(19) United States
(12) Patent Application Publication
(10) Pub. No.: US 2018/0110796 A1
(43) Pub. Date: Apr. 26, 2018

(54) COMPOSITIONS AND METHODS FOR THE TREATMENT OF HBV INFECTION

(71) Applicant: Spring Bank Pharmaceuticals, Inc., Milford, MA (US)

(72) Inventor: Radhakrishnan P. Iyer, Shrewsbury, MA (US)

(21) Appl. No.: 15/565,046

(22) PCT Filed: Apr. 7, 2016
(86) PCT No.: PCT/US16/26498

§ 371 Date: Oct. 6, 2017

Related U.S. Application Data
(60) Provisional application No. 62/279,382, filed on Jan. 15, 2016.
(60) Provisional application No. 62/220,406, filed on Sep. 18, 2015.
(60) Provisional application No. 62/144,300, filed on Apr. 7, 2015.

(51) Int. Cl. A61K 31/7084; A61K 31/522; A61P 31/12

(52) U.S. Cl. CPC A61K 31/7084; A61P 31/12; A61K 31/522

(57) ABSTRACT

This invention relates to methods useful in the treatment of a hepatitis infection.

### RELATED APPLICATIONS

This application claims priority to U.S. Provisional Application No. 62/279,382, filed on Jan. 15, 2016; U.S. Provisional Application No. 62/220,406, filed on Sep. 18, 2015; and U.S. Provisional Application No. 62/144,300, filed on Apr. 7, 2015. The entire disclosures of each of the foregoing applications are incorporated herein by reference.

### STATEMENT REGARDING FEDERALLY SPONSORED RESEARCH OF DEVELOPMENT

This invention was made with government support under grant number R01AI094469 awarded by the National Institutes of Health. The government has certain rights in the invention.

### FIELD OF INVENTION

This invention relates to compositions and methods useful in the treatment of a viral infection or infections.

### BACKGROUND OF INVENTION

Chronic infection with hepatitis B virus (HBV) is a major public health problem and is responsible for approximately 1.2 million deaths per year worldwide due to HBV-associated liver diseases, such as hepatic cirrhosis, and hepatocellular carcinoma (HCC) (Levanchy, D. J Viral Hepatol (2004) 11:97-107). It is estimated that more than 2 billion people have serological evidence of previous or current HBV infection, and that over 350 million individuals are chronic carriers of HBV (Levanchy, D. J Viral Hepatol (2004) 11:97-107; Kwon H., Lok. A. S. Nat Rev Gastroenterol Hepatol (2011) 8:275-284). Although safe and effective prophylactic vaccines against HBV are available, improvements in therapeutics for treatment of chronic HBV infection are still urgently needed. Current antiviral therapies for chronic hepatitis B (CHB) are limited, and include nucleoside and nucleotide analogs and interferon (IFN) treatment. While administration of nucleosides and nucleotides may reduce viral load and improve the long-term outcome of CHB, prolonged use rarely leads to a cure. Only 2-3% of treated patients per year experience a loss of measurable biomarkers of HBV infection, namely durable loss of HBV surface antigen (HBsAg) and seroconversion to antibodies against HBsAg (anti-HBs) (Kwon H., Lok. A. S. Nat Rev Gastroenterol Hepatol (2011) 8:275-284). Long-term IFN administration is also associated with treatment-limiting adverse effects and variability in treatment response, and while the rate of durable HBsAg loss is higher than with nucleoside and nucleotide analogs, it still only occurs in less than 10% of patients.

Further, a major obstacle for treatment of chronic HBV infection relates to the emergence of drug resistant variants that occurs upon extended use of currently available nucleoside and nucleotide analogs, many of which target the viral DNA polymerase. In addition, current treatments require persistent and long-term use, which often results in unwarranted side effects and the risk of relapse upon treatment discontinuation. Accordingly, there is a critical need for a new generation of therapies to combat chronic HBV infection.

### SUMMARY OF INVENTION

In one aspect, the present invention features a method of treating a subject infected with the Hepatitis B virus, the method comprising administering to the subject a pharmaceutical composition comprising a compound of Formula (I) at a dosage of about 0.5 mg/kg to about 100 mg/kg, wherein the compound is selected from:

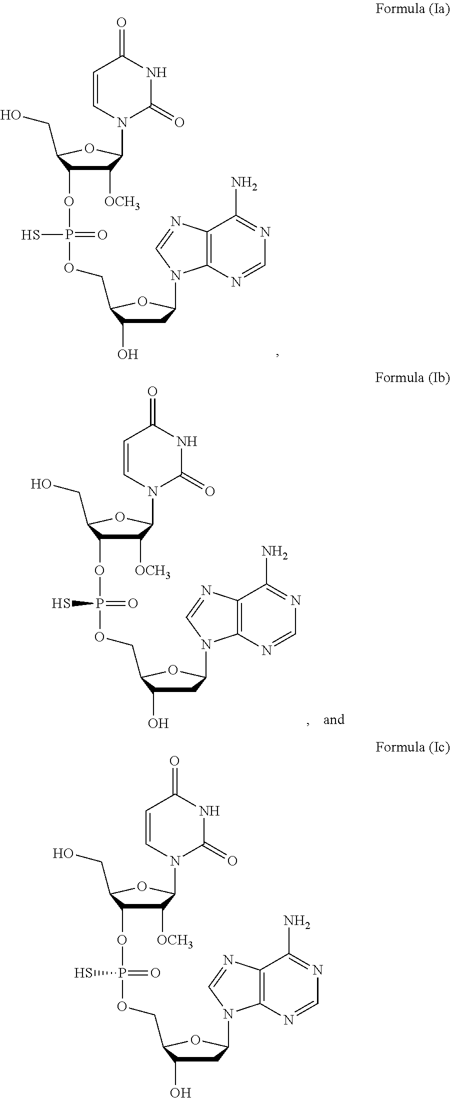

or a prodrug or pharmaceutically acceptable salt thereof to thereby treat the subject. In some embodiments, the prodrug of Formula (I) is a compound of Formula (II), wherein the compound is selected from:

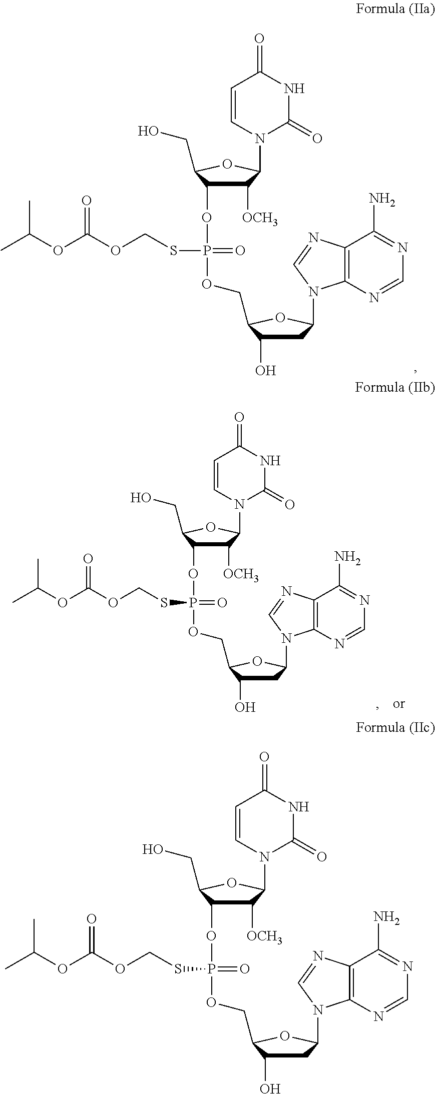

or a pharmaceutically acceptable salt thereof.

In some embodiments, the composition comprises a mixture of compounds of Formula (I). In some embodiments, the composition comprises a mixture of Formula (Ib) and Formula (Ic). In some embodiments, the mixture comprises a ratio of Formula (Ib) to Formula (Ic) of about 1:1 (e.g., a racemic mixture). In some embodiments, the mixture comprises a ratio of Formula (Ib) to Formula (Ic) of about 51:49, about 52:48, about 53:47, about 54:46, about 55:45, about 60:40, about 65:35, about 70:30, about 75:25, about 80:20, about 85:15, about 90:10, about 95:5, or about 99:1. In some embodiments, the mixture comprises a ratio of Formula (Ic) to Formula (Ib) of about 51:49, about 52:48, about 53:47, about 54:46, about 55:45, about 60:40, about 65:35, about 70:30, about 75:25, about 80:20, about 85:15, about 90:10, about 95:5, or about 99:1.

In some embodiments, the composition comprises Formula (Ib) and comprises less than about 5% of Formula (Ic), e.g., less than about 4%, less than about 3%, less than about 2%, less than about 1%, less than about 0.5%, or less than about 0.1% of Formula (Ic), or is substantially free of Formula (Ic). In some embodiments, the composition comprises Formula (Ic) and comprises less than about 5% of Formula (Ib), e.g., less than about 4%, less than about 3%, less than about 2%, less than about 1%, less than about 0.5%, or less than about 0.1% of Formula (Ib), or is substantially free of Formula (Ib).

In some embodiments, the composition comprises a mixture of compounds of Formula (II). In some embodiments, the composition comprises a mixture of Formula (IIb) and Formula (IIc). In some embodiments, the mixture comprises a ratio of Formula (IIb) to Formula (IIc) of about 1:1 (e.g., a racemic mixture). In some embodiments, the mixture comprises a ratio of Formula (IIb) to Formula (IIc) of about 51:49, about 52:48, about 53:47, about 54:46, about 55:45, about 60:40, about 65:35, about 70:30, about 75:25, about 80:20, about 85:15, about 90:10, about 95:5, or about 99:1. In some embodiments, the mixture comprises a ratio of Formula (IIc) to Formula (IIb) of about 51:49, about 52:48, about 53:47, about 54:46, about 55:45, about 60:40, about 65:35, about 70:30, about 75:25, about 80:20, about 85:15, about 90:10, about 95:5, or about 99:1.

In some embodiments, the composition comprises Formula (IIb) and comprises less than about 5% of Formula (IIc), e.g., less than about 4%, less than about 3%, less than about 2%, less than about 1%, less than about 0.5%, or less than about 0.1% of Formula (IIc), or is substantially free of Formula (IIc). In some embodiments, the composition comprises Formula (IIc) and comprises less than about 5% of Formula (IIb), e.g., less than about 4%, less than about 3%, less than about 2%, less than about 1%, less than about 0.5%, or less than about 0.1% of Formula (IIb), or is substantially free of Formula (IIb).

In some embodiments, the composition is administered orally. In some embodiments, the compound of Formula (I) or Formula (II) is administered orally. In some embodiments, the compound of Formula (II) is administered orally. In some embodiments, the composition is administered parenterally (e.g., intraperitoneally). In some embodiments, the compound of Formula (I) or Formula (II) is administered parenterally (e.g., intraperitoneally). In some embodiments, the compound of Formula (II) is administered parenterally (e.g., intraperitoneally).

In some embodiments, the subject is a mammal. In some embodiments, the subject is a human. In some embodiments the subject is a non-human animal, e.g., a woodchuck (e.g., Eastern woodchuck).

In some embodiments, the method comprises daily administration of said dosage. In some embodiments, the administration is once daily. In some embodiments, the administration is greater than once daily, e.g., twice daily, three times daily, four times daily.

In some embodiments, the method comprises administration of said dosage at a frequency less than once a day, e.g., once every 36 hours, once every other day, or once a week.

In some embodiments, the dosage comprises about 0.5 mg/kg to about 100 mg/kg. In some embodiments, the dosage comprises about 0.5 mg/kg to about 95 mg/kg, about 90 mg/kg, about 85 mg/kg, about 80 mg/kg, about 75 mg/kg, about 70 mg/kg, about 65 mg/kg about 60 mg/kg, about 55 mg/kg, about 50 mg/kg, about 45 mg/kg, about 40 mg/kg, about 35 mg/kg, about 30 mg/kg, about 25 mg/kg, about 20 mg/kg, about 15 mg/kg, or about 10 mg/kg. In some embodiments, the dosage comprises about 0.5 mg/kg to about 50 mg/kg. In some embodiments, the dosage comprises about 0.5 mg/kg to about 40 mg/kg.

In some embodiments, the dosage comprises greater than about 0.5 mg/kg, e.g., about 1.0 mg/kg, about 1.5 mg/kg, about 2 mg/kg, about 3 mg/kg, about 4 mg/kg, about 5 mg/kg, about 10 mg/kg about 15 mg/kg, about 20 mg/kg, about 25 mg/kg, about 30 mg/kg, about 35 mg/kg, about 40 mg/kg, about 50 mg/kg, about 55 mg/kg, about 60 mg/kg, about 65 mg/kg, about 70 mg/kg, about 75 mg/kg, about 80 mg/kg, about 85 mg/kg, or about 90 mg/kg up to about 100 mg/kg. In some embodiments, the dosage comprises about 5 mg/kg to about 50 mg/kg. In some embodiments, the dosage comprises about 10 mg/kg to about 50 mg/kg. In some embodiments, the dosage comprises about 15 mg/kg to about 50 mg/kg.

In some embodiments, the dosage comprises a liquid or a solid dosage form. In some embodiments, the liquid dosage form comprises a suspension, a solution, a linctus, an emulsion, a drink, an elixir, or a syrup. In some embodiments, the solid dosage form comprises a capsule, tablet, dragé, or powder. In some embodiments, the liquid or solid dosage form is orally administered. In some embodiments, the liquid or solid form is parenterally (e.g., intraperitoneally) administered.

In some embodiments, the method further comprises the administration of an additional agent. In some embodiments, the method further comprises the administration of a therapeutically effective amount of an additional agent. In some embodiments, the additional agent is an antiviral agent or an anticancer agent. In some embodiments, the antiviral agent comprises an interferon, a nucleoside analog, a non-nucleoside antiviral, or a non-interferon immune enhancer. In some embodiments, the interferon comprises interferon alfa-2a, interferon alfa-2b, interferon alfa-n1, interferon alfacon-1, or a pegylated interferon (e.g., peginterferon alfa-2a, peginterferon alfa-2b). In some embodiments, the nucleoside analog comprises lamivudine, adefovir dipivoxil, entecavir, telbivudine, clevudine, ribavarin, tenofovir, tenofovir dipivoxil, tenofovir alafenamide, besifovir, or AGX-1009. In some embodiments, the antiviral agent is entecavir. In some embodiments, the antiviral compound comprises NOV-225, BAM 205, Myrcludex B, ARC-520, BAY 41-4109, REP 9AC, Alinia (nitazoxanide), Dd-RNAi, NVR-121 (NVR 3-778), BSBI-25, NVP-018, TKM-HBV, or ALN-HBV. In some embodiments, the non-interferon immune enhancer comprises zadaxin (thymosin alpha-1), GS-4774, CYT107 (interleukin-7), Dv-601, HBV core antigen vaccine, or GS-9620. In some embodiments, the antiviral agent is a capsid inhibitor, an entry inhibitor, a secretion inhibitor, a microRNA, an antisense RNA agent, an RNAi agent, or other agent designed to inhibit viral RNA. In some embodiments, the anticancer agent is selected from methotrexate, 5-fluorouracil, doxorubicin, vincristine, bleomycin, vinblastine, dacarbazine, toposide, cisplatin, epirubicin, and sorafenib tosylate.

In some embodiments, in a method described herein, the subject is treatment naïve. In some embodiments, the subject has previously been treated for HBV infection. In some embodiments, the previous treatment for HBV infection has failed. In some embodiments, the subject has relapsed.

In some embodiments, the subject has been previously been treated with an anti-HBV agent other than a compound of Formula (I) or Formula (II) or a pharmaceutically acceptable salt thereof (e.g., an interferon, ribavirin) and is suffering from a relapsed HBV infection.

In some embodiments, the subject is suffering from a co-infection with Hepatitis B virus (HDV). In some embodiments, the subject has been diagnosed with an HBV infection. In some embodiments, the subject has been diagnosed with an HDV infection. In some embodiments, the subject has been diagnosed with a co-infection of HBV and HDV.

In some embodiments, in a method described herein, the subject has been diagnosed with cirrhosis of the liver. In some embodiments, the subject has been diagnosed with hepatocellular carcinoma. In some embodiments, the subject has been diagnosed with hepatocellular carcinoma and is awaiting liver transplantation. In some embodiments, the subject is non-cirrhotic or has not been diagnosed with hepatocellular carcinoma.

In some embodiments, in a method described herein, the subject has been further diagnosed with an HIV infection. In some embodiments, the strain of HIV infection is known. In some embodiments, the subject is infected with HIV-1 or HIV-2 (e.g., strain 1 or strain 2).

In some embodiments, the methods described herein further comprise analyzing or receiving analysis of the body weight and temperature of the subject at least once a week until the end of treatment.

In some embodiments, the methods described herein further comprise analyzing or receiving analysis of a blood sample from the subject at least once prior to the end of treatment. In some embodiments, the blood sample is analyzed for viral load and surface antigen levels. In some embodiments, the blood sample is analyzed for the expression level of interferon (e.g., interferon alfa or interferon beta), an interferon stimulating protein (e.g., ISG15, CXCL10, OAS 1), or other cytokines. In some embodiments, the blood sample is analyzed for the presence of anti-WHS antibodies and anti-WHc antibodies.

In some embodiments, the methods described herein further comprise analyzing or receiving analysis of a liver biopsy specimen from the subject at least once prior to the end of treatment. In some embodiments, the liver biopsy specimen is analyzed for the levels of viral DNA, viral RNA, viral antigens, and cccDNA. In some embodiments, the liver biopsy specimen is analyzed for the expression level of interferon (e.g., interferon alfa or interferon beta), an interferon stimulating protein (e.g., ISG15, CXCL10, OAS 1), or other cytokines. In some embodiments, the liver biopsy specimen is analyzed for the presence of anti-WHS antibodies and anti-WHc antibodies. In some embodiments, the liver biopsy specimen is analyzed for the reduction of liver inflammation, necrosis, steatosis, or fibrosis.

In another aspect, the present invention features a method of treating Hepatitis B virus in a subject, the method comprising administering to the subject a compound of Formula (I), wherein the compound is selected from:

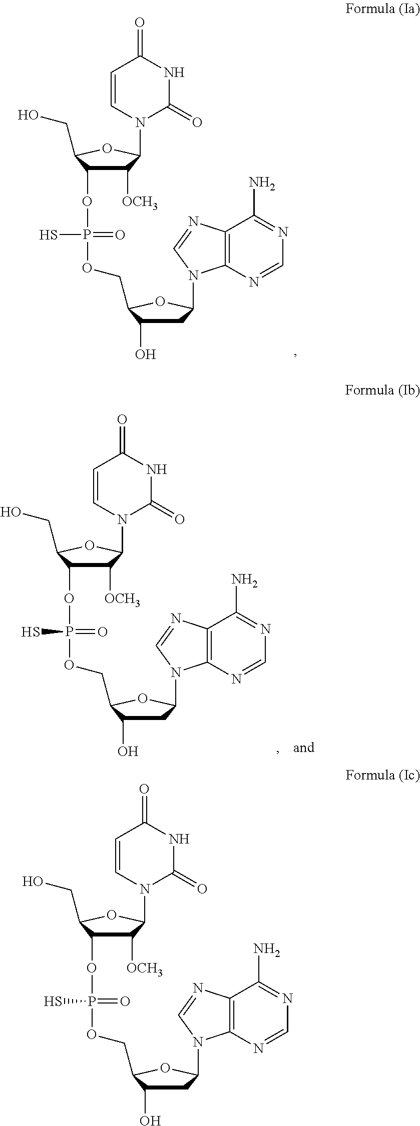

or a prodrug or pharmaceutically acceptable salt thereof in combination with entecavir or a pharmaceutically acceptable salt thereof to thereby treat the subject.

In another aspect, the present invention features a method of treating Hepatitis B virus in a subject comprising administering to the subject a course of entecavir or a pharmaceutically acceptable salt thereof, wherein the subject has previously been treated with a course of compound of Formula (I), wherein the compound is selected from:

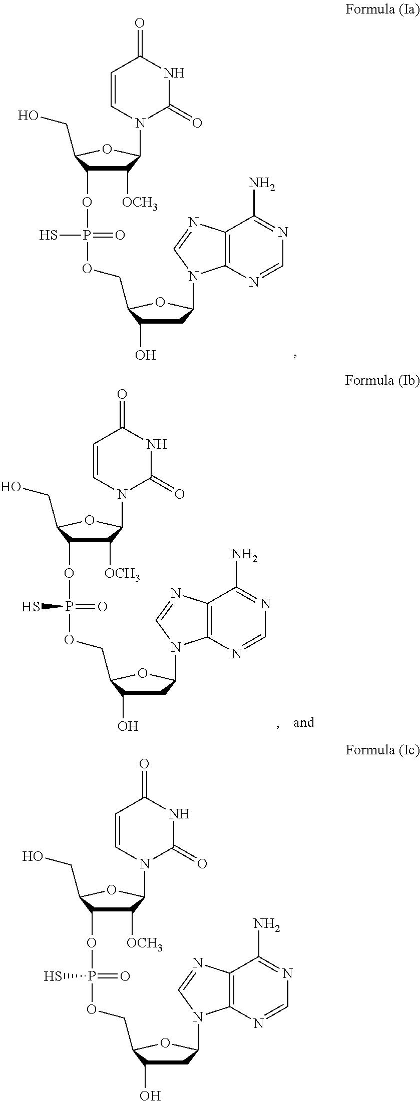

or a prodrug or pharmaceutically acceptable salt thereof to thereby treat the subject.

In another aspect, the present invention features a method of treating Hepatitis B virus in a subject, wherein the subject has previously been treated with a course of entecavir or a pharmaceutically acceptable salt thereof, the method comprising administering to the subject a course of compound of Formula (I), wherein the compound is selected from:

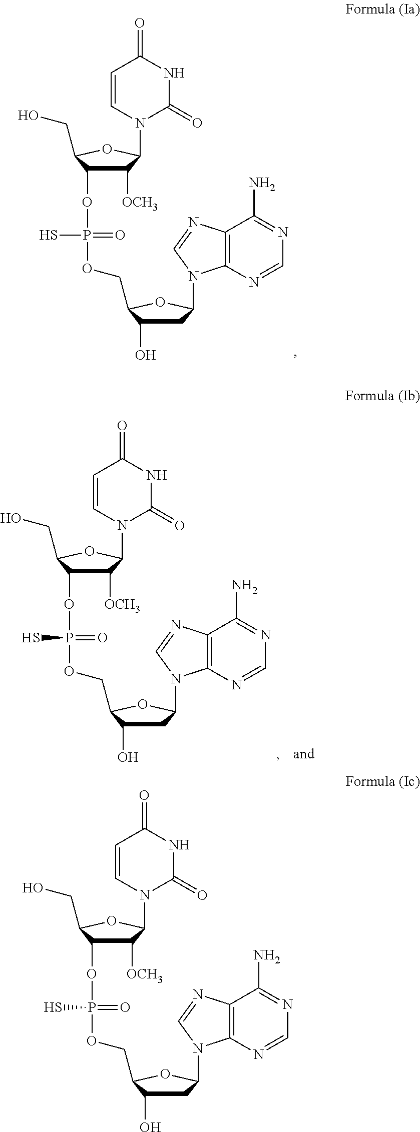

or a prodrug or pharmaceutically acceptable salt thereof to thereby treat subject.

In another aspect, the present invention features a method of treating Hepatitis B virus in a subject, the method comprising first administering a course of entecavir or a pharmaceutically acceptable salt thereof to the subject, and subsequently administering to the subject a course of a compound of Formula (I), wherein the compound is selected from:

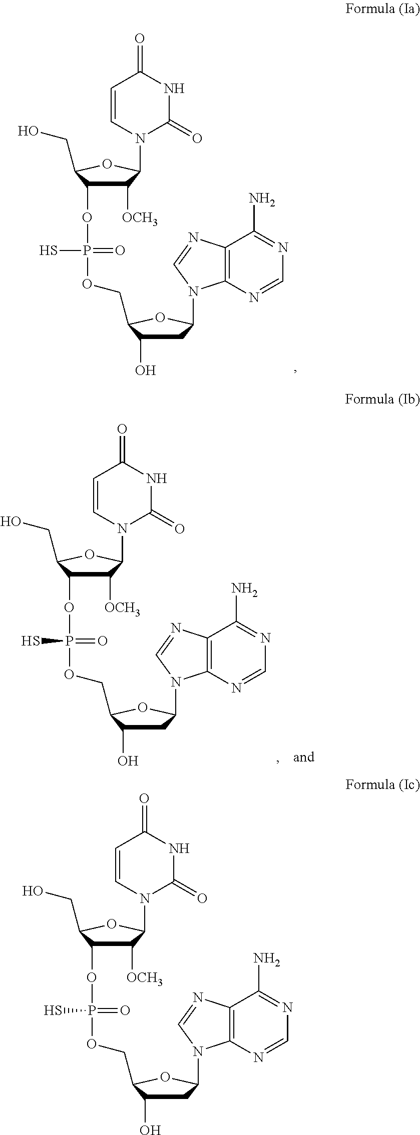

or a prodrug or pharmaceutically acceptable salt thereof to thereby treat the subject.

In another aspect, the present invention features a method of treating Hepatitis B virus in a subject, the method comprising first administering to the subject a course of a compound of Formula (I), wherein the compound is selected from:

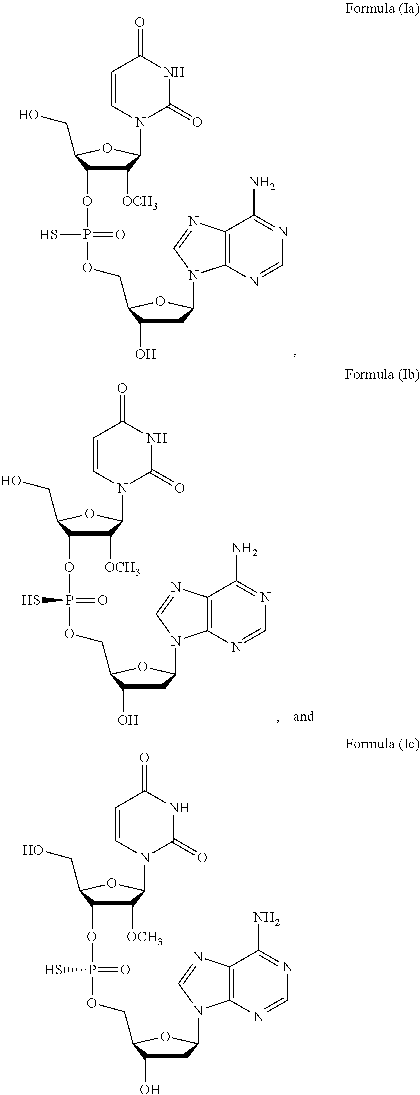

or a prodrug or pharmaceutically acceptable salt thereof, and subsequently administering to the subject a course of entecavir or a pharmaceutically acceptable salt thereof to thereby treat the subject.

In any of the aforementioned aspects of the invention, the prodrug of Formula (I) is a compound of Formula (II), wherein the compound is selected from:

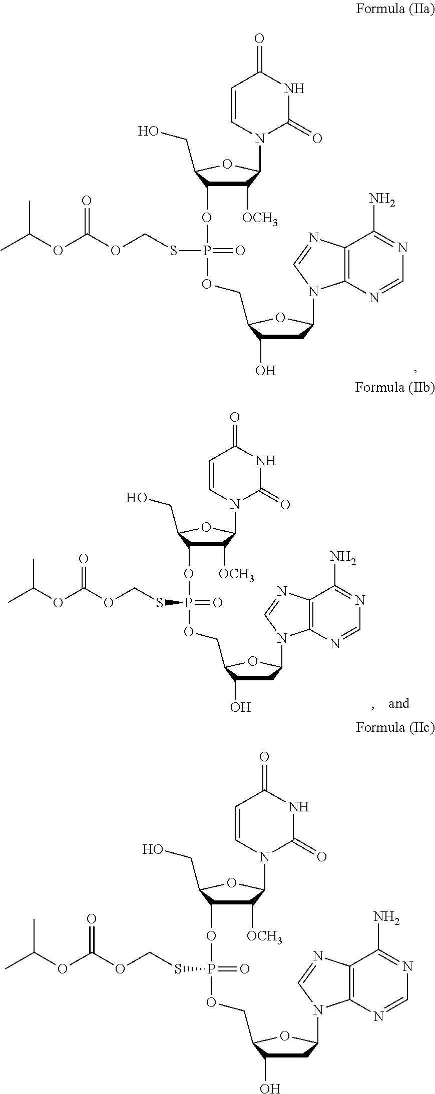

or a pharmaceutically acceptable salt thereof.

In some embodiments, a course of a compound of Formula (I) or Formula (II) is between about 1 day to about 24 weeks. In some embodiments, the compound of Formula (I) or Formula (II) is administered at least weekly (e.g., once a week, twice a week, three times a week, four times a week, five times a week, six times a week, 7 times a week) throughout a course of treatment. In some embodiments, the compound of Formula (I) or Formula (II) is administered daily throughout a course of treatment.

In some embodiments, the course of entecavir is between about 1 day to about 12 weeks. In some embodiments, entecavir is administered at least weekly (e.g., once a week, twice a week, three times a week, four times a week, five times a week, six times a week, 7 times a week) throughout a course of treatment. In some embodiments, entecavir is administered daily throughout a course of treatment.

In some embodiments, the dosage of the compound of Formula (I) or Formula (II) is between about 5 mg/kg to about 100 mg/kg (e.g., about 5 mg/kg, about 10 mg/kg, about 15 mg/kg, about 20 mg/kg, about 30 mg/kg, about 40 mg/kg, about 50 mg/kg, about 60 mg/kg, about 70 mg/kg, about 80 mg/kg, about 90 mg/kg, or about 100 mg/kg). In some embodiments, the dosage of the compound of Formula (I) or Formula (II) is between about 10 mg/kg to about 50 mg/kg (e.g., about 10 mg/kg, about 15 mg/kg, about 20 mg/kg, about 25 mg/kg, about 30 mg/kg, about 35 mg/kg, about 40 mg/kg, about 45 mg/kg, or about 50 mg/kg).

In some embodiments, the dosage of entecavir is between about 0.1 mg to about 5 mg (e.g., about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.25 mg, about 1.5 mg, about 1.75 mg, about 2 mg, about 2.5 mg, about 3 mg, about 3.5 mg, about 4 mg, about 4.5 mg, or about 5 mg). In some embodiments, the dosage of entecavir is between about 0.01 mg/kg to about 10 mg/kg (e.g., about 0.01 mg/kg, about 0.025 mg/kg, about 0.05 mg/kg, about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg). In some embodiments, the dosage of entecavir is between about 0.1 mg/kg to about 5 mg/kg (e.g., about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.25 mg/kg, about 1.5 mg/kg, about 1.75 mg/kg, about 2 mg/kg, about 2.5 mg/kg, about 3 mg/kg, about 3.5 mg/kg, about 4 mg/kg, about 4.5 mg/kg, or about 5 mg/kg).

In some embodiments, the dosage comprises a liquid or a solid dosage form. In some embodiments, the liquid dosage form comprises a suspension, a solution, a linctus, an emulsion, a drink, an elixir, or a syrup. In some embodiments, the solid dosage form comprises a capsule, tablet, dragée, or powder.

In some embodiments, the compound of Formula (I) or Formula (II) or entecavir is administered orally (e.g., the compound of Formula (I) or Formula (II) is administered orally, or entecavir is administered orally, or both the compound of Formula (I) or Formula (II) and entecavir are administered orally). In some embodiments, the compound of Formula (I) or Formula (II) or entecavir is administered parenterally (e.g., the compound of Formula (II) is administered parenterally). In some embodiments, the compound of Formula (I) or Formula (II) is administered parenterally and entecavir is administered orally. In some embodiments, compound of Formula (I) or Formula (II) is formulated as a fixed dose combination with entecavir (e.g., as a liquid dosage form or solid dosage form, e.g., a capsule or tablet). In some embodiments, compound of Formula (I) or Formula (II) is formulated as a fixed dose combination with entecavir (e.g., as a liquid dosage form or solid dosage form, e.g., a capsule or tablet) for oral administration.

In some embodiments, the administration of a compound of Formula (I) or Formula (II) and entecavir has a synergistic or additive effect. In some embodiments, the administration of a compound of Formula (I) or Formula (II) and entecavir has an additive effect. In some embodiments, the administration of a compound of Formula (I) or Formula (II) and entecavir has a synergistic effect.

In some embodiments, the composition comprises a mixture of compounds of Formula (I), e.g., Formula (Ib) and Formula (Ic). In some embodiments, the composition comprises Formula (Ib) and comprises less than about 5% of Formula (Ic) (e.g., less than about 4%, less than about 3%, less than about 2%, less than about 1%, less than about 0.5%, or less than about 0.1% of Formula (Ic)), or is substantially free of Formula (Ic). In some embodiments, the composition comprises Formula (Ic) and comprises less than about 5% of Formula (Ib) (e.g., less than about 4%, less than about 3%, less than about 2%, less than about 1%, less than about 0.5%, or less than about 0.1% of Formula (Ib), or is substantially free of Formula (Ib)).

In some embodiments, the composition comprises a mixture of compounds of Formula (II), e.g., Formula (IIb) and Formula (IIc). In some embodiments, the composition comprises

Formula (IIb) and comprises less than about 5% of Formula (IIc) (e.g., less than about 4%, less than about 3%, less than about 2%, less than about 1%, less than about 0.5%, or less than about 0.1% of Formula (IIc), or is substantially free of Formula (IIc)). In some embodiments, the composition comprises Formula (IIc) and comprises less than about 5% of Formula (IIb) (e.g., less than about 4%, less than about 3%, less than about 2%, less than about 1%, less than about 0.5%, or less than about 0.1% of Formula (IIb), or is substantially free of Formula (IIb)).

In some embodiments, the subject is a mammal. In some embodiments, the subject is a human. In some embodiments the subject is a non-human animal, e.g., a woodchuck (e.g., Eastern woodchuck).

In some embodiments, the method further comprises the administration of a therapeutically effective amount of an additional agent. In some embodiments, the additional agent is an antiviral agent or an anticancer agent. In some embodiments, the antiviral agent comprises an interferon, a nucleoside analog, a non-nucleoside antiviral, or a non-interferon immune enhancer. In some embodiments, the interferon comprises interferon alfa-2a, interferon alfa-2b, interferon alfa-n1, interferon alfacon-1, or a pegylated interferon (e.g., peginterferon alfa-2a, peginterferon alfa-2b). In some embodiments, the nucleoside analog comprises lamivudine, adefovir dipivoxil, telbivudine, clevudine, ribavarin, tenofovir, tenofovir dipivoxil, tenofovir alafenamide, besifovir, or AGX-1009. In some embodiments, the antiviral agent is tenofovir (e.g., tenofovir dipivoxil, tenofovir alafenamide). In some embodiments, the antiviral compound comprises NOV-225, BAM 205, Myrcludex B, ARC-520, BAY 41-4109, REP 9AC, Alinia (nitazoxanide), Dd-RNAi, NVR-121 (NVR 3-778), BSBI-25, NVP-018, TKM-HBV, or ALN-HBV. In some embodiments, the non-interferon immune enhancer comprises zadaxin (thymosin alpha-1), GS-4774, CYT107 (interleukin-7), Dv-601, HBV core antigen vaccine, or GS-9620. In some embodiments, the antiviral agent is a capsid inhibitor, an entry inhibitor, a secretion inhibitor, a microRNA, an antisense RNA agent, an RNAi agent, or other agent designed to inhibit viral RNA. In some embodiments, the anticancer agent is selected from methotrexate, 5-fluorouracil, doxorubicin, vincristine, bleomycin, vinblastine, dacarbazine, toposide, cisplatin, epirubicin, and sorafenib tosylate.

In some embodiments, in a method described herein, the subject is treatment naïve. In some embodiments, the subject has previously been treated for HBV infection. In some embodiments, the previous treatment for HBV infection has failed. In some embodiments, the subject has relapsed.

In some embodiments, the subject has been previously been treated with an anti-HBV agent other than a compound of Formula (I) or Formula (II) or a pharmaceutically acceptable salt thereof (e.g., an interferon, ribavirin) and is suffering from a relapsed HBV infection.

In some embodiments, the subject is suffering from a co-infection with Hepatitis B virus (HDV). In some embodiments, the subject has been diagnosed with an HBV infection. In some embodiments, the subject has been diagnosed with an HDV infection. In some embodiments, the subject has been diagnosed with a co-infection of HBV and HDV.

In some embodiments, in a method described herein, the subject has been diagnosed with cirrhosis of the liver. In some embodiments, the subject has been diagnosed with hepatocellular carcinoma. In some embodiments, the subject has been diagnosed with hepatocellular carcinoma and is awaiting liver transplantation. In some embodiments, the subject is non-cirrhotic or has not been diagnosed with hepatocellular carcinoma.

In some embodiments, in a method described herein, the subject has been further diagnosed with an HIV infection. In some embodiments, the strain of HIV infection is known. In some embodiments, the subject is infected with HIV-1 or HIV-2 (e.g., strain 1 or strain 2).

In some embodiments, the methods described herein further comprise analyzing or receiving analysis of the body weight and temperature of the subject at least once a week until the end of treatment.

In some embodiments, the methods described herein further comprise analyzing or receiving analysis of a blood sample from the subject at least once prior to the end of treatment. In some embodiments, the blood sample is analyzed for viral load and surface antigen levels. In some embodiments, the blood sample is analyzed for the expression level of interferon (e.g., interferon alfa or interferon beta), an interferon stimulating protein (e.g., ISG15, CXCL10, OAS 1), or other cytokines. In some embodiments, the blood sample is analyzed for the presence of anti-WHS antibodies and anti-WHc antibodies.

In some embodiments, the methods described herein further comprise analyzing or receiving analysis of a liver biopsy specimen from the subject at least once prior to the end of treatment. In some embodiments, the liver biopsy specimen is analyzed for the levels of viral DNA, viral RNA, viral antigens, and cccDNA. In some embodiments, the liver biopsy specimen is analyzed for the expression level of interferon (e.g., interferon alfa or interferon beta), an interferon stimulating protein (e.g., ISG15, CXCL10, OAS 1), or other cytokines. In some embodiments, the liver biopsy specimen is analyzed for the presence of anti-WHS antibodies and anti-WHc antibodies. In some embodiments, the liver biopsy specimen is analyzed for the reduction of liver inflammation, necrosis, steatosis, or fibrosis.

In any and all embodiments, the method features a pharmaceutical composition for use in treating Hepatitis B virus in a subject, the composition comprising a compound of Formula (I) (e.g., Formula (Ia), Formula (Ib), or Formula (Ic)), a compound of Formula (II) (e.g., Formula (IIa), Formula (IIb), or Formula (IIc)), or a pharmaceutically acceptable salt thereof in combination with entecavir to thereby treat the subject.

In another aspect, the present invention features a method of treating Hepatitis B virus in a subject, the method comprising administering to the subject a compound of Formula (I), wherein the compound is selected from:

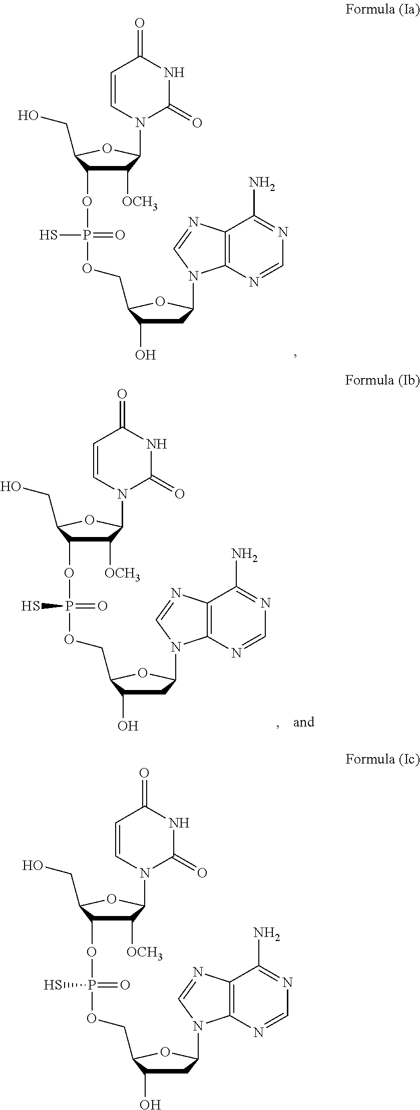

or a prodrug or pharmaceutically acceptable salt thereof in combination with tenofovir (e.g., tenofovir dipivoxil or tenofovir alafenamide) or a pharmaceutically acceptable salt thereof to thereby treat the subject.

In another aspect, the present invention features a method of treating Hepatitis B virus in a subject comprising administering to the subject a course of tenofovir (e.g., tenofovir dipivoxil or tenofovir alafenamide) or a pharmaceutically acceptable salt thereof, wherein the subject has previously been treated with a course of compound of Formula (I), wherein the compound is selected from:

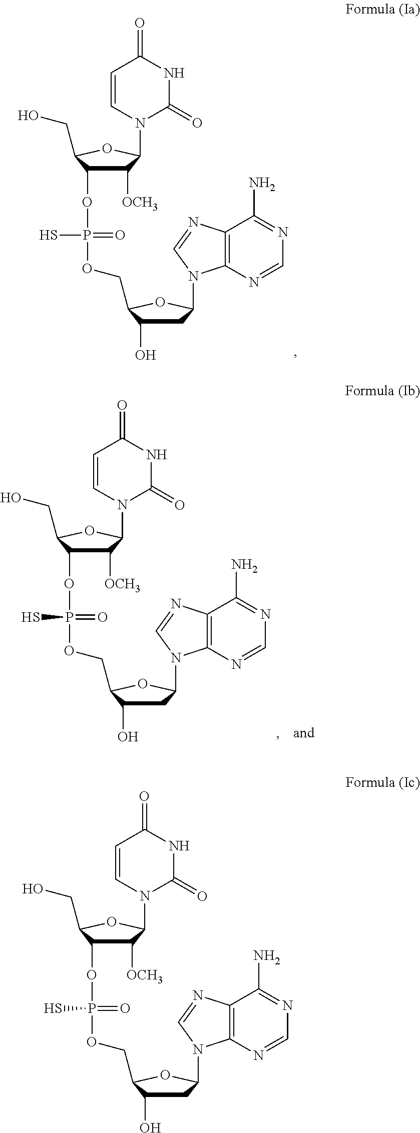

or a prodrug or pharmaceutically acceptable salt thereof to thereby treat the subject.

In another aspect, the present invention features a method of treating Hepatitis B virus in a subject, wherein the subject has previously been treated with a course of tenofovir (e.g., tenofovir dipivoxil or tenofovir alafenamide) or a pharmaceutically acceptable salt thereof, the method comprising administering to the subject a course of compound of Formula (I), wherein the compound is selected from:

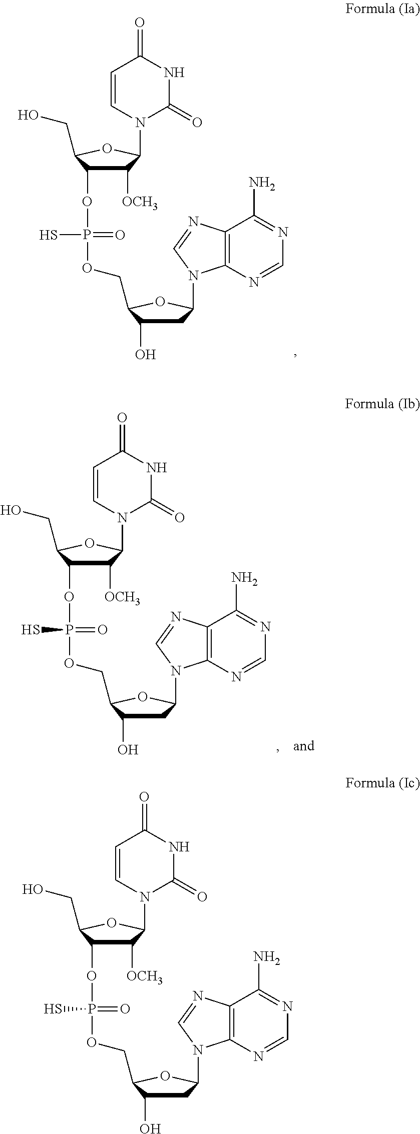

or a prodrug or pharmaceutically acceptable salt thereof to thereby treat subject.

In another aspect, the present invention features a method of treating Hepatitis B virus in a subject, the method comprising first administering a course of tenofovir (e.g., tenofovir dipivoxil or tenofovir alafenamide) or a pharmaceutically acceptable salt thereof to the subject, and subsequently administering to the subject a course of a compound of Formula (I), wherein the compound is selected from:

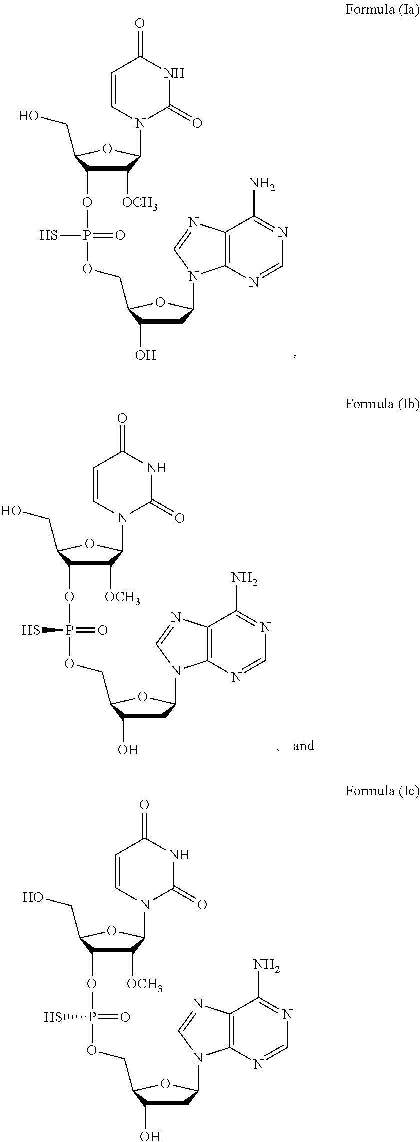

or a prodrug or pharmaceutically acceptable salt thereof to thereby treat the subject.

In another aspect, the present invention features a method of treating Hepatitis B virus in a subject, the method comprising first administering to the subject a course of a compound of Formula (I), wherein the compound is selected from:

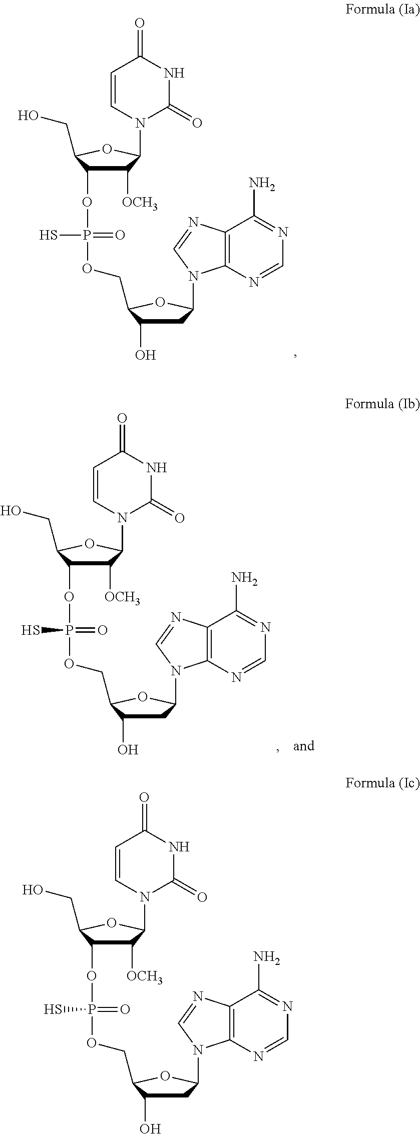

or a prodrug or pharmaceutically acceptable salt thereof, and subsequently administering to the subject a course of tenofovir (e.g., tenofovir dipivoxil or tenofovir alafenamide) or a pharmaceutically acceptable salt thereof to thereby treat the subject.

In any of the aforementioned aspects of the invention, the prodrug of Formula (I) is a compound of Formula (II), wherein the compound is selected from:

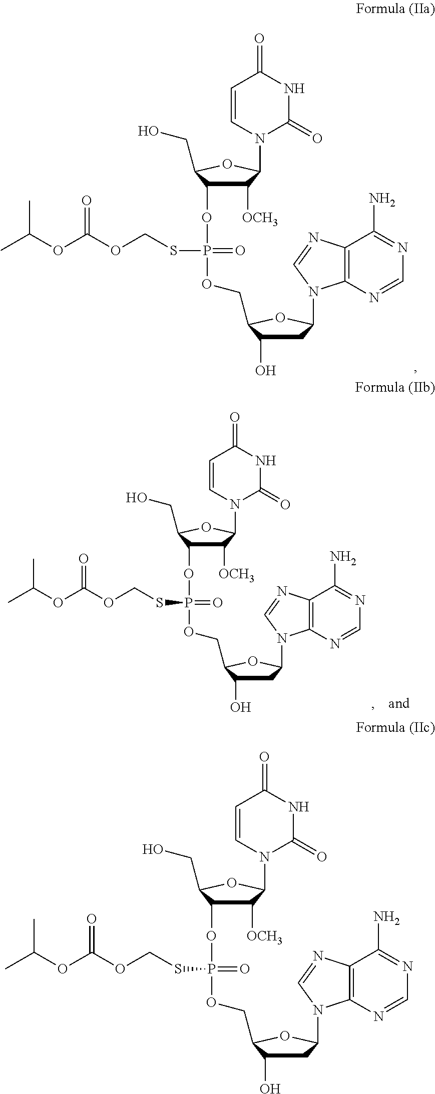

or a pharmaceutically acceptable salt thereof.

In some embodiments, a course of a compound of Formula (I) or Formula (II) is between about 1 day to about 24 weeks. In some embodiments, the compound of Formula (I) or Formula (II) is administered at least weekly (e.g., once a week, twice a week, three times a week, four times a week, five times a week, six times a week, 7 times a week) throughout a course of treatment. In some embodiments, the compound of Formula (I) or Formula (II) is administered daily throughout a course of treatment.

In some embodiments, the course of tenofovir (e.g., tenofovir dipivoxil or tenofovir alafenamide) is between about 1 day to about 12 weeks. In some embodiments, tenofovir (e.g., tenofovir dipivoxil or tenofovir alafenamide) is administered at least weekly (e.g., once a week, twice a week, three times a week, four times a week, five times a week, six times a week, 7 times a week) throughout a course of treatment. In some embodiments, tenofovir (e.g., tenofovir dipivoxil or tenofovir alafenamide) is administered daily throughout a course of treatment.

In some embodiments, the dosage of the compound of Formula (I) or Formula (II) is between about 5 mg/kg to about 100 mg/kg (e.g., about 5 mg/kg, about 10 mg/kg, about 15 mg/kg, about 20 mg/kg, about 30 mg/kg, about 40 mg/kg, about 50 mg/kg, about 60 mg/kg, about 70 mg/kg, about 80 mg/kg, about 90 mg/kg, or about 100 mg/kg). In some embodiments, the dosage of the compound of Formula (I) or Formula (II) is between about 10 mg/kg to about 50 mg/kg (e.g., about 10 mg/kg, about 15 mg/kg, about 20 mg/kg, about 25 mg/kg, about 30 mg/kg, about 35 mg/kg, about 40 mg/kg, about 45 mg/kg, or about 50 mg/kg).

In some embodiments, the dosage of tenofovir (e.g., tenofovir dipivoxil or tenofovir alafenamide) is between about 10 mg to about 500 mg (e.g., about 25 mg, about 50 mg, about 75 mg, about 100 mg, about 150 mg, about 200 mg, about 250 mg, or about 300 mg). In some embodiments, the dosage of tenofovir (e.g., tenofovir dipivoxil or tenofovir alafenamide) is between about 0.01 mg/kg to about 20 mg/kg (e.g., about 0.01 mg/kg, about 0.025 mg/kg, about 0.05 mg/kg, about 0.1 mg/kg, about 0.5 mg/kg, about 1 mg/kg, about 2 mg/kg, about 5 mg/kg, about 7.5 mg/kg, about 10 mg/kg, about 12.5 mg/kg, about 15 mg/kg, about 17.5 mg/kg, or about 20 mg/kg). In some embodiments, the dosage of tenofovir (e.g., tenofovir dipivoxil or tenofovir alafenamide) is between about 1 mg/kg to about 20 mg/kg (e.g., about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 12.5 mg/kg, about 15 mg/kg, about 17.5 mg/kg, or about 20 mg/kg).

In some embodiments, the dosage comprises a liquid or a solid dosage form. In some embodiments, the liquid dosage form comprises a suspension, a solution, a linctus, an emulsion, a drink, an elixir, or a syrup. In some embodiments, the solid dosage form comprises a capsule, tablet, dragée, or powder.

In some embodiments, the compound of Formula (I) or Formula (II) or tenofovir (e.g., tenofovir dipivoxil or tenofovir alafenamide) is administered orally (e.g., the compound of Formula (I) or Formula (II) is administered orally, or tenofovir (e.g., tenofovir dipivoxil or tenofovir alafenamide) is administered orally, or both the compound of Formula (I) or Formula (II) and entecavir are administered orally). In some embodiments, the compound of Formula (I) or Formula (II) or tenofovir (e.g., tenofovir dipivoxil or tenofovir alafenamide) is administered parenterally (e.g., the compound of Formula (II) is administered parenterally). In some embodiments, the compound of Formula (I) or Formula (II) is administered parenterally and tenofovir (e.g., tenofovir dipivoxil or tenofovir alafenamide) is administered orally. In some embodiments, compound of Formula (I) or Formula (II) is formulated as a fixed dose combination with tenofovir (e.g., tenofovir dipivoxil or tenofovir alafenamide), e.g., as a liquid dosage form or solid dosage form (e.g., a capsule or tablet). In some embodiments, compound of Formula (I) or Formula (II) is formulated as a fixed dose combination with tenofovir (e.g., tenofovir dipivoxil or tenofovir alafenamide), e.g., as a liquid dosage form or solid dosage form (e.g., a capsule or tablet) for oral administration.

In some embodiments, the administration of a compound of Formula (I) or Formula (II) and tenofovir (e.g., tenofovir dipivoxil or tenofovir alafenamide) has a synergistic or additive effect. In some embodiments, the administration of a compound of Formula (I) or Formula (II) and tenofovir (e.g., tenofovir dipivoxil or tenofovir alafenamide) has an additive effect. In some embodiments, the administration of a compound of Formula (I) or Formula (II) and tenofovir (e.g., tenofovir dipivoxil or tenofovir alafenamide) has a synergistic effect.

In some embodiments, the composition comprises a mixture of compounds of Formula (I), e.g., Formula (Ib) and Formula (Ic). In some embodiments, the composition comprises Formula (Ib) and comprises less than about 5% of Formula (Ic) (e.g., less than about 4%, less than about 3%, less than about 2%, less than about 1%, less than about 0.5%, or less than about 0.1% of Formula (Ic)), or is substantially free of Formula (Ic). In some embodiments, the composition comprises Formula (Ic) and comprises less than about 5% of Formula (Ib) (e.g., less than about 4%, less than about 3%, less than about 2%, less than about 1%, less than about 0.5%, or less than about 0.1% of Formula (Ib), or is substantially free of Formula (Ib)).

In some embodiments, the composition comprises a mixture of compounds of Formula (II), e.g., Formula (IIb) and Formula (IIc). In some embodiments, the composition comprises Formula (IIb) and comprises less than about 5% of Formula (IIc) (e.g., less than about 4%, less than about 3%, less than about 2%, less than about 1%, less than about 0.5%, or less than about 0.1% of Formula (IIc), or is substantially free of Formula (IIc)). In some embodiments, the composition comprises Formula (IIc) and comprises less than about 5% of Formula (IIb) (e.g., less than about 4%, less than about 3%, less than about 2%, less than about 1%, less than about 0.5%, or less than about 0.1% of Formula (IIb), or is substantially free of Formula (IIb)).

In some embodiments, the subject is a mammal. In some embodiments, the subject is a human. In some embodiments the subject is a non-human animal, e.g., a woodchuck (e.g., Eastern woodchuck).

In some embodiments, the method further comprises the administration of a therapeutically effective amount of an additional agent. In some embodiments, the additional agent is an antiviral agent or an anticancer agent. In some embodiments, the antiviral agent comprises an interferon, a nucleoside analog, a non-nucleoside antiviral, or a non-interferon immune enhancer. In some embodiments, the interferon comprises interferon alfa-2a, interferon alfa-2b, interferon alfa-n1, interferon alfacon-1, or a pegylated interferon (e.g., peginterferon alfa-2a, peginterferon alfa-2b). In some embodiments, the nucleoside analog comprises lamivudine, adefovir dipivoxil, telbivudine, clevudine, ribavarin, entecavir, besifovir, or AGX-1009. In some embodiments, the antiviral agent is entecavir. In some embodiments, the antiviral compound comprises NOV-225, BAM 205, Myrcludex B, ARC-520, BAY 41-4109, REP 9AC, Alinia (nitazoxanide), Dd-RNAi, NVR-121 (NVR 3-778), BSBI-25, NVP-018, TKM-HBV, or ALN-HBV. In some embodiments, the non-interferon immune enhancer comprises zadaxin (thymosin alpha-1), GS-4774, CYT107 (interleukin-7), Dv-601, HBV core antigen vaccine, or GS-9620. In some embodiments, the antiviral agent is a capsid inhibitor, an entry inhibitor, a secretion inhibitor, a microRNA, an antisense RNA agent, an RNAi agent, or other agent designed to inhibit viral RNA. In some embodiments, the anticancer agent is selected from methotrexate, 5-fluorouracil, doxorubicin, vincristine, bleomycin, vinblastine, dacarbazine, toposide, cisplatin, epirubicin, and sorafenib tosylate.

In some embodiments, in a method described herein, the subject is treatment naïve. In some embodiments, the subject has previously been treated for HBV infection. In some embodiments, the previous treatment for HBV infection has failed. In some embodiments, the subject has relapsed.

In some embodiments, the subject has been previously been treated with an anti-HBV agent other than a compound of Formula (I) or Formula (II) or a pharmaceutically acceptable salt thereof (e.g., an interferon, ribavirin) and is suffering from a relapsed HBV infection.

In some embodiments, the subject is suffering from a co-infection with Hepatitis B virus (HDV). In some embodiments, the subject has been diagnosed with an HBV infection. In some embodiments, the subject has been diagnosed with an HDV infection. In some embodiments, the subject has been diagnosed with a co-infection of HBV and HDV.

In some embodiments, in a method described herein, the subject has been diagnosed with cirrhosis of the liver. In some embodiments, the subject has been diagnosed with hepatocellular carcinoma. In some embodiments, the subject has been diagnosed with hepatocellular carcinoma and is awaiting liver transplantation. In some embodiments, the subject is non-cirrhotic or has not been diagnosed with hepatocellular carcinoma.

In some embodiments, in a method described herein, the subject has been further diagnosed with an HIV infection. In some embodiments, the strain of HIV infection is known. In some embodiments, the subject is infected with HIV-1 or HIV-2 (e.g., strain 1 or strain 2).

In some embodiments, the methods described herein further comprise analyzing or receiving analysis of the body weight and temperature of the subject at least once a week until the end of treatment.

In some embodiments, the methods described herein further comprise analyzing or receiving analysis of a blood sample from the subject at least once prior to the end of treatment. In some embodiments, the blood sample is analyzed for viral load and surface antigen levels. In some embodiments, the blood sample is analyzed for the expression level of interferon (e.g., interferon alfa or interferon beta), an interferon stimulating protein (e.g., ISG15, CXCL10, OAS 1), or other cytokines. In some embodiments, the blood sample is analyzed for the presence of anti-WHS antibodies and anti-WHc antibodies.

In some embodiments, the methods described herein further comprise analyzing or receiving analysis of a liver biopsy specimen from the subject at least once prior to the end of treatment. In some embodiments, the liver biopsy specimen is analyzed for the levels of viral DNA, viral RNA, viral antigens, and cccDNA. In some embodiments, the liver biopsy specimen is analyzed for the expression level of interferon (e.g., interferon alfa or interferon beta), an interferon stimulating protein (e.g., ISG15, CXCL10, OAS 1), or other cytokines. In some embodiments, the liver biopsy specimen is analyzed for the presence of anti-WHS antibodies and anti-WHc antibodies. In some embodiments, the liver biopsy specimen is analyzed for the reduction of liver inflammation, necrosis, steatosis, or fibrosis.

In any and all embodiments, the method features a pharmaceutical composition for use in treating Hepatitis B virus in a subject, the composition comprising a compound of Formula (I) (e.g., Formula (Ia), Formula (Ib), or Formula (Ic)), a compound of Formula (II) (e.g., Formula (IIa), Formula (IIb), or Formula (IIc)), or a pharmaceutically acceptable salt thereof in combination with tenofovir (e.g., tenofovir dipivoxil or tenofovir alafenamide) to thereby treat the subject.

In another aspect, the present invention features a method of treating a subject infected with a drug-resistant strain of the Hepatitis B virus (HBV), the method comprising administering to the subject a compound of Formula (I), wherein the compound is selected from:

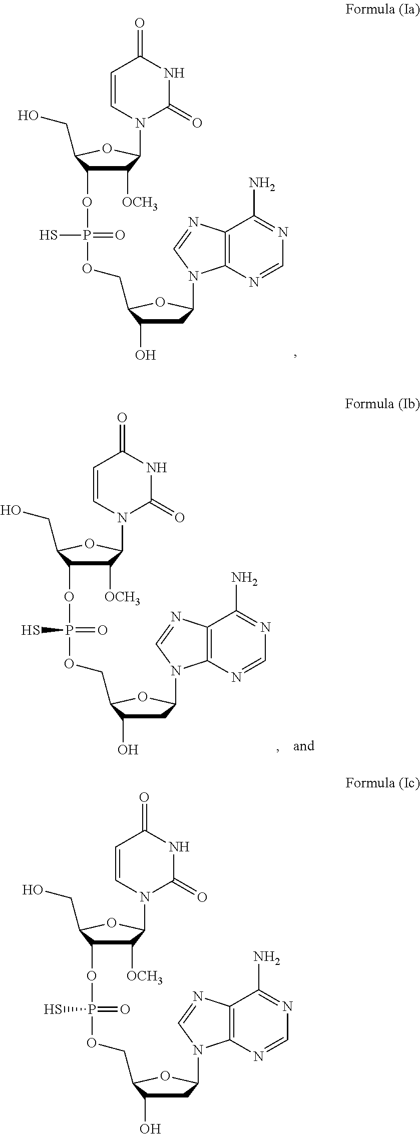

or a prodrug or pharmaceutically acceptable salt thereof to thereby treat the subject, and wherein the level of an HBV biomarker is not substantially reduced in the subject upon administration of an anti-HBV agent other than a compound of Formula (I). In some embodiments, the prodrug of Formula (I) is a compound of Formula (II), wherein the compound is selected from:

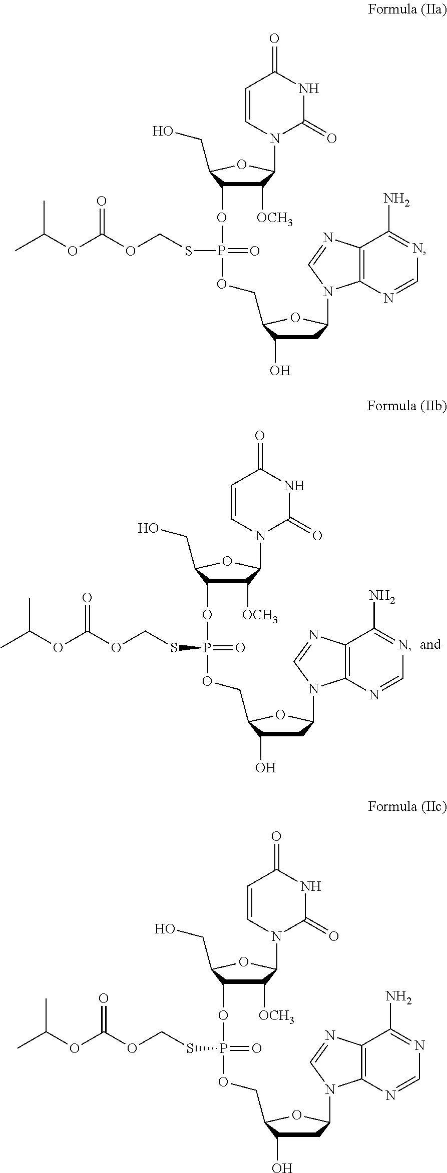

or a pharmaceutically acceptable salt thereof. In some embodiments, the method comprises administering to the subject a compound of Formula (I) or a pharmaceutically acceptable salt thereof. In some embodiments, the method comprises administering to the subject a compound of Formula (II) or a pharmaceutically acceptable salt thereof.

In some embodiments, the HBV biomarker comprises the viral load, HBsAg level, HBeAg level, or cccDNA level. In some embodiments, the viral load of the drug-resistant strain of HBV is not substantially reduced by exposure to an anti-HBV agent other than a compound of Formula (I) or Formula (II) or a pharmaceutically acceptable salt thereof. In some embodiments, the viral load of the drug-resistant strain of HBV is reduced by less than about 50%, about 40%, about 30%, about 20%, about 15%, about 10%, about 5%, about 2.5%, about 1%, about 0.5%, about 0.1%, or less upon exposure to an anti-HBV agent other than a compound of Formula (I) or Formula (II) or a pharmaceutically acceptable salt thereof. In some embodiments, the viral load of the drug-resistant strain of HBV is reduced by less than about 2 log units, about 1.5 log units, about 1 log unit, about 0.5 log units, about 0.1 log units, or less upon administration of an anti-HBV agent other than a compound of Formula (I) or Formula (II) or a pharmaceutically acceptable salt thereof.

In some embodiments, the viral load of the drug-resistant strain of HBV is substantially reduced by a compound of Formula (I) or Formula (II) or a pharmaceutically acceptable salt thereof. In some embodiments, the viral load of the drug-resistant strain of HBV is reduced by more than about 10%, about 20%, about 30%, about 40%, about 50%, about 60%, about 70%, about 80%, about 90%, about 95%, about 99%, about 99.9%, or about 99.99% or more upon administration of a compound of Formula (I) or Formula (II) or a pharmaceutically acceptable salt thereof. In some embodiments, the viral load of the drug-resistant strain of HBV is reduced by more than about 1 log unit, about 1.5 log units, about 2 log units, about 2.5 log units, about 3 log units, about 3.5 log units, about 4 log units, about 4.5 log units, about 5 log units, or more upon administration to a compound of Formula (I) or Formula (II) or a pharmaceutically acceptable salt thereof.

In some embodiments, the HBsAg level of the drug-resistant strain of HBV is not substantially reduced by exposure to an anti-HBV agent other than a compound of Formula (I) or Formula (II) or a pharmaceutically acceptable salt thereof. In some embodiments, the HBsAg level of the drug-resistant strain of HBV is reduced by less than about 50%, about 40%, about 30%, about 20%, about 15%, about 10%, about 5%, about 2.5%, about 1%, about 0.5%, about 0.1%, or less upon exposure to an anti-HBV agent other than a compound of Formula (I) or Formula (II) or a pharmaceutically acceptable salt thereof. In some embodiments, the HBsAg level of the drug-resistant strain of HBV is reduced by less than about 2 log units, about 1.5 log units, about 1 log unit, about 0.5 log units, about 0.1 log units, or less upon administration of an anti-HBV agent other than a compound of Formula (I) or Formula (II) or a pharmaceutically acceptable salt thereof.

In some embodiments, the HBsAg level of the drug-resistant strain of HBV is substantially reduced by a compound of Formula (I) or Formula (II) or a pharmaceutically acceptable salt thereof. In some embodiments, the HBsAg level of the drug-resistant strain of HBV is reduced by more than about 10%, about 20%, about 30%, about 40%, about 50%, about 60%, about 70%, about 80%, about 90%, about 95%, about 99%, about 99.9%, or about 99.99% or more upon administration of a compound of Formula (I) or Formula (II) or a pharmaceutically acceptable salt thereof. In some embodiments, the HBsAg level of the drug-resistant strain of HBV is reduced by more than about 1 log unit, about 1.5 log units, about 2 log units, about 2.5 log units, about 3 log units, about 3.5 log units, about 4 log units, about 4.5 log units, about 5 log units, or more upon administration to a compound of Formula (I) or Formula (II) or a pharmaceutically acceptable salt thereof.

In some embodiments, the HBeAg level of the drug-resistant strain of HBV is not substantially reduced by exposure to an anti-HBV agent other than a compound of Formula (I) or Formula (II) or a pharmaceutically acceptable salt thereof. In some embodiments, the HBeAg level of the drug-resistant strain of HBV is reduced by less than about 50%, about 40%, about 30%, about 20%, about 15%, about 10%, about 5%, about 2.5%, about 1%, about 0.5%, about 0.1%, or less upon exposure to an anti-HBV agent other than a compound of Formula (I) or Formula (II) or a pharmaceutically acceptable salt thereof. In some embodiments, the HBeAg level of the drug-resistant strain of HBV is reduced by less than about 2 log units, about 1.5 log units, about 1 log unit, about 0.5 log units, about 0.1 log units, or less upon administration of an anti-HBV agent other than a compound of Formula (I) or Formula (II) or a pharmaceutically acceptable salt thereof.

In some embodiments, the HBeAg level of the drug-resistant strain of HBV is substantially reduced by a compound of Formula (I) or Formula (II) or a pharmaceutically acceptable salt thereof. In some embodiments, the HBeAg level of the drug-resistant strain of HBV is reduced by more than about 10%, about 20%, about 30%, about 40%, about 50%, about 60%, about 70%, about 80%, about 90%, about 95%, about 99%, about 99.9%, or about 99.99% or more upon administration of a compound of Formula (I) or Formula (II) or a pharmaceutically acceptable salt thereof. In some embodiments, the HBeAg level of the drug-resistant strain of HBV is reduced by more than about 1 log unit, about 1.5 log units, about 2 log units, about 2.5 log units, about 3 log units, about 3.5 log units, about 4 log units, about 4.5 log units, about 5 log units, or more upon administration to a compound of Formula (I) or Formula (II) or a pharmaceutically acceptable salt thereof.

In some embodiments, the cccDNA level of the drug-resistant strain of HBV is not substantially reduced by exposure to an anti-HBV agent other than a compound of Formula (I) or Formula (II) or a pharmaceutically acceptable salt thereof. In some embodiments, the cccDNA level of the drug-resistant strain of HBV is reduced by less than about 50%, about 40%, about 30%, about 20%, about 15%, about 10%, about 5%, about 2.5%, about 1%, about 0.5%, about 0.1%, or less upon exposure to an anti-HBV agent other than a compound of Formula (I) or Formula (II) or a pharmaceutically acceptable salt thereof. In some embodiments, the cccDNA level of the drug-resistant strain of HBV is reduced by less than about 2 log units, about 1.5 log units, about 1 log unit, about 0.5 log units, about 0.1 log units, or less upon administration of an anti-HBV agent other than a compound of Formula (I) or Formula (II) or a pharmaceutically acceptable salt thereof.

In some embodiments, the cccDNA level of the drug-resistant strain of HBV is substantially reduced by a compound of Formula (I) or Formula (II) or a pharmaceutically acceptable salt thereof. In some embodiments, the cccDNA level of the drug-resistant strain of HBV is reduced by more than about 10%, about 20%, about 30%, about 40%, about 50%, about 60%, about 70%, about 80%, about 90%, about 95%, about 99%, about 99.9%, or about 99.99% or more upon administration of a compound of Formula (I) or Formula (II) or a pharmaceutically acceptable salt thereof. In some embodiments, the cccDNA level of the drug-resistant strain of HBV is reduced by more than about 1 log unit, about 1.5 log units, about 2 log units, about 2.5 log units, about 3 log units, about 3.5 log units, about 4 log units, about 4.5 log units, about 5 log units, or more upon administration to a compound of Formula (I) or Formula (II) or a pharmaceutically acceptable salt thereof.

In some embodiments, the drug-resistant strain of HBV is resistant to an anti-HBV agent other than a compound of Formula (I) or Formula (II) or a pharmaceutically acceptable salt thereof, and the anti-HBV agent is an interferon, a nucleoside analog, a non-nucleoside antiviral, an immune enhancer, or a direct-acting antiviral agent. In some embodiments, the interferon comprises interferon alfa-2a, interferon alfa-2b, interferon alfa-n1, interferon alfacon-1, or a pegylated interferon (e.g., peginterferon alfa-2a, peginterferon alfa-2b). In some embodiments, the nucleoside analog comprises lamivudine, adefovir dipivoxil, entecavir, telbivudine, clevudine, ribavarin, tenofovir, tenofovir alafenamide, besifovir, or AGX-1009. In some embodiments, non-nucleoside antiviral agent comprises NOV-225, BAM 205, Myrcludex B, ARC-520, BAY 41-4109, REP 9AC, Alinia (nitazoxanide), Dd-RNAi, NVR-121 (NVR 3-778), BSBI-25, NVP-018, TKM-HBV, or ALN-HBV. In some embodiments, the immune enhancer comprises zadaxin (thymosin alpha-1), GS-4774, CYT107 (interleukin-7), Dv-601, HBV core antigen vaccine, or GS-9620.

In some embodiments, the drug-resistant HBV strain is an HBV variant strain or HBV mutant strain. In some embodiments, the drug-resistant HBV strain comprises a variant or mutant form of the HBsAg, HBcAg, HBeAg, L, M, P, or X proteins. In some embodiments, the drug-resistant HBV variant comprises an amino acid mutation (e.g., an amino acid substitution, addition, or deletion) in the sequence of the HBsAg, HBcAg, HBeAg, L, M, P, or X proteins, e.g., as compared with a reference sequence.

In some embodiments, the drug-resistant HBV variant comprises an amino acid mutation (e.g., an amino acid substitution, addition, or deletion) in the sequence of the HBsAg protein, e.g., as compared with a reference sequence. In some embodiments, the amino acid mutation (e.g., an amino acid substitution, addition, or deletion) in the HBsAg protein sequence comprises a mutation located between amino acid position 100 and amino acid position 200, e.g., as compared with a reference sequence. In some embodiments, the amino acid mutation (e.g., an amino acid substitution, addition, or deletion) in the HBsAg protein sequence comprises a mutation at amino acid positions 115, 118, 120, 123, 126, 129, 131, 133, 134, 142, 143, 144, 145, or 154, e.g., as compared with a reference sequence. In some embodiments, the amino acid mutation (e.g., an amino acid substitution, addition, or deletion) in the HBsAg protein sequence comprises a T115N, T118V, P120L, P120Q, T126S, Q129H, T131K, M133I, M133L, F134N, F134H, P142L, P142S, T143L, D144A, D144V, G145R, or S154P mutation.

In some embodiments, the amino acid mutation (e.g., an amino acid substitution, addition, or deletion) in the HBsAg protein sequence comprises a mutation located between amino acid position 150 and amino acid position 200, e.g., as compared with a reference sequence. In some embodiments, the amino acid mutation (e.g., an amino acid substitution, addition, or deletion) in the HBsAg protein sequence comprises a mutation at amino acid positions 161, 172, 173, 175, 176, 193, 194, or 196, e.g., as compared with a reference sequence. In some embodiments, the amino acid mutation (e.g., an amino acid substitution, addition, or deletion) in the HBsAg protein sequence comprises a F161H, F161L, W172L, W172*, L173F, L175F, L176V, L176*, S193L, V194F, V194S, I195M, W196L, W196S, or W196* mutation, e.g., as compared to a reference or consensus sequence, wherein “*” represents a stop codon.

In some embodiments, the drug-resistant HBV variant comprises an amino acid mutation (e.g., an amino acid substitution, addition, or deletion) in the sequence of the P protein, e.g., as compared with a reference sequence. In some embodiments, the amino acid mutation (e.g., an amino acid substitution, addition, or deletion) in the P protein sequence comprises a mutation located between amino acid position 60 and amino acid position 275, e.g., as compared with a reference sequence. In some embodiments, the amino acid mutation (e.g., an amino acid substitution, addition, or deletion) in the P protein sequence comprises a mutation at amino acid positions 80, 169, 173, 180, 181, 184, 169, 202, 204, 215, 233, 236, or 250, e.g., as compared with a reference sequence. In some embodiments, the amino acid mutation (e.g., an amino acid substitution, addition, or deletion) in the P protein sequence comprises a N169T, I169T, V173L, L180M, A181T, A181V, T184A, T184C, T184G, T184I, T184L, T184M, T184S, S202C, S202G, S202I, M204I, M204V, N236T, M250I, or M250V mutation. In some embodiments, the amino acid mutation (e.g., an amino acid substitution, addition, or deletion) in the P protein sequence comprises a L180M, M204I, M204V, or N236T mutation.

In some embodiments, the method described herein comprises administering to the subject a mixture of compounds of Formula (I) or pharmaceutically acceptable salts thereof. In some embodiments, the method described herein comprises administering to the subject a mixture of compounds of Formula (Ib) and Formula (Ic) or pharmaceutically acceptable salts thereof. In some embodiments, the mixture comprises a ratio of Formula (Ib) to Formula (Ic) of about 1:1 (e.g., a racemic mixture). In some embodiments, the mixture comprises a ratio of Formula (Ib) to Formula (Ic) of about 51:49, about 52:48, about 53:47, about 54:46, about 55:45, about 60:40, about 65:35, about 70:30, about 75:25, about 80:20, about 85:15, about 90:10, about 95:5, or about 99:1. In some embodiments, the mixture comprises a ratio of Formula (Ic) to Formula (Ib) of about 51:49, about 52:48, about 53:47, about 54:46, about 55:45, about 60:40, about 65:35, about 70:30, about 75:25, about 80:20, about 85:15, about 90:10, about 95:5, or about 99:1.

In some embodiments, the method described herein comprises administering to the subject a mixture of compounds of Formula (I) comprising Formula (Ib) and less than about 5% of Formula (Ic), e.g., less than about 4%, less than about 3%, less than about 2%, less than about 1%, less than about 0.5%, or less than about 0.1% of Formula (Ic). In some embodiments, the method described herein comprises administering to the subject a compound of Formula (I) comprising Formula (Ib) or a pharmaceutically acceptable salt thereof that is substantially free of Formula (Ic). In some embodiments, the method described herein comprises administering to the subject a mixture of compounds of Formula (I) comprising Formula (Ic) and less than about 5% of Formula (Ib), e.g., less than about 4%, less than about 3%, less than about 2%, less than about 1%, less than about 0.5%, or less than about 0.1% of Formula (Ib). In some embodiments, the method described herein comprises administering to the subject a compound of Formula (I) comprising Formula (Ic) or a pharmaceutically acceptable salt thereof that is substantially free of Formula (Ib).

In some embodiments, the method described herein comprises administering to the subject a mixture of compounds of Formula (II) or pharmaceutically acceptable salts thereof. In some embodiments, the method described herein comprises administering to the subject a mixture of Formula (IIb) and Formula (IIc) or pharmaceutically acceptable salts thereof. In some embodiments, the mixture comprises a ratio of Formula (IIb) to Formula (IIc) of about 1:1 (e.g., a racemic mixture). In some embodiments, the mixture comprises a ratio of Formula (IIb) to Formula (IIc) of about 51:49, about 52:48, about 53:47, about 54:46, about 55:45, about 60:40, about 65:35, about 70:30, about 75:25, about 80:20, about 85:15, about 90:10, about 95:5, or about 99:1. In some embodiments, the mixture comprises a ratio of Formula (IIc) to Formula (IIb) of about 51:49, about 52:48, about 53:47, about 54:46, about 55:45, about 60:40, about 65:35, about 70:30, about 75:25, about 80:20, about 85:15, about 90:10, about 95:5, or about 99:1.

In some embodiments, the method described herein comprises administering to the subject a mixture of compounds of Formula (II) comprising Formula (IIb) and less than about 5% of Formula (IIc), e.g., less than about 4%, less than about 3%, less than about 2%, less than about 1%, less than about 0.5%, or less than about 0.1% of Formula (IIc). In some embodiments, the method described herein comprises administering to the subject a compound of Formula (II) comprising Formula (IIb) or a pharmaceutically acceptable salt thereof that is substantially free of Formula (IIc). In some embodiments, the method described herein comprises administering to the subject a mixture of compounds of Formula (II) comprising Formula (IIc) and less than about 5% of Formula (IIb), e.g., less than about 4%, less than about 3%, less than about 2%, less than about 1%, less than about 0.5%, or less than about 0.1% of Formula (IIb). In some embodiments, the method described herein comprises administering to the subject a compound of Formula (II) comprising Formula (IIc) or a pharmaceutically acceptable salt thereof that is substantially free of Formula (IIb).

In some embodiments, in a method described herein, the IC₅₀ value of a compound of Formula (I) or Formula (II) is less than 10 μM (e.g., a compound of Formula (II) is less than 10 μM). In some embodiments, the IC₅₀ value of a compound of Formula (I) or Formula (II) is less than 1 μM (e.g., a compound of Formula (II) is less than 1 μM). In some embodiments, the IC₅₀ value of a compound of Formula (I) or Formula (II) is less than 0.1 μM (e.g., the IC₅₀ value of a compound of Formula (II) is less than 0.1 μM). In some embodiments, the IC₅₀ value of a compound of Formula (I) or Formula (II) is less than 0.01 μM (e.g., the IC₅₀ value of a compound of Formula (II) is less than 0.1 μM).

In some embodiments, in a method described herein, the compound of Formula (I) or Formula (II) is administered orally. In some embodiments, the compound of Formula (I) is administered orally. In some embodiments, the compound of Formula (II) is administered orally. In some embodiments, the compound of Formula (I) or Formula (II) is administered parenterally. In some embodiments, the compound of Formula (I) is administered parenterally. In some embodiments, the compound of Formula (II) is administered parenterally. In some embodiments, the compound of Formula (I) or Formula (II) is administered intravenously. In some embodiments, the compound of Formula (I) is administered intravenously. In some embodiments, the compound of Formula (II) is administered intravenously.

In some embodiments, the compound of Formula (I) or Formula (II) is formulated a liquid or solid dosage form. In some embodiments, the liquid dosage form comprises a suspension, a solution, a linctus, an emulsion, a drink, an elixir, or a syrup. In some embodiments, the solid dosage form comprises a capsule, tablet, pill, dragée, powder, or microencapsulated dose form.

In some embodiments, the compound (e.g., a compound of Formula (I) or Formula (II)) is administered at a dosage between about 0.5 mg/kg and about 1000 mg/kg. In some embodiments, the compound (e.g., a compound of Formula (I) or Formula (II)) is administered at a dosage between about 0.5 mg/kg and about 1000 mg/kg, about 900 mg/kg, about 800 mg/kg, about 700 mg/kg, about 600 mg/kg, about 500 mg/kg, about 400 mg/kg, about 300 mg/kg, about 250 mg/kg about 200 mg/kg, about 150 mg/kg, about 100 mg/kg, about 75 mg/kg, about 50 mg/kg, about 25 mg/kg, about 10 mg/kg, about 5 mg/kg, about 2.5 mg/kg, or less. In some embodiments, the compound (e.g., a compound of Formula (I) or Formula (II)) is administered at a dosage between about 5 mg/kg and about 500 mg/kg. In some embodiments, the compound (e.g., a compound of Formula (I) or Formula (II)) is administered at a dosage between about 5 mg/kg and about 500 mg/kg, 450 mg/kg, about 400 mg/kg, about 350 mg/kg about 300 mg/kg, about 250 mg/kg, about 200 mg/kg, about 150 mg/kg, about 100 mg/kg, about 75 mg/kg, about 50 mg/kg, about 25 mg/kg, about 10 mg/kg, or less.

In some embodiments, the dosage of Formula (I) or Formula (II) is between about 10 mg and about 1500 mg, about 1250 mg, about 1000 mg, about 900 mg, about 800 mg, about 700 mg, about 600 mg, about 500 mg, about 400 mg, about 300 mg, about 250 mg, about 200 mg, about 150 mg, about 100 mg, about 75 mg, about 50 mg, about 25 mg, or less. In some embodiments, the dosage of Formula (I) or Formula (II) is between about 10 mg, about 25 mg, about 50 mg, about 75 mg, about 100 mg, about 150 mg, about 200 mg, about 250 mg, about 300 mg, about 400 mg, about 500 mg, about 600 mg, about 700 mg, about 800 mg, about 900 mg, about 1000 mg, about 1250 mg, and about 1500 mg. In some embodiments, the dosage of Formula (I) or Formula (II) is between about 50 mg and about 1000 mg. In some embodiments, the dosage of Formula (I) or Formula (II) is between about 200 mg and about 1000 mg.

In some embodiments, in a method described herein, the compound of Formula (I) or Formula (II) is administered daily. In some embodiments, the compound of Formula (I) or Formula (II) is administered once daily. In some embodiments, the compound of Formula (I) or Formula (II) is administered more than once a day, e.g., twice a day, three times a day, four times a day. In some embodiments, the compound of Formula (I) or Formula (II) is administered every other day, every 2 days, every 3 days, every 4 days, or more. In some embodiments, thecompound of Formula (I) or Formula (II) is administered once a week, twice a week, three times a week, four times a week, five times a week, or six times a week.

In some embodiments, in a method described herein, the duration of the method is one day. In some embodiments, the duration of the method is greater than 1 day, e.g., about 2 days, about 3 days, about 4 days, about 5 days, about 6 days, about 7 days, about 8 days, about 9 days, about 10 days, about 11 days, about 12 days, about 13 days, about 14 days, about 2 weeks, about 3 weeks, about 4 weeks, about 1 month, about 1.5 months, about 2 months, about 3 months, about 4 months, about 5 months, about 6 months. In some embodiments, the duration of the method is between about 1 day and about 2 weeks. In some embodiments, the duration of the method is between 6 days and 14 days. In some embodiments, the duration of the method is for one week. In some embodiments, the duration of the method lasts until the subject is cured of HBV infection (e.g., until the subject presents an undetectable level of HBV RNA).

In some embodiments, in a method described herein, a compound of Formula (I) or Formula (II) is formulated as a pharmaceutical composition. In some embodiments, the pharmaceutical composition further comprises a pharmaceutically acceptable carrier or excipient.

In some embodiments, in a method described herein, the subject is a mammal. In some embodiments, the subject is a human. In some embodiments, the subject has been diagnosed with HBV infection. In some embodiments, the subject is diagnosed with chronic hepatitis B (CHB). In some embodiments, the genotype of the HBV infection is known. In some embodiments, the subject is infected with HBV genotype A (e.g., HBV-A1-7), HBV genotype B (e.g., HBV-B2-5), HBV genotype C (e.g., HBV-C1-16), HBV genotype D (e.g., HBV-D1-7), HBV genotype E, HBV genotype F (e.g., HBV-F1-4), HBV genotype G, HBV genotype H, HBV genotype I, or HBV genotype J. In some embodiments, a compound of Formula (I) or Formula (II) has pan-genotypic activity.

In some embodiments, in a method described herein, the subject is treatment naïve. In some embodiments, the subject has previously been treated for HBV infection. In some embodiments, the previous treatment for HBV infection has failed. In some embodiments, the subject has relapsed.

In some embodiments, the subject has been previously been treated with an anti-HBV agent other than a compound of Formula (I) or Formula (II) or a pharmaceutically acceptable salt thereof (e.g., an interferon, ribavirin) and is suffering from a relapsed HBV infection.

In some embodiments, the subject is suffering from a co-infection with Hepatitis B virus (HDV). In some embodiments, the subject has been diagnosed with an HBV infection. In some embodiments, the subject has been diagnosed with an HDV infection. In some embodiments, the subject has been diagnosed with a co-infection of HBV and HDV.

In some embodiments, in a method described herein, the subject has been diagnosed with cirrhosis of the liver. In some embodiments, the subject has been diagnosed with hepatocellular carcinoma. In some embodiments, the subject has been diagnosed with hepatocellular carcinoma and is awaiting liver transplantation. In some embodiments, the subject is non-cirrhotic or has not been diagnosed with hepatocellular carcinoma.

In some embodiments, in a method described herein, the subject has been further diagnosed with an HIV infection. In some embodiments, the strain of HIV infection is known. In some embodiments, the subject is infected with HIV-1 or HIV-2 (e.g., strain 1 or strain 2).

In some embodiments, the subject has been previously been treated with an anti-HBV agent other than a compound of Formula (I) or Formula (II) or a pharmaceutically acceptable salt thereof (e.g., an interferon, ribavirin) and is suffering from a relapsed HBV infection.

In some embodiments, in a method described herein, the subject is further administered an additional agent or treatment or a pharmaceutically acceptable salt thereof. In some embodiments, the additional agent is an interferon, a nucleoside analog, a non-nucleoside antiviral, a non-interferon immune enhancer, or a direct-acting antiviral. In some embodiments, the additional agent is an interferon, e.g., peg-interferon alfa (e.g., peg-interferon alfa-2a or peg-interferon alfa-2b). In some embodiments, the additional agent is a nucleoside analog, e.g., lamivudine, adefovir dipivoxil, entecavir, telbivudine, clevudine, ribavarin, tenofovir (e.g., tenofovir dipivoxil or tenofovir alafenamide), besifovir, or AGX-1009. In some embodiments, the non-nucleoside antiviral agent comprises NOV-225, BAM 205, Myrcludex B, ARC-520, BAY 41-4109, REP 9AC, Alinia (nitazoxanide), Dd-RNAi, NVR-121 (NVR 3-778), BSBI-25, NVP-018, TKM-HBV, or ALN-HBV. In some embodiments, the immune enhancer comprises zadaxin (thymosin alpha-1), GS-4774, CYT107 (interleukin-7), Dv-601, HBV core antigen vaccine, or GS-9620. In some embodiments, the additional agent is entecavir.

In any and all embodiments, the method features a pharmaceutical composition for use in treating a drug-resistant strain of the Hepatitis B virus in a subject, the composition comprising a compound of Formula (I) (e.g., Formula (Ia), Formula (Ib), or Formula (Ic)), a compound of Formula (II) (e.g., Formula (IIa), Formula (IIb), or Formula (IIc)), or a pharmaceutically acceptable salt thereof to thereby treat the subject.

In another aspect, the present invention features a method of treating a subject infected with a drug-resistant strain of the Hepatitis B virus (HBV), the method comprising administering to the subject a compound of Formula (I), wherein the compound is selected from:

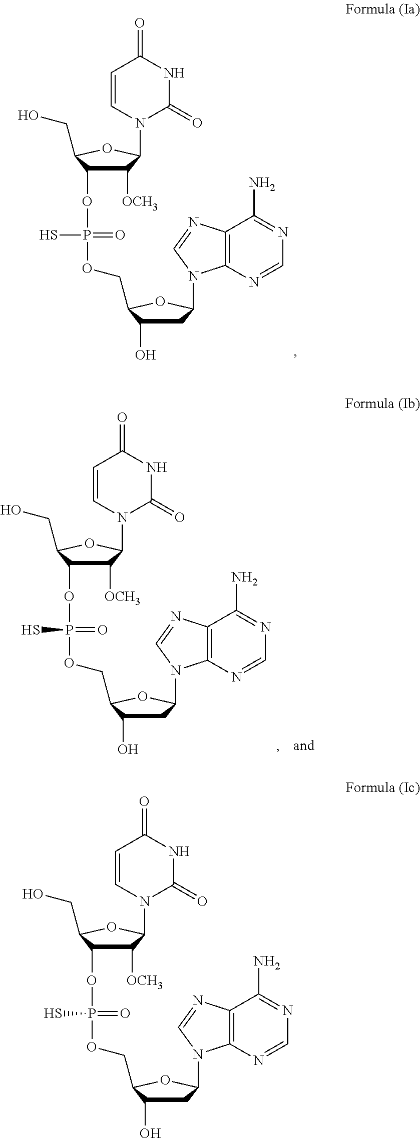

or a prodrug or pharmaceutically acceptable salt thereof to thereby treat the subject, and wherein the drug-resistant strain of HBV is resistant to an anti-HBV agent other than a compound of Formula (I). In some embodiments, the prodrug of Formula (I) is a compound of Formula (II), wherein the compound is selected from:

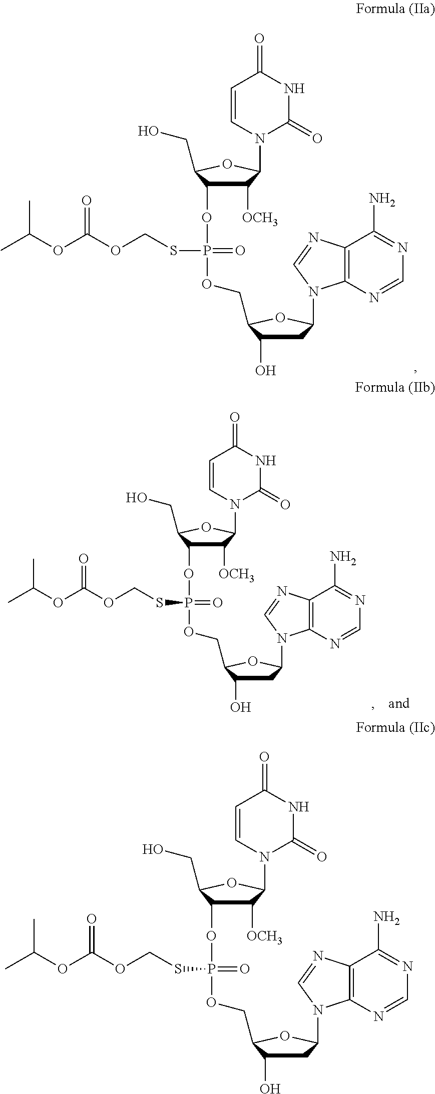

or a pharmaceutically acceptable salt thereof. In some embodiments, the method comprises administering to the subject a compound of Formula (I) or a pharmaceutically acceptable salt thereof. In some embodiments, the method comprises administering to the subject a compound of Formula (II) or a pharmaceutically acceptable salt thereof.

In some embodiments, the level of an HBV biomarker in the subject is not substantially reduced by exposure to an anti-HBV agent other than a compound of Formula (I) or Formula (II). In some embodiments, the level of an HBV biomarker is not substantially reduced by exposure to an anti-HBV agent other than a compound of Formula (I). In some embodiments, the level of an HBV biomarker is not substantially reduced by exposure to an anti-HBV agent other than a compound of Formula (II).

In some embodiments, the HBV biomarker comprises the viral load, HBsAg level, HBeAg level, or cccDNA level. In some embodiments, the viral load of the drug-resistant strain of HBV is not substantially reduced by exposure to an anti-HBV agent other than a compound of Formula (I) or Formula (II) or a pharmaceutically acceptable salt thereof. In some embodiments, the viral load of the drug-resistant strain of HBV is reduced by less than about 50%, about 40%, about 30%, about 20%, about 15%, about 10%, about 5%, about 2.5%, about 1%, about 0.5%, about 0.1%, or less upon exposure to an anti-HBV agent other than a compound of Formula (I) or Formula (II) or a pharmaceutically acceptable salt thereof. In some embodiments, the viral load of the drug-resistant strain of HBV is reduced by less than about 2 log units, about 1.5 log units, about 1 log unit, about 0.5 log units, about 0.1 log units, or less upon administration of an anti-HBV agent other than a compound of Formula (I) or Formula (II) or a pharmaceutically acceptable salt thereof.

In some embodiments, the viral load of the drug-resistant strain of HBV is substantially reduced by a compound of Formula (I) or Formula (II) or a pharmaceutically acceptable salt thereof. In some embodiments, the viral load of the drug-resistant strain of HBV is reduced by more than about 10%, about 20%, about 30%, about 40%, about 50%, about 60%, about 70%, about 80%, about 90%, about 95%, about 99%, about 99.9%, or about 99.99% or more upon administration of a compound of Formula (I) or Formula (II) or a pharmaceutically acceptable salt thereof. In some embodiments, the viral load of the drug-resistant strain of HBV is reduced by more than about 1 log unit, about 1.5 log units, about 2 log units, about 2.5 log units, about 3 log units, about 3.5 log units, about 4 log units, about 4.5 log units, about 5 log units, or more upon administration to a compound of Formula (I) or Formula (II) or a pharmaceutically acceptable salt thereof.

In some embodiments, the HBsAg level of the drug-resistant strain of HBV is not substantially reduced by exposure to an anti-HBV agent other than a compound of Formula (I) or Formula (II) or a pharmaceutically acceptable salt thereof. In some embodiments, the HBsAg level of the drug-resistant strain of HBV is reduced by less than about 50%, about 40%, about 30%, about 20%, about 15%, about 10%, about 5%, about 2.5%, about 1%, about 0.5%, about 0.1%, or less upon exposure to an anti-HBV agent other than a compound of Formula (I) or Formula (II) or a pharmaceutically acceptable salt thereof. In some embodiments, the HBsAg level of the drug-resistant strain of HBV is reduced by less than about 2 log units, about 1.5 log units, about 1 log unit, about 0.5 log units, about 0.1 log units, or less upon administration of an anti-HBV agent other than a compound of Formula (I) or Formula (II) or a pharmaceutically acceptable salt thereof.

In some embodiments, the HBsAg level of the drug-resistant strain of HBV is substantially reduced by a compound of Formula (I) or Formula (II) or a pharmaceutically acceptable salt thereof. In some embodiments, the HBsAg level of the drug-resistant strain of HBV is reduced by more than about 10%, about 20%, about 30%, about 40%, about 50%, about 60%, about 70%, about 80%, about 90%, about 95%, about 99%, about 99.9%, or about 99.99% or more upon administration of a compound of Formula (I) or Formula (II) or a pharmaceutically acceptable salt thereof. In some embodiments, the HBsAg level of the drug-resistant strain of HBV is reduced by more than about 1 log unit, about 1.5 log units, about 2 log units, about 2.5 log units, about 3 log units, about 3.5 log units, about 4 log units, about 4.5 log units, about 5 log units, or more upon administration to a compound of Formula (I) or Formula (II) or a pharmaceutically acceptable salt thereof.

In some embodiments, the HBeAg level of the drug-resistant strain of HBV is not substantially reduced by exposure to an anti-HBV agent other than a compound of Formula (I) or Formula (II) or a pharmaceutically acceptable salt thereof. In some embodiments, the HBeAg level of the drug-resistant strain of HBV is reduced by less than about 50%, about 40%, about 30%, about 20%, about 15%, about 10%, about 5%, about 2.5%, about 1%, about 0.5%, about 0.1%, or less upon exposure to an anti-HBV agent other than a compound of Formula (I) or Formula (II) or a pharmaceutically acceptable salt thereof. In some embodiments, the HBeAg level of the drug-resistant strain of HBV is reduced by less than about 2 log units, about 1.5 log units, about 1 log unit, about 0.5 log units, about 0.1 log units, or less upon administration of an anti-HBV agent other than a compound of Formula (I) or Formula (II) or a pharmaceutically acceptable salt thereof.

In some embodiments, the HBeAg level of the drug-resistant strain of HBV is substantially reduced by a compound of Formula (I) or Formula (II) or a pharmaceutically acceptable salt thereof. In some embodiments, the HBeAg level of the drug-resistant strain of HBV is reduced by more than about 10%, about 20%, about 30%, about 40%, about 50%, about 60%, about 70%, about 80%, about 90%, about 95%, about 99%, about 99.9%, or about 99.99% or more upon administration of a compound of Formula (I) or Formula (II) or a pharmaceutically acceptable salt thereof. In some embodiments, the HBeAg level of the drug-resistant strain of HBV is reduced by more than about 1 log unit, about 1.5 log units, about 2 log units, about 2.5 log units, about 3 log units, about 3.5 log units, about 4 log units, about 4.5 log units, about 5 log units, or more upon administration to a compound of Formula (I) or Formula (II) or a pharmaceutically acceptable salt thereof.

In some embodiments, the cccDNA level of the drug-resistant strain of HBV is not substantially reduced by exposure to an anti-HBV agent other than a compound of Formula (I) or Formula (II) or a pharmaceutically acceptable salt thereof. In some embodiments, the cccDNA level of the drug-resistant strain of HBV is reduced by less than about 50%, about 40%, about 30%, about 20%, about 15%, about 10%, about 5%, about 2.5%, about 1%, about 0.5%, about 0.1%, or less upon exposure to an anti-HBV agent other than a compound of Formula (I) or Formula (II) or a pharmaceutically acceptable salt thereof. In some embodiments, the cccDNA level of the drug-resistant strain of HBV is reduced by less than about 2 log units, about 1.5 log units, about 1 log unit, about 0.5 log units, about 0.1 log units, or less upon administration of an anti-HBV agent other than a compound of Formula (I) or Formula (II) or a pharmaceutically acceptable salt thereof.

In some embodiments, the cccDNA level of the drug-resistant strain of HBV is substantially reduced by a compound of Formula (I) or Formula (II) or a pharmaceutically acceptable salt thereof. In some embodiments, the cccDNA level of the drug-resistant strain of HBV is reduced by more than about 10%, about 20%, about 30%, about 40%, about 50%, about 60%, about 70%, about 80%, about 90%, about 95%, about 99%, about 99.9%, or about 99.99% or more upon administration of a compound of Formula (I) or Formula (II) or a pharmaceutically acceptable salt thereof. In some embodiments, the cccDNA level of the drug-resistant strain of HBV is reduced by more than about 1 log unit, about 1.5 log units, about 2 log units, about 2.5 log units, about 3 log units, about 3.5 log units, about 4 log units, about 4.5 log units, about 5 log units, or more upon administration to a compound of Formula (I) or Formula (II) or a pharmaceutically acceptable salt thereof.

In some embodiments, the drug-resistant strain of HBV is resistant to an anti-HBV agent other than a compound of Formula (I) or Formula (II) or a pharmaceutically acceptable salt thereof, and the anti-HBV agent is an interferon, a nucleoside analog, a non-nucleoside antiviral, an immune enhancer, or a direct-acting antiviral agent. In some embodiments, the interferon comprises interferon alfa-2a, interferon alfa-2b, interferon alfa-n1, interferon alfacon-1, or a pegylated interferon (e.g., peginterferon alfa-2a, peginterferon alfa-2b). In some embodiments, the nucleoside analog comprises lamivudine, adefovir dipivoxil, entecavir, telbivudine, clevudine, ribavarin, tenofovir (e.g., tenofovir dipivoxil or tenofovir alafenamide) besifovir, or AGX-1009. In some embodiments, non-nucleoside antiviral agent comprises NOV-225, BAM 205, Myrcludex B, ARC-520, BAY 41-4109, REP 9AC, Alinia (nitazoxanide), Dd-RNAi, NVR-121 (NVR 3-778), BSBI-25, NVP-018, TKM-HBV, or ALN-HBV. In some embodiments, the immune enhancer comprises zadaxin (thymosin alpha-1), GS-4774, CYT107 (interleukin-7), Dv-601, HBV core antigen vaccine, or GS-9620.

In some embodiments, the drug-resistant HBV strain is an HBV variant strain or HBV mutant strain. In some embodiments, the drug-resistant HBV strain comprises a variant or mutant form of the HBsAg, HBcAg, HBeAg, L, M, P, or X proteins. In some embodiments, the drug-resistant HBV variant comprises an amino acid mutation (e.g., an amino acid substitution, addition, or deletion) in the sequence of the HBsAg, HBcAg, HBeAg, L, M, P, or X proteins, e.g., as compared with a reference sequence.

In some embodiments, the drug-resistant HBV variant comprises an amino acid mutation (e.g., an amino acid substitution, addition, or deletion) in the sequence of the HBsAg protein, e.g., as compared with a reference sequence. In some embodiments, the amino acid mutation (e.g., an amino acid substitution, addition, or deletion) in the HBsAg protein sequence comprises a mutation located between amino acid position 100 and amino acid position 200, e.g., as compared with a reference sequence. In some embodiments, the amino acid mutation (e.g., an amino acid substitution, addition, or deletion) in the HBsAg protein sequence comprises a mutation at amino acid positions 115, 118, 120, 123, 126, 129, 131, 133, 134, 142, 143, 144, 145, or 154, e.g., as compared with a reference sequence. In some embodiments, the amino acid mutation (e.g., an amino acid substitution, addition, or deletion) in the HBsAg protein sequence comprises a T115N, T118V, P120L, P120Q, T126S, Q129H, T131K, M133I, M133L, F134N, F134H, P142L, P142S, T143L, D144A, D144V, G145R, or S154P mutation.

In some embodiments, the amino acid mutation (e.g., an amino acid substitution, addition, or deletion) in the HBsAg protein sequence comprises a mutation located between amino acid position 150 and amino acid position 200, e.g., as compared with a reference sequence. In some embodiments, the amino acid mutation (e.g., an amino acid substitution, addition, or deletion) in the HBsAg protein sequence comprises a mutation at amino acid positions 161, 172, 173, 175, 176, 193, 194, or 196, e.g., as compared with a reference sequence. In some embodiments, the amino acid mutation (e.g., an amino acid substitution, addition, or deletion) in the HBsAg protein sequence comprises a 161H, F161L, W172L, W172*, L173F, L175F, L176V, L176*, S193L, V194F, V194S, I195M, W196L, W196S, or W196* mutation, e.g., as compared to a reference or consensus sequence, wherein “*” represents a stop codon.

In some embodiments, the drug-resistant HBV variant comprises an amino acid mutation (e.g., an amino acid substitution, addition, or deletion) in the sequence of the P protein, e.g., as compared with a reference sequence. In some embodiments, the amino acid mutation (e.g., an amino acid substitution, addition, or deletion) in the P protein sequence comprises a mutation located between amino acid position 60 and amino acid position 275, e.g., as compared with a reference sequence. In some embodiments, the amino acid mutation (e.g., an amino acid substitution, addition, or deletion) in the P protein sequence comprises a mutation at amino acid positions 80, 169, 173, 180, 181, 184, 169, 202, 204, 215, 233, 236, or 250, e.g., as compared with a reference sequence. In some embodiments, the amino acid mutation (e.g., an amino acid substitution, addition, or deletion) in the P protein sequence comprises a N169T, I169T, V173L, L180M, A181T, A181V, T184A, T184C, T184G, T184I, T184L, T184M, T184S, S202C, S202G, S202I, M204I, M204V, N236T, M250I, or M250V mutation. In some embodiments, the amino acid mutation (e.g., an amino acid substitution, addition, or deletion) in the P protein sequence comprises a L180M, M204I, M204V, or N236T mutation.

In some embodiments, the method described herein comprises administering to the subject a mixture of compounds of Formula (I) or pharmaceutically acceptable salts thereof. In some embodiments, the method described herein comprises administering to the subject a mixture of compounds of Formula (Ib) and Formula (Ic) or pharmaceutically acceptable salts thereof. In some embodiments, the mixture comprises a ratio of Formula (Ib) to Formula (Ic) of about 1:1 (e.g., a racemic mixture). In some embodiments, the mixture comprises a ratio of Formula (Ib) to Formula (Ic) of about 51:49, about 52:48, about 53:47, about 54:46, about 55:45, about 60:40, about 65:35, about 70:30, about 75:25, about 80:20, about 85:15, about 90:10, about 95:5, or about 99:1. In some embodiments, the mixture comprises a ratio of Formula (Ic) to Formula (Ib) of about 51:49, about 52:48, about 53:47, about 54:46, about 55:45, about 60:40, about 65:35, about 70:30, about 75:25, about 80:20, about 85:15, about 90:10, about 95:5, or about 99:1.

In some embodiments, the method described herein comprises administering to the subject a mixture of compounds of Formula (I) comprising Formula (Ib) and less than about 5% of Formula (Ic), e.g., less than about 4%, less than about 3%, less than about 2%, less than about 1%, less than about 0.5%, or less than about 0.1% of Formula (Ic). In some embodiments, the method described herein comprises administering to the subject a compound of Formula (I) comprising Formula (Ib) or a pharmaceutically acceptable salt thereof that is substantially free of Formula (Ic). In some embodiments, the method described herein comprises administering to the subject a mixture of compounds of Formula (I) comprising Formula (Ic) and less than about 5% of Formula (Ib), e.g., less than about 4%, less than about 3%, less than about 2%, less than about 1%, less than about 0.5%, or less than about 0.1% of Formula (Ib). In some embodiments, the method described herein comprises administering to the subject a compound of Formula (I) comprising Formula (Ic) or a pharmaceutically acceptable salt thereof that is substantially free of Formula (Ib).

In some embodiments, the method described herein comprises administering to the subject a mixture of compounds of Formula (II) or pharmaceutically acceptable salts thereof. In some embodiments, the method described herein comprises administering to the subject a mixture of Formula (IIb) and Formula (IIc) or pharmaceutically acceptable salts thereof. In some embodiments, the mixture comprises a ratio of Formula (IIb) to Formula (IIc) of about 1:1 (e.g., a racemic mixture). In some embodiments, the mixture comprises a ratio of Formula (IIb) to Formula (IIc) of about 51:49, about 52:48, about 53:47, about 54:46, about 55:45, about 60:40, about 65:35, about 70:30, about 75:25, about 80:20, about 85:15, about 90:10, about 95:5, or about 99:1. In some embodiments, the mixture comprises a ratio of Formula (IIc) to Formula (IIb) of about 51:49, about 52:48, about 53:47, about 54:46, about 55:45, about 60:40, about 65:35, about 70:30, about 75:25, about 80:20, about 85:15, about 90:10, about 95:5, or about 99:1.

In some embodiments, the method described herein comprises administering to the subject a mixture of compounds of Formula (II) comprising Formula (IIb) and less than about 5% of Formula (IIc), e.g., less than about 4%, less than about 3%, less than about 2%, less than about 1%, less than about 0.5%, or less than about 0.1% of Formula (IIc). In some embodiments, the method described herein comprises administering to the subject a compound of Formula (II) comprising Formula (IIb) or a pharmaceutically acceptable salt thereof that is substantially free of Formula (IIc). In some embodiments, the method described herein comprises administering to the subject a mixture of compounds of Formula (II) comprising Formula (IIc) and less than about 5% of Formula (IIb), e.g., less than about 4%, less than about 3%, less than about 2%, less than about 1%, less than about 0.5%, or less than about 0.1% of Formula (IIb). In some embodiments, the method described herein comprises administering to the subject a compound of Formula (II) comprising Formula (IIc) or a pharmaceutically acceptable salt thereof that is substantially free of Formula (IIb).

In some embodiments, in a method described herein, the IC₅₀ value of a compound of Formula (I) or Formula (II) is less than 10 μM (e.g., a compound of Formula (II) is less than 10 μM). In some embodiments, the IC₅₀ value of a compound of Formula (I) or Formula (II) is less than 1 μM (e.g., a compound of Formula (II) is less than 1 μM). In some embodiments, the IC₅₀ value of a compound of Formula (I) or Formula (II) is less than 0.1 μM (e.g., the IC₅₀ value of a compound of Formula (II) is less than 0.1 μM). In some embodiments, the IC₅₀ value of a compound of Formula (I) or Formula (II) is less than 0.01 μM (e.g., the IC₅₀ value of a compound of Formula (II) is less than 0.1 μM).

In some embodiments, in a method described herein, the compound of Formula (I) or Formula (II) is administered orally. In some embodiments, the compound of Formula (I) is administered orally. In some embodiments, the compound of Formula (II) is administered orally. In some embodiments, the compound of Formula (I) or Formula (II) is administered parenterally. In some embodiments, the compound of Formula (I) is administered parenterally. In some embodiments, the compound of Formula (II) is administered parenterally. In some embodiments, the compound of Formula (I) or Formula (II) is administered intravenously. In some embodiments, the compound of Formula (I) is administered intravenously. In some embodiments, the compound of Formula (II) is administered intravenously.

In some embodiments, the compound of Formula (I) or Formula (II) is formulated a liquid or solid dosage form. In some embodiments, the liquid dosage form comprises a suspension, a solution, a linctus, an emulsion, a drink, an elixir, or a syrup. In some embodiments, the solid dosage form comprises a capsule, tablet, pill, dragée, powder, or microencapsulated dose form.

In some embodiments, the compound (e.g., a compound of Formula (I) or Formula (II)) is administered at a dosage between about 0.5 mg/kg and about 1000 mg/kg. In some embodiments, the compound (e.g., a compound of Formula (I) or Formula (II)) is administered at a dosage between about 0.5 mg/kg and about 1000 mg/kg, about 900 mg/kg, about 800 mg/kg, about 700 mg/kg, about 600 mg/kg, about 500 mg/kg, about 400 mg/kg, about 300 mg/kg, about 250 mg/kg about 200 mg/kg, about 150 mg/kg, about 100 mg/kg, about 75 mg/kg, about 50 mg/kg, about 25 mg/kg, about 10 mg/kg, about 5 mg/kg, about 2.5 mg/kg, or less. In some embodiments, the compound (e.g., a compound of Formula (I) or Formula (II)) is administered at a dosage between about 5 mg/kg and about 500 mg/kg. In some embodiments, the compound (e.g., a compound of Formula (I) or Formula (II)) is administered at a dosage between about 5 mg/kg and about 500 mg/kg, 450 mg/kg, about 400 mg/kg, about 350 mg/kg about 300 mg/kg, about 250 mg/kg, about 200 mg/kg, about 150 mg/kg, about 100 mg/kg, about 75 mg/kg, about 50 mg/kg, about 25 mg/kg, about 10 mg/kg, or less.

In some embodiments, the dosage of Formula (I) or Formula (II) is between about 10 mg and about 1500 mg, about 1250 mg, about 1000 mg, about 900 mg, about 800 mg, about 700 mg, about 600 mg, about 500 mg, about 400 mg, about 300 mg, about 250 mg, about 200 mg, about 150 mg, about 100 mg, about 75 mg, about 50 mg, about 25 mg, or less. In some embodiments, the dosage of Formula (I) or Formula (II) is between about 10 mg, about 25 mg, about 50 mg, about 75 mg, about 100 mg, about 150 mg, about 200 mg, about 250 mg, about 300 mg, about 400 mg, about 500 mg, about 600 mg, about 700 mg, about 800 mg, about 900 mg, about 1000 mg, about 1250 mg, and about 1500 mg. In some embodiments, the dosage of Formula (I) or Formula (II) is between about 50 mg and about 1000 mg. In some embodiments, the dosage of Formula (I) or Formula (II) is between about 200 mg and about 1000 mg.

In some embodiments, in a method described herein, the compound of Formula (I) or Formula (II) is administered daily. In some embodiments, the compound of Formula (I) or Formula (II) is administered once daily. In some embodiments, the compound of Formula (I) or Formula (II) is administered more than once a day, e.g., twice a day, three times a day, four times a day. In some embodiments, the compound of Formula (I) or Formula (II) is administered every other day, every 2 days, every 3 days, every 4 days, or more. In some embodiments, thecompound of Formula (I) or Formula (II) is administered once a week, twice a week, three times a week, four times a week, five times a week, or six times a week.

In some embodiments, in a method described herein, the duration of the method is one day. In some embodiments, the duration of the method is greater than 1 day, e.g., about 2 days, about 3 days, about 4 days, about 5 days, about 6 days, about 7 days, about 8 days, about 9 days, about 10 days, about 11 days, about 12 days, about 13 days, about 14 days, about 2 weeks, about 3 weeks, about 4 weeks, about 1 month, about 1.5 months, about 2 months, about 3 months, about 4 months, about 5 months, about 6 months. In some embodiments, the duration of the method is between about 1 day and about 2 weeks. In some embodiments, the duration of the method is between 6 days and 14 days. In some embodiments, the duration of the method is for one week. In some embodiments, the duration of the method lasts until the subject is cured of HBV infection (e.g., until the subject presents an undetectable level of HBV RNA).

In some embodiments, in a method described herein, a compound of Formula (I) or Formula (II) is formulated as a pharmaceutical composition. In some embodiments, the pharmaceutical composition further comprises a pharmaceutically acceptable carrier or excipient.

In some embodiments, in a method described herein, the subject is a mammal. In some embodiments, the subject is a human. In some embodiments, the subject has been diagnosed with HBV infection. In some embodiments, the subject is diagnosed with chronic hepatitis B (CHB). In some embodiments, the genotype of the HBV infection is known. In some embodiments, the subject is infected with HBV genotype A (e.g., HBV-A1-7), HBV genotype B (e.g., HBV-B2-5), HBV genotype C (e.g., HBV-C1-16), HBV genotype D (e.g., HBV-D1-7), HBV genotype E, HBV genotype F (e.g., HBV-F1-4), HBV genotype G, HBV genotype H, HBV genotype I, or HBV genotype J. In some embodiments, a compound of Formula (I) or Formula (II) has pan-genotypic activity.

In some embodiments, in a method described herein, the subject is treatment naïve. In some embodiments, the subject has previously been treated for HBV infection. In some embodiments, the previous treatment for HBV infection has failed. In some embodiments, the subject has relapsed.

In some embodiments, the subject has been previously been treated with an anti-HBV agent other than a compound of Formula (I) or Formula (II) or a pharmaceutically acceptable salt thereof (e.g., an interferon, ribavirin) and is suffering from a relapsed HBV infection.

In some embodiments, the subject is suffering from a co-infection with Hepatitis B virus (HDV). In some embodiments, the subject has been diagnosed with an HBV infection. In some embodiments, the subject has been diagnosed with an HDV infection. In some embodiments, the subject has been diagnosed with a co-infection of HBV and HDV.

In some embodiments, in a method described herein, the subject has been diagnosed with cirrhosis of the liver. In some embodiments, the subject has been diagnosed with hepatocellular carcinoma. In some embodiments, the subject has been diagnosed with hepatocellular carcinoma and is awaiting liver transplantation. In some embodiments, the subject is non-cirrhotic or has not been diagnosed with hepatocellular carcinoma.

In some embodiments, in a method described herein, the subject has been further diagnosed with an HIV infection. In some embodiments, the strain of HIV infection is known. In some embodiments, the subject is infected with HIV-1 or HIV-2 (e.g., strain 1 or strain 2).

In some embodiments, the subject has been previously been treated with an anti-HBV agent other than a compound of Formula (I) or Formula (II) or a pharmaceutically acceptable salt thereof (e.g., an interferon, ribavirin) and is suffering from a relapsed HBV infection.

In some embodiments, in a method described herein, the subject is further administered an additional agent or treatment or a pharmaceutically acceptable salt thereof. In some embodiments, the additional agent is an interferon, a nucleoside analog, a non-nucleoside antiviral, a non-interferon immune enhancer, or a direct-acting antiviral. In some embodiments, the additional agent is an interferon, e.g., peg-interferon alfa (e.g., peg-interferon alfa-2a or peg-interferon alfa-2b). In some embodiments, the additional agent is a nucleoside analog, e.g., lamivudine, adefovir dipivoxil, entecavir, telbivudine, clevudine, ribavarin, tenofovir (e.g., tenofovir dipivoxil or tenofovir alafenamide), besifovir, or AGX-1009. In some embodiments, the non-nucleoside antiviral agent comprises NOV-225, BAM 205, Myrcludex B, ARC-520, BAY 41-4109, REP 9AC, Alinia (nitazoxanide), Dd-RNAi, NVR-121 (NVR 3-778), BSBI-25, NVP-018, TKM-HBV, or ALN-HBV. In some embodiments, the immune enhancer comprises zadaxin (thymosin alpha-1), GS-4774, CYT107 (interleukin-7), Dv-601, HBV core antigen vaccine, or GS-9620.

In any and all embodiments, the method features a pharmaceutical composition for use in treating a drug-resistant strain of the Hepatitis B virus in a subject, the composition comprising a compound of Formula (I) (e.g., Formula (Ia), Formula (Ib), or Formula (Ic)), a compound of Formula (II) (e.g., Formula (IIa), Formula (IIb), or Formula (IIc)), or a pharmaceutically acceptable salt thereof to thereby treat the subject.

In another aspect, the present invention features a method of treating a subject infected with the Hepatitis B virus (HBV) that has previously been administered an anti-HBV agent, the method comprising administering to the subject a compound of Formula (I), wherein the compound is selected from:

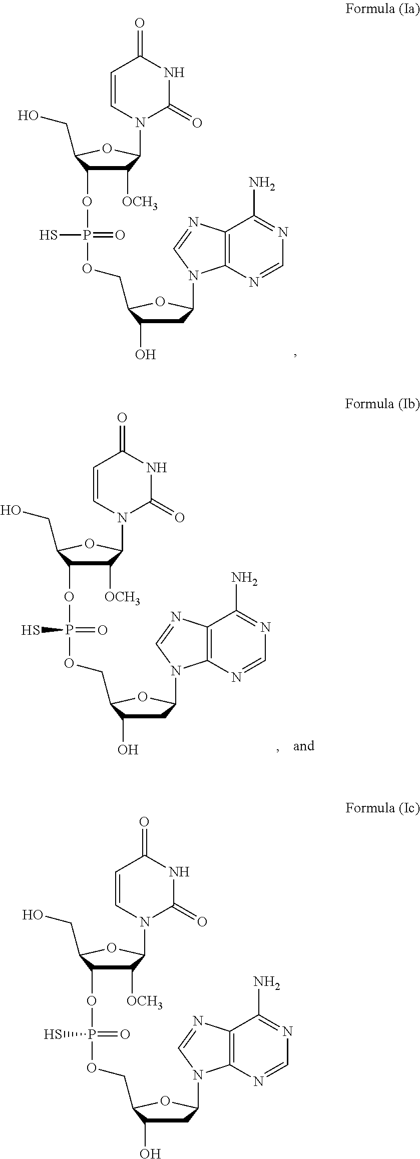

or a prodrug or pharmaceutically acceptable salt thereof to thereby treat the subject. In some embodiments, the prodrug of Formula (I) is a compound of Formula (II), wherein the compound is selected from:

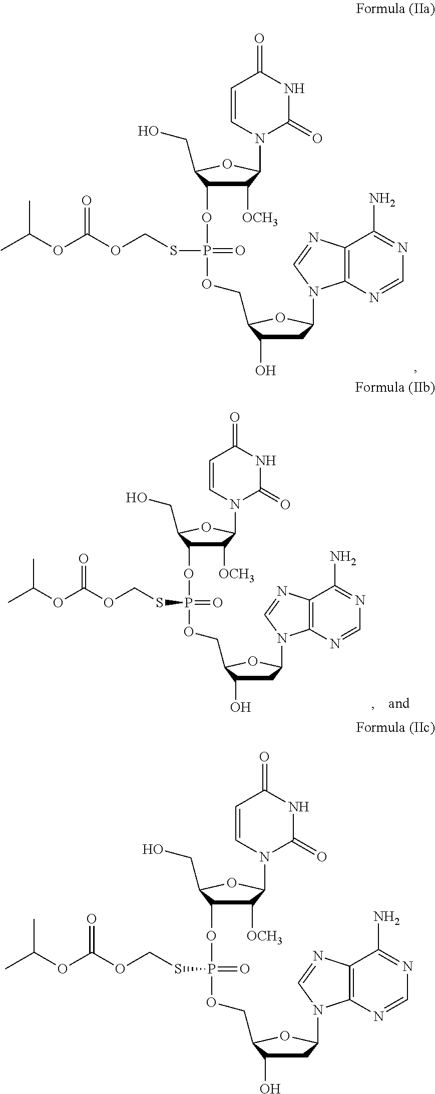

or a pharmaceutically acceptable salt thereof. In some embodiments, the method comprises administering to the subject a compound of Formula (I) or a pharmaceutically acceptable salt thereof. In some embodiments, the method comprises administering to the subject a compound of Formula (II) or a pharmaceutically acceptable salt thereof.

In some embodiments, the anti-HBV agent is an anti-HBV agent other than a compound of Formula (I) or Formula (II) or a pharmaceutically acceptable salt thereof. In some embodiments, the anti-HBV agent is an interferon, a nucleoside analog, a non-nucleoside antiviral, an immune enhancer, or a direct-acting antiviral agent. In some embodiments, the interferon comprises interferon alfa-2a, interferon alfa-2b, interferon alfa-n1, interferon alfacon-1, or a pegylated interferon (e.g., peginterferon alfa-2a, peginterferon alfa-2b). In some embodiments, the nucleoside analog comprises lamivudine, adefovir dipivoxil, entecavir, telbivudine, clevudine, ribavarin, tenofovir (e.g., tenofovir dipivoxil or tenofovir alafenamide), besifovir, or AGX-1009. In some embodiments, non-nucleoside antiviral agent comprises NOV-225, BAM 205, Myrcludex B, ARC-520, BAY 41-4109, REP 9AC, Alinia (nitazoxanide), Dd-RNAi, NVR-121 (NVR 3-778), BSBI-25, NVP-018, TKM-HBV, or ALN-HBV. In some embodiments, the immune enhancer comprises zadaxin (thymosin alpha-1), GS-4774, CYT107 (interleukin-7), Dv-601, HBV core antigen vaccine, or GS-9620.

In some embodiments, the HBV strain is a drug-resistant HBV strain. In some embodiments, the drug-resistant strain of HBV is resistant to an anti-HBV agent other than a compound of Formula (I) or Formula (II). In some embodiments, the drug-resistant strain of HBV is resistant to an anti-HBV agent other than a compound of Formula (I). In some embodiments, the drug-resistant strain of HBV is resistant to an anti-HBV agent other than a compound of Formula (II).

In some embodiments, the level of an HBV biomarker in the subject is not substantially reduced by exposure to an anti-HBV agent other than a compound of Formula (I) or Formula (II). In some embodiments, the level of an HBV biomarker is not substantially reduced by exposure to an anti-HBV agent other than a compound of Formula (I). In some embodiments, the level of an HBV biomarker is not substantially reduced by exposure to an anti-HBV agent other than a compound of Formula (II).

In some embodiments, the HBV biomarker comprises the viral load, HBsAg level, HBeAg level, or cccDNA level. In some embodiments, the viral load of the drug-resistant strain of HBV is not substantially reduced by exposure to an anti-HBV agent other than a compound of Formula (I) or Formula (II) or a pharmaceutically acceptable salt thereof. In some embodiments, the viral load of the drug-resistant strain of HBV is reduced by less than about 50%, about 40%, about 30%, about 20%, about 15%, about 10%, about 5%, about 2.5%, about 1%, about 0.5%, about 0.1%, or less upon exposure to an anti-HBV agent other than a compound of Formula (I) or Formula (II) or a pharmaceutically acceptable salt thereof. In some embodiments, the viral load of the drug-resistant strain of HBV is reduced by less than about 2 log units, about 1.5 log units, about 1 log unit, about 0.5 log units, about 0.1 log units, or less upon administration of an anti-HBV agent other than a compound of Formula (I) or Formula (II) or a pharmaceutically acceptable salt thereof.

In some embodiments, the viral load of the drug-resistant strain of HBV is substantially reduced by a compound of Formula (I) or Formula (II) or a pharmaceutically acceptable salt thereof. In some embodiments, the viral load of the drug-resistant strain of HBV is reduced by more than about 10%, about 20%, about 30%, about 40%, about 50%, about 60%, about 70%, about 80%, about 90%, about 95%, about 99%, about 99.9%, or about 99.99% or more upon administration of a compound of Formula (I) or Formula (II) or a pharmaceutically acceptable salt thereof. In some embodiments, the viral load of the drug-resistant strain of HBV is reduced by more than about 1 log unit, about 1.5 log units, about 2 log units, about 2.5 log units, about 3 log units, about 3.5 log units, about 4 log units, about 4.5 log units, about 5 log units, or more upon administration to a compound of Formula (I) or Formula (II) or a pharmaceutically acceptable salt thereof.

In some embodiments, the HBsAg level of the drug-resistant strain of HBV is not substantially reduced by exposure to an anti-HBV agent other than a compound of Formula (I) or Formula (II) or a pharmaceutically acceptable salt thereof. In some embodiments, the HBsAg level of the drug-resistant strain of HBV is reduced by less than about 50%, about 40%, about 30%, about 20%, about 15%, about 10%, about 5%, about 2.5%, about 1%, about 0.5%, about 0.1%, or less upon exposure to an anti-HBV agent other than a compound of Formula (I) or Formula (II) or a pharmaceutically acceptable salt thereof. In some embodiments, the HBsAg level of the drug-resistant strain of HBV is reduced by less than about 2 log units, about 1.5 log units, about 1 log unit, about 0.5 log units, about 0.1 log units, or less upon administration of an anti-HBV agent other than a compound of Formula (I) or Formula (II) or a pharmaceutically acceptable salt thereof.

In some embodiments, the HBsAg level of the drug-resistant strain of HBV is substantially reduced by a compound of Formula (I) or Formula (II) or a pharmaceutically acceptable salt thereof. In some embodiments, the HBsAg level of the drug-resistant strain of HBV is reduced by more than about 10%, about 20%, about 30%, about 40%, about 50%, about 60%, about 70%, about 80%, about 90%, about 95%, about 99%, about 99.9%, or about 99.99% or more upon administration of a compound of Formula (I) or Formula (II) or a pharmaceutically acceptable salt thereof. In some embodiments, the HBsAg level of the drug-resistant strain of HBV is reduced by more than about 1 log unit, about 1.5 log units, about 2 log units, about 2.5 log units, about 3 log units, about 3.5 log units, about 4 log units, about 4.5 log units, about 5 log units, or more upon administration to a compound of Formula (I) or Formula (II) or a pharmaceutically acceptable salt thereof.

In some embodiments, the HBeAg level of the drug-resistant strain of HBV is not substantially reduced by exposure to an anti-HBV agent other than a compound of Formula (I) or Formula (II) or a pharmaceutically acceptable salt thereof. In some embodiments, the HBeAg level of the drug-resistant strain of HBV is reduced by less than about 50%, about 40%, about 30%, about 20%, about 15%, about 10%, about 5%, about 2.5%, about 1%, about 0.5%, about 0.1%, or less upon exposure to an anti-HBV agent other than a compound of Formula (I) or Formula (II) or a pharmaceutically acceptable salt thereof. In some embodiments, the HBeAg level of the drug-resistant strain of HBV is reduced by less than about 2 log units, about 1.5 log units, about 1 log unit, about 0.5 log units, about 0.1 log units, or less upon administration of an anti-HBV agent other than a compound of Formula (I) or Formula (II) or a pharmaceutically acceptable salt thereof.

In some embodiments, the HBeAg level of the drug-resistant strain of HBV is substantially reduced by a compound of Formula (I) or Formula (II) or a pharmaceutically acceptable salt thereof. In some embodiments, the HBeAg level of the drug-resistant strain of HBV is reduced by more than about 10%, about 20%, about 30%, about 40%, about 50%, about 60%, about 70%, about 80%, about 90%, about 95%, about 99%, about 99.9%, or about 99.99% or more upon administration of a compound of Formula (I) or Formula (II) or a pharmaceutically acceptable salt thereof. In some embodiments, the HBeAg level of the drug-resistant strain of HBV is reduced by more than about 1 log unit, about 1.5 log units, about 2 log units, about 2.5 log units, about 3 log units, about 3.5 log units, about 4 log units, about 4.5 log units, about 5 log units, or more upon administration to a compound of Formula (I) or Formula (II) or a pharmaceutically acceptable salt thereof.

In some embodiments, the cccDNA level of the drug-resistant strain of HBV is not substantially reduced by exposure to an anti-HBV agent other than a compound of Formula (I) or Formula (II) or a pharmaceutically acceptable salt thereof. In some embodiments, the cccDNA level of the drug-resistant strain of HBV is reduced by less than about 50%, about 40%, about 30%, about 20%, about 15%, about 10%, about 5%, about 2.5%, about 1%, about 0.5%, about 0.1%, or less upon exposure to an anti-HBV agent other than a compound of Formula (I) or Formula (II) or a pharmaceutically acceptable salt thereof. In some embodiments, the cccDNA level of the drug-resistant strain of HBV is reduced by less than about 2 log units, about 1.5 log units, about 1 log unit, about 0.5 log units, about 0.1 log units, or less upon administration of an anti-HBV agent other than a compound of Formula (I) or Formula (II) or a pharmaceutically acceptable salt thereof.

In some embodiments, the cccDNA level of the drug-resistant strain of HBV is substantially reduced by a compound of Formula (I) or Formula (II) or a pharmaceutically acceptable salt thereof. In some embodiments, the cccDNA level of the drug-resistant strain of HBV is reduced by more than about 10%, about 20%, about 30%, about 40%, about 50%, about 60%, about 70%, about 80%, about 90%, about 95%, about 99%, about 99.9%, or about 99.99% or more upon administration of a compound of Formula (I) or Formula (II) or a pharmaceutically acceptable salt thereof. In some embodiments, the cccDNA level of the drug-resistant strain of HBV is reduced by more than about 1 log unit, about 1.5 log units, about 2 log units, about 2.5 log units, about 3 log units, about 3.5 log units, about 4 log units, about 4.5 log units, about 5 log units, or more upon administration to a compound of Formula (I) or Formula (II) or a pharmaceutically acceptable salt thereof.

In some embodiments, the drug-resistant strain of HBV is resistant to an anti-HBV agent other than a compound of Formula (I) or Formula (II) or a pharmaceutically acceptable salt thereof, and the anti-HBV agent is an interferon, a nucleoside analog, a non-nucleoside antiviral, an immune enhancer, or a direct-acting antiviral agent. In some embodiments, the interferon comprises interferon alfa-2a, interferon alfa-2b, interferon alfa-n1, interferon alfacon-1, or a pegylated interferon (e.g., peginterferon alfa-2a, peginterferon alfa-2b). In some embodiments, the nucleoside analog comprises lamivudine, adefovir dipivoxil, entecavir, telbivudine, clevudine, ribavarin, tenofovir (e.g., tenofovir dipivoxil or tenofovir alafenamide), besifovir, or AGX-1009. In some embodiments, non-nucleoside antiviral agent comprises NOV-225, BAM 205, Myrcludex B, ARC-520, BAY 41-4109, REP 9AC, Alinia (nitazoxanide), Dd-RNAi, NVR-121 (NVR 3-778), BSBI-25, NVP-018, TKM-HBV, or ALN-HBV. In some embodiments, the immune enhancer comprises zadaxin (thymosin alpha-1), GS-4774, CYT107 (interleukin-7), Dv-601, HBV core antigen vaccine, or GS-9620.

In some embodiments, the drug-resistant HBV strain is an HBV variant strain or HBV mutant strain. In some embodiments, the drug-resistant HBV strain comprises a variant or mutant form of the HBsAg, HBcAg, HBeAg, L, M, P, or X proteins. In some embodiments, the drug-resistant HBV variant comprises an amino acid mutation (e.g., an amino acid substitution, addition, or deletion) in the sequence of the HBsAg, HBcAg, HBeAg, L, M, P, or X proteins, e.g., as compared with a reference sequence.

In some embodiments, the drug-resistant HBV variant comprises an amino acid mutation (e.g., an amino acid substitution, addition, or deletion) in the sequence of the HBsAg protein, e.g., as compared with a reference sequence. In some embodiments, the amino acid mutation (e.g., an amino acid substitution, addition, or deletion) in the HBsAg protein sequence comprises a mutation located between amino acid position 100 and amino acid position 200, e.g., as compared with a reference sequence. In some embodiments, the amino acid mutation (e.g., an amino acid substitution, addition, or deletion) in the HBsAg protein sequence comprises a mutation at amino acid positions 115, 118, 120, 123, 126, 129, 131, 133, 134, 142, 143, 144, 145, or 154, e.g., as compared with a reference sequence. In some embodiments, the amino acid mutation (e.g., an amino acid substitution, addition, or deletion) in the HBsAg protein sequence comprises a T115N, T118V, P120L, P120Q, T126S, Q129H, T131K, M133I, M133L, F134N, F134H, P142L, P142S, T143L, D144A, D144V, G145R, or S154P mutation.

In some embodiments, the amino acid mutation (e.g., an amino acid substitution, addition, or deletion) in the HBsAg protein sequence comprises a mutation located between amino acid position 150 and amino acid position 200, e.g., as compared with a reference sequence. In some embodiments, the amino acid mutation (e.g., an amino acid substitution, addition, or deletion) in the HBsAg protein sequence comprises a mutation at amino acid positions 161, 172, 173, 175, 176, 193, 194, or 196, e.g., as compared with a reference sequence. In some embodiments, the amino acid mutation (e.g., an amino acid substitution, addition, or deletion) in the HBsAg protein sequence comprises a F161H, F161L, W172L, W172*, L173F, L175F, L176V, L176*, S193L, V194F, V194S, I195M, W196L, W196S, or W196* mutation, e.g., as compared to a reference or consensus sequence, wherein “*” represents a stop codon.

In some embodiments, the drug-resistant HBV variant comprises an amino acid mutation (e.g., an amino acid substitution, addition, or deletion) in the sequence of the P protein, e.g., as compared with a reference sequence. In some embodiments, the amino acid mutation (e.g., an amino acid substitution, addition, or deletion) in the P protein sequence comprises a mutation located between amino acid position 60 and amino acid position 275, e.g., as compared with a reference sequence. In some embodiments, the amino acid mutation (e.g., an amino acid substitution, addition, or deletion) in the P protein sequence comprises a mutation at amino acid positions 80, 169, 173, 180, 181, 184, 169, 202, 204, 215, 233, 236, or 250, e.g., as compared with a reference sequence. In some embodiments, the amino acid mutation (e.g., an amino acid substitution, addition, or deletion) in the P protein sequence comprises a N169T, I169T, V173L, L180M, A181T, A181V, T184A, T184C, T184G, T184I, T184L, T184M, T184S, S202C, S202G, S202I, M204I, M204V, N236T, M250I, or M250V mutation. In some embodiments, the amino acid mutation (e.g., an amino acid substitution, addition, or deletion) in the P protein sequence comprises a L180M, M204I, M204V, or N236T mutation.

In some embodiments, the method described herein comprises administering to the subject a mixture of compounds of Formula (I) or pharmaceutically acceptable salts thereof. In some embodiments, the method described herein comprises administering to the subject a mixture of compounds of Formula (Ib) and Formula (Ic) or pharmaceutically acceptable salts thereof. In some embodiments, the mixture comprises a ratio of Formula (Ib) to Formula (Ic) of about 1:1 (e.g., a racemic mixture). In some embodiments, the mixture comprises a ratio of Formula (Ib) to Formula (Ic) of about 51:49, about 52:48, about 53:47, about 54:46, about 55:45, about 60:40, about 65:35, about 70:30, about 75:25, about 80:20, about 85:15, about 90:10, about 95:5, or about 99:1. In some embodiments, the mixture comprises a ratio of Formula (Ic) to Formula (Ib) of about 51:49, about 52:48, about 53:47, about 54:46, about 55:45, about 60:40, about 65:35, about 70:30, about 75:25, about 80:20, about 85:15, about 90:10, about 95:5, or about 99:1.

In some embodiments, the method described herein comprises administering to the subject a mixture of compounds of Formula (I) comprising Formula (Ib) and less than about 5% of Formula (Ic), e.g., less than about 4%, less than about 3%, less than about 2%, less than about 1%, less than about 0.5%, or less than about 0.1% of Formula (Ic). In some embodiments, the method described herein comprises administering to the subject a compound of Formula (I) comprising Formula (Ib) or a pharmaceutically acceptable salt thereof that is substantially free of Formula (Ic). In some embodiments, the method described herein comprises administering to the subject a mixture of compounds of Formula (I) comprising Formula (Ic) and less than about 5% of Formula (Ib), e.g., less than about 4%, less than about 3%, less than about 2%, less than about 1%, less than about 0.5%, or less than about 0.1% of Formula (Ib). In some embodiments, the method described herein comprises administering to the subject a compound of Formula (I) comprising Formula (Ic) or a pharmaceutically acceptable salt thereof that is substantially free of Formula (Ib).

In some embodiments, the method described herein comprises administering to the subject a mixture of compounds of Formula (II) or pharmaceutically acceptable salts thereof. In some embodiments, the method described herein comprises administering to the subject a mixture of Formula (IIb) and Formula (IIc) or pharmaceutically acceptable salts thereof. In some embodiments, the mixture comprises a ratio of Formula (IIb) to Formula (IIc) of about 1:1 (e.g., a racemic mixture). In some embodiments, the mixture comprises a ratio of Formula (IIb) to Formula (IIc) of about 51:49, about 52:48, about 53:47, about 54:46, about 55:45, about 60:40, about 65:35, about 70:30, about 75:25, about 80:20, about 85:15, about 90:10, about 95:5, or about 99:1. In some embodiments, the mixture comprises a ratio of Formula (IIc) to Formula (IIb) of about 51:49, about 52:48, about 53:47, about 54:46, about 55:45, about 60:40, about 65:35, about 70:30, about 75:2, about 80:20, about 85:15, about 90:10, about 95:5, or about 99:1.

In some embodiments, the method described herein comprises administering to the subject a mixture of compounds of Formula (II) comprising Formula (IIb) and less than about 5% of Formula (IIc), e.g., less than about 4%, less than about 3%, less than about 2%, less than about 1%, less than about 0.5%, or less than about 0.1% of Formula (IIc). In some embodiments, the method described herein comprises administering to the subject a compound of Formula (II) comprising Formula (IIb) or a pharmaceutically acceptable salt thereof that is substantially free of Formula (IIc). In some embodiments, the method described herein comprises administering to the subject a mixture of compounds of Formula (II) comprising Formula (IIc) and less than about 5% of Formula (IIb), e.g., less than about 4%, less than about 3%, less than about 2%, less than about 1%, less than about 0.5%, or less than about 0.1% of Formula (IIb). In some embodiments, the method described herein comprises administering to the subject a compound of Formula (II) comprising Formula (IIc) or a pharmaceutically acceptable salt thereof that is substantially free of Formula (IIb).

In some embodiments, in a method described herein, the IC₅₀ value of a compound of Formula (I) or Formula (II) is less than 10 μM (e.g., a compound of Formula (II) is less than 10 μM). In some embodiments, the IC₅₀ value of a compound of Formula (I) or Formula (II) is less than 1 μM (e.g., a compound of Formula (II) is less than 1 μM). In some embodiments, the IC₅₀ value of a compound of Formula (I) or Formula (II) is less than 0.1 μM (e.g., the IC₅₀ value of a compound of Formula (II) is less than 0.1 μM). In some embodiments, the IC₅₀ value of a compound of Formula (I) or Formula (II) is less than 0.01 μM (e.g., the IC₅₀ value of a compound of Formula (II) is less than 0.1 μM).

In some embodiments, in a method described herein, the compound of Formula (I) or Formula (II) is administered orally. In some embodiments, the compound of Formula (I) is administered orally. In some embodiments, the compound of Formula (II) is administered orally. In some embodiments, the compound of Formula (I) or Formula (II) is administered parenterally. In some embodiments, the compound of Formula (I) is administered parenterally. In some embodiments, the compound of Formula (II) is administered parenterally. In some embodiments, the compound of Formula (I) or Formula (II) is administered intravenously. In some embodiments, the compound of Formula (I) is administered intravenously. In some embodiments, the compound of Formula (II) is administered intravenously.

In some embodiments, the compound of Formula (I) or Formula (II) is formulated a liquid or solid dosage form. In some embodiments, the liquid dosage form comprises a suspension, a solution, a linctus, an emulsion, a drink, an elixir, or a syrup. In some embodiments, the solid dosage form comprises a capsule, tablet, pill, dragée, powder, or microencapsulated dose form.

In some embodiments, the compound (e.g., a compound of Formula (I) or Formula (II)) is administered at a dosage between about 0.5 mg/kg and about 1000 mg/kg. In some embodiments, the compound (e.g., a compound of Formula (I) or Formula (II)) is administered at a dosage between about 0.5 mg/kg and about 1000 mg/kg, about 900 mg/kg, about 800 mg/kg, about 700 mg/kg, about 600 mg/kg, about 500 mg/kg, about 400 mg/kg, about 300 mg/kg, about 250 mg/kg about 200 mg/kg, about 150 mg/kg, about 100 mg/kg, about 75 mg/kg, about 50 mg/kg, about 25 mg/kg, about 10 mg/kg, about 5 mg/kg, about 2.5 mg/kg, or less. In some embodiments, the compound (e.g., a compound of Formula (I) or Formula (II)) is administered at a dosage between about 5 mg/kg and about 500 mg/kg. In some embodiments, the compound (e.g., a compound of Formula (I) or Formula (II)) is administered at a dosage between about 5 mg/kg and about 500 mg/kg, 450 mg/kg, about 400 mg/kg, about 350 mg/kg about 300 mg/kg, about 250 mg/kg, about 200 mg/kg, about 150 mg/kg, about 100 mg/kg, about 75 mg/kg, about 50 mg/kg, about 25 mg/kg, about 10 mg/kg, or less.

In some embodiments, the dosage of Formula (I) or Formula (II) is between about 10 mg and about 1500 mg, about 1250 mg, about 1000 mg, about 900 mg, about 800 mg, about 700 mg, about 600 mg, about 500 mg, about 400 mg, about 300 mg, about 250 mg, about 200 mg, about 150 mg, about 100 mg, about 75 mg, about 50 mg, about 25 mg, or less. In some embodiments, the dosage of Formula (I) or Formula (II) is between about 10 mg, about 25 mg, about 50 mg, about 75 mg, about 100 mg, about 150 mg, about 200 mg, about 250 mg, about 300 mg, about 400 mg, about 500 mg, about 600 mg, about 700 mg, about 800 mg, about 900 mg, about 1000 mg, about 1250 mg, and about 1500 mg. In some embodiments, the dosage of Formula (I) or Formula (II) is between about 50 mg and about 1000 mg. In some embodiments, the dosage of Formula (I) or Formula (II) is between about 200 mg and about 1000 mg.

In some embodiments, in a method described herein, the compound of Formula (I) or Formula (II) is administered daily. In some embodiments, the compound of Formula (I) or Formula (II) is administered once daily. In some embodiments, the compound of Formula (I) or Formula (II) is administered more than once a day, e.g., twice a day, three times a day, four times a day. In some embodiments, the compound of Formula (I) or Formula (II) is administered every other day, every 2 days, every 3 days, every 4 days, or more. In some embodiments, thecompound of Formula (I) or Formula (II) is administered once a week, twice a week, three times a week, four times a week, five times a week, or six times a week.

In some embodiments, in a method described herein, the duration of the method is one day. In some embodiments, the duration of the method is greater than 1 day, e.g., about 2 days, about 3 days, about 4 days, about 5 days, about 6 days, about 7 days, about 8 days, about 9 days, about 10 days, about 11 days, about 12 days, about 13 days, about 14 days, about 2 weeks, about 3 weeks, about 4 weeks, about 1 month, about 1.5 months, about 2 months, about 3 months, about 4 months, about 5 months, about 6 months. In some embodiments, the duration of the method is between about 1 day and about 2 weeks. In some embodiments, the duration of the method is between 6 days and 14 days. In some embodiments, the duration of the method is for one week. In some embodiments, the duration of the method lasts until the subject is cured of HBV infection (e.g., until the subject presents an undetectable level of HBV RNA).

In some embodiments, in a method described herein, a compound of Formula (I) or Formula (II) is formulated as a pharmaceutical composition. In some embodiments, the pharmaceutical composition further comprises a pharmaceutically acceptable carrier or excipient.

In some embodiments, in a method described herein, the subject is a mammal. In some embodiments, the subject is a human. In some embodiments, the subject has been diagnosed with HBV infection. In some embodiments, the subject is diagnosed with chronic hepatitis B (CHB). In some embodiments, the genotype of the HBV infection is known. In some embodiments, the subject is infected with HBV genotype A (e.g., HBV-A1-7), HBV genotype B (e.g., HBV-B2-5), HBV genotype C (e.g., HBV-C1-16), HBV genotype D (e.g., HBV-D1-7), HBV genotype E, HBV genotype F (e.g., HBV-F1-4), HBV genotype G, HBV genotype H, HBV genotype I, or HBV genotype J. In some embodiments, a compound of Formula (I) or Formula (II) has pan-genotypic activity.

In some embodiments, in a method described herein, the subject is treatment naïve. In some embodiments, the subject has previously been treated for HBV infection. In some embodiments, the previous treatment for HBV infection has failed. In some embodiments, the subject has relapsed.

In some embodiments, the subject has been previously been treated with an anti-HBV agent other than a compound of Formula (I) or Formula (II) or a pharmaceutically acceptable salt thereof (e.g., an interferon, ribavirin) and is suffering from a relapsed HBV infection.

In some embodiments, the subject is suffering from a co-infection with Hepatitis B virus (HDV). In some embodiments, the subject has been diagnosed with an HBV infection. In some embodiments, the subject has been diagnosed with an HDV infection. In some embodiments, the subject has been diagnosed with a co-infection of HBV and HDV.

In some embodiments, in a method described herein, the subject has been diagnosed with cirrhosis of the liver. In some embodiments, the subject has been diagnosed with hepatocellular carcinoma. In some embodiments, the subject has been diagnosed with hepatocellular carcinoma and is awaiting liver transplantation. In some embodiments, the subject is non-cirrhotic or has not been diagnosed with hepatocellular carcinoma.

In some embodiments, in a method described herein, the subject has been further diagnosed with an HIV infection. In some embodiments, the strain of HIV infection is known. In some embodiments, the subject is infected with HIV-1 or HIV-2 (e.g., strain 1 or strain 2).

In some embodiments, in a method described herein, the subject is further administered an additional agent or treatment or a pharmaceutically acceptable salt thereof. In some embodiments, the additional agent is an interferon, a nucleoside analog, a non-nucleoside antiviral, a non-interferon immune enhancer, or a direct-acting antiviral. In some embodiments, the additional agent is an interferon, e.g., peg-interferon alfa (e.g., peg-interferon alfa-2a or peg-interferon alfa-2b). In some embodiments, the additional agent is a nucleoside analog, e.g., lamivudine, adefovir dipivoxil, entecavir, telbivudine, clevudine, ribavarin, tenofovir (e.g., tenofovir dipivoxil or tenofovir alafenamide), besifovir, or AGX-1009. In some embodiments, the non-nucleoside antiviral agent comprises NOV-225, BAM 205, Myrcludex B, ARC-520, BAY 41-4109, REP 9AC, Alinia (nitazoxanide), Dd-RNAi, NVR-121 (NVR 3-778), BSBI-25, NVP-018, TKM-HBV, or ALN-HBV. In some embodiments, the immune enhancer comprises zadaxin (thymosin alpha-1), GS-4774, CYT107 (interleukin-7), Dv-601, HBV core antigen vaccine, or GS-9620. In some embodiments, the additional agent is entecavir or tenofovir (e.g., tenofovir dipivoxil or tenofovir alafenamide).

In any and all embodiments, the method features a pharmaceutical composition for use in treating a subject infected with the Hepatitis B virus (HBV) that has previously been administered an anti-HBV agent, the composition comprising a compound of Formula (I) (e.g., Formula (Ia), Formula (Ib), or Formula (Ic)), a compound of Formula (II) (e.g., Formula (IIa), Formula (IIb), or Formula (IIc)), or a pharmaceutically acceptable salt thereof to thereby treat the subject.

In another aspect, the present invention features a method of treating Hepatitis D virus in a subject, the method comprising administering to the subject a compound of Formula (I), wherein the compound is selected from:

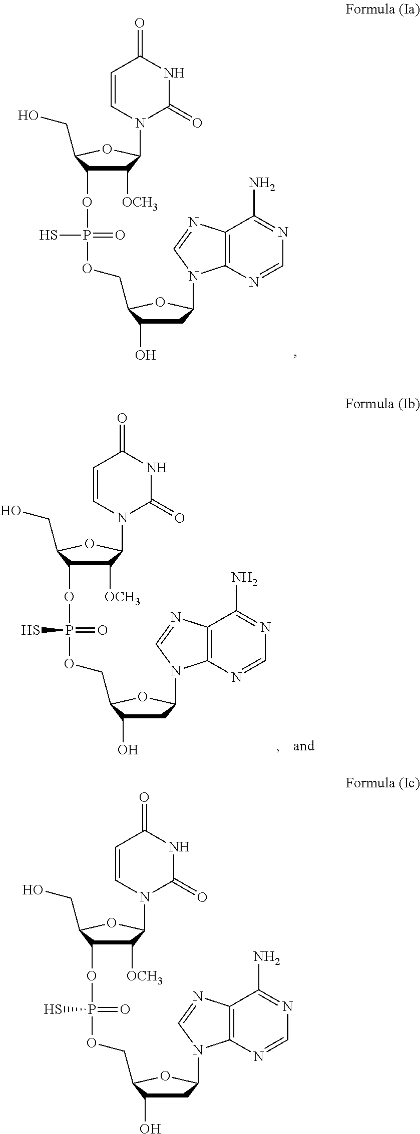

or a prodrug or pharmaceutically acceptable salt thereof in combination with entecavir or a pharmaceutically acceptable salt thereof to thereby treat the subject.

In another aspect, the present invention features a method of treating Hepatitis D virus in a subject comprising administering to the subject a course of entecavir or a pharmaceutically acceptable salt thereof, wherein the subject has previously been treated with a course of compound of Formula (I), wherein the compound is selected from:

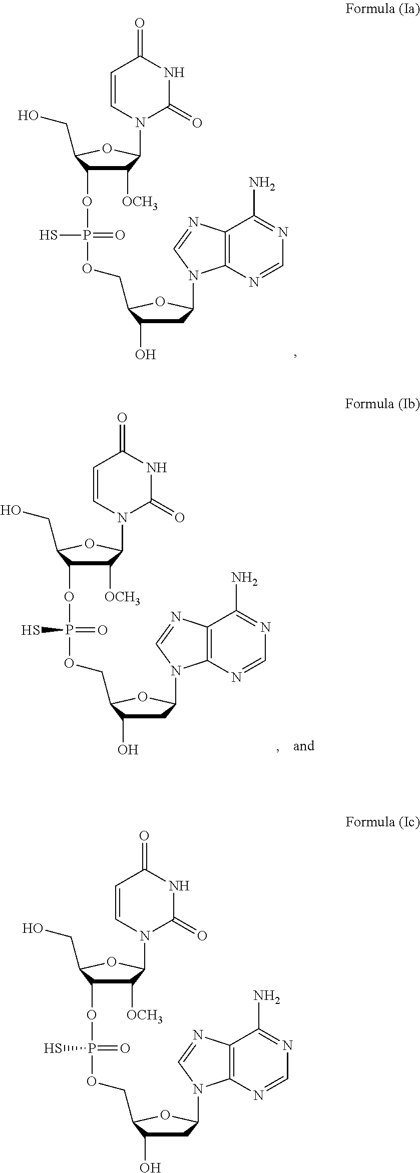

or a prodrug or pharmaceutically acceptable salt thereof to thereby treat the subject.

In another aspect, the present invention features a method of treating Hepatitis D virus in a subject, wherein the subject has previously been treated with a course of entecavir or a pharmaceutically acceptable salt thereof, the method comprising administering to the subject a course of compound of Formula (I), wherein the compound is selected from:

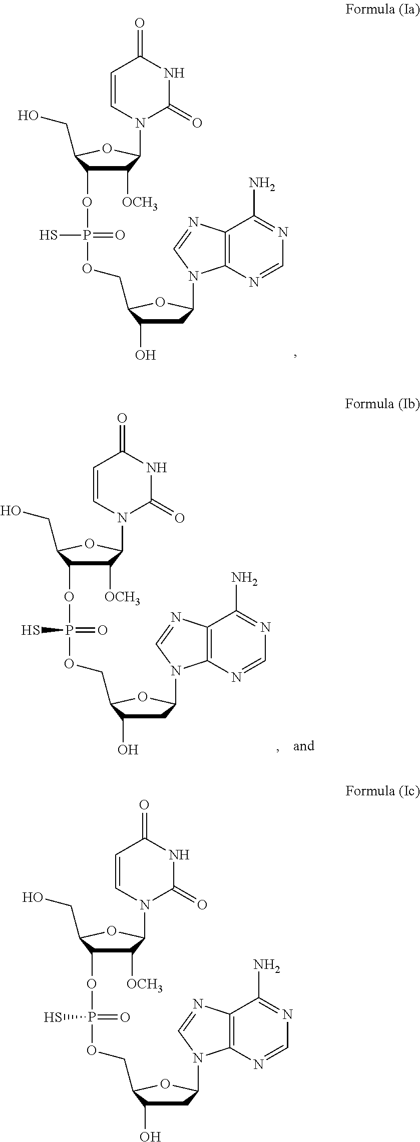

or a prodrug or pharmaceutically acceptable salt thereof to thereby treat subject.

In another aspect, the present invention features a method of treating Hepatitis D virus in a subject, the method comprising first administering a course of entecavir or a pharmaceutically acceptable salt thereof to the subject, and subsequently administering to the subject a course of a compound of Formula (I), wherein the compound is selected from:

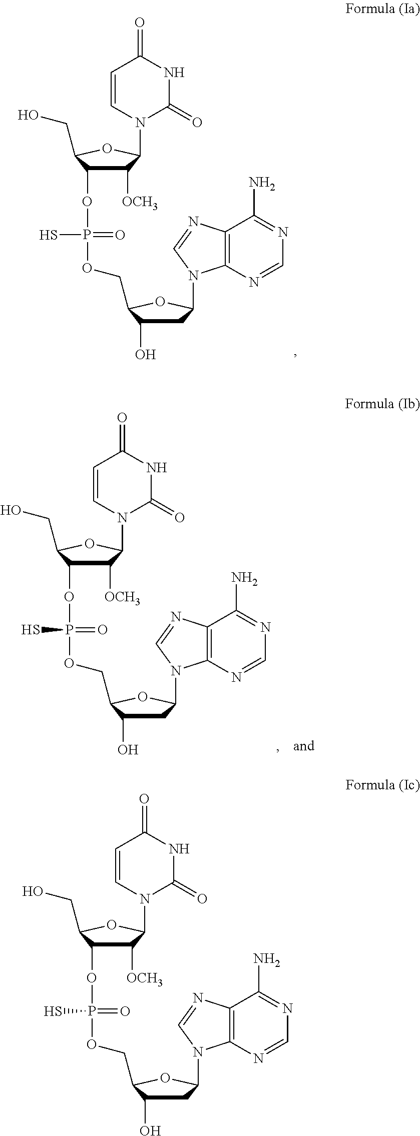

or a prodrug or pharmaceutically acceptable salt thereof to thereby treat the subject.

In another aspect, the present invention features a method of treating Hepatitis D virus in a subject, the method comprising first administering to the subject a course of a compound of Formula (I), wherein the compound is selected from:

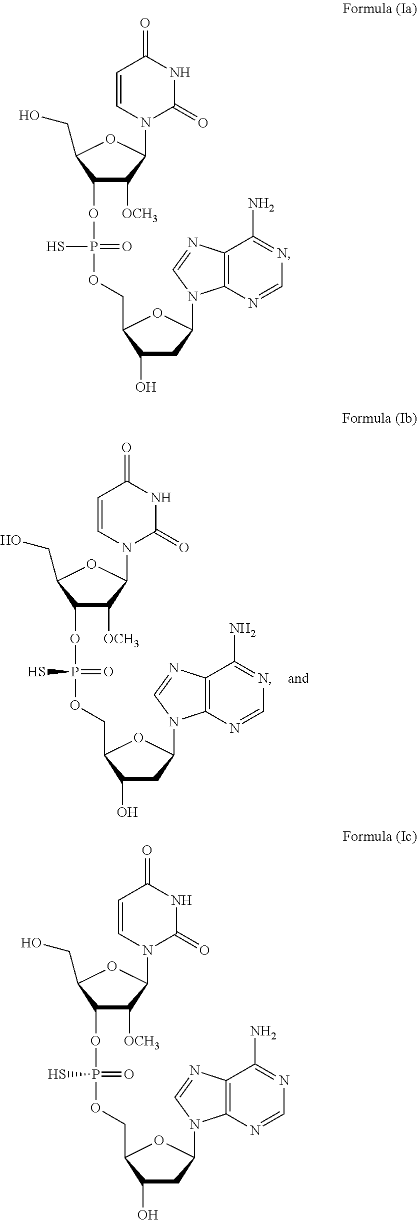

or a prodrug or pharmaceutically acceptable salt thereof, and subsequently administering to the subject a course of entecavir or a pharmaceutically acceptable salt thereof to thereby treat the subject.

In any or all of the preceding embodiments, the prodrug of Formula (I) is a compound of Formula (II), wherein the compound is selected from:

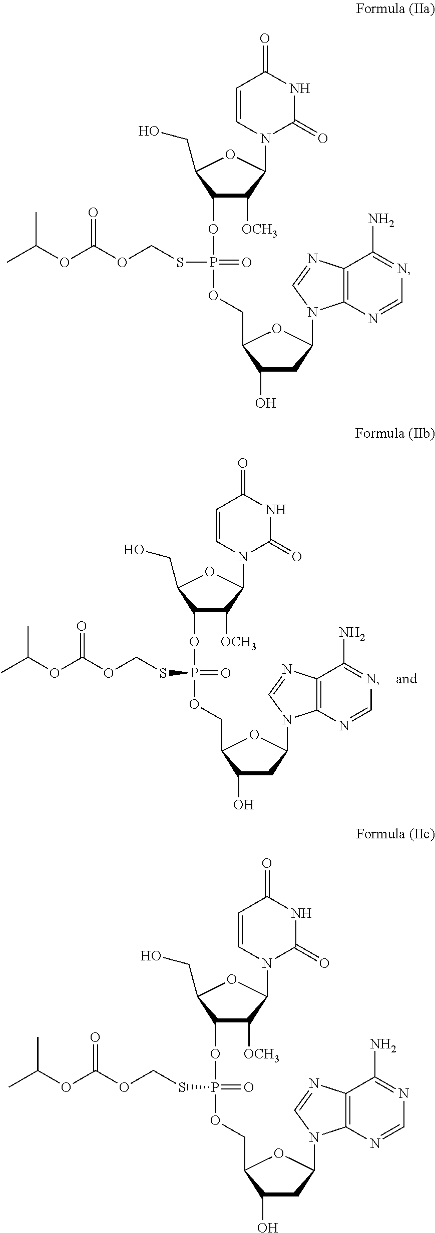

or a pharmaceutically acceptable salt thereof.

In some embodiments, a course of a compound of Formula (I) or Formula (II) is between about 1 day to about 24 weeks. In some embodiments, the compound of Formula (I) or Formula (II) is administered at least weekly (e.g., once a week, twice a week, three times a week, four times a week, five times a week, six times a week, 7 times a week) throughout a course of treatment. In some embodiments, the compound of Formula (I) or Formula (II) is administered daily throughout a course of treatment.

In some embodiments, the course of entecavir is between about 1 day to about 12 weeks. In some embodiments, entecavir is administered at least weekly (e.g., once a week, twice a week, three times a week, four times a week, five times a week, six times a week, 7 times a week) throughout a course of treatment. In some embodiments, entecavir is administered daily throughout a course of treatment.

In some embodiments, the dosage of the compound of Formula (I) or Formula (II) is between about 5 mg/kg to about 100 mg/kg (e.g., about 5 mg/kg, about 10 mg/kg, about 15 mg/kg, about 20 mg/kg, about 30 mg/kg, about 40 mg/kg, about 50 mg/kg, about 60 mg/kg, about 70 mg/kg, about 80 mg/kg, about 90 mg/kg, or about 100 mg/kg). In some embodiments, the dosage of the compound of Formula (I) or Formula (II) is between about 10 mg/kg to about 50 mg/kg (e.g., about 10 mg/kg, about 15 mg/kg, about 20 mg/kg, about 25 mg/kg, about 30 mg/kg, about 35 mg/kg, about 40 mg/kg, about 45 mg/kg, or about 50 mg/kg).

In some embodiments, the dosage of entecavir is between about 0.1 mg to about 5 mg (e.g., about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.25 mg, about 1.5 mg, about 1.75 mg, about 2 mg, about 2.5 mg, about 3 mg, about 3.5 mg, about 4 mg, about 4.5 mg, or about 5 mg). In some embodiments, the dosage of entecavir is between about 0.01 mg/kg to about 10 mg/kg (e.g., about 0.01 mg/kg, about 0.025 mg/kg, about 0.05 mg/kg, about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg). In some embodiments, the dosage of entecavir is between about 0.1 mg/kg to about 5 mg/kg (e.g., about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.25 mg/kg, about 1.5 mg/kg, about 1.75 mg/kg, about 2 mg/kg, about 2.5 mg/kg, about 3 mg/kg, about 3.5 mg/kg, about 4 mg/kg, about 4.5 mg/kg, or about 5 mg/kg).

In some embodiments, the dosage comprises a liquid or a solid dosage form. In some embodiments, the liquid dosage form comprises a suspension, a solution, a linctus, an emulsion, a drink, an elixir, or a syrup. In some embodiments, the solid dosage form comprises a capsule, tablet, dragée, or powder.

In some embodiments, the compound of Formula (I) or Formula (II) or entecavir is administered orally (e.g., the compound of Formula (I) or Formula (II) is administered orally, or entecavir is administered orally, or both the compound of Formula (I) or Formula (II) and entecavir are administered orally). In some embodiments, the compound of Formula (I) or Formula (II) or entecavir is administered parenterally (e.g., the compound of Formula (II) is administered parenterally). In some embodiments, the compound of Formula (I) or Formula (II) is administered parenterally and entecavir is administered orally. In some embodiments, compound of Formula (I) or Formula (II) is formulated as a fixed dose combination with entecavir (e.g., as a liquid dosage form or solid dosage form, e.g., a capsule or tablet). In some embodiments, compound of Formula (I) or Formula (II) is formulated as a fixed dose combination with entecavir (e.g., as a liquid dosage form or solid dosage form, e.g., a capsule or tablet) for oral administration.

In some embodiments, the administration of a compound of Formula (I) or Formula (II) and entecavir has a synergistic or additive effect. In some embodiments, the administration of a compound of Formula (I) or Formula (II) and entecavir has an additive effect. In some embodiments, the administration of a compound of Formula (I) or Formula (II) and entecavir has a synergistic effect.

In some embodiments, the composition comprises a mixture of compounds of Formula (I), e.g., Formula (Ib) and Formula (Ic). In some embodiments, the composition comprises Formula (Ib) and comprises less than about 5% of Formula (Ic) (e.g., less than about 4%, less than about 3%, less than about 2%, less than about1%, less than about 0.5%, or less than about 0.1% of Formula (Ic)), or is substantially free of Formula (Ic). In some embodiments, the composition comprises Formula (Ic) and comprises less than about 5% of Formula (Ib) (e.g., less than about 4%, less than about 3%, less than about 2%, less than about 1%, less than about 0.5%, or less than about 0.1% of Formula (Ib), or is substantially free of Formula (Ib)).

In some embodiments, the composition comprises a mixture of compounds of Formula (II), e.g., Formula (IIb) and Formula (IIc). In some embodiments, the composition comprises Formula (IIb) and comprises less than about 5% of Formula (IIc) (e.g., less than about 4%, less than about 3%, less than about 2%, less than about 1%, less than about 0.5%, or less than about 0.1% of Formula (IIc), or is substantially free of Formula (IIc)). In some embodiments, the composition comprises Formula (IIc) and comprises less than about 5% of Formula (IIb) (e.g., less than about 4%, less than about 3%, less than about 2%, less than about 1%, less than about 0.5%, or less than about 0.1% of Formula (IIb), or is substantially free of Formula (IIb)).

In some embodiments, the subject is a mammal. In some embodiments, the subject is a human. In some embodiments the subject is a non-human animal, e.g., a woodchuck (e.g., Eastern woodchuck).

In some embodiments, the method further comprises the administration of a therapeutically effective amount of an additional agent. In some embodiments, the additional agent is an antiviral agent or an anticancer agent. In some embodiments, the antiviral agent comprises an interferon, a nucleoside analog, a non-nucleoside antiviral, or a non-interferon immune enhancer. In some embodiments, the interferon comprises interferon alfa-2a, interferon alfa-2b, interferon alfa-n1, interferon alfacon-1, or a pegylated interferon (e.g., peginterferon alfa-2a, peginterferon alfa-2b). In some embodiments, the nucleoside analog comprises lamivudine, adefovir dipivoxil, telbivudine, clevudine, ribavarin, tenofovir, tenofovir dipivoxil, tenofovir alafenamide, besifovir, or AGX-1009. In some embodiments, the antiviral agent is tenofovir (e.g., tenofovir dipivoxil, tenofovir alafenamide). In some embodiments, the antiviral compound comprises NOV-225, BAM 205, Myrcludex B, ARC-520, BAY 41-4109, REP 9AC, Alinia (nitazoxanide), Dd-RNAi, NVR-121 (NVR 3-778), BSBI-25, NVP-018, TKM-HBV, or ALN-HBV. In some embodiments, the non-interferon immune enhancer comprises zadaxin (thymosin alpha-1), GS-4774, CYT107 (interleukin-7), Dv-601, HBV core antigen vaccine, or GS-9620. In some embodiments, the antiviral agent is a capsid inhibitor, an entry inhibitor, a secretion inhibitor, a microRNA, an antisense RNA agent, an RNAi agent, or other agent designed to inhibit viral RNA. In some embodiments, the anticancer agent is selected from methotrexate, 5-fluorouracil, doxorubicin, vincristine, bleomycin, vinblastine, dacarbazine, toposide, cisplatin, epirubicin, and sorafenib tosylate.

In some embodiments, in a method described herein, the subject is treatment naïve. In some embodiments, the subject has previously been treated for HBV infection. In some embodiments, the previous treatment for HBV infection has failed. In some embodiments, the subject has relapsed.

In some embodiments, the subject has been previously been treated with an anti-HBV agent other than a compound of Formula (I) or Formula (II) or a pharmaceutically acceptable salt thereof (e.g., an interferon, ribavirin) and is suffering from a relapsed HBV infection.

In some embodiments, the subject is suffering from a co-infection with Hepatitis B virus (HDV). In some embodiments, the subject has been diagnosed with an HBV infection. In some embodiments, the subject has been diagnosed with an HDV infection. In some embodiments, the subject has been diagnosed with a co-infection of HBV and HDV.

In some embodiments, in a method described herein, the subject has been diagnosed with cirrhosis of the liver. In some embodiments, the subject has been diagnosed with hepatocellular carcinoma. In some embodiments, the subject has been diagnosed with hepatocellular carcinoma and is awaiting liver transplantation. In some embodiments, the subject is non-cirrhotic or has not been diagnosed with hepatocellular carcinoma.

In some embodiments, in a method described herein, the subject has been further diagnosed with an HIV infection. In some embodiments, the strain of HIV infection is known. In some embodiments, the subject is infected with HIV-1 or HIV-2 (e.g., strain 1 or strain 2).

In some embodiments, the methods described herein further comprise analyzing or receiving analysis of the body weight and temperature of the subject at least once a week until the end of treatment.

In some embodiments, the methods described herein further comprise analyzing or receiving analysis of a blood sample from the subject at least once prior to the end of treatment. In some embodiments, the blood sample is analyzed for viral load and surface antigen levels. In some embodiments, the blood sample is analyzed for the expression level of interferon (e.g., interferon alfa or interferon beta), an interferon stimulating protein (e.g., ISG15, CXCL10, OAS 1), or other cytokines. In some embodiments, the blood sample is analyzed for the presence of anti-WHS antibodies and anti-WHc antibodies.

In some embodiments, the methods described herein further comprise analyzing or receiving analysis of a liver biopsy specimen from the subject at least once prior to the end of treatment. In some embodiments, the liver biopsy specimen is analyzed for the levels of viral DNA, viral RNA, viral antigens, and cccDNA. In some embodiments, the liver biopsy specimen is analyzed for the expression level of interferon (e.g., interferon alfa or interferon beta), an interferon stimulating protein (e.g., ISG15, CXCL10, OAS 1), or other cytokines. In some embodiments, the liver biopsy specimen is analyzed for the presence of anti-WHS antibodies and anti-WHc antibodies. In some embodiments, the liver biopsy specimen is analyzed for the reduction of liver inflammation, necrosis, steatosis, or fibrosis.

In any and all embodiments, the method features a pharmaceutical composition for use in treating a subject infected with the Hepatitis D virus (HDV), the composition comprising a compound of Formula (I) (e.g., Formula (Ia), Formula (Ib), or Formula (Ic)), a compound of Formula (II) (e.g., Formula (IIa), Formula (IIb), or Formula (IIc)), or a pharmaceutically acceptable salt thereof, in combination with entecavir to thereby treat the subject.

In another aspect, the present invention features a pharmaceutical composition comprising a compound of Formula (I) wherein the compound is selected from:

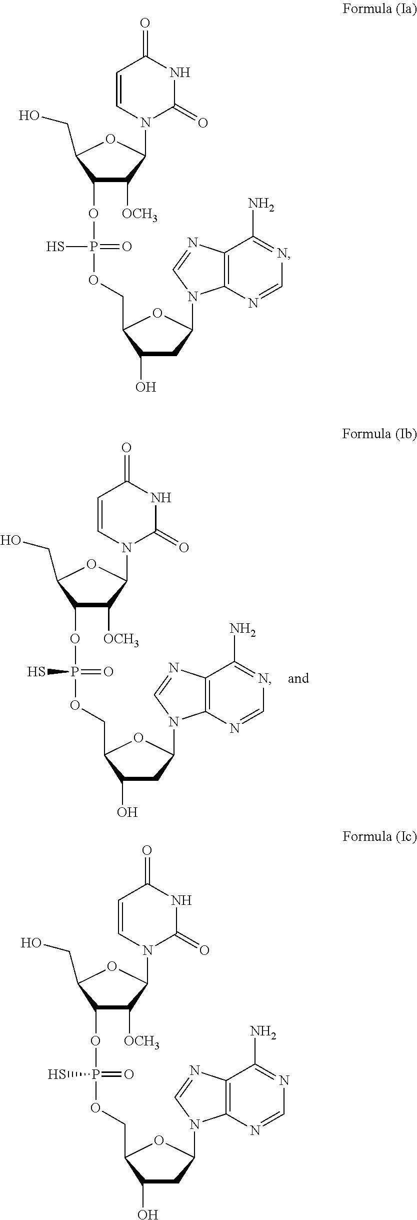

or a prodrug or pharmaceutically acceptable salt thereof and entecavir or a pharmaceutically acceptable salt thereof.

In some embodiments, the prodrug of Formula (I) is a compound of Formula (II) and the compound is selected from:

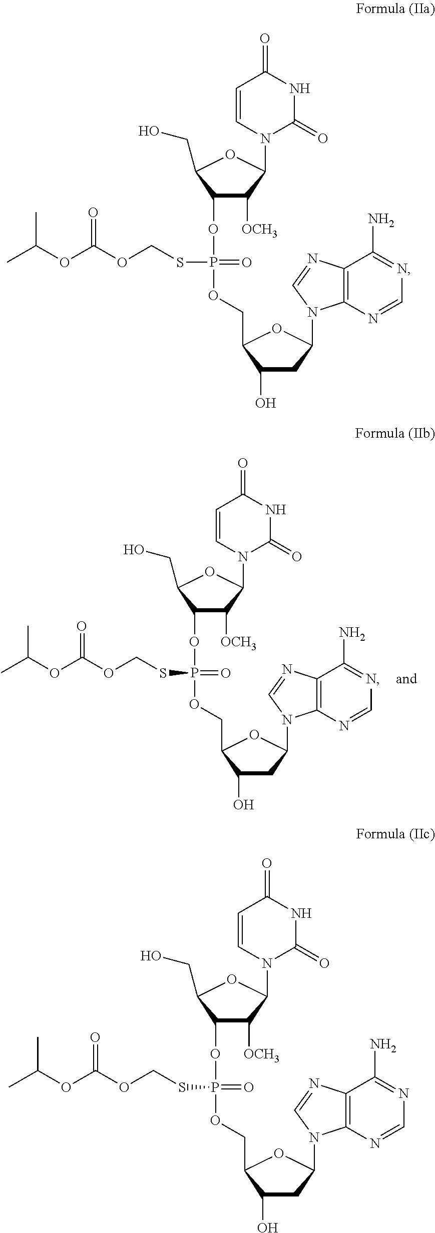

or a pharmaceutically acceptable salt thereof.

In some embodiments, the pharmaceutical composition comprises an oral dosage form. In some embodiments, the oral dosage form is a liquid dosage form or a solid dosage form. In some embodiments, the liquid dosage form comprises a suspension, a solution, a linctus, an emulsion, a drink, an elixir, or a syrup. In some embodiments, the solid dosage form comprises a capsule, tablet, dragée, or powder.

In some embodiments, the dosage of the compound of Formula (I) or Formula (II) is between about 5 mg/kg to about 100 mg/kg (e.g., about 5 mg/kg, about 10 mg/kg, about 15 mg/kg, about 20 mg/kg, about 30 mg/kg, about 40 mg/kg, about 50 mg/kg, about60 mg/kg, about 70 mg/kg, about 80 mg/kg, about 90 mg/kg, or about 100 mg/kg). In some embodiments, the dosage of the compound of Formula (I) or Formula (II) is between about 10 mg/kg to about 50 mg/kg (e.g., about 10 mg/kg, about 15 mg/kg, about 20 mg/kg, about 25 mg/kg, about 30 mg/kg, about 35 mg/kg, about 40 mg/kg, about 45 mg/kg, or about 50 mg/kg).

In some embodiments, the dosage of entecavir is between about 0.1 mg to about 5 mg (e.g., about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.25 mg, about 1.5 mg, about 1.75 mg, about 2 mg, about 2.5 mg, about 3 mg, about 3.5 mg, about 4 mg, about 4.5 mg, or about 5 mg). In some embodiments, the dosage of entecavir is between about 0.01 mg/kg to about 10 mg/kg (e.g., about 0.01 mg/kg, about 0.025 mg/kg, about 0.05 mg/kg, about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg). In some embodiments, the dosage of entecavir is between about 0.1 mg/kg to about 5 mg/kg (e.g., about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.25 mg/kg, about 1.5 mg/kg, about 1.75 mg/kg, about 2 mg/kg, about 2.5 mg/kg, about 3 mg/kg, about 3.5 mg/kg, about 4 mg/kg, about 4.5 mg/kg, or about 5 mg/kg).

In some embodiments, the dosage comprises a liquid or a solid dosage form. In some embodiments, the liquid dosage form comprises a suspension, a solution, a linctus, an emulsion, a drink, an elixir, or a syrup. In some embodiments, the solid dosage form comprises a capsule, tablet, dragée, or powder.

In some embodiments, the compound of Formula (I) or Formula (II) or entecavir is administered orally (e.g., the compound of Formula (I) or Formula (II) is administered orally, or entecavir is administered orally, or both the compound of Formula (I) or Formula (II) and entecavir are administered orally). In some embodiments, the compound of Formula (I) or Formula (II) or entecavir is administered parenterally (e.g., the compound of Formula (II) is administered parenterally). In some embodiments, the compound of Formula (I) or Formula (II) is administered parenterally and entecavir is administered orally. In some embodiments, compound of Formula (I) or Formula (II) is formulated as a fixed dose combination with entecavir (e.g., as a liquid dosage form or solid dosage form, e.g., a capsule or tablet). In some embodiments, compound of Formula (I) or Formula (II) is formulated as a fixed dose combination with entecavir (e.g., as a liquid dosage form or solid dosage form, e.g., a capsule or tablet) for oral administration.

In some embodiments, the administration of a compound of Formula (I) or Formula (II) and entecavir has a synergistic or additive effect. In some embodiments, the administration of a compound of Formula (I) or Formula (II) and entecavir has an additive effect. In some embodiments, the administration of a compound of Formula (I) or Formula (II) and entecavir has a synergistic effect.

In some embodiments, the composition comprises a mixture of compounds of Formula (I), e.g., Formula (Ib) and Formula (Ic). In some embodiments, the composition comprises Formula (Ib) and comprises less than about 5% of Formula (Ic) (e.g., less than about 4%, less than about 3%, less than about 2%, less than about 1%, less than about 0.5%, or less than about 0.1% of Formula (Ic)), or is substantially free of Formula (Ic). In some embodiments, the composition comprises Formula (Ic) and comprises less than about 5% of Formula (Ib) (e.g., less than about 4%, less than about 3%, less than about 2%, less than about 1%, less than about 0.5%, or less than about 0.1% of Formula (Ib), or is substantially free of Formula (Ib)).

In some embodiments, the composition comprises a mixture of compounds of Formula (II), e.g., Formula (IIb) and Formula (IIc). In some embodiments, the composition comprises Formula (IIb) and comprises less than about 5% of Formula (IIc) (e.g., less than about 4%, less than about 3%, less than about 2%, less than about 1%, less than about 0.5%, or less than about 0.1% of Formula (IIc), or is substantially free of Formula (IIc)). In some embodiments, the composition comprises Formula (IIc) and comprises less than about 5% of Formula (IIb) (e.g., less than about 4%, less than about 3%, less than about 2%, less than about 1%, less than about 0.5%, or less than about 0.1% of Formula (IIb), or is substantially free of Formula (IIb)).

In another aspect, the present invention features a pharmaceutical composition comprising a compound of Formula (I) wherein the compound is selected from:

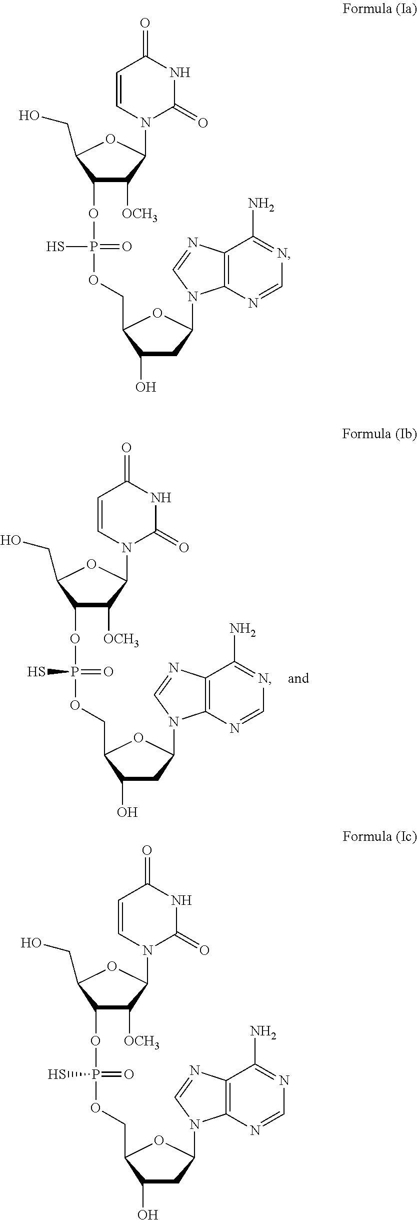

or a prodrug or pharmaceutically acceptable salt thereof and tenofovir (e.g., tenofovir dipivoxil or tenofovir alafenamide) or a pharmaceutically acceptable salt thereof.

In some embodiments, the prodrug of Formula (I) is a compound of Formula (II) and the compound is selected from:

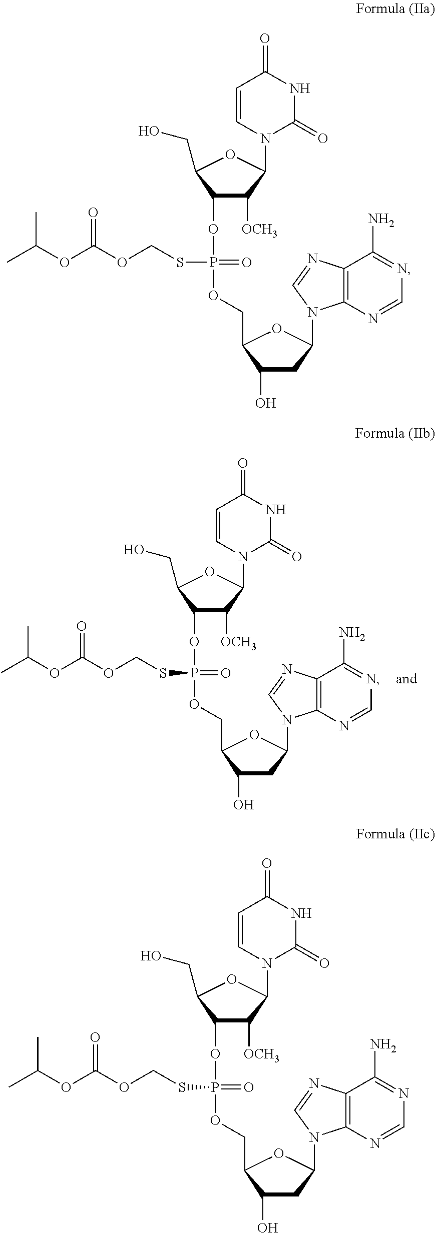

or a pharmaceutically acceptable salt thereof.

In some embodiments, the pharmaceutical composition comprises an oral dosage form. In some embodiments, the oral dosage form is a liquid dosage form or a solid dosage form. In some embodiments, the liquid dosage form comprises a suspension, a solution, a linctus, an emulsion, a drink, an elixir, or a syrup. In some embodiments, the solid dosage form comprises a capsule, tablet, dragée, or powder.

In some embodiments, the dosage of the compound of Formula (I) or Formula (II) is between about 5 mg/kg to about 100 mg/kg (e.g., about 5 mg/kg, about 10 mg/kg, about 15 mg/kg, about 20 mg/kg, about 30 mg/kg, about 40 mg/kg, about 50 mg/kg, about 60 mg/kg, about 70 mg/kg, about 80 mg/kg, about 90 mg/kg, or about 100 mg/kg). In some embodiments, the dosage of the compound of Formula (I) or Formula (II) is between about 10 mg/kg to about 50 mg/kg (e.g., about 10 mg/kg, about 15 mg/kg, about 20 mg/kg, about 25 mg/kg, about 30 mg/kg, about 35 mg/kg, about 40 mg/kg, about 45 mg/kg, or about 50 mg/kg).

In some embodiments, the dosage of tenofovir (e.g., tenofovir dipivoxil or tenofovir alafenamide) is between about 10 mg to about 500 mg (e.g., about 25 mg, about 50 mg, about 75 mg, about 100 mg, about 150 mg, about 200 mg, about 250 mg, or about 300 mg). In some embodiments, the dosage of tenofovir (e.g., tenofovir dipivoxil or tenofovir alafenamide) is between about 0.01 mg/kg to about 20 mg/kg (e.g., about 0.01 mg/kg, about 0.025 mg/kg, about 0.05 mg/kg, about 0.1 mg/kg, about 0.5 mg/kg, about 1 mg/kg, about 2 mg/kg, about 5 mg/kg, about 7.5 mg/kg, about 10 mg/kg, about 12.5 mg/kg, about 15 mg/kg, about 17.5 mg/kg, or about 20 mg/kg). In some embodiments, the dosage of tenofovir (e.g., tenofovir dipivoxil or tenofovir alafenamide) is between about 1 mg/kg to about 20 mg/kg (e.g., about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 12.5 mg/kg, about 15 mg/kg, about 17.5 mg/kg, or about 20 mg/kg).

In some embodiments, the compound of Formula (I) or Formula (II) or tenofovir (e.g., tenofovir dipivoxil or tenofovir alafenamide) is administered orally (e.g., the compound of Formula (I) or Formula (II) is administered orally, or tenofovir (e.g., tenofovir dipivoxil or tenofovir alafenamide) is administered orally, or both the compound of Formula (I) or Formula (II) and entecavir are administered orally). In some embodiments, the compound of Formula (I) or Formula (II) or tenofovir (e.g., tenofovir dipivoxil or tenofovir alafenamide) is administered parenterally (e.g., the compound of Formula (II) is administered parenterally). In some embodiments, the compound of Formula (I) or Formula (II) is administered parenterally and tenofovir (e.g., tenofovir dipivoxil or tenofovir alafenamide) is administered orally. In some embodiments, compound of Formula (I) or Formula (II) is formulated as a fixed dose combination with tenofovir (e.g., tenofovir dipivoxil or tenofovir alafenamide), e.g., as a liquid dosage form or solid dosage form, e.g., a capsule or tablet. In some embodiments, compound of Formula (I) or Formula (II) is formulated as a fixed dose combination with tenofovir (e.g., tenofovir dipivoxil or tenofovir alafenamide), e.g., as a liquid dosage form or solid dosage form, e.g., a capsule or tablet for oral administration.

In some embodiments, the administration of a compound of Formula (I) or Formula (II) and tenofovir (e.g., tenofovir dipivoxil or tenofovir alafenamide) has a synergistic or additive effect. In some embodiments, the administration of a compound of Formula (I) or Formula (II) and tenofovir (e.g., tenofovir dipivoxil or tenofovir alafenamide) has an additive effect. In some embodiments, the administration of a compound of Formula (I) or Formula (II) and tenofovir (e.g., tenofovir dipivoxil or tenofovir alafenamide) has a synergistic effect.

In some embodiments, the composition comprises a mixture of compounds of Formula (I), e.g., Formula (Ib) and Formula (Ic). In some embodiments, the composition comprises Formula (Ib) and comprises less than about 5% of Formula (Ic) (e.g., less than about 4%, less than about 3%, less than about 2%, less than about 1%, less than about 0.5%, or less than about 0.1% of Formula (Ic)), or is substantially free of Formula (Ic). In some embodiments, the composition comprises Formula (Ic) and comprises less than about 5% of Formula (Ib) (e.g., less than about 4%, less than about 3%, less than about 2%, less than about 1%, less than about 0.5%, or less than about 0.1% of Formula (Ib), or is substantially free of Formula (Ib)).

In some embodiments, the composition comprises a mixture of compounds of Formula (II), e.g., Formula (IIb) and Formula (IIc). In some embodiments, the composition comprises Formula (IIb) and comprises less than about 5% of Formula (IIc) (e.g., less than about 4%, less than about 3%, less than about 2%, less than about 1%, less than about 0.5%, or less than about 0.1% of Formula (IIc), or is substantially free of Formula (IIc)). In some embodiments, the composition comprises Formula (IIc) and comprises less than about 5% of Formula (IIb) (e.g., less than about 4%, less than about 3%, less than about 2%, less than about 1%, less than about 0.5%, or less than about 0.1% of Formula (IIb), or is substantially free of Formula (IIb)).

### BRIEF DESCRIPTION OF THE DRAWINGS

FIG. 1 is a chart depicting the antiviral activity of Formula (Ia) in chronically WHV-infected woodchucks through week 16 after intraperitoneal administration of Formula (Ia).

FIG. 2A-2F are graphs depicting the dose-dependent, transient suppression of serum viremia and antigenemia in chronic WHV carrier woodchucks upon Formula (IIa) treatment. Changes in levels of serum WHV DNA (FIGS. 2A-2C) and WHsAg (FIGS. 2D-2F) relative to T₀ (pretreatment baseline) during daily, oral treatment with Formula (IIa) for 12 weeks in individual woodchucks administered a low (15 mg/kg, FIGS. 2A, 2D) or high dose (30 mg/kg, FIGS. 2B, 2E), and mean of each group (FIGS. 2C, 2F, open circles: low dose; closed circles: high dose). Error bars represent the standard error of the mean.

FIGS. 3A-3C are graphs describing the dose-dependent, transient reduction in hepatic levels of WHV nucleic acids upon Formula (IIa) treatment. Changes in mean hepatic levels of WHV cccDNA (FIG. 3A), WHV RI DNA (FIG. 3B), and WHV RNA (FIG. 3C) relative to week 1 (pretreatment baseline) in response to Formula (IIa) treatment at a low (15 mg/kg, open circles) or high dose (30 mg/kg, closed circles). Error bars represent the standard error of the mean.

FIGS. 4A-4E are graphs depicting the effect of Formula (IIa) dosage on WHV replication in chronic WHV carrier woodchucks. Maximum reductions in serum WHV DNA (FIG. 4A) and WHsAg (FIG. 4B) and in hepatic WHV cccDNA (FIG. 4C), WHV RI DNA (FIG. 4D), and WHV RNA (FIG. 4E) in response to Formula (IIa) treatment at a low (LD; 15 mg/kg) or high dose (HD; 30 mg/kg) were observed. Changes in serum and hepatic viral parameters were calculated relative to T₀ or week −1 (pretreatment baseline), respectively. The bar height indicates the mean of each group, and the errors bars represent the standard error of the mean. The asterisks immediately below the bars denote the level of statistical significance relative to pretreatment baseline: **p<0.01 and ***p<0.001. The p-values below the horizontal lines indicate the level of statistical significance between both groups. Per the sampling scheme described herein, the following data was included in the analyses: maximum reduction in serum WHV DNA and WHsAg at weeks 0-12 and maximum reduction in hepatic WHV cccDNA, RI DNA, and RNA at weeks 6 and 12.

FIGS. 5A-5D are graphs depicting the effect of Formula (IIa) treatment on WHV antigen expression and inflammation in the liver of chronic WHV carrier woodchucks. Changes in mean scores for cytoplasmic WHcAg expression (FIGS. 5A, 5B) and liver histology (FIGS. 5C, 5D) in response to Formula (IIa) treatment at a low (15 mg/kg, FIGS. 5A, 5C) or high dose (30 mg/kg, FIGS. 5B, 5D) are shown. Changes in mean serum WHV DNA (open circles) relative to T₀ (pretreatment baseline) is plotted on the left y-axis. The mean immunohistochemistry (IHC) score for cytoplasmic WHcAg expression in liver (FIGS. 5A, 5B) and the mean liver histology score for portal and lobular sinusoidal hepatitis (FIGS. 5C, 5D) are plotted on the right y-axis. The IHC score was derived from the mean of the stained hepatocyte percentage score combined with the mean of the staining intensity score. A composite IHC score of 0 indicates absence of WHcAg staining in all hepatocytes (0%) whereas 8 indicates presence of strong WHcAg staining in 81-100% of hepatocytes. Specifically, the percentage of WHcAg stained hepatocytes was scored on a 0-5 scale, where 0 indicates 0%, 1 indicates 1-20%, 2 indicates 21-40%, 3 indicates 41-60%, 4 indicates 61-80%, and 5 indicates 81-100% of cells stained. The intensity of WHcAg staining was scored on a 0-3 scale, where 0 indicates no staining, 1 indicates weak staining, 2 indicates moderate staining, and 3 indicates strong staining. The liver histology score was derived from the mean of the lobular sinusoidal hepatitis score combined with the mean of the portal hepatitis score (n=1-5 portal tracts examined). A composite histology score of 0 indicates absent hepatitis, >0-2 indicates mild hepatitis, >2-4 indicates moderate hepatitis and >4 indicates marked to severe hepatitis. Error bars represent the standard error of the mean.

FIGS. 6A-6B are graphs depicting the effect of Formula (IIa) treatment of chronic WHV carrier woodchucks on liver enzyme levels. Changes in mean serum levels of SDH, AST and ALT in response to Formula (IIa) treatment at a low (15 mg/kg, FIG. 6A) or high dose (30 mg/kg, FIG. 6B) were observed. Changes in mean serum WHV DNA (open circles) relative to T₀ (pretreatment baseline) is plotted on the left y-axis. Changes in mean serum SDH, AST, and ALT are all plotted on the right y-axis. Error bars represent the standard error of the mean.

FIGS. 7A-7D are graphs depicting the effect of Formula (IIa) treatment on expression levels of type I IFNs, cytokine, and ISGs in peripheral blood of chronic WHV carrier woodchucks. Changes in mean blood transcript levels of IFN-α, IFN-β, and IL-6 (FIGS. 7A, 7B) and of CXCL10, OAS1 and ISG15 (FIGS. 7C, 7D) are shown in response to Formula (IIa) treatment at a low (15 mg/kg, FIG. 7A, 7C) or high dose (30 mg/kg, FIGS. 7B, 7D). Changes in mean serum WHV DNA (open circles) relative to T₀ (pretreatment baseline) is plotted on the left y-axis. Changes in mean blood IFN-α, IFN-β, and IL-6 are all plotted on the right y-axis in the top panels. Changes in mean blood CXCL10, OAS1, and ISG15 are all plotted on the right y-axis in the bottom panels. Error bars represent the standard error of the mean.

FIGS. 8A-8D are graphs depicting the effect of Formula (IIa) treatment on expression levels of type I IFNs, cytokine, and ISGs in the liver of chronic WHV carrier woodchucks. Changes in mean liver transcript levels of IFN-α, IFN-β, and IL-6 (FIGS. 8A, 8B) and of CXCL10, OAS1 and ISG15 (FIGS. 8C, 8D) are shown in response to Formula (IIa) treatment at a low (15 mg/kg, FIGS. 8A, 8C) or high dose (30 mg/kg, FIGS. 8B, 8D). Changes in mean serum WHV DNA (black open circles) relative to T₀ (pretreatment baseline) is plotted on the left y-axis. Changes in mean liver IFN-α, IFN-β, and IL-6 are all plotted on the right y-axis in the top panels. Changes in mean liver CXCL10, OAS1, and ISG15 are all plotted on the right y-axis in the bottom panels. Error bars represent the standard error of the mean.

FIGS. 9A-9B are graphs showing the long-lasting activation of the RIG-I/NOD2 pathway upon treatment with Formula (IIa). Changes in mean liver transcript levels of RIG-I, NOD2, TMEM173 (i.e., STING), and IRF3 in a subset of woodchucks in response to Formula (IIa) treatment are shown in response to Formula (IIa) treatment at a low (15 mg/kg, n=3, FIG. 9A) or high dose (30 mg/kg, n=3, FIG. 9B).

FIGS. 10A-10F are graphs further showing the long-lasting activation of the RIG-I/NOD2 pathway upon Formula (IIa) treatment. Changes in the IHC scores for RIG-I (FIG. 10A) and NOD2 (FIG. 10B) expression in the liver are shown over the course of the 20 week treatment. Pictures of RIG-I (FIGS. 10C, 10E) and NOD2 (FIGS. 10D, 10F) stained hepatocytes from two woodchucks, each treated with Formula (IIa) as part of high dose group, are shown at pre-treatment, as well as at the 6, 12, and 20 week time points.

FIGS. 11A-11B are graphs showing the changes in mean serum levels of WHV DNA relative to T₀ (pretreatment baseline) and in mean plasma levels of Formula (Ia) during daily, oral treatment with Formula (IIa) for 12 weeks in woodchucks administered a low (15 mg/kg, FIG. 9A) or high dose (30 mg/kg, FIG. 9B). Serum WHV DNA is plotted on the left y-axis. Plasma Formula (Ia) is plotted on the right y-axis. Error bars represent the standard error of the mean.

FIGS. 12A-12B are graphs depicting individual changes in serum levels of WHV DNA relative to T₀ (pretreatment baseline) and in plasma levels of Formula (Ia) during daily, oral treatment with Formula (IIa) for 12 weeks in individual woodchucks administered a low (15 mg/kg, FIG. 10A) or high dose (30 mg/kg, FIG. 10B). Serum WHV DNA (black open circles) is plotted on the left y-axis. Formula (Ia) (red circles) is plotted on the right y-axis.

FIGS. 13A-13F are graphs depicting hepatic levels of WHV cccDNA (FIGS. 11A, 11B), WHV RI DNA (FIGS. 11C, 11D) and WHV RNA (FIGS. 11E, 11F) relative to week-1 (pretreatment baseline) during daily, oral treatment with Formula (IIa) for 12 weeks in individual woodchucks administered a low (15 mg/kg, FIGS. 11A, 11C, 11E) or high dose (30 mg/kg, FIGS. 11B, 11D, 11F).

FIGS. 14A-14B are graphs showing individual changes in serum levels of WHV DNA relative to T₀ (pretreatment baseline) and immunohistochemistry (IHC) scores for cytoplasmic WHcAg expression in liver during daily, oral treatment with Formula (IIa) for 12 weeks in individual woodchucks administered a low (15 mg/kg, FIG. 12A) or high dose (30 mg/kg, FIG. 12B). Serum WHV DNA (open circles) is plotted on the left y-axis and the IHC score is plotted on the right y-axis. The IHC score was derived from the mean of the stained hepatocyte percentage score combined with the mean of the staining intensity score. A composite IHC score of 0 indicates absence of WHcAg staining in all hepatocytes (0%) whereas 8 indicates presence of strong WHcAg staining in 81-100% of hepatocytes. Specifically, the percentage of WHcAg stained hepatocytes was scored on a 0-5 scale, where 0 indicates 0%, 1 indicates 1-20%, 2 indicates 21-40%, 3 indicates 41-60%, 4 indicates 61-80%, and 5 indicates 81-100% of cells stained. The intensity of WHcAg staining was scored on a 0-3 scale, where 0 indicates no staining, 1 indicates weak staining, 2 indicates moderate staining, and 3 indicates strong staining. ND=not determined as liver biopsy tissue was not collected.

FIGS. 15A-15B are graphs depicting individual changes in serum levels of WHV DNA relative to T₀ (pretreatment baseline) and scores for liver histology during daily, oral treatment with Formula (IIa) for 12 weeks in individual woodchucks administered a (a) low (15 mg/kg) or (b) high dose (30 mg/kg). Serum WHV DNA (black open circles) is plotted on the left y-axis and the liver histology score (brown bars) is plotted on the right y-axis. The liver histology score was derived from the mean of the lobular sinusoidal hepatitis score combined with the mean of the portal hepatitis score (n=1-5 portal tracts examined). A composite histology score of 0 indicates absent hepatitis, >0-2 indicates mild hepatitis, >2-4 indicates moderate hepatitis and >4 indicates marked to severe hepatitis. ND=not determined as liver biopsy tissue was not collected.

FIGS. 16A-16E are graphs showing changes in group serum levels of liver enzymes during and following completion of Formula (IIa) treatment in relation to pretreatment levels. Maximum increases in serum SDH (FIG. 14A), AST (FIG. 14B), ALT (FIG. 14C), ALP (FIG. 14D), and GGT (FIG. 14E) in response to Formula (IIa) treatment at a low (LD; 15 mg/kg) or high dose (HD; 30 mg/kg). Changes in serum levels of liver enzymes were calculated relative to T₀ (pretreatment baseline). The bar height indicates the mean of each group, and the errors bars represent the standard error of the mean. The asterisk immediately above the bar denotes the level of statistical significance relative to pretreatment baseline: *p<0.05. The p-values above the horizontal lines indicate the level of statistical significance between both groups and of individual groups between treatment and follow-up. Per the sampling scheme described herein, the following data for liver enzymes was included in the analyses: maximum increases during treatment at weeks 4, 8 and 12, and maximum increases during follow-up at weeks 16 and 20. The T₀ (pretreatment baseline) levels for SDH, AST, ALT, ALP, and GGT were 73.0, 57.6, 7.2, 47.4, 5.2 IU/mL in the low dose group and 94.6, 69.8, 7.6, 24.2, 5.4 IU/mL in the high dose group.

FIGS. 17A-17B are graphs showing individual changes in serum levels of WHV DNA relative to T₀ (pretreatment baseline) and in serum levels of SDH, AST and ALT during daily, oral treatment with Formula (IIa) for 12 weeks in individual woodchucks administered a low (15 mg/kg, FIG. 15A) or high dose (30 mg/kg, FIG. 15B). Serum WHV DNA (open circles) is plotted on the left y-axis. SDH, AST, and ALT are all plotted on the right y-axis.

FIGS. 18A-18F are graphs showing changes in group expression levels of type I IFNs, cytokine, and ISGs in peripheral blood during and following completion of Formula (IIa) treatment in relation to pretreatment levels. Maximum increases in blood transcripts of IFN-α (FIG. 16A), IFN-β (FIG. 16B), IL-6 (FIG. 16C), CXCL10 (FIG. 16D), OAS1 (FIG. 16E), and ISG15 (FIG. 16F) in response to Formula (IIa) treatment at a low (LD; 15 mg/kg) or high dose (HD; 30 mg/kg). Changes in transcript levels of host innate immune response genes were calculated relative to T₀ (pretreatment baseline). The bar height indicates the mean of each group, and the errors bars represent the standard error of the mean. The asterisks immediately above the bars denote the level of statistical significance relative to pretreatment baseline: *p<0.05, **p<0.01 and ***p<0.001. The p-values above the horizontal lines indicate the level of statistical significance between both dose groups and of individual dose groups between treatment and follow-up. Per the sampling scheme described in the experimental procedures, the following data for transcript levels of immune response genes was included in the analyses: maximum increases during treatment at weeks 6 and 12, and maximum increase during follow-up at week 18.

FIGS. 19A-19B are graphs showing changes in serum levels of WHV DNA relative to T₀ (pretreatment baseline) and in blood transcript levels of IFN-α, IFN-β, and IL-6 during daily, oral treatment with Formula (IIa) for 12 weeks in individual woodchucks administered a low (15 mg/kg, FIG. 17A) or high dose (30 mg/kg, FIG. 17B). Serum WHV DNA (open circles) is plotted on the left y-axis. IFN-α, IFN-β, and IL-6 are all plotted on the right y-axis.

FIGS. 20A-20B are graphs showing individual changes in serum levels of WHV DNA relative to T₀ (pretreatment baseline) and in blood transcript levels of CXCL10, OAS1 and ISG15 during daily, oral treatment with Formula (IIa) for 12 weeks in individual woodchucks administered a low (15 mg/kg, FIG. 18A) or high dose (30 mg/kg, FIG. 18B). Serum WHV DNA (open circles) is plotted on the left y-axis. CXCL10, OAS1, and ISG15 are all plotted on the right y-axis in the bottom panels.

FIGS. 21A-21F are graphs showing a comparison of basal expression levels of type I IFNs, cytokine, and ISGs in peripheral blood of age-matched, untreated chronic WHV carrier woodchucks with pretreatment levels in Formula (IIa) treated woodchucks. Mean levels of blood transcripts of IFN-α (FIG. 21A), IFN-α (FIG. 21B), IL-6 FIG. 21C), CXCL10 (FIG. 21D), OAS1 (FIG. 21E), and ISG15 (FIG. 21F) in five untreated control woodchucks and in ten woodchucks of the combined low dose (LD) and high dose (HD) groups are shown. Transcript levels of host innate immune response genes for woodchucks of the LD and HD groups were obtained at the pre-treatment baseline (T₀). The bar height indicates the mean of each group. The p values above the horizontal lines indicate the level of statistical significance between groups.

FIGS. 22A-22F are graphs showing changes in group expression levels of type I IFNs, cytokine, and ISGs in liver during and following completion of Formula (IIa) treatment in relation to pretreatment levels. Maximum increases in liver transcripts of IFN-α (FIG. 19A), IFN-β (FIG. 19B), IL-6 (FIG. 19C), CXCL10 (FIG. 19D), OAS1 (FIG. 19E), and ISG15 (FIG. 19F) in response to Formula (IIa) treatment at a low (LD; 15 mg/kg) or high dose (HD; 30 mg/kg). Changes in transcript levels of host immune response genes were calculated relative to week-1 (pretreatment baseline). The bar height indicates the mean of each group, and the errors bars represent the standard error of the mean. The asterisks immediately above the bars denote the level of statistical significance relative to pretreatment baseline: *p<0.05, **p<0.01 and ***p<0.001. The p-values above the horizontal lines indicate the level of statistical significance between both groups and of individual groups between treatment and follow-up. Per the sampling scheme described in the experimental procedures, the following data for transcript levels of immune response genes was included in the analyses: maximum increases during treatment at weeks 6 and 12, and maximum increase during follow-up at week 20.

FIGS. 23A-23B are graphs showing changes in serum levels of WHV DNA relative to T₀ (pretreatment baseline) and in liver transcript levels of IFN-α, IFN-β, and IL-6 during daily, oral treatment with Formula (IIa) for 12 weeks in individual woodchucks administered a low (15 mg/kg, FIG. 20A) or high dose (30 mg/kg, FIG. 20B). Serum WHV DNA (open circles) is plotted on the left y-axis. IFN-α, IFN-β, and IL-6 are all plotted on the right y-axis.

FIGS. 24A-24B are graphs showing individual changes in serum levels of WHV DNA relative to T₀ (pretreatment baseline) and in liver transcript levels of CXCL10, OAS1 and ISG15 during daily, oral treatment with Formula (IIa) for 12 weeks in individual woodchucks administered a low (15 mg/kg, FIG. 21A) or high dose (30 mg/kg, FIG. 21B). Serum WHV DNA (open circles) is plotted on the left y-axis. CXCL10, OAS1, and ISG15 are all plotted on the right y-axis in the bottom panels.

FIGS. 25A-25F are graphs showing a comparison of basal levels of type I IFNs, cytokine, and ISGs in livers of age-matched, untreated chronic WHV carrier woodchucks with pretreatment levels in Formula (IIa) treated woodchucks. Mean levels of liver transcripts of IFN-α (FIG. 25A), IFN-β (FIG. 25B), IL-6 (FIG. 25C), CXCL10 (FIG. 25D), OAS1 (FIG. 25E), and ISG15 (FIG. 25F) in five untreated control woodchucks and in ten woodchucks of the combined low dose (15 mg/kg) and high dose (30 mg/kg) groups are shown. Transcript levels of host response genes for woodchucks of the low dose and high dose groups were obtained at T₀ (pretreatment baseline), and the bar height indicates the mean of each group. The p-values above the horizontal lines refer to the levels of statistical significance between the groups.

FIG. 26 is a table summarizing the oligonucleotides used for determinations of host immune response in blood and liver. F: forward primer; R: reverse primer; P: probe.

FIGS. 27A-27B are graphs comparing the decline of serum WHV DNA in two groups of five chronically WHV-infected woodchucks as described in Example 3. Entecavir (0.5 mg/kg) was either administered before (group 2, FIG. 27A) or after treatment with Formula (IIa) (30 mg/kg, Group 2, FIG. 27B) for 16 weeks, and the effect on serum WHV DNA monitored.

FIGS. 28A-28B are graphs comparing the decline of serum WHV DNA in two groups of five chronically WHV-infected woodchucks as described in Example 3. Entecavir (0.5 mg/kg) was either administered before (group 2, FIG. 28A) or after treatment with Formula (IIa) (30 mg/kg, Group 2, FIG. 28B) for 16 weeks, and the effect on serum WHV DNA monitored.

FIG. 29 is a graph comparing the effects of treatment with Formula (IIa) alone (15 mg/kg/day and 30 mg/kg/day) with the effects of treatment of Formula (IIa) (30 mg/kg/day) followed by entecavir (0.5 mg/kg/day) on serum WHV DNA (log₁₀) levels in chronically WHV-infected woodchucks.

FIG. 30 is a graph comparing the effects of treatment with Formula (IIa) alone (15 mg/kg/day and 30 mg/kg/day) with the effects of treatment of Formula (IIa) (30 mg/kg/day) followed by entecavir (0.5 mg/kg/day) on serum WHsAg levels in chronically WHV-infected woodchucks.

FIG. 31 depicts a table summarizing the in vitro activity (EC₅₀, μM) of Formula (IIa) and the antiviral nucleoside analogs lamivudine (3TC) and adefovir dipivoxil (ADV) in assays using six different cell samples chronically infected with HBV. In each assay, Formula (IIa), 3TC, or ADV was added to the cells daily for nine consecutive days. Each cell sample was infected with either wild type HBV or a variant HBV strain comprising a mutation in the HBV polymerase (P), e.g., M204V, M204I, L180M, L180M/M204V, or N236T.

### DETAILED DESCRIPTION OF THE INVENTION

The present invention relates to methods of treating a subject infected with the Hepatitis B virus, the method comprising administration of a compound of Formula (I) (e.g., Formula (Ia), Formula (Ib), or Formula (Ic)) or a prodrug (e.g., a compound of Formula (II), e.g., Formula (IIa), Formula (IIb), or Formula (IIc)) or pharmaceutically acceptable salt thereof.

### Definitions

As used herein, the articles “a” and “an” refer to one or to more than one (e.g., to at least one) of the grammatical object of the article.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the term “acquire” or “acquiring” as the terms are used herein, refer to obtaining possession of a physical entity (e.g., a sample, e.g., blood sample or liver biopsy specimen), or a value, e.g., a numerical value, by “directly acquiring” or “indirectly acquiring” the physical entity or value. “Directly acquiring” means performing a process (e.g., an analytical method) to obtain the physical entity or value. “Indirectly acquiring” refers to receiving the physical entity or value from another party or source (e.g., a third party laboratory that directly acquired the physical entity or value). Directly acquiring a value includes performing a process that includes a physical change in a sample or another substance, e.g., performing an analytical process which includes a physical change in a substance, e.g., a sample, performing an analytical method, e.g., a method as described herein, e.g., by sample analysis of bodily fluid, such as blood by, e.g., mass spectroscopy (e.g. LC-MS), or PCR (e.g., RT-PCR).

As used herein, an amount of a compound, conjugate, or substance effective to treat a disorder (e.g., a disorder described herein), “therapeutically effective amount,” “effective amount” or “effective course” refers to an amount of the compound, substance, or composition which is effective, upon single or multiple dose administration(s) to a subject, in treating a subject, or in curing, alleviating, relieving or improving a subject with a disorder (e.g., an HBV infection or HBV/HDV co-infection) beyond that expected in the absence of such treatment.

As used herein, the terms “prevent” or “preventing” as used in the context of a disorder or disease, refer to administration of an agent to a subject, e.g., the administration of a compound of the present invention (e.g., compound of Formula (I) (e.g., Formula (Ia), Formula (Ib), or Formula (Ic)) or a prodrug (e.g., a compound of Formula (II), e.g., Formula (IIa), Formula (IIb), or Formula (IIc)) to a subject, such that the onset of at least one symptom of the disorder or disease is delayed as compared to what would be seen in the absence of administration of said agent.

As used herein, the term “prodrug” refers to a compound which, when metabolized (e.g., in vivo or in vitro), yields an active compound. In some embodiments, the prodrug may be inactive, or possess less activity that the free drug, but may provide advantageous handling, administration, or metabolic properties. Exemplary prodrug moieties of the present invention may be linked to the free drug through the hydroxyl, amino, phosphate, or phosphorothioate backbone of the nucleotide, and may comprise an ester, a carbamate, a carbonyl, a thioester, amide, isocyanate, urea, thiourea, or other physiologically acceptable metabolically labile moiety. In some embodiments, a prodrug is activated through enzymatic hydrolysis.

As used herein, the term “resistant” or “resistance” refers to a strain of HBV that is not substantially diminished or inactivated upon administration with an anti-HBV agent. In some embodiments, a resistant HBV strain comprises a protein (e.g., an HBsAg, HBcAg, HBeAg, L, M, P, or X protein) that substantially maintains its activity, function, or structure in the presence of an anti-HBV agent known to inhibit, bind to, or alter said protein. In some embodiments, a resistant HBV strain comprises a protein bearing an amino acid mutation (e.g., an amino acid substitution, addition, or deletion) compared with a reference sequence of said protein. In some embodiments, an HBV protein bearing an amino acid mutation (e.g., an amino acid substitution, addition, or deletion) may result in aberrant function of said protein or affect the inhibition of said protein with an anti-HBV agent. In some embodiments, the level of resistance may be determined through a measurement of viral load or other biomarker in a sample (e.g., a serum sample), or through the determination of the IC₅₀ value of a specific antiviral agent other agents beyond a compound of Formula (I) or Formula (II) in a sample (e.g., a serum sample).

As used herein, the term “subject” is intended to include human and non-human animals. Exemplary human subjects include a human patient having a disorder, e.g., a disorder described herein, or a normal subject. The term “non-human animals” includes all vertebrates, e.g., non-mammals (such as chickens, amphibians, reptiles) and mammals, such as non-human primates, domesticated and/or agriculturally useful animals, e.g., sheep, dogs, cats, cows, pigs, etc. In exemplary embodiments of the invention, the subject is a woodchuck (e.g., an Eastern woodchuck (Marmota monax)).

As used herein, the terms “treat” or “treating” a subject having a disorder or disease refer to subjecting the subject to a regimen, e.g., the administration of a compound of Formula (I) or a prodrug (e.g., a compound of Formula (II)) or pharmaceutically acceptable salt thereof, or a composition comprising Formula (I) or a prodrug (e.g., a compound of Formula (II)) or pharmaceutically acceptable salt thereof, such that at least one symptom of the disorder or disease is cured, healed, alleviated, relieved, altered, remedied, ameliorated, or improved. Treating includes administering an amount effective to alleviate, relieve, alter, remedy, ameliorate, improve or affect the disorder or disease, or the symptoms of the disorder or disease. The treatment may inhibit deterioration or worsening of a symptom of a disorder or disease.

Numerous ranges, e.g., ranges for the amount of a drug administered per day, are provided herein. In some embodiments, the range includes both endpoints. In other embodiments, the range excludes one or both endpoints. By way of example, the range can exclude the lower endpoint. Thus, in such an embodiment, a range of 250 to 400 mg/day, excluding the lower endpoint, would cover an amount greater than 250 that is less than or equal to 400 mg/day.

“Co-administration”, “co-administering” or “co-providing”, as used herein in the context of the administration of therapies, refers to administration at the same time, administration of one therapy before (e.g., immediately before, less than about 5, about 10, about 15, about 30, about 45, about 60 minutes, about 1, about 2, about 3, about 4, about 6, about 8, about 10, about 12, about 16, about 20, about 24, about 48, about 72 or more hours before) administration of a secondary therapy.

A “course” or “course of therapy,” as referred to herein, comprises one or more separate administrations of a therapeutic agent (e.g., a compound of Formula (I)) or a prodrug (e.g., a compound of Formula (II)) or pharmaceutically acceptable salt thereof, in combination with entecavir). A course of therapy can comprise one or more cycles of a therapeutic agent. In some embodiments, a therapeutic agent is administered to a subject at least once, at least twice, at least three times, at least four times, or more over a course of treatment. A subject may be administered with one or more courses of treatment. In some embodiments, rest periods may be interposed between courses of treatment. For example, a rest period may be about 1, about 2, about 4, about 6, about 8, about 10, about 12, about 16, about 20, or about 24 hours; or about 1, about 2, about 3, about 4, about 5, about 6, or about 7 days; or about 1, about 2, about 3, about 4 or more weeks in length.

A “cycle”, as used herein in the context of a cycle of administration of a drug, refers to a period of time for which a drug is administered to a patient. For example, if a drug is administered for a cycle of 4 weeks days, the periodic administration, e.g., daily or twice daily, is given for 4 weeks. A drug can be administered for more than one cycle. In some embodiments, the first and second or subsequent cycles are the same in terms of one or both of duration and periodic administration. In embodiments, a first and second or subsequent cycle differs in terms of one or both of duration and periodic administration. Rest periods may be interposed between cycles. A rest cycle may be about 1, about 2, about 4, about 6, about 8, about 10, about 12, about 16, about 20, or about 24 hours; or about 1, about 2, about 3, about 4, about 5, about 6, or about 7 days; or about 1, about 2, about 3, about 4 or more weeks in length.

### Compounds and Therapeutic Agents

The present invention features methods for treatment of a subject infected with HBV or a resistant variant thereof comprising administration of a composition comprising a compound of Formula (I) or a prodrug or pharmaceutically acceptable salt thereof. The active agent is Formula (I), which may be described by any one of Formula (Ia), Formula (Ib), and Formula (Ic), or a combination thereof:

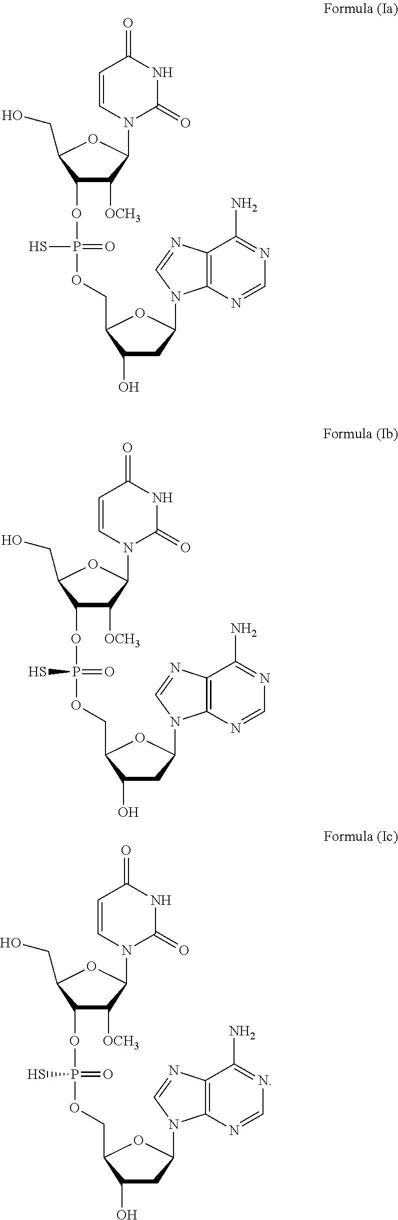

The composition of the present invention may comprise a prodrug of Formula (I), wherein the prodrug moiety comprises a hydroxyl, amino, phosphate, ester, carbamate, carbonyl, thioester, amide, isocyanate, urea, thiourea, or other physiologically acceptable metabolically labile moiety. In some embodiments, a prodrug is activated through enzymatic hydrolysis.

In certain embodiments, said prodrug is a compound of Formula (II). The prodrug thereof (e.g., the compound of Formula (II)) may be described by any one of Formula (IIa), Formula (IIb), and Formula (IIc), or a combination thereof:

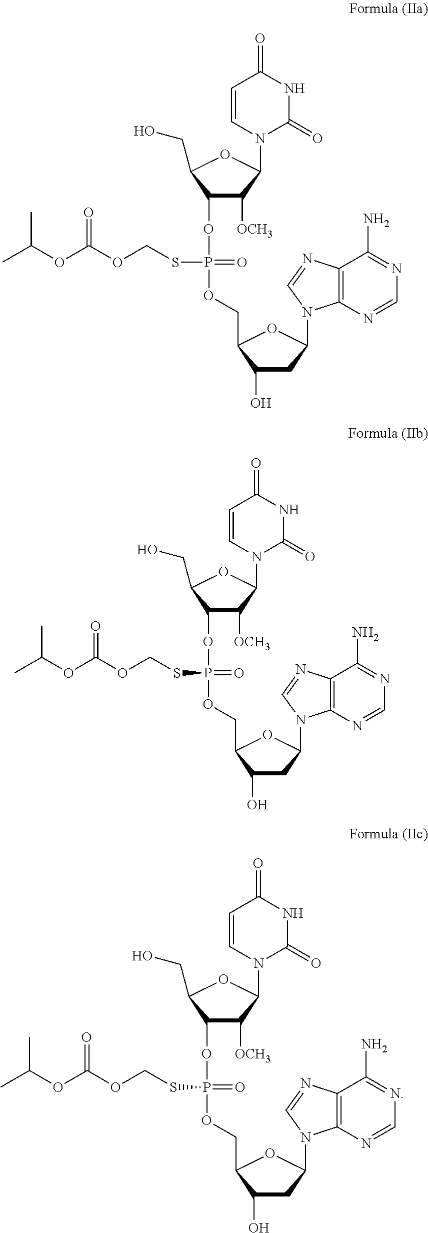

Formula (I) and its prodrug Formula (II) are small molecule nucleic acid hybrid (dinucleotide) compounds that combine both antiviral and immune modulating activities. The latter activity mediates controlled apoptosis of virus-infected hepatocytes via stimulation of the innate immune response, similar to what is also achieved by IFN-α therapy in HBV-infected patients.

Without wishing to be bound by theory, the mechanism of action of Formula (I) and its prodrug Formula (II) may be dissected into two components. The first component entails the host immune stimulating activity of Formula (I), which induces endogenous IFNs via the activation of viral sensor proteins, e.g., retinoic acid-inducible gene 1 (RIG-I) and nucleotide-binding oligomerization domain-containing protein 2 (NOD2) (Takeuchi, 0. and Akira S. Cell (2010) 140:805-820; Sato, S. et al. Immunity (2015) 42:123-132; Sabbah, A. et al. Nat Immunol (2009) 10:1073-1080). Activation may occur by binding of Formula (I) to the RIG-I/NOD2 proteins at their nucleotide binding domain. The RIG-I and NOD2 proteins are located in the cytosol of cells, including hepatocytes, and usually recognize signature patterns of foreign nucleic acids such as the pathogen associated molecular pattern (PAMP). Once PAMP within viral RNA or DNA is recognized, RIG-I and NOD2 may become activated and trigger the IFN signaling cascade that then results in IFN and interferon-stimulated gene (ISG) production and induction of an antiviral state in cells. In the case of HBV, the PAMP is believed to be the pre-genomic RNA which has a significant double-stranded RNA structure known as epsilon structure.

The second component of the mechanism of action of Formula (I) and its prodrug Formula (II) involves its direct antiviral activity, which inhibits the synthesis of viral nucleic acids by steric blockage of the viral polymerase. The block may be achieved by interaction Formula (I) with RIG-I and NOD2 as described above that then in turn may prevent the polymerase enzyme from engaging with the viral nucleic acid template for replication (i.e, HBV pre-genomic RNA). The cytotoxic potential of Formula (II) (e.g., Formula (IIa)) has been initially evaluated using a panel of cell lines. Similar to the parental drug, Formula (II) demonstrated an excellent safety profile, with a 50% cytotoxic concentration (CC50) of greater than 1000 μM (Coughlin, J. E. et al. Bioorg Med Chem Lett (2010) 20:1783-1786). Formula (II) has been further evaluated for anti-HBV activity in a cell-based assay against wild-type HBV and against lamivudine-(3TC) and adefovir-(ADV) resistant mutant HBV. Formula (II) was found to have antiviral activity against wild-type HBV, with a potency that was in the range of ADV (but less than that of 3TC).

In some embodiments, the method described herein comprises administration of a compound of Formula (I), e.g., Formula (Ia), Formula (Ib), or Formula (Ic), or a pharmaceutically acceptable salt thereof. In other embodiments, the method described herein comprises administration of prodrug of Formula (I) (e.g., a compound of Formula (II), e.g., Formula (IIa), Formula (IIb), or Formula (IIc)) or a pharmaceutically acceptable salt thereof. In other embodiments, the method herein describes administration of a composition comprised of a combination of a compound of Formula (I) (e.g., Formula (Ia), Formula (Ib), or Formula (Ic)) and a compound of Formula (II) (e.g., Formula (Ia), Formula (Ib), or Formula (Ic)) or pharmaceutically acceptable salts thereof. It is well established that the prodrug Formula (I) has been shown to be converted to the active drug Formula (I) (e.g., the Rp- and Sp-Formula (I) isomers) upon administration.

The compounds provided herein may contain one or more asymmetric centers and thus occur as racemates and racemic mixtures, single enantiomers, individual diastereomers and diastereomeric mixtures. All such isomeric forms of these compounds are expressly included within the scope. Unless otherwise indicated when a compound is named or depicted by a structure without specifying the stereochemistry and has one or more chiral centers, it is understood to represent all possible stereoisomers of the compound. The compounds provided herewith may also contain linkages (e.g., carbon-carbon bonds, phosphorus-oxygen bonds, or phosphorus-sulfur bonds) or substituents that can restrict bond rotation, e.g. restriction resulting from the presence of a ring or double bond.

### HBV Infection

The present invention relates to methods for treating a subject infected with HBV through administration of Formula (I) or the prodrug Formula (II), or a pharmaceutically acceptable salt thereof. HBV is an enveloped DNA virus classified as the species type Orthohepadnavirus, which contains three other species, the woodchuck hepatitis virus (WHV), the woolly monkey hepatitis B virus, and the ground squirrel hepatitis virus. The virus is characterized into four major serotypes (adr, adw, ayr, ayw) based upon the antigenic epitopes present on the viral envelope proteins and eight genotypes (genotypes A-H) according to the overall nucleotide sequence of the viral genome. In some embodiments, the methods described herein are used to treat a subject suffering from any known form of HBV infection (e.g., any genotype or serotype of HBV or a combination thereof).

While effective antiviral therapy exists for chronic HBV infection, the infected patient often requires prolonged or lifelong therapy. There are five nucleoside and nucleotide analogs commercially available for treatment of HBV (e.g., lamivudine, adefovir, tenofovir, telbivudine, and entecavir), but their use is limited due to the emergence of drug resistant variants during treatment, the risk of relapse upon treatment discontinuation, and unwarranted side effects. A major challenge of current HBV therapy is to clear the viral, covalently closed circular (ccc) DNA molecule within the nucleus of hepatocytes, which is representing the HBV genome and that is used by the virus as a template for synthesizing the pre-genomic RNA needed for replication. Drugs that target directly HBV cccDNA are currently not available for use in patients. Indirect evidence for treatment-induced reduction of this viral molecule includes the loss of HBV surface antigen (HBsAg), but even after 5 years of therapy with currently available nucleoside and nucleotide analogs, clearance of HBsAg and subsequent seroconversion to antibodies against HBsAg (anti-HBs) are rare events and only achieved in less than 10% of treated patients. In addition, successfully treated patients with antiviral response still exhibit significant levels of HBV-induced liver disease above those in uninfected individuals.

Interferons (e.g., IFN-α) and alternate formulations (e.g., pegylated IFN-α) are also licensed for therapy of HBV but their use is limited because of unwanted side effects. In addition, variability in treatment response of chronic HBV carriers is still a common observation with IFN-α, administered alone or in combination with nucleoside and/or nucleotide analogs, but overall approximately 25-30% of such patients achieve a sustained antiviral response after 2 years of drug administration, including the loss of HBsAg. Therefore, one goal of current HBV therapy is to develop new antiviral compounds that can mimic the benefits of IFN-α therapy but induce suppression of HBV replication, clearance of HBsAg, and seroconversion to anti-HBs in more than one-third of treated patients.

### HBV and Drug-Resistance

The present invention further relates to methods for treating a subject infected with a resistant variant of HBV through administration of Formula (I) or the prodrug Formula (II), or a pharmaceutically acceptable salt thereof. The HBV genome is comprised of circular, partially duplexed DNA that encodes four known genes termed C, X, P, and S. Multiple open-reading frames and/or proteolytic processing of the resulting gene products give rise to HBV proteins including the surface antigen (HBsAg), core protein (HBcAg or C), E antigen (HBeAg or pre-C), long surface protein (L), middle surface protein (M), polymerase (P), and X protein.

Naturally, HBV exists within a host as a population of genetically distinct but closely related virions, due in part to the low fidelity of the viral reverse transcriptase, or polymerase P (Locarnini, S. and Warner, N. Antivir Ther (2007) 12 Suppl 3:H15-H23; Coleman, P. F. Ener Infect Dis (2006) 12:198-203). Treatment with standard anti-HBV agents may eliminate some or nearly all of the HBV population, and readily select out a small and possibly undetectable HBV population that is resistant to said treatment and capable of developing into a chronic infection. Drug-resistance is further affected by other factors including, but not limited to, the viral mutation frequency, the mutability of the antiviral target site, the particular selective pressure applied by the antiviral agent, and the overall replication fitness of the resistant strain (Locarnini, S. and Warner, N. Antivir Ther (2007) 12 Suppl 3:H15-H23). HBV strains resistant to a number of standard anti-HBV agents have been reported, including lamivudine and adefovir dipivoxil.

Without being bound by any particular theory, a drug-resistant strain of HBV may comprise an amino acid mutation (e.g., an amino acid substitution, addition, or deletion) in a particular protein that may result in a structural change, e.g., a conformational or steric change, that affects the ability of an anti-HBV agent from binding to said protein and modulating its activity, e.g., through inhibiting HBV replication or pathogenicity. Particularly, amino acids in and around the active site or close to the inhibitor binding site may be mutated such that the activity of the protein is impacted. In some instances, the amino acid mutation (e.g., an amino acid substitution, addition, or deletion) may be conservative and may not substantially impact the structure or function of a protein. For example, in certain cases, the substitution of a serine residue with a threonine residue may not significantly impact the function of a protein. In other cases, the amino acid mutation may be more dramatic, such as the substitution of a charged amino acid (e.g., aspartic acid or lysine) with a large, nonpolar amino acid (e.g., phenylalanine or tryptophan) and therefore may have a substantial impact on protein function. The nature of the mutations that render the HBV strain resistant to one or more antiviral agents can be readily identified using standard sequencing techniques, e.g., deep sequencing techniques, that are well known in the art.

In some embodiments, the drug-resistant HBV strain comprises a variant or mutant form of the HBsAg, HBcAg, HBeAg, L, M, P, or X proteins. In some embodiments, the drug-resistant HBV strain comprises a variant or mutant form of the HBsAg, HBcAg, HBeAg, L, M, P, or X proteins compared with the accepted consensus sequence of said proteins.

In some embodiments, the drug-resistant HBV variant comprises an amino acid mutation (e.g., an amino acid substitution, addition, or deletion) in the sequence of the HBsAg, HBcAg, HBeAg, L, M, P, or X proteins, e.g., as compared to a reference or consensus sequence. In some embodiments, the amino acid mutation in the sequence of the HBsAg, HBcAg, HBeAg, L, M, P, or X proteins is an amino acid substitution. In some embodiments, the amino acid mutation in the sequence of the HBsAg, HBcAg, HBeAg, L, M, P, or X proteins is an amino acid addition. In some embodiments, the amino acid mutation in the sequence of the HBsAg, HBcAg, HBeAg, L, M, P, or X proteins is an amino acid deletion.

In some embodiments, the amino acid mutation in the sequence of the HBsAg, HBcAg, HBeAg, L, M, P, or X proteins comprises an amino acid substitution of the wild type amino acid residue present at a particular position in the sequence with another amino acid selected from one of the naturally occurring amino acids. In some embodiments, the amino acid mutation in the sequence of the HBsAg, HBcAg, HBeAg, L, M, P, or X proteins comprises an amino acid substitution of the wild type amino acid residue present at a particular position in the sequence with an alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, or valine residue.

In some embodiments, the amino acid mutation in the sequence of the HBsAg, HBcAg, HBeAg, L, M, P, or X proteins comprises an amino acid addition to the wild type sequence at a particular position of an amino acid selected from one of the naturally occurring amino acids. In some embodiments, the amino acid mutation in the sequence of the HBsAg, HBcAg, HBeAg, L, M, P, or X proteins comprises an amino acid addition to the wild type sequence at a particular position selected from an alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine residue.

In some embodiments, the amino acid mutation in the sequence of the HBsAg, HBcAg, HBeAg, L, M, P, or X proteins comprises an amino acid deletion at a particular position of the wild type sequence. In some embodiments, the amino acid deletion in the sequence of the HBsAg, HBcAg, HBeAg, L, M, P, or X proteins comprises an amino acid deletion of an alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, or valine residue.

In some embodiments, the drug-resistant HBV variant comprises an amino acid mutation (e.g., an amino acid substitution, addition, or deletion) in the sequence of the HBsAg protein, e.g., as compared to a reference or consensus sequence. In some embodiments, the amino acid mutation (e.g., an amino acid substitution, addition, or deletion) in the HBsAg protein sequence comprises a mutation from amino acid position 100 to amino acid position 200, e.g., as compared to a reference or consensus sequence. In some embodiments, the amino acid mutation (e.g., an amino acid substitution, addition, or deletion) in the HBsAg protein sequence comprises a mutation from amino acid position 105 to amino acid position 160 e.g., as compared to a reference or consensus sequence. In some embodiments, the amino acid mutation (e.g., an amino acid substitution, addition, or deletion) in the HBsAg protein sequence comprises a mutation from amino acid position 115 to amino acid position 155, e.g., as compared to a reference or consensus sequence. In some embodiments, the amino acid mutation (e.g., an amino acid substitution, addition, or deletion) in the HBsAg protein sequence comprises a mutation at amino acid positions 115, 118, 120, 123, 126, 129, 131, 133, 134, 142, 143, 144, 145, or 154, e.g., as compared to a reference or consensus sequence. In some embodiments, the amino acid mutation (e.g., an amino acid substitution, addition, or deletion) in the HBsAg protein sequence comprises a T115N, T118V, P120L, P120Q, T126S, Q129H, T131K, M133I, M133L, F134N, F134H, P142L, P142S, T143L, D144A, D144V, G145R, or S154P mutation.

In some embodiments, the amino acid mutation (e.g., an amino acid substitution, addition, or deletion) in the HBsAg protein sequence comprises a mutation from amino acid position 150 to amino acid position 200 e.g., as compared to a reference or consensus sequence. In some embodiments, the amino acid mutation (e.g., an amino acid substitution, addition, or deletion) in the HBsAg protein sequence comprises a mutation from amino acid position 160 to amino acid position 200, e.g., as compared to a reference or consensus sequence. In some embodiments, the amino acid mutation (e.g., an amino acid substitution, addition, or deletion) in the HBsAg protein sequence comprises a mutation at amino acid positions 161, 172, 173, 175, 176, 193, 194, or 196, e.g., as compared to a reference or consensus sequence. In some embodiments, the amino acid mutation (e.g., an amino acid substitution, addition, or deletion) in the HBsAg protein sequence comprises a F161H, F161L, W172L, W172*, L173F, L175F, L176V, L176*, S193L, V194F, V194S, I195M, W196L, W196S, or W196* mutation, e.g., as compared to a reference or consensus sequence, wherein “*” represents a stop codon.

In some embodiments, the drug-resistant HBV variant comprises an amino acid mutation (e.g., an amino acid substitution, addition, or deletion) in the sequence of the P protein, e.g., as compared to a reference or consensus sequence. In some embodiments, the amino acid mutation (e.g., an amino acid substitution, addition, or deletion) in the P protein sequence comprises a mutation from amino acid position 60 to amino acid position 275, e.g., as compared to a reference or consensus sequence. In some embodiments, the amino acid mutation (e.g., an amino acid substitution, addition, or deletion) in the P protein sequence comprises a mutation from amino acid position 80 to amino acid position 250, e.g., as compared to a reference or consensus sequence. In some embodiments, the amino acid mutation (e.g., an amino acid substitution, addition, or deletion) in the P protein sequence comprises a mutation at amino acid positions 80, 169, 173, 180, 181, 184, 169, 202, 204, 215, 233, 236, or 250, e.g., as compared to a reference or consensus sequence. In some embodiments, the amino acid mutation (e.g., an amino acid substitution, addition, or deletion) in the P protein sequence comprises a mutation at amino acid positions 180, 204, or 236, e.g., as compared to a reference or consensus sequence.

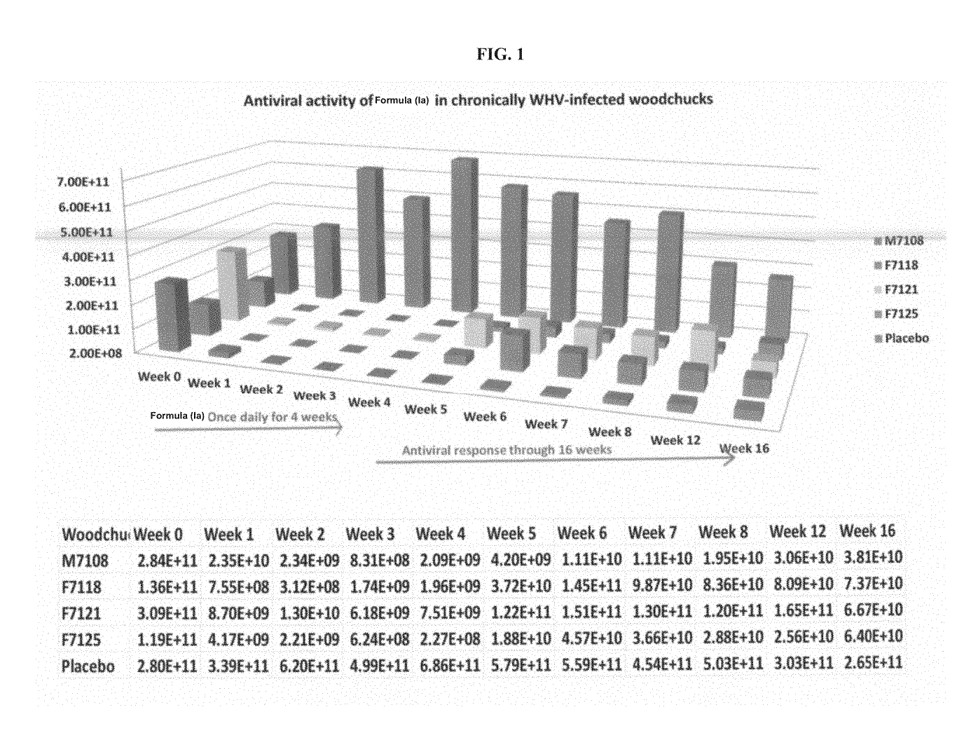

In some embodiments, the amino acid mutation (e.g., an amino acid substitution, addition, or deletion) in the P protein sequence comprises a N169T, I169T, V173L, L180M, A181T, A181V, T184A, T184C, T184G, T184I, T184L, T184M, T184S, S202C, S202G, S202I, M204I, M204V, N236T, M250I, or M250V mutation. In some embodiments, the amino acid mutation (e.g., an amino acid substitution, addition, or deletion) in the P protein sequence comprises a L180M, M204I, M204V, or N236T mutation, e.g., as depicted in FIG. 1. In some embodiments, the amino acid mutation (e.g., an amino acid substitution, addition, or deletion) in the P protein sequence comprises an L180M mutation, e.g., as depicted in FIG. 1. In some embodiments, the amino acid mutation (e.g., an amino acid substitution, addition, or deletion) in the P protein sequence comprises an M204I mutation, e.g., as depicted in FIG. 1. In some embodiments, the amino acid mutation (e.g., an amino acid substitution, addition, or deletion) in the P protein sequence comprises an M204V mutation, e.g., as depicted in FIG. 1. In some embodiments, the amino acid mutation (e.g., an amino acid substitution, addition, or deletion) in the P protein sequence comprises an L180M and an M204V mutation, e.g., as depicted in FIG. 1. In some embodiments, the amino acid mutation (e.g., an amino acid substitution, addition, or deletion) in the P protein sequence comprises an N236T mutation, e.g., as depicted in FIG. 1.

In some embodiments, the amino acid mutation (e.g., an amino acid substitution, addition, or deletion) in the P protein sequence comprises an L180M, M204V/I, I169T, V173L, and M250V mutation. In some embodiments, the amino acid mutation (e.g., an amino acid substitution, addition, or deletion) in the P protein sequence comprises an L180M, M204V/I, T184G, and S202I/G mutation.

In some embodiments, the drug-resistant HBV variant comprises an amino acid mutation (e.g., an amino acid substitution, addition, or deletion) in the sequences of both the HBsAg and P proteins, e.g., as compared to reference or consensus sequences.

In some embodiments, the drug-resistant HBV variant comprises an amino acid mutation (e.g., an amino acid substitution, addition, or deletion) in the sequence of the HBcAg protein. In some embodiments, the drug-resistant HBV variant comprises an amino acid mutation (e.g., an amino acid substitution, addition, or deletion) in the sequence of the HBeAg protein. In some embodiments, the drug-resistant HBV variant comprises an amino acid mutation (e.g., an amino acid substitution, addition, or deletion) in the sequence of the L protein. In some embodiments, the drug-resistant HBV variant comprises an amino acid mutation (e.g., an amino acid substitution, addition, or deletion) in the sequence of the M protein. In some embodiments, the drug-resistant HBV variant comprises an amino acid mutation (e.g., an amino acid substitution, addition, or deletion) in the sequence of the X protein.

In some embodiments, the drug-resistant HBV variant comprises more than one amino acid mutation (e.g., an amino acid substitution, addition, or deletion) in the sequence of the HBsAg, HBcAg, HBeAg, L, M, P, or X proteins. In some embodiments, the drug-resistant HBV variant comprises at least 2, at least 3, at least 4, at least 5, at least 6, at least 7, at least 8, at least 9, at least 10, at least 12, at least 15, at least 20, at least 25, at least 30, at least 35, at least 40, at least 45, at least 50 or more amino acid mutations (e.g., an amino acid substitution, addition, or deletion) in the sequence of the HBsAg, HBcAg, HBeAg, L, M, P, or X proteins. In some embodiments, the drug-resistant HBV variant comprises an amino acid mutation (e.g., an amino acid substitution, addition, or deletion) in the sequence of the only one of the HBsAg, HBcAg, HBeAg, L, M, P, or X proteins. In some embodiments, the drug-resistant HBV variant comprises an amino acid mutation (e.g., an amino acid substitution, addition, or deletion) in the sequence of at least 2, at least 3, at least 4, at least 5, at least 6, at least 7, or all of the HBsAg, HBcAg, HBeAg, L, M, P, or X proteins. In some embodiments, the drug-resistant HBV variant may comprise an amino acid mutation in a protein other than the HBsAg, HBcAg, HBeAg, L, M, P, or X proteins.

In the above embodiments, the amino acid mutation (e.g., an amino acid substitution, addition, or deletion) in the drug-resistant HBV strain comprises a variant or mutant form of the HBsAg, HBcAg, HBeAg, L, M, P, or X proteins compared with the accepted consensus sequence or a reference sequence of said proteins.

In some embodiments, the drug-resistant variant of HBV is resistant to an anti-HBV agent other than a compound other than Formula (I) or Formula (II) or a pharmaceutically acceptable salt thereof. In some embodiments, the drug-resistant variant of HBV is resistant to an interferon, a nucleoside analog, a non-nucleoside antiviral, a non-interferon immune enhancer, or a direct-acting antiviral, each of which does not include a compound of Formula (I) or Formula (II) or a pharmaceutically acceptable salt thereof. In some embodiments, the drug-resistant variant of HBV is resistant to interferon (e.g., peg-interferon), ribavirin, lamivudine, adefovir dipivoxil, entecavir, telbivudine, clevudine, tenofovir, tenofovir alafenamide, besifovir, or AGX-1009 or a combination thereof. In some embodiments, the drug-resistant variant of HBV is resistant to an interferon (e.g., peg-interferon). In some embodiments, the drug-resistant variant of HBV is resistant to ribavirin. In some embodiments, the drug-resistant variant of HBV is resistant to an interferon (e.g., peg-interferon) and ribavirin. In some embodiments, the drug-resistant variant of HBV is resistant to lamivudine, adefovir dipivoxil, entecavir, telbivudine, clevudine, tenofovir, tenofovir alafenamide, besifovir. In some embodiments, the drug-resistant variant of HBV is resistant to lamivudine, adefovir dipivoxil, or entecavir. In some embodiments, the drug-resistant HBV variant is resistant to more than one anti-HBV agent. In some embodiments, the IC₅₀ of an anti-HBV agent other than a compound of Formula (I) or Formula (II) in a sample infected with a drug-resistant variant of HBV is higher than the IC₅₀ of a compound of Formula (I) or Formula (II) or a pharmaceutically acceptable salt thereof. In some embodiments, the IC₅₀ of an anti-HBV agent other than a compound of Formula (I) or Formula (II) is more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 95% higher than the IC₅₀ of a compound of Formula (I) or Formula (II) or a pharmaceutically acceptable salt thereof. In some embodiments, the IC₅₀ of an anti-HBV agent other than a compound of Formula (I) or Formula (II) is more than about 1.5 fold, about 2 fold, about 2.5 fold, about 3 fold, about 3.5 fold, about 4 fold, about 4.5 fold, about 5 fold, about 10 fold, about 15 fold, about 20 fold, about 25 fold, about 35 fold, or about 50 fold higher than the IC₅₀ of a compound of Formula (I) or Formula (II) or a pharmaceutically acceptable salt thereof.

### HDV Infection

The present invention further relates to methods for treating a subject suffering from a HDV (e.g., a co-infection with HBV and HDV) through administration of Formula (I) or the prodrug Formula (II), or a pharmaceutically acceptable salt thereof, in combination with entcavir or tenofovir (e.g., tenofovir dipivoxil, tenofovir alafenamide). Hepatitis D (HDV) is small circular enveloped RNA virus and is the sole member of the Delta virus genus. The circular genome comprises 1,700 nucleotides and encodes only a single protein, the HDV surface antigen (HDAg). As HDV does not produce envelope proteins, the virus is unable to generate progeny viral particles on its own and requires the co-infection of the host cell with HBV to complete the viral replication. The viral replication machinery utilizes the HBV-derived envelope proteins to produce and package mature virions to propagate virulence. HDV is characterized into eight major serotypes (HDV-1, HDV-2, HDV-3, HDV-4, HDV-5, HDV-6, HDV-7, and HDV-8) according to the overall nucleotide sequence of the viral genome. In some embodiments, the methods described herein are used to treat a subject suffering from a co-infection of HBV and HDV in combination with entecavir or tenofovir (e.g., tenofovir dipivoxil, tenofovir alafenamide). The HBV and HDV may comprise any genotype of HBV or HDV, or a combination of varying gentotypes of HBV and HDV.

### Pharmaceutical Compositions

The present invention features methods for treating a subject infected with HBV, the methods comprising administering a compound of Formula (I) (e.g., Formula (Ia), Formula (Ib), or Formula (Ic)) or a prodrug thereof (e.g., a compound of Formula (II), e.g., Formula (IIa), Formula (IIb), or Formula (IIc)), or a pharmaceutically acceptable salt thereof. An HBV infection may comprise infection with one or more resistant strains of HBV. The present invention further includes methods for treating a subject infected with HBV or HDV (e.g., a co-infection of HBV and HDV), the methods comprising administering a compound of Formula (I) (e.g., Formula (Ia), Formula (Ib), or Formula (Ic)) or a prodrug thereof (e.g., a compound of Formula (II), e.g., Formula (IIa), Formula (IIb), or Formula (IIc)), or a pharmaceutically acceptable salt thereof, in combination with entecavir or tenofovir (e.g., tenofovir dipivoxil, tenofovir alafenamide).

While it is possible for the compound of the present invention (e.g., a compound of Formula (I), or a prodrug thereof (e.g., a compound of Formula (II)) to be administered alone, it is preferable to administer said compound as a pharmaceutical composition or formulation, where the compounds are combined with one or more pharmaceutically acceptable diluents, excipients or carriers. The compounds according to the invention may be formulated for administration in any convenient way for use in human or veterinary medicine. In certain embodiments, the compounds included in the pharmaceutical preparation may be active itself, or may be a prodrug, e.g., capable of being converted to an active compound in a physiological setting (e.g., a compound of Formula (II), e.g., Formula (IIa), Formula (IIb), or Formula (IIc)). Regardless of the route of administration selected, the compounds of the present invention, which may be used in a suitable hydrated form, and/or the pharmaceutical compositions of the present invention, are formulated into a pharmaceutically acceptable dosage form such as described below or by other conventional methods known to those of skill in the art.

The amount and concentration of compounds of the present invention (e.g., a compound of Formula (I), or a prodrug thereof (e.g., a compound of Formula (II)) in the pharmaceutical compositions, as well as the quantity of the pharmaceutical composition administered to a subject, can be selected based on clinically relevant factors, such as medically relevant characteristics of the subject (e.g., age, weight, gender, other medical conditions, and the like), the solubility of compounds in the pharmaceutical compositions, the potency and activity of the compounds, and the manner of administration of the pharmaceutical compositions. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

Thus, another aspect of the present invention provides pharmaceutically acceptable compositions comprising a therapeutically effective amount or prophylacticaly effective amount of a compound described herein (e.g., a compound of Formula (I) or a prodrug thereof (e.g., a compound of Formula (II)), formulated together with one or more pharmaceutically acceptable carriers (additives) and/or diluents. As described in detail below, the pharmaceutical compositions of the present invention may be specially formulated for administration in solid or liquid form, including those adapted for oral or parenteral administration, for example, by oral dosage, or by subcutaneous, intramuscular or intravenous injection as, for example, a sterile solution or suspension. However, in certain embodiments the subject compounds may be simply dissolved or suspended in sterile water. In certain embodiments, the pharmaceutical preparation is non-pyrogenic, i.e., does not elevate the body temperature of a patient.

The phrases “systemic administration,” “administered systemically,” “peripheral administration” and “administered peripherally” as used herein mean the administration of the compound other than directly into the central nervous system, such that it enters the patient's system and, thus, is subject to metabolism and other like processes, for example, subcutaneous administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, stabilizing agent, excipient, solvent or encapsulating material, involved in carrying or transporting the subject antagonists from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include, but are not limited to: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) ascorbic acid; (17) pyrogen-free water; (18) isotonic saline; (19) Ringer's solution; (20) ethyl alcohol; (21) phosphate buffer solutions; (22) cyclodextrins such as Captisol®; and (23) other non-toxic compatible substances such as antioxidants and antimicrobial agents employed in pharmaceutical formulations.

As set out above, certain embodiments of the compounds described herein may contain a basic functional group, such as an amine, and are thus capable of forming pharmaceutically acceptable salts with pharmaceutically acceptable acids. The term “pharmaceutically acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (see, for example, Berge et al. (1977) “Pharmaceutical Salts”, J. Pharm. Sci. 66:1-19).

In other cases, the compounds of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of the compound of the present invention (e.g., a compound of Formula (I), or a prodrug thereof (e.g., a compound of Formula (II)). These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

Wetting agents, emulsifiers, and lubricants, such as sodium lauryl sulfate and magnesium stearate, as well as coloring agents, release agents, coating agents, sweetening, flavoring and perfuming agents, preservatives and antioxidants can also be present in the compositions. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutically acceptable carriers, as well as wetting agents, emulsifiers, lubricants, coloring agents, release agents, coating agents, sweetening, flavoring agents, perfuming agents, preservatives, antioxidants, and other additional components may be present in an amount between about 0.001% and 99% of the composition described herein. For example, said pharmaceutically acceptable carriers, as well as wetting agents, emulsifiers, lubricants, coloring agents, release agents, coating agents, sweetening, flavoring agents, perfuming agents, preservatives, antioxidants, and other additional components may be present from about 0.005%, about 0.01%, about 0.05%, about 0.1%, about 0.25%, about 0.5%, about 0.75%, about 1%, about 1.5%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 85%, about 90%, about 95%, or about 99% of the composition described herein.

Pharmaceutical compositions of the present invention may be in a form suitable for oral administration, e.g., a liquid or solid oral dosage form. In some embodiments, the liquid dosage form comprises a suspension, a solution, a linctus, an emulsion, a drink, an elixir, or a syrup. In some embodiments, the solid dosage form comprises a capsule, tablet, powder, dragée, or powder. The pharmaceutical composition may be in unit dosage forms suitable for single administration of precise dosages. Pharmaceutical compositions may comprise, in addition to the compound described herein (e.g., a compound of Formula (I) or a prodrug thereof (e.g., a compound of Formula (II)) or a pharmaceutically acceptable salt thereof, a pharmaceutically acceptable carrier, and may optionally further comprise one or more pharmaceutically acceptable excipients, such as, for example, stabilizers (e.g., a binder, e.g., polymer, e.g., a precipitation inhibitor, diluents, binders, and lubricants.

In some embodiments, the composition described herein comprises a liquid dosage form for oral administration, e.g., a solution or suspension. In other embodiments, the composition described herein comprises a solid dosage form for oral administration capable of being directly compressed into a tablet. In addition, said tablet may include other medicinal or pharmaceutical agents, carriers, and or adjuvants. Exemplary pharmaceutical compositions include compressed tablets (e.g., directly compressed tablets), e.g., comprising a compound of the present invention (e.g., a compound of Formula (I) or a prodrug thereof (e.g., a compound of Formula (II)) or a pharmaceutically acceptable salt thereof.

Formulations of the present invention include those suitable for parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about 99 percent of active ingredient, preferably from about 5 percent to about 70 percent, most preferably from about 10 percent to about 30 percent. Pharmaceutical compositions of this invention suitable for parenteral administration comprise compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

In some embodiments, a compound of the present invention (e.g., a compound of Formula (I) or a prodrug thereof (e.g., a compound of Formula (II)) is provided as a composition in combination with an additional agent (e.g., entecavir or tenofovir). For example, a compound of may be prepared as a fixed dose composition in combination with entecavir or tenofovir (e.g., tenofovir dipivoxil or tenofovir alafenamide). The fixed dose composition may be formulated for oral administration, e.g., as a solid dosage form or a liquid dosage form. In some embodiments, the liquid dosage form comprises a suspension, a solution, a linctus, an emulsion, a drink, an elixir, or a syrup. In some embodiments, the solid dosage form comprises a capsule, tablet, dragée, or powder.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a compound of the present invention (e.g., a compound of Formula (I) or a prodrug thereof (e.g., a compound of Formula (II)), it may be desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered form of the compound of the present invention is accomplished by dissolving or suspending compound in an oil vehicle.

In some embodiments, it may be advantageous to administer the compound of the present invention (e.g., a compound of Formula (I) or a prodrug thereof (e.g., a compound of Formula (II)) in a sustained fashion. It will be appreciated that any formulation that provides a sustained absorption profile may be used. In certain embodiments, sustained absorption may be achieved by combining a compound of the present invention with other pharmaceutically acceptable ingredients, diluents, or carriers that slow its release properties into systemic circulation.

### Routes of Administration

The compounds and compositions used in the methods described herein may be administered to a subject in a variety of forms depending on the selected route of administration, as will be understood by those skilled in the art. Exemplary routes of administration of the compositions used in the methods described herein include topical, enteral, or parenteral applications. Topical applications include but are not limited to epicutaneous, inhalation, enema, eye drops, ear drops, and applications through mucous membranes in the body. Enteral applications include oral administration, rectal administration, vaginal administration, and gastric feeding tubes. Parenteral administration includes intravenous, intraarterial, intracapsular, intraorbital, intracardiac, intradermal, transtracheal, subcuticular, intraarticular, subcapsular, subarachnoid, intraspinal, epidural, intrastemal, intraperitoneal, subcutaneous, intramuscular, transepithelial, nasal, intrapulmonary, intrathecal, rectal, and topical modes of administration. Parenteral administration may be by continuous infusion over a selected period of time. In certain embodiments of the invention, the compositions described herein comprising a compound of Formula (I) or a prodrug thereof (e.g., a compound of Formula (II)) is administered orally. In other embodiments of the invention, the compositions described herein comprising a compound of Formula (I) or a prodrug thereof (e.g., a compound of Formula (II)) is administered intravenously.

In an embodiment, the compositions described herein comprising a compound of Formula (I) or a prodrug thereof (e.g., a compound of Formula (II)) is administered orally in combination with entecavir or tenofovir (e.g., tenofovir dipivoxil or tenofovir alafenamide). In an embodiment, the compositions described herein comprising a compound of Formula (I) or a prodrug thereof (e.g., a compound of Formula (II)) is administered orally prior to or after oral administration of entecavir or tenofovir (e.g., tenofovir dipivoxil or tenofovir alafenamide).

In other embodiments of the invention, the compositions described herein comprising a compound of Formula (I) or a prodrug thereof (e.g., a compound of Formula (II)) is administered parenterally (e.g., intraperitoneally). In an embodiment, the compositions described herein comprising a compound of Formula (I) or a prodrug thereof (e.g., a compound of Formula (II)) is administered parenterally in combination with entecavir or tenofovir (e.g., tenofovir dipivoxil or tenofovir alafenamide). In an embodiment, the compositions described herein comprising a compound of Formula (I) or a prodrug thereof (e.g., a compound of Formula (II)) is administered parenterally prior to or after oral administration of entecavir or tenofovir (e.g., tenofovir dipivoxil or tenofovir alafenamide).

For intravenous, intraperitoneal, or intrathecal delivery or direct injection, the composition must be sterile and fluid to the extent that the composition is deliverable by syringe. In addition to water, the carrier can be an isotonic buffered saline solution, ethanol, polyol (for example, glycerol, propylene glycol, and liquid polyetheylene glycol, and the like), and suitable mixtures thereof. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

The choice of the route of administration will depend on whether a local or systemic effect is to be achieved. For example, for local effects, the composition can be formulated for topical administration and applied directly where its action is desired. For systemic, long term effects, the composition can be formulated for enteral administration and given via the digestive tract. For systemic, immediate and/or short term effects, the composition can be formulated for parenteral administration and given by routes other than through the digestive tract.

### Dosages

The compositions of the present invention are formulated into acceptable dosage forms by conventional methods known to those of skill in the art. Actual dosage levels of the active ingredients in the compositions of the present invention (e.g., a compound of Formula (I) or a prodrug thereof (e.g., a compound of Formula (II)) may be varied so as to obtain an amount of the active ingredient which is effective to achieve the desired therapeutic response for a particular subject, composition, and mode of administration, without being toxic to the subject. The selected dosage level will depend upon a variety of pharmacokinetic factors including the activity of the particular compositions of the present invention employed, the route of administration, the time of administration, the rate of absorption of the particular agent being employed, the duration of the treatment, other drugs, substances, and/or materials used in combination with the particular compositions employed, the age, sex, weight, condition, general health and prior medical history of the subject being treated, and like factors well known in the medical arts. A physician or veterinarian having ordinary skill in the art can readily determine and prescribe the effective amount of the composition required. For example, the physician or veterinarian can start doses of the substances of the invention employed in the composition at levels lower than that required in order to achieve the desired therapeutic effect and gradually increase the dosage until the desired effect is achieved. In general, a suitable daily dose of a composition of the invention will be that amount of the substance which is the lowest dose effective to produce a therapeutic effect. Such an effective dose will generally depend upon the factors described above. Preferably, the effective daily dose of a therapeutic composition may be administered as two, three, four, five, six or more sub-doses administered separately at appropriate intervals throughout the day, optionally, in unit dosage forms.

Preferred therapeutic dosage levels are between about 0.1 mg/kg to about 1000 mg/kg (e.g., about 0.2 mg/kg, 0.5 mg/kg, 1.0 mg/kg, 1.5 mg/kg, 2 mg/kg, 3 mg/kg, 4 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 350 mg/kg, 400 mg/kg, 450 mg/kg, 500 mg/kg, 600 mg/kg, 700 mg/kg, 800 mg/kg, 900 mg/kg, or 1000 mg/kg) of the composition per day administered (e.g., orally or intraperitoneally) to a subject afflicted with the disorders described herein (e.g., HBV infection). Preferred prophylactic dosage levels are between about 0.1 mg/kg to about 1000 mg/kg (e.g., about 0.2 mg/kg, 0.5 mg/kg, 1.0 mg/kg, 1.5 mg/kg, 2 mg/kg, 3 mg/kg, 4 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 350 mg/kg, 400 mg/kg, 450 mg/kg, 500 mg/kg, 600 mg/kg, 700 mg/kg, 800 mg/kg, 900 mg/kg, or 1000 mg/kg) of the composition per day administered (e.g., orally or intraperitoneally) to a subject. The dose may also be titrated (e.g., the dose may be escalated gradually until signs of toxicity appear, such as headache, diarrhea, or nausea).

The frequency of treatment may also vary. The subject can be treated one or more times per day (e.g., once, twice, three, four or more times) or every so-many hours (e.g., about every 2, 4, 6, 8, 12, or 24 hours). The composition can be administered 1 or 2 times per 24 hours. The time course of treatment may be of varying duration, e.g., for two, three, four, five, six, seven, eight, nine, ten, or more days, two weeks, 1 month, 2 months, 4 months, 6 months, 8 months, 10 months, or more than one year. For example, the treatment can be twice a day for three days, twice a day for seven days, twice a day for ten days. Treatment cycles can be repeated at intervals, for example weekly, bimonthly or monthly, which are separated by periods in which no treatment is given. The treatment can be a single treatment or can last as long as the life span of the subject (e.g., many years).

### Patient Selection and Monitoring

The methods of the present invention described herein entail administration of a compound of Formula (I) or a prodrug thereof (e.g., a compound of Formula (II)) or a pharmaceutically acceptable salt thereof for the treatment of HBV infection (e.g., a resistant HBV infection). The methods described herein further entail administration of a compound of Formula (I) or a prodrug thereof (e.g., a compound of Formula (II)) or a pharmaceutically acceptable salt thereof for the treatment of a subject infected with HBV or HDV (e.g., a co-infection of HBV and HDV) in combination with entecavir or tenofovir (e.g., tenofovir dipivoxil or tenofovir alafenamide). Accordingly, a patient and/or subject can be selected for treatment using a compound of Formula (I) or a prodrug thereof (e.g., a compound of Formula (II)) or a pharmaceutically acceptable salt thereof by first evaluating the patient and/or subject to determine whether the subject is infected with HBV or HDV and determination of the serotypic and genotypic classification of the virus. A subject can be evaluated as infected with HBV or HDV using methods known in the art. The subject can also be monitored, for example, subsequent to administration of a compound described herein (e.g., a compound of Formula (I) or a prodrug thereof (e.g., a compound of Formula (II)) or a pharmaceutically acceptable salt thereof.

In some embodiments, the subject is a mammal. In some embodiments, the subject is a human. In some embodiments, the subject is an adult. In some embodiments, the subject is suffering from an acute form of HBV infection. In some embodiments, the subject is suffering from a chronic form of HBV infection. In some embodiments, the subject has been diagnosed with hepatitis B (e.g., acute or chronic hepatitis B).

In some embodiments, the genotype of the HBV infection is known. In some embodiments, the subject is infected with HBV genotype A (e.g., HBV-A1-7), HBV genotype B (e.g., HBV-B2-5), HBV genotype C (e.g., HBV-C1-16), HBV genotype D (e.g., HBV-D1-7), HBV genotype E, HBV genotype F (e.g., HBV-F1-4), HBV genotype G, HBV genotype H, HBV genotype I, or HBV genotype J. In some embodiments, the subject is infected with HBV genotype A (e.g., HBV-A1-7), HBV genotype B (e.g., HBV-B2-5), HBV genotype C (e.g., HBV-C1-16), HBV genotype D (e.g., HBV-D1-7), HBV genotype E, HBV genotype F (e.g., HBV-F1-4), HBV genotype G, or HBV genotype H. In some embodiments, the subject is infected with HBV genotype A (e.g., HBV-A1-7). In some embodiments, the subject is infected with HBV genotype B (e.g., HBV-B2-5). In some embodiments, the subject is infected with HBV genotype C (e.g., HBV-C1-16). In some embodiments, the subject is infected with HBV genotype D (e.g., HBV-D1-7). In some embodiments, the subject is infected with HBV genotype E. In some embodiments, the subject is infected with HBV genotype F (e.g., HBV-F1-4). In some embodiments, the subject is infected with HBV genotype G. In some embodiments, the subject is infected with HBV genotype H. In some embodiments, the subject is infected with HBV genotype I. In some embodiments, the subject is infected with HBV genotype J.

In some embodiments, the drug-resistant strain of HBV comprises HBV genotype A, (e.g., HBV-A1-7), HBV genotype B (e.g., HBV-B2-5), HBV genotype C (e.g., HBV-C1-16), HBV genotype D (e.g., HBV-D1-7), HBV genotype E, HBV genotype F (e.g., HBV-F1-4), HBV genotype G, HBV genotype H, HBV genotype I, or HBV genotype J.

In some embodiments, the subject is a non-human mammal. In some embodiments, the subject is a woodchuck, e.g., the eastern woodchuck. The eastern woodchuck (Marmota monax) is naturally infected with the woodchuck hepatitis virus (WHV), a hepadnavirus which is genetically closely related to human HBV. Neonatal infection of woodchucks with WHV parallels the main route of human (vertical) transmission for chronic HBV infection and displays a disease course similar to that in HBV-infected patients. Thus, chronic WHV infection in woodchucks is a fully immunocompetent model for studying CHB and HBV-induced HCC, and chronic WHV carriers have extensively been used to evaluate efficacy and safety of current and new HBV therapeutics. The recent comparison of hepatic transcriptional profiles in woodchucks and humans with acute self-limiting and chronic hepadnaviral infections identified important parallels in the antiviral immune responses and demonstrated molecular similarities in HCC induced by WHV and HBV. As these studies have established the translational value of this animal model for CHB, woodchucks with chronic WHV infection may used to evaluate antiviral efficacy, safety and pharmacodynamics associated with treatment.

In some embodiments, the subject is treatment naïve. In some embodiments, the subject has previously been treated for HBV infection. In some embodiments, the subject is suffering from a relapsed HBV infection. In some embodiments, the subject has been treated with an anti-HBV agent other than a compound of Formula (I) or Formula (II) or a pharmaceutically acceptable salt thereof and is suffering from a relapsed HBV infection. In some embodiments, the subject has been treated with an interferon, a nucleoside analog, a non-nucleoside antiviral, or an immune enhancer and is suffering from a relapsed HBV infection. In some embodiments, the subject has been treated with an interferon, e.g., peg-interferon alfa (e.g., peg-interferon alfa-2a or peg-interferon alfa-2b) and is suffering from a relapsed HBV infection. In some embodiments, the subject has been treated with ribavirin and is suffering from a relapsed HBV infection. In some embodiments, the subject has been treated with a nucleoside analog, e.g., lamivudine, adefovir dipivoxil, entecavir, telbivudine, clevudine, ribavarin, tenofovir, tenofovir alafenamide, besifovir, or AGX-1009, and is suffering from a relapsed HBV infection. In some embodiments, the subject has been treated with a non-nucleoside antiviral agent, e.g., NOV-225, BAM 205, Myrcludex B, ARC-520, BAY 41-4109, REP 9AC, Alinia (nitazoxanide), Dd-RNAi, NVR-121 (NVR 3-778), BSBI-25, NVP-018, TKM-HBV, or ALN-HBV, and is suffering from a relapsed HBV infection. In some embodiments, the subject has been treated with a immune enhancer, e.g., zadaxin (thymosin alpha-1), GS-4774, CYT107 (interleukin-7), Dv-601, HBV core antigen vaccine, or GS-9620, and is suffering from a relapsed HBV infection.

In some embodiments, the subject has been diagnosed with cirrhosis of the liver. In some embodiments, the subject has been diagnosed with hepatocellular carcinoma. In some embodiments, the subject has been diagnosed with hepatocellular carcinoma and is awaiting liver transplantation.

In some embodiments, the subject has been further diagnosed with an HIV infection. In some embodiments, the strain of HIV infection is known. In some embodiments, the subject is infected with HIV-1 or HIV-2 (e.g., strain 1 or strain 2).

In some embodiments, the subject is suffering from an HBV infection and an HDV infection (e.g., an HBV and HDV co-infection). In some embodiments, the subject is suffering from a chronic form of HBV or HDV infection. In some embodiments, the subject has been diagnosed with hepatitis B (e.g., acute or chronic hepatitis B, e.g., a resistant variant of acute or chronic hepatitis B). In some embodiments, the subject has been diagnosed with hepatitis D (e.g., acute or chronic hepatitis D). In some embodiments, the genotype of the HDV infection is known. In some embodiments, the subject is treatment naïve. In some embodiments, the subject has received previous treatment for HDV.

### Combination Therapies

In some embodiments, additional therapeutic agents may be administered with compositions of the present invention for the treatment of HBV or any symptom or associated condition thereof. When combination therapy is employed, the additional therapeutic agent(s) can be administered as a separate formulation or may be combined with any of the compositions described herein.

For example, any of the methods described herein may further comprise the administration of a therapeutically effective amount of an additional agent in conjunction with a compound of Formula (I) or Formula (II). In some embodiments, the additional agent is an antiviral agent or an anticancer agent. In some embodiments, the antiviral agent comprises an interferon, a nucleoside analog, a non-nucleoside antiviral, or a non-interferon immune enhancer. In some embodiments, the interferon comprises interferon alfa-2a, interferon alfa-2b, interferon alfa-n1, interferon alfacon-1, or a pegylated interferon (e.g., peginterferon alfa-2a, peginterferon alfa-2b). In some embodiments, the nucleoside analog comprises lamivudine, adefovir dipivoxil, entecavir, telbivudine, clevudine, ribavarin, tenofovir, tenofovir dipivoxil, tenofovir alafenamide, besifovir, or AGX-1009. In some embodiments, the antiviral agent is entecavir. In some embodiments, the antiviral agent is tenofovir (e.g., tenofovir dipivoxil or tenofovir alafenamide). In some embodiments, the antiviral compound comprises NOV-225, BAM 205, Myrcludex B, ARC-520, BAY 41-4109, REP 9AC, Alinia (nitazoxanide), Dd-RNAi, NVR-121 (NVR 3-778), BSBI-25, NVP-018, TKM-HBV, or ALN-HBV. In some embodiments, the non-interferon immune enhancer comprises zadaxin (thymosin alpha-1), GS-4774, CYT107 (interleukin-7), Dv-601, HBV core antigen vaccine, or GS-9620. In some embodiments, the antiviral agent is a capsid inhibitor, an entry inhibitor, a secretion inhibitor, a microRNA, an antisense RNA agent, an RNAi agent, or other agent designed to inhibit viral RNA. In some embodiments, the anticancer agent is selected from methotrexate, 5-fluorouracil, doxorubicin, vincristine, bleomycin, vinblastine, dacarbazine, toposide, cisplatin, epirubicin, and sorafenib tosylate.

Administration in combination can proceed by any technique apparent to those of skill in the art including, for example, separate, sequential, concurrent, and alternating administration. As used herein, “administered in combination” or a combined administration of two or more agents means that two or more agents (e.g., compounds described herein) are administered to a subject at the same time or within an interval such that there is overlap of an effect of each agent on the patient. Preferably they are administered within 15, 10, 5, or 1 minute of one another. In some embodiments, the combination of a compound of Formula (I) or Formula (II) and the additional agent has a synergistic or additive effect. In some embodiments, the term “additive” refers to an outcome wherein when two agents are used in combination, the combination of the agents acts in a manner equal to but not greater than the sum of the individual anti-HBV activities of each agent.

In some embodiments, the terms “synergy” or “synergistic” refer to an outcome wherein when two agents are used in combination, the combination of the agents acts so as to require a lower concentration of each individual agent than the concentration required to be efficacious in the absence of the other agent. In some embodiments, a synergistic effect results in a reduced in a reduced minimum inhibitory concentration of one or both agents, such that the effect is greater than the sum of the effects. A synergistic effect is greater than an additive effect. In some embodiments, the agents in the composition herein may exhibit a synergistic effect, wherein the anti-HBV activity at a particular concentration is greater than at least about 1.25, 1.5, 1.75, 2, 2.5, 3, 4, 5, 10, 12, 15, 20, 25, 50, or 100 times the anti-HBV activity or anti-HDV activity of either agent alone. Preferably the administrations of the agents are spaced sufficiently close together such that a combinatorial (e.g., a synergistic) effect is achieved.

The combinations can have synergistic effect when used to treat a subject suffering from an HBV infection, a resistant HBV infection, or an HBV/HDV co-infection. The agents can be administered simultaneously, for example in a combined unit dose (providing simultaneous delivery of both agents). Alternatively, the agents can be administered at a specified time interval, for example, an interval of minutes, hours, days or weeks. Generally, the agents are concurrently bioavailable, e.g., detectable, in the subject.

In another aspect, the present invention features methods for treating a subject infected with HBV or an HBV/HDV through administration of a compound of compound of Formula (I) or Formula (II) or a pharmaceutically acceptable salt thereof, in combination with entecavir or tenofovir (e.g., tenofovir dipivoxil or tenofovir alafenamide). In some embodiments, the combination of a compound of Formula (II) and entecavir or tenofovir has a synergistic or additive effect. In some embodiments, the term “additive” refers to an outcome wherein when two agents are used in combination, the combination of the agents acts in a manner equal to but not greater than the sum of the individual anti-HBV or anti-HDV activities of each agent.

### EXAMPLES

### Example 1

### Antiviral Activity of Formula (Ia) in the Woodchuck Model of HBV Infection

### Experimental Study

Three groups of five chronic WHV carrier woodchucks were used in this in this study. Formula (Ia) was administered intraperitoneally to two groups for a total of 4 weeks. Groups for drug treatment received Formula (Ia) at either 10 mg/kg/day or 30 mg/kg/day. The third group received physiological saline intraperitoneally and served as placebo-treated controls.

Blood samples for WHV serology, serum HBV DNA, and hematological and biochemical profiles were obtained prior to the start of treatment and taken thereafter according to the study design. The body weight of each subject was recorded when the woodchucks were anesthetized and bled. The recorded body weights of the drug-treated woodchucks were compared to the placebo-treated control woodchucks to assess possible drug toxicity. WHsAg and anti-WHs and anti-WHc antibodies were determined qualitatively using established ELISA protocols. Dilutions of serum were used to ensure the detection of these markers under saturation conditions.

WHV DNA in serum was initially measured by dot blot hybridization and quantified by comparing signals of test specimens and standards of known WHV DNA concentration using a homologous probe. The limit of detection of this assay is 1×10⁷ viral genomic equivalents/mL. Quantitative WHV nucleic acid analyses were also performed using a real-time PCR assay with samples containing serum WHV DNA below the limit of detection by dot blot analysis.

Liver biopsies were obtained under general anesthesia using 16-gauge disposable biopsy needles directed by ultrasound imaging according to the study schedule. The needle was inserted at a site near the ventral midline caudal to the xiphoid cartilage and directed dorsolaterally and cranially into the left lateral lobe of the liver. The biopsy specimen was processed for histopathological examination using standard conditions. Histological sections were stained by immunohisochemistry for WHcAg and for membrane-bound and cytoplasmic WHsAg, and the results of these studies were expressed as percentage of counted cells. Nucleic acid analysis was also carried out. Woodchucks that showed clinical signs of life-threatening illness were euthanized.

### Study Results

WHV DNA and WHV Antigens in the Liver. Liver biopsies were obtained prior to treatment with Formula (Ia), after weeks of treatment with Formula (Ia), and then at 8 weeks and 16 weeks after the study during a follow up period. These liver biopsy specimens demonstrated apparent differences in scores for portal hepatitis and lobular hepatitis/necrosis between woodchucks treated and untreated.

The staining intensity of cytoplasmic WHcAg I liver tissues treated with the higher dose of Formula (Ia) (30 mg/kg) was significantly decreased compared to placebo controls at the end of the treatment period at week 4 (p, 0.05). Individual woodchucks in each of the Formula (Ia)-treated groups showed transient decreases in the hepatic expression of WHcAg and WHsAg during the treatment period. Following drug withdrawal, both viral markers increased in surviving woodchuck and were similar to placebo. Woodchucks with transient declines in the hepatic expression of WHcAg and WHsAg during drug treatment were the same that also had more pronounced transient declines in serum WHsAg and/or serum WHV DNA.

WHV DNA and WHV Antigens in the Serum. Treatment with Formula (Ia) for 4 weeks with a dose of 10 mg/kg/day showed declines in the WHV DNA between 1.7 and 2.7 logs. Serum WHV viremia was significantly reduced compared to placebo-treated controls during treatment at weeks 1, 2, 3, and 4 (p<0.05). Formula (Ia) treatment with a dose of 30 mg/kg/day induced a more rapid effect on serum WHV DNA in all woodchucks in this group with decreases between 2.4 and 4.2 logs. Slow recrudescence of viral load was noted following drug withdrawal. No significant changes in the concentration of serum WHV DNA were observed in the placebo-treated control woodchucks during the study period.

The majority of woodchucks exhibited no apparent changes in serum WHsAg during treatment. No changes in the anti-WHc antibody responses were observed in any of the woodchucks during treatment.

The results of this study suggest that Formula (Ia) monotherapy induces antiviral responses in chronic WHV carrier woodchucks at the treated doses and for the duration of treatment. Consistent reductions in serum WHsAg and in hepatic expression of WHcAg and WHsAg were not observed on a dose-response basis, however individual woodchucks across both dosing groups had significant declines during treatment.

### Example 2

### Antiviral Efficacy and Immunity Associated with Response to Oral Administration of Formula (IIa) in a Woodchuck Model of Chronic Hepatitis B

### Experimental Procedures

Doses of Formula (IIa) with excipient were dry mixed with woodchuck diet powder (Dyets, Bethlehem, Pa.) and the blended drug material subsequently suspended in ultrapure water (high performance liquid chromatography (HPLC) water from J. T. Baker). Formula (IIa) was orally administered to woodchucks within ½ hour after drug preparation.

All woodchucks used in this study were born in captivity and infected at 3 days of age with the cWHV7P2a inoculum containing WHV strain 7-11 (WHV7). cWHV7P2a has the same biological and virological characteristics as the parent cWHV7P2 inoculum as both were derived from cWHV7P1. Chronically infected animals were confirmed positive for serum WHV DNA and WHV surface antigen (WHsAg) and had undetectable antibodies against WHsAg (anti-WHs) at approximately 1 year post-infection. Absence of liver tumors in woodchucks with low gamma-glutamyl transferase (GGT) was confirmed by ultrasonography. Chronic WHV carrier woodchucks were assigned and stratified by gender, body weight, and by pretreatment serum markers (WHV DNA and WHsAg loads and serum GGT and sorbitol-dehydrogenase (SDH) levels) into two groups (n=5 each). Woodchucks were treated daily, orally with either a low (15 mg/kg) or high dose (30 mg/kg) of Formula (IIa) for 12 weeks. For select assays, blood and liver samples from five age-matched chronic WHV carrier woodchucks were included for comparison of basal expression levels of immune response genes in untreated animals with pretreated levels in Formula (IIa) treated animals.

Plasma levels of Formula (Ia) were evaluated at pretreatment (T₀) and then bi-weekly throughout the study at approximately 2 hours post-dose. Woodchuck plasma was analyzed by LC-MS and quantified using isotopically enriched internal standards.

Depending on the serum concentration, WHV DNA was quantified weekly by either dot blot hybridization or real time PCR assay on a 7500 Real Time PCR System instrument (Applied Biosystems, Foster City, Calif.) as described previously (Menne, S. et al. Antimicrob Agents Chemother (2008) 52:3617-3632). Serum levels of WHsAg and anti-WHs were measured weekly by WHV-specific enzyme immunoassays as described previously (Cote, P. J. et al. Viral Immunol (1993) 6:161-169). Dilutions of serum samples were used to ensure detection of these markers under saturating conditions. Both markers were quantified against a standard curve of woodchuck sera with known concentration of WHsAg (lower sensitivity: 50 ng/mL serum) or anti-WHs (lower sensitivity: 100 Standard (Std) U/mL serum), respectively.

Hepatic levels of WHV nucleic acids were determined in liver biopsy samples collected at pretreatment (week-1), during treatment (week 6), at the end of treatment (week 12), and at the end of the study (week 20). WHV RNA was measured quantitatively by Northern blot hybridization as previously described (Peek, S. F. et al Hepatalogy (2001) 33:254-266). WHV DNA replicative intermediates (RI) and WHV covalently-closed circular (ccc) DNA were quantitatively determined by Southern blot hybridization as previously described (Jacob, J. R. et al. Antiviral Res (2004) 63:115-121). Paraffin sections of formalin-fixed liver biopsy samples were stained with hematoxylin and eosin (H&E) and immunostained with an antibody against WHV core antigen (WHcAg) using a 1:350 dilution as previously described (Cote, P. J. et al Hepatalogy (2000) 31:190-200; Peek, S. F. et al Hepatalogy (2001) 33:254-266). Histopathological examination and evaluation of WHV antigen expression was performed using woodchuck-specific criteria as previously described (Peek, S. F. et al Hepatalogy (2001) 33:254-266). From two woodchucks of the HD group, livers were also immunostained with cross-reactive antibodies against RIG-I and NOD2 (Origene Technologies, Rockville, Md.) following the manufacturer's instructions, using a 1:125 or 1:200 dilution, respectively.

Immune responses associated with Formula (IIa) treatment were determined by changes in RNA transcript levels of IFN-α, IFN-β, IFN-γ induced protein 10 (IP-10 or CXCL10), interleukin 6 (IL-6), interferon-induced 17 kDa protein (ISG15), and 2′-5′-oligoadenylate synthetase 1 (OAS1) in blood and liver using PCR techniques. Whole blood was collected into PAXgene blood tubes (Qiagen, Redwood City, Calif.) at pretreatment (week-1 and T₀), during treatment (weeks 6 and 12), and during follow-up (week 18) and stored at −70° C. until use. Total RNA was isolated with on-column DNase I digestion using the PAXgene Blood miRNA Kit (Qiagen). Total RNA was further isolated from liver biopsy samples collected at pretreatment (week-1), during treatment (weeks 6 and 12), and at the end of the study (week 20) using the RNeasy Mini Kit (Qiagen) with on-column DNase I digestion using the RNase-Free DNase Set (Qiagen). Following reverse transcription of mRNA with the High Capacity cDNA Reverse Transcription Kit (Applied Biosystems) using oligo(dT), complementary (c) DNA samples were amplified on a 7500 Real Time PCR System instrument (Applied Biosystems) using TaqMan Gene Expression Master Mix (Applied Biosystems) and woodchuck-specific primers and probes (Supplementary Table 1). Woodchuck 18S rRNA expression was used to normalize target gene expression. Transcription levels of target genes were calculated as a fold-change relative to pretreatment level at week-1 (liver) or at T₀ (blood) using the formula 2^{-ΔΔCt}. Samples from a subset of woodchucks from the LD and HD groups were analyzed for expression changes of genes involved in the RIG-I/NOD2 pathway induced by Formula (IIa), including RIG-I, NOD2, transmembrane protein 173 (TMEM173 or STING), interferon regulatory factor 3 (IRF3), and IRF7.

Various measurements (body weight, body temperature, clinical chemistry, and hematology) were obtained weekly to monthly throughout the study to monitor drug safety. Serum and hepatic WHV parameters, host immune response parameters, and drug safety parameters were compared to the values at pretreatment and between both dose groups using unpaired Student's t-test with equal variance. P values of <0.05 were considered statistically significant.

### Results

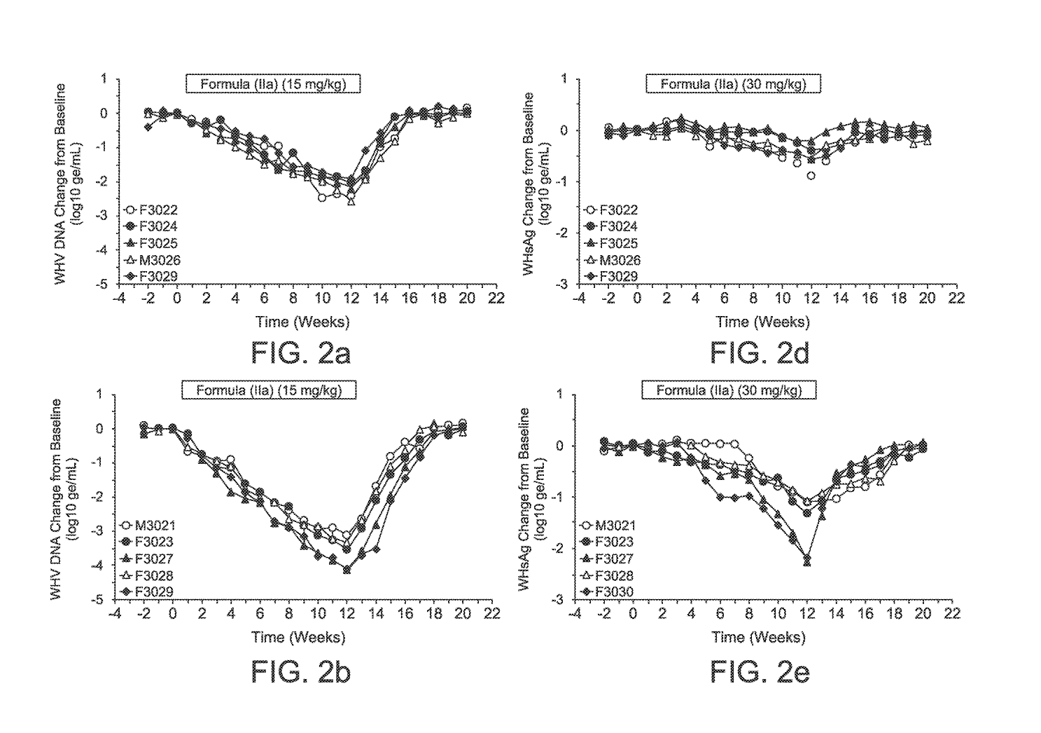

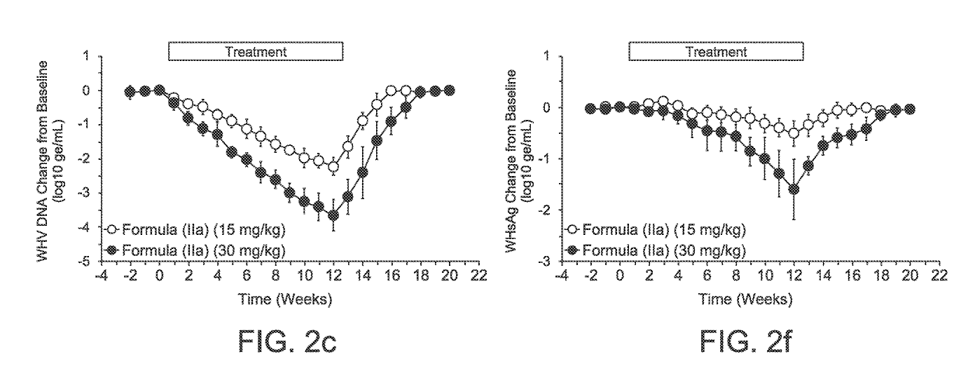

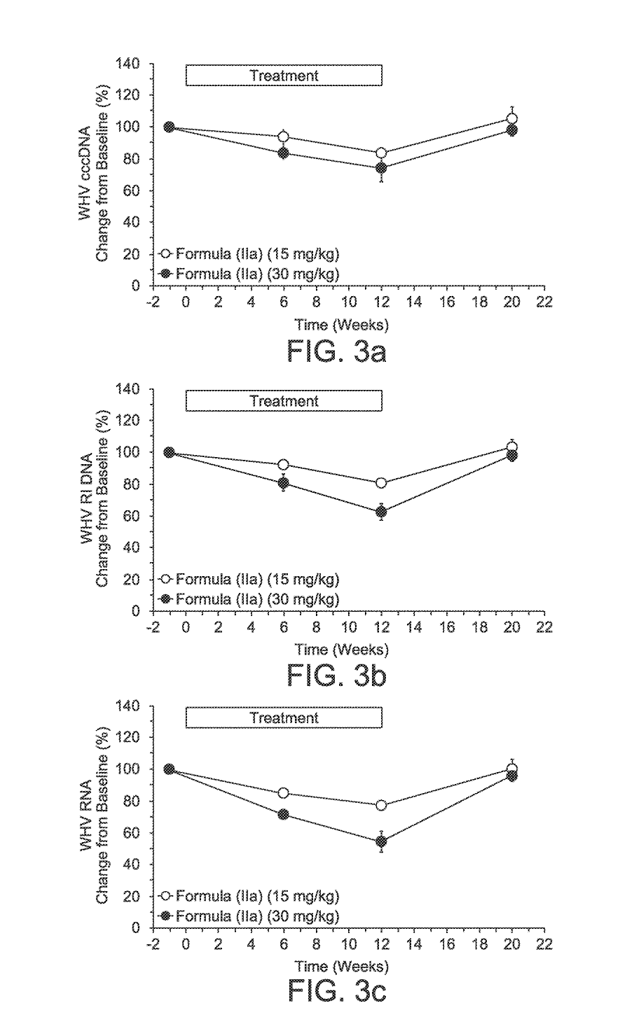

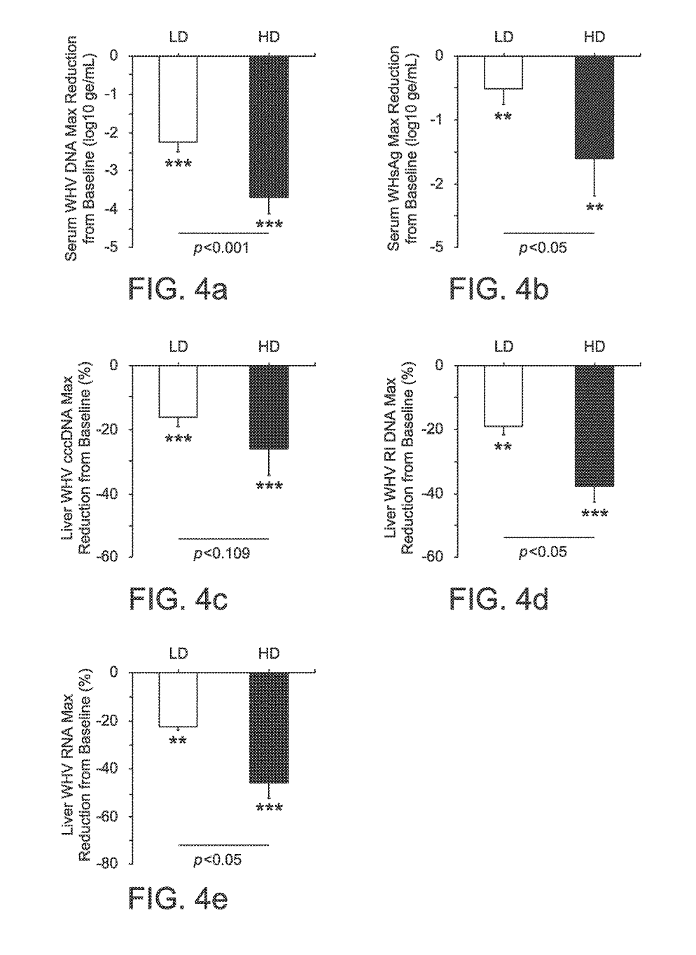

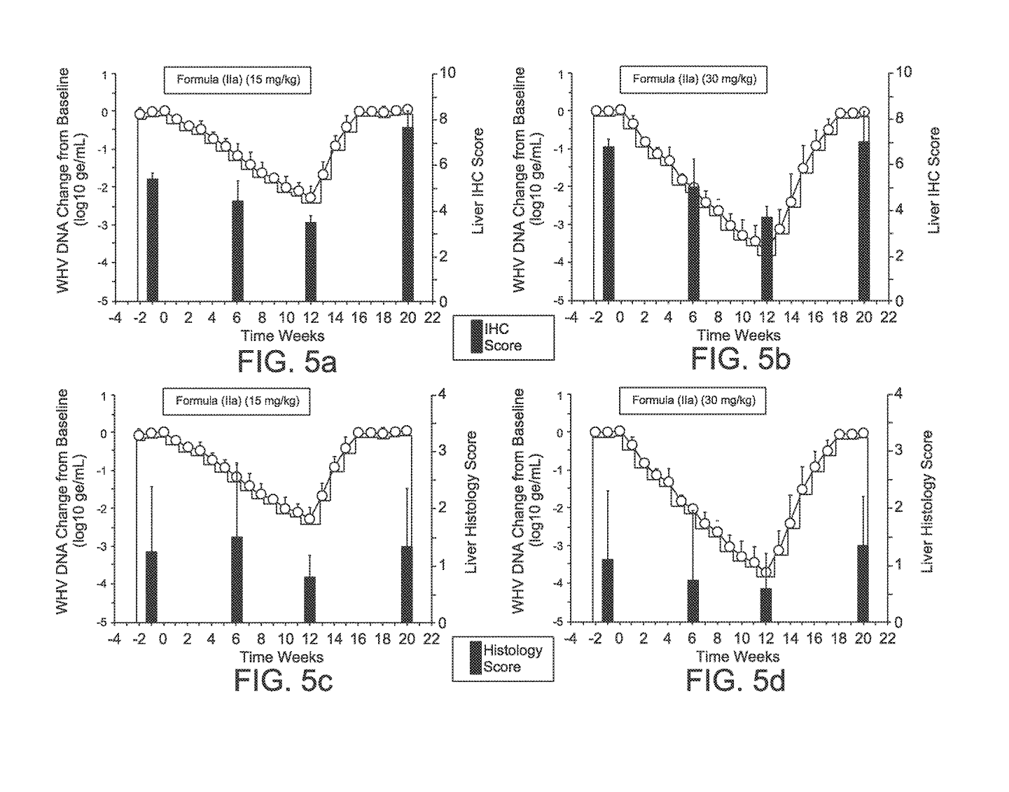

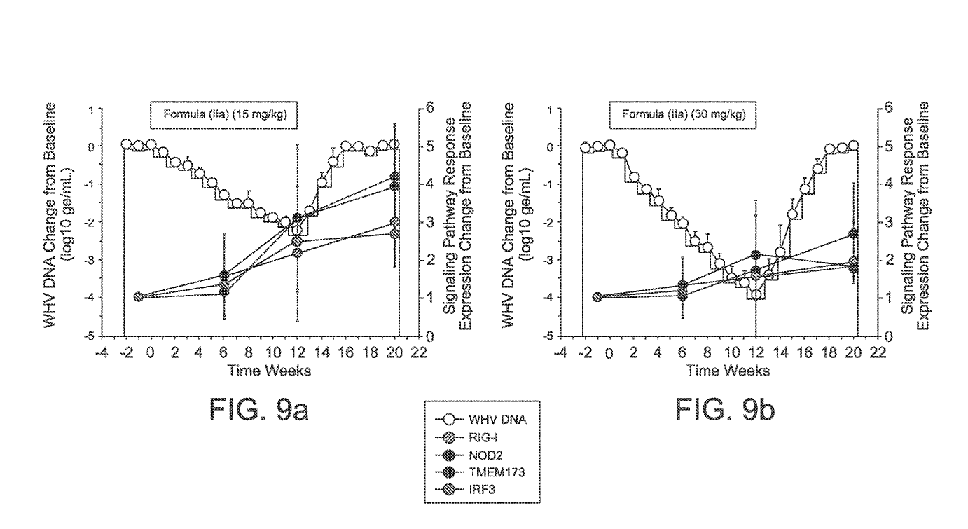

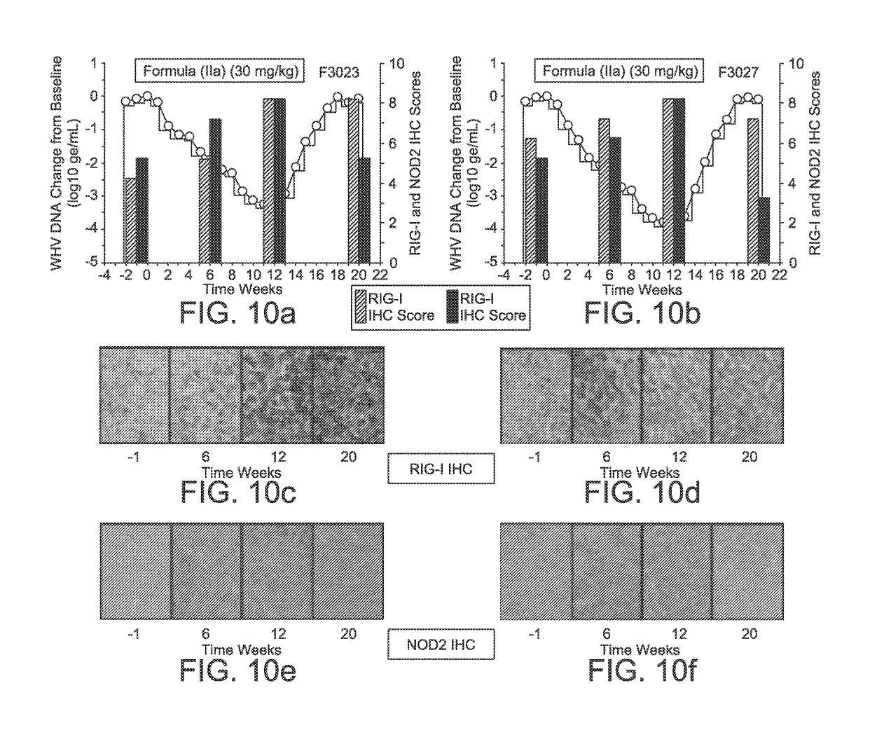

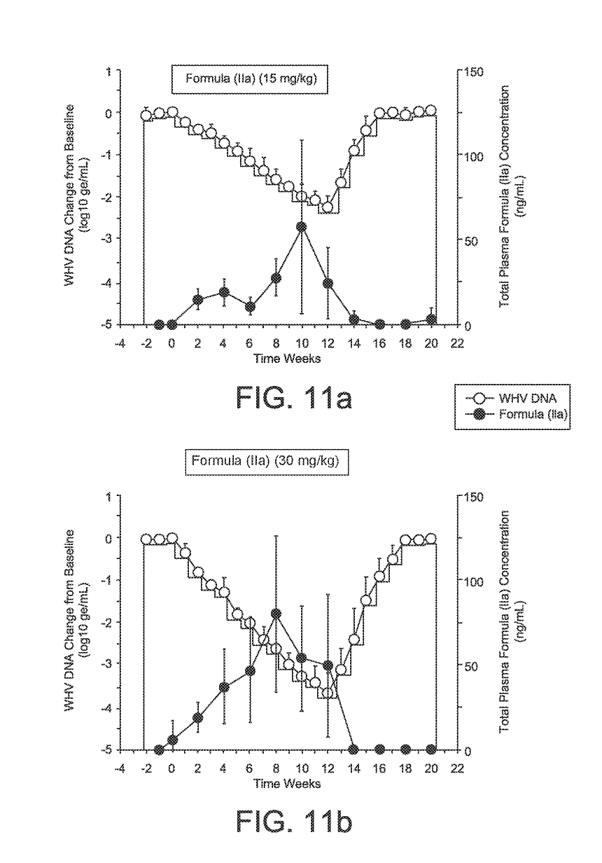

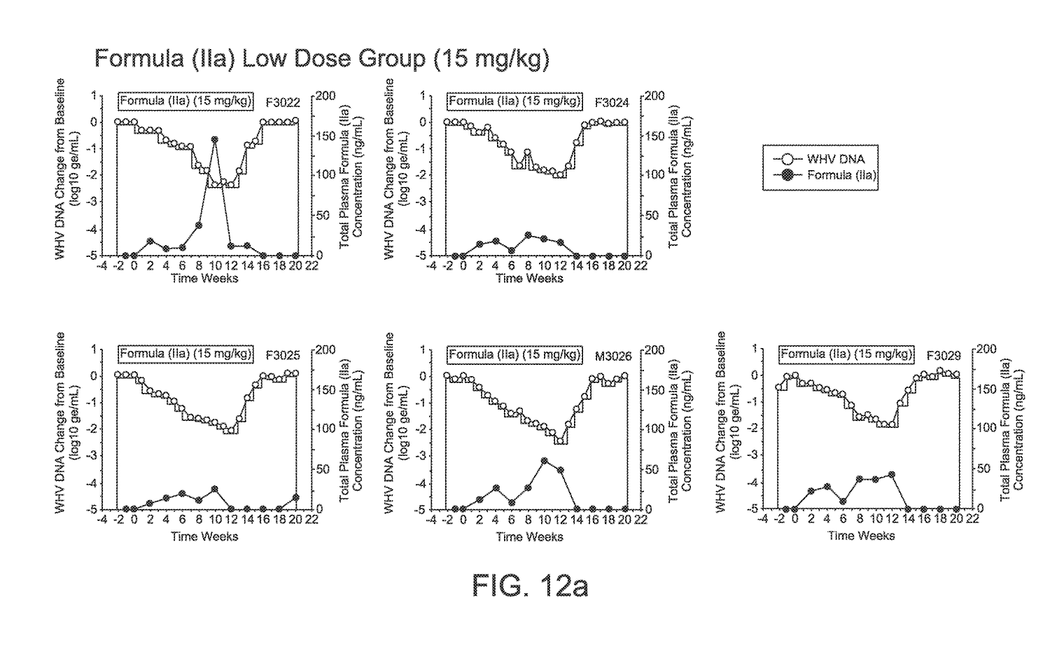

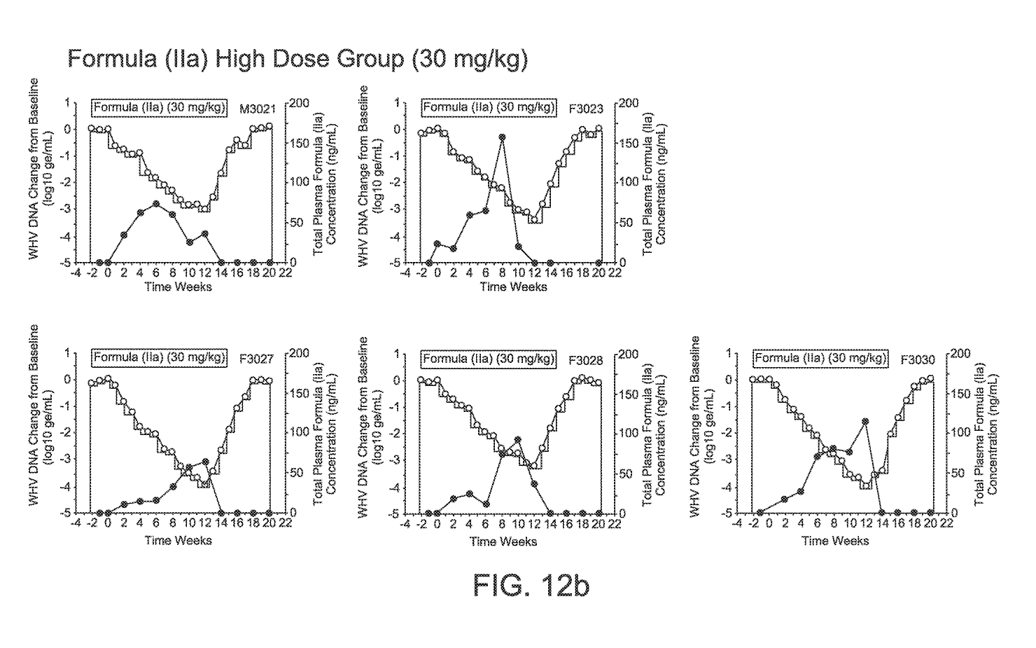

Formula (IIa) efficacy study in chronic WHV carrier woodchucks. The antiviral efficacy of Formula (IIa) was evaluated in a single agent, repeat-dose efficacy study in adult woodchucks chronically infected with WHV. For modeling vertical transmission in humans, chronic infection in these animals was established by neonatal WHV infection. Two groups of 5 woodchucks each were treated daily, orally with either a low (15 mg/kg) or high dose (30 mg/kg) of Formula (IIa) for 12 weeks. Following cessation of treatment, animals were followed for additional 8 weeks until the end of the study at week 20 (time of scheduled euthanasia).
Treatment of chronic WHV carrier woodchucks resulted in dose-dependent plasma exposure of Formula (IIa). The plasma exposure of Formula (IIa) following oral administration at 15 and 30 mg/kg was dose-dependent and statistically significant increased (p<0.05) during treatment when compared to pretreatment level, and also in the high dose group when compared to the low dose group (FIGS. 9 and 10).
Formula (IIa) treatment of chronic WHV carriers induced dose-dependent suppression of serum viremia. Formula (IIa) treatment induced marked reductions in serum WHV DNA from pretreatment level at T₀ that were noted as early as the first week of treatment (FIGS. 2A-2C). Formula (IIa) induced reductions in viremia were observed in all treated woodchucks but were more pronounced and sustained in animals administered the higher dose than in animals treated with the lower dose. Dose-dependent declines in WHV DNA occurred uniformly in all woodchucks but some variability in antiviral response was noted for two animals treated with the higher dose (F3027 and F3030). Reductions in mean viral load during the 12-week treatment period occurred gradually, and WHV DNA declined by approximately 1 log₁₀ every 6 or 3 weeks, respectively, in the low and high dose groups. At the end of treatment at week 12, the average reduction of WHV DNA in the low and high dose groups was 2.2 or 3.7 log₁₀, respectively (FIGS. 2C and FIG. 4A). After the completion of treatment, rebound in viral load was observed in all woodchucks and WHV DNA increased gradually to pretreatment level during the 8-week follow-up period. In woodchucks treated with the low dose, WHV DNA returned to pretreatment level within 3-5 weeks following Formula (IIa) withdrawal but animals administered the higher dose had a delay in relapse by 2-4 weeks as pretreatment level was reached 5-7 weeks after drug withdrawal. The two woodchucks of the high dose group with the most pronounced WHV DNA reduction (F3027 and F3030) also had the most delayed rebound in viral load. Mean viral load in the low and high dose groups during weeks 1-15 or weeks 1-17, respectively, were significantly reduced compared to pretreatment level at T₀ (all p<0.05). In addition, the mean viral load of the high dose group during weeks 2-17 was significantly lower than in the low dose group (all p<0.05).
Formula (IIa) treatment induced dose-dependent reduction in serum antigenemia but no seroconversion. Formula (IIa) administration caused dose-dependent reductions in serum WHsAg from pretreatment level at T₀ in all treated woodchucks (FIG. 2D-2E), and also in serum WHV e antigen (WHeAg). Declines in antigenemia were observed as early as the second week of treatment and were more marked and durable in woodchucks treated with the higher dose, especially in the two animals with the more pronounced reduction in viremia (F3027 and F3030). The maximum reduction of WHsAg observed in the low and high dose groups was 0.5 or 1.6 log₁₀, respectively, after 12 weeks of treatment (FIG. 2F and FIG. 4B). Following withdrawal of Formula (IIa), gradual rebound in antigen load to pretreatment level was noted. In woodchucks treated with the lower dose, WHsAg returned to pretreatment level within 1-5 weeks after the completion of treatment. In animals administered the higher dose, a 1-7 week delay in antigen rebound was observed, and pretreatment level was reached 6-8 weeks after the end of treatment. Mean antigen load was significantly reduced in the low and high dose groups during weeks 10-14 or weeks 9-16, respectively, when compared to the pretreatment level at T₀ (all p<0.05), and in the high dose group during weeks 11-16, when compared to the low dose group (all p<0.05). As 12 weeks of Formula (IIa) administration at the doses applied was unable to produce complete loss of detectable WHsAg (and WHV DNA) in treated woodchucks, seroconversion to anti-WHs antibodies and to antibodies against WHeAg (anti-WHe antibodies) was not observed (data not shown).
Formula (IIa) treatment induced dose-dependent reduction in hepatic levels of WHV nucleic acids. Compared to pretreatment level at week-1, Formula (IIa) administration resulted in dose-dependent reductions in the levels of hepatic WHV covalently-closed circular (ccc) DNA, WHV DNA replicative intermediates (RI), and WHV RNA (FIG. 3, FIG. 10). Although liver biopsies could not be collected from all woodchucks at the end of treatment, the declines in these viral markers correlated well with the reductions in serum viremia and antigenemia (compare FIG. 2 and FIG. 3). After 12 weeks of treatment, the maximum reduction of WHV cccDNA, WHV DNA RI and WHV RNA from pretreatment level in the low and high dose groups was 16%, 19% and 22% or 25%, 38% and 45%, respectively, indicating that the antiviral effect of Formula (IIa) was most pronounced for viral RNA (FIGS. 4C-E). Following cessation of treatment, rebound in the hepatic levels of these viral markers was observed in all woodchucks at the end of the study. When compared to pretreatment level, mean WHV cccDNA, WHV DNA RI and WHV RNA levels were significantly reduced in the low dose group at weeks 6 and 12, 12, or 6 and 12 (all p<0.05), respectively, whereas the levels of all three WHV molecules were significantly reduced in the high dose group at weeks 6 and 12 (all p<0.05). Compared to the low dose group, mean WHV cccDNA, WHV DNA RI and WHV RNA levels in the high dose group were significantly lower at weeks 6, 12, or 6 and 12, respectively (all p<0.05).
Formula (IIa) treatment induced decline in hepatic WHV antigen expression and was associated with reduced liver inflammation. Formula (IIa) treatment resulted in dose-dependent, transient reductions in hepatic expression scores of cytoplasmic WHcAg from pretreatment level at week-1 in all treated woodchucks (FIG. 5A-5B, FIG. 11). Declines in WHcAg expression were already observed after 6 weeks of treatment. WHcAg expression continued to decline during the remainder of treatment, and the reductions at week 12 were most pronounced in woodchucks treated with the higher dose. Following completion of treatment, increases in the hepatic expression scores of WHcAg were noted for all woodchucks at the end of the study. Mean scores were significantly reduced in the low and high dose groups at weeks 6 and 12 (all p<0.05) when compared to pretreatment; however, on a group level the difference in hepatic WHcAg expression was not statistically significant.

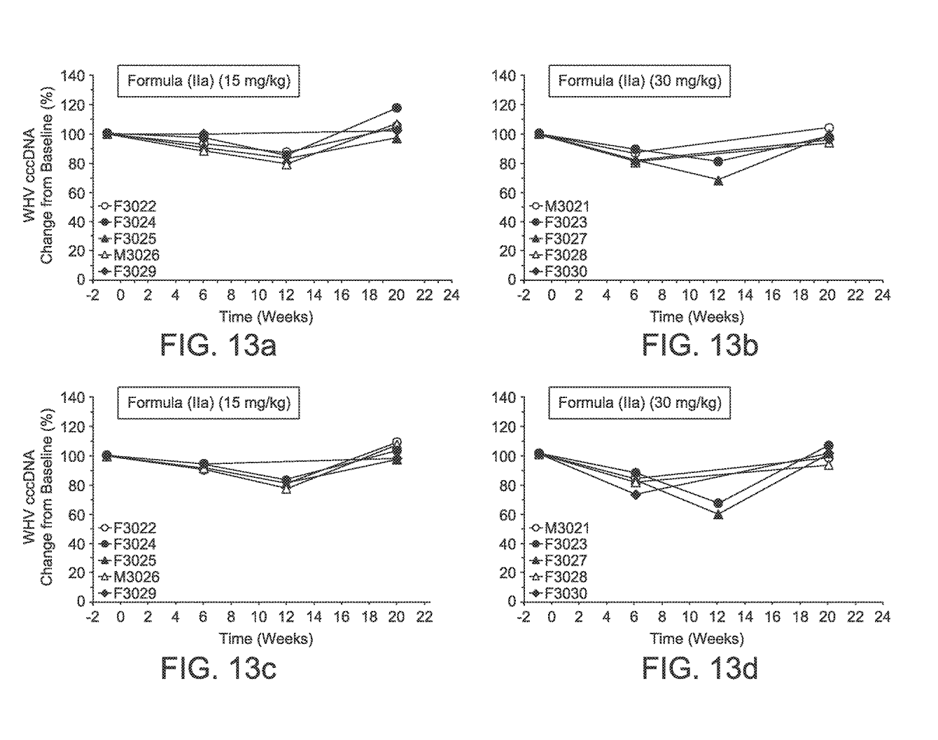

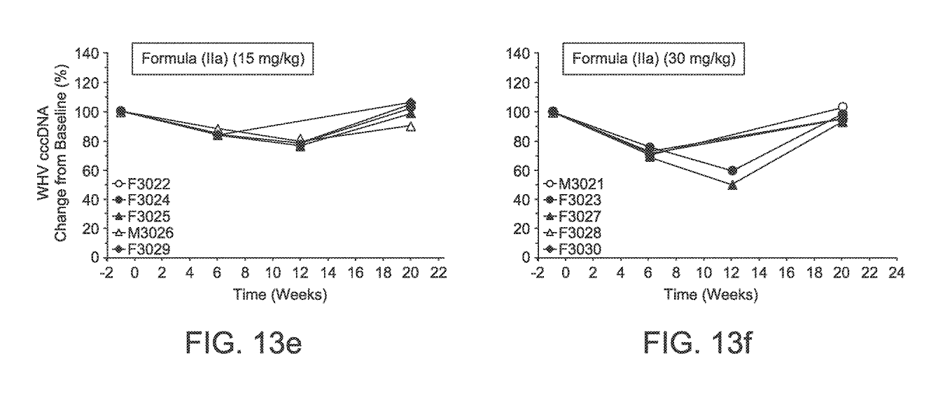

Formula (IIa) administration further correlated temporally with unchanged or even reduced scores of liver inflammation in all treated woodchucks during the 12-week treatment period (FIG. 5C-5D, FIG. 13). After cessation of treatment, the composite scores for lobular sinusoidal and portal hepatitis increased in most (although not all) animals at the end of the study. The trend to reduced liver inflammation was comparable in woodchucks treated with the lower or higher dose of Formula (IIa), with no statistically significant differences when compared to pretreatment or between the groups.

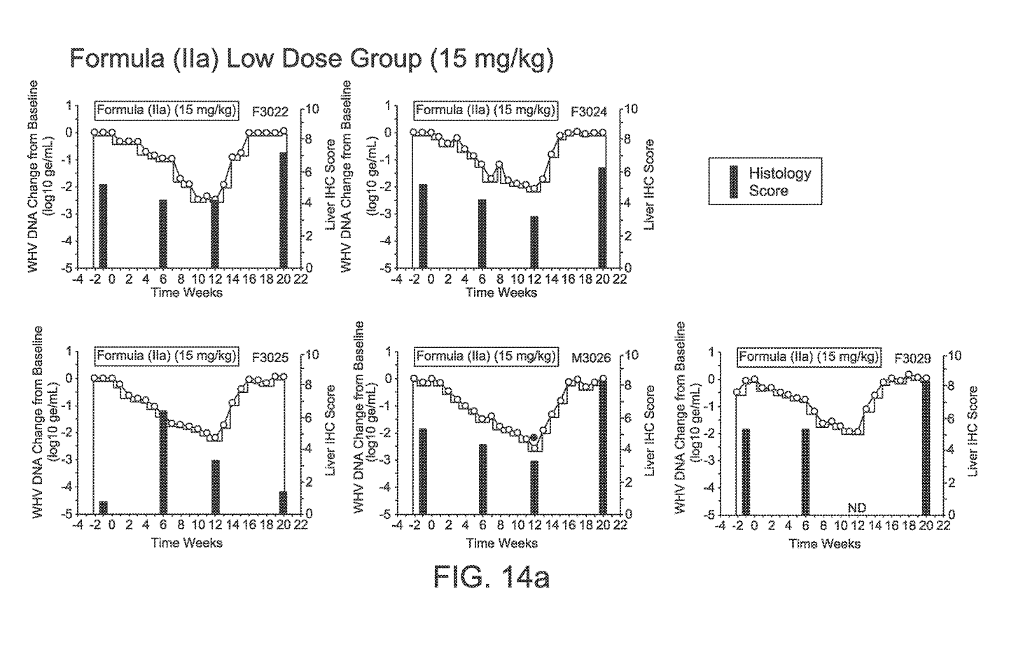

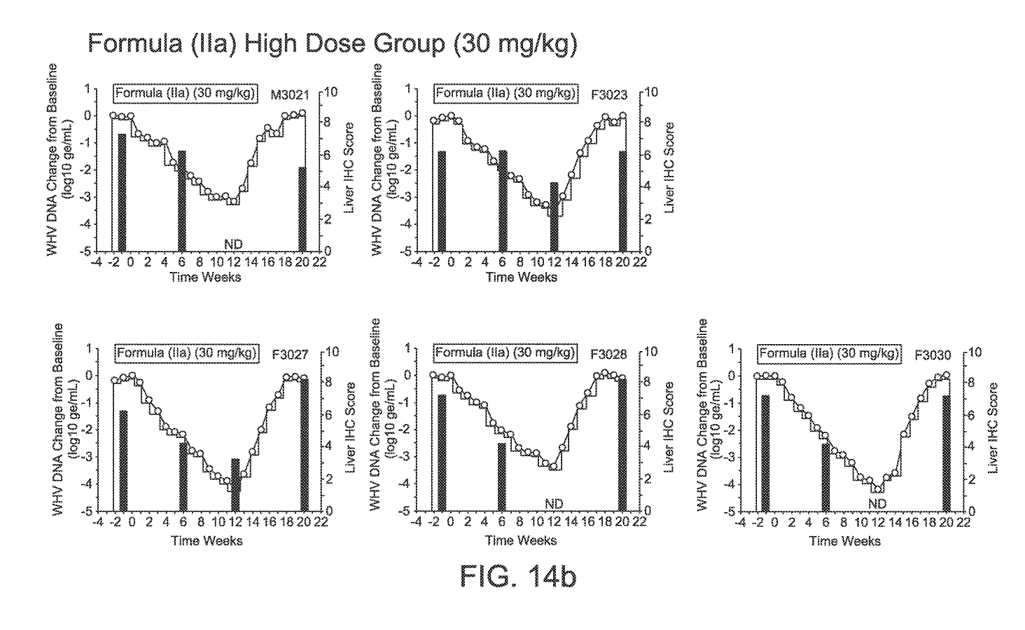

Tolerability of Formula (IIa) treatment in chronic WHV carrier woodchucks. Formula (IIa) treatment was well-tolerated in woodchucks, and there were no signs of overt toxicity based on gross observations, body weights, body temperatures, hematology, or clinical chemistry (data not shown), and no mortality was observed during the study. There was a tendency in the high dose group towards lower estimated tumor volumes (data not shown) and towards reduced levels of GGT, an established oncofetal marker of liver tumor development in woodchucks with chronic WHV infection (FIG. 14).

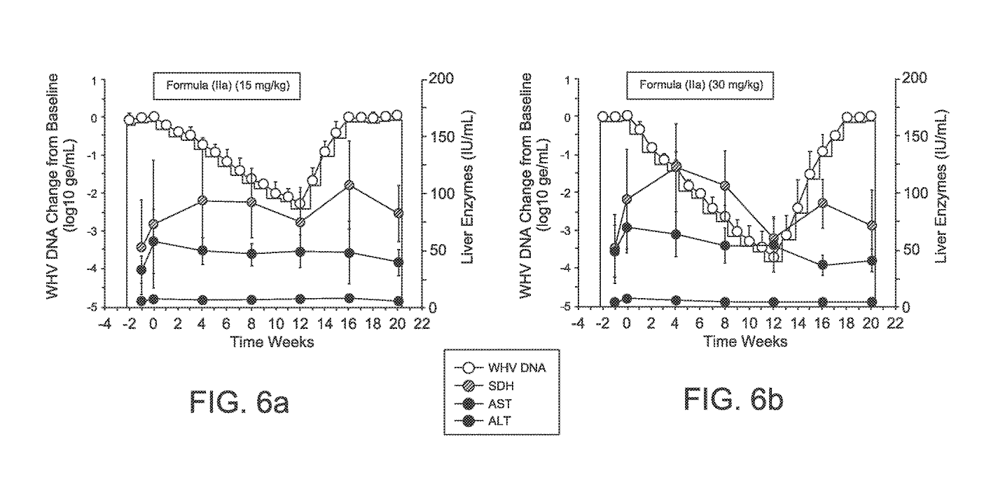

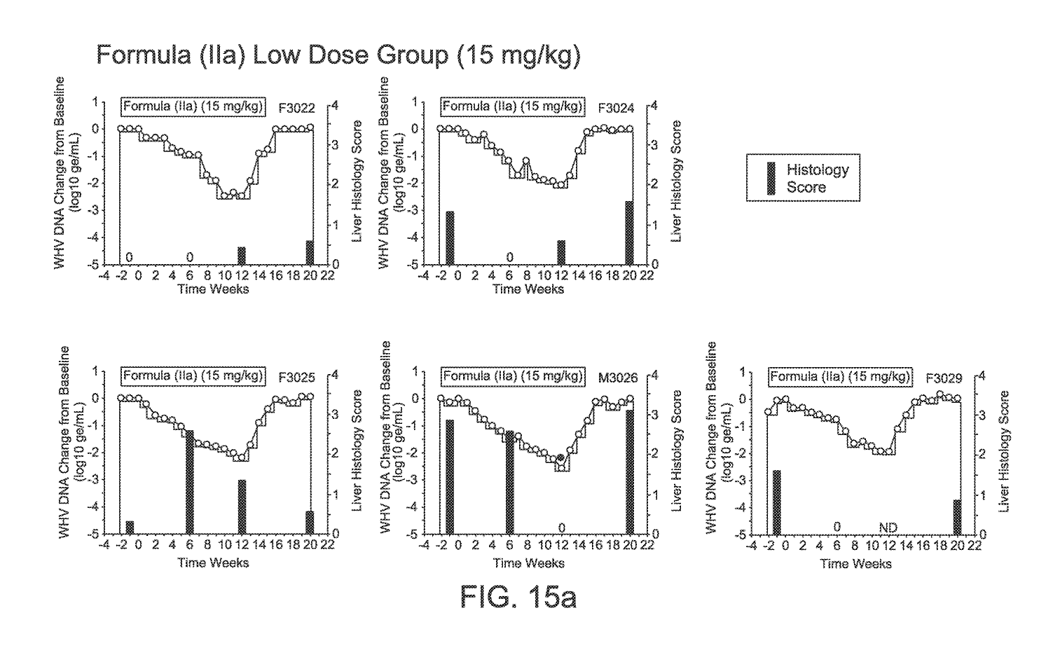

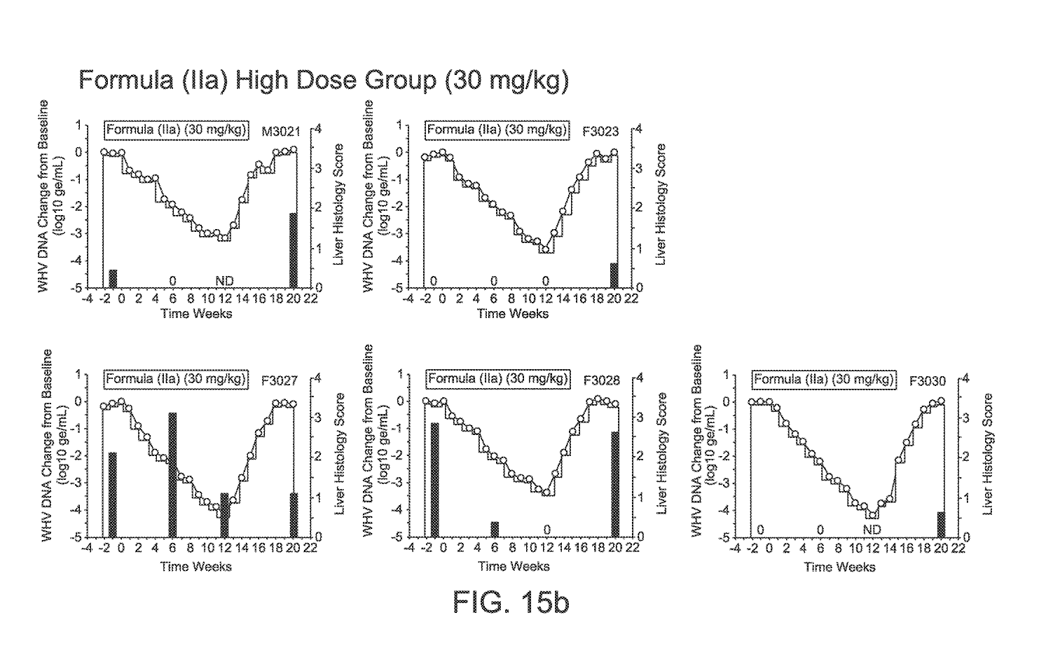

There was further a trend towards elevated serum SDH levels during Formula (IIa) administration, especially during the initial 4-8 weeks of treatment, and elevations were more pronounced in the high dose group than in the low dose group (FIG. 6, FIG. 14). Conversely, at the end of treatment at week 12, and at the time of peak antiviral response in both groups, serum SDH levels declined, and the reductions were again more pronounced in the high dose group. Following cessation of treatment, serum SDH levels became elevated again at the time of initial relapse of viremia and antigenemia. Serum levels of alanine aminotransferase (ALT) and aspartate aminotransferases (AST) essentially remained unchanged or declined slightly during and following treatment in woodchucks of both groups. On a group level, however, these overall differences were not statistically significant when compared to pretreatment or between the groups, with the only exception of serum AST that was significantly reduced in the high dose group during follow-up when compared to pretreatment and treatment levels (FIG. 14). The temporal association between elevation of SDH (and unchanged or reduced ALT, AST and alkaline phosphatase (ALP)) and initial decline of viral and antigen loads during Formula (IIa) treatment, and again during viral relapse following completion of treatment, was also observed on an individual level (FIG. 15). Furthermore on an individual animal level, there was a temporal association between peak antiviral response and decline of SDH that also correlated temporally with reduced liver inflammation. This biphasic kinetic of SDH during treatment when correlated to the mounting antiviral response and the declining liver inflammation may be indicative of the host immune response induced by Formula (IIa).

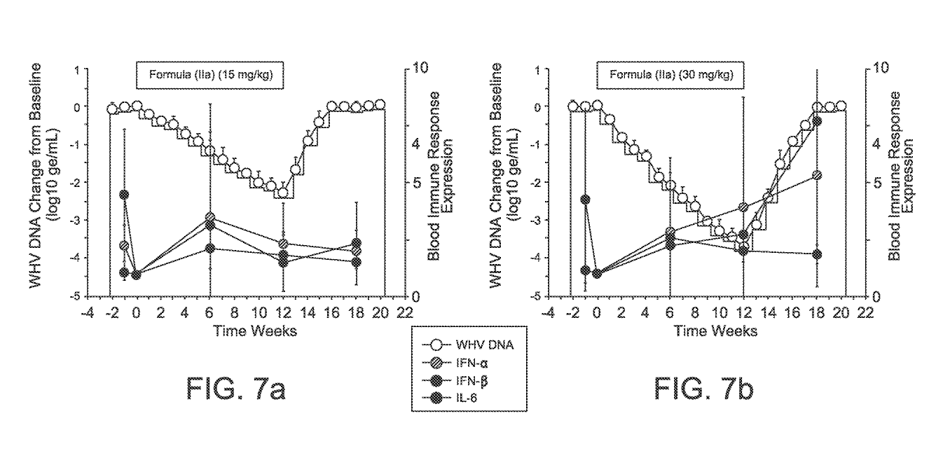

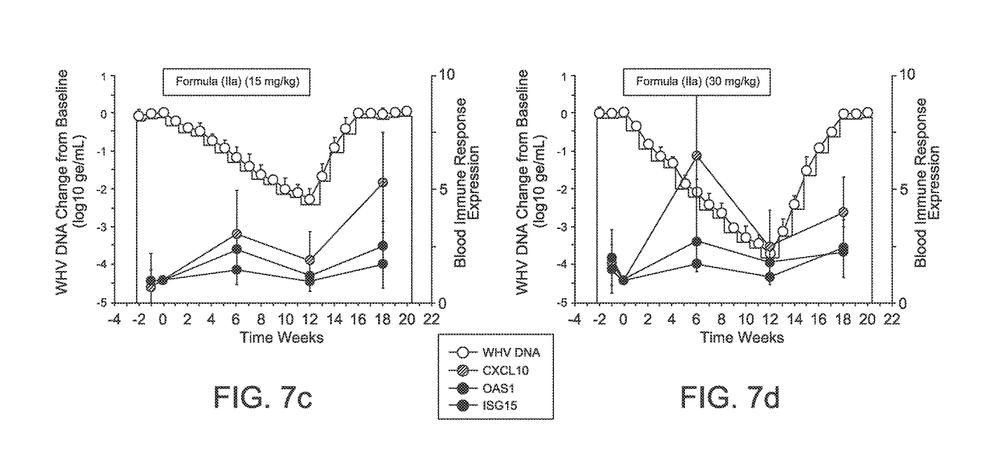

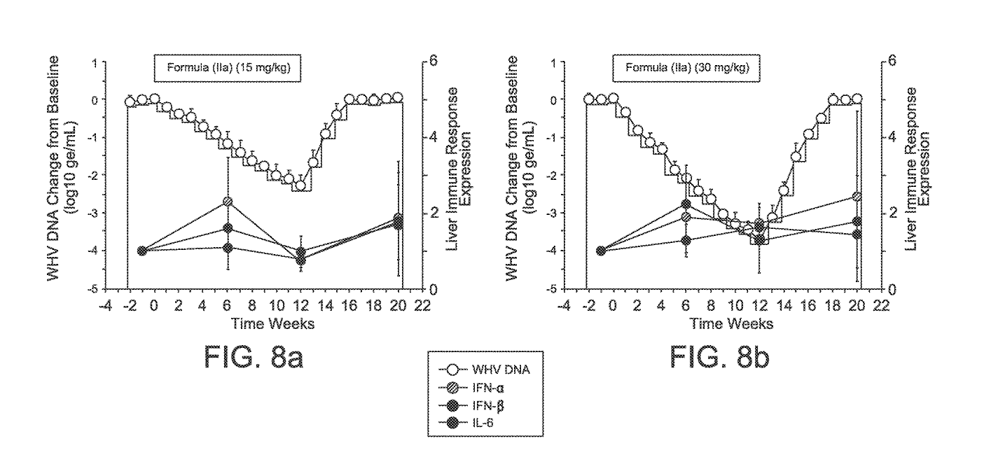

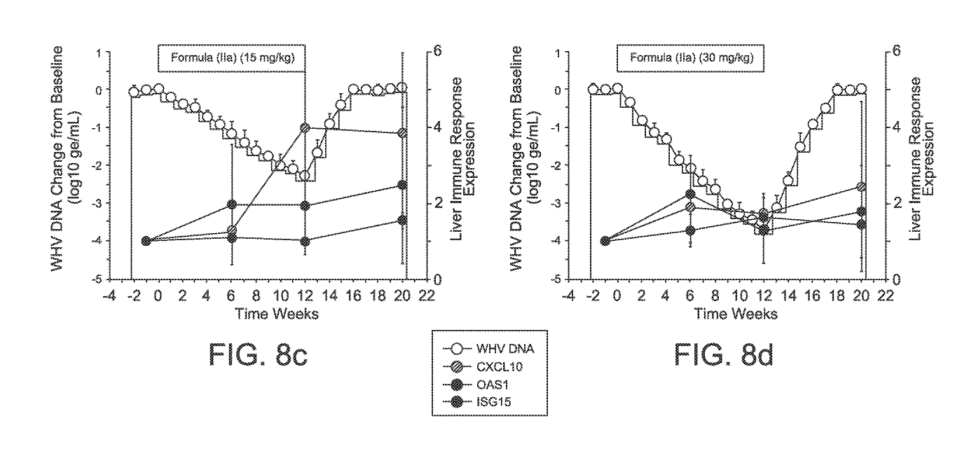

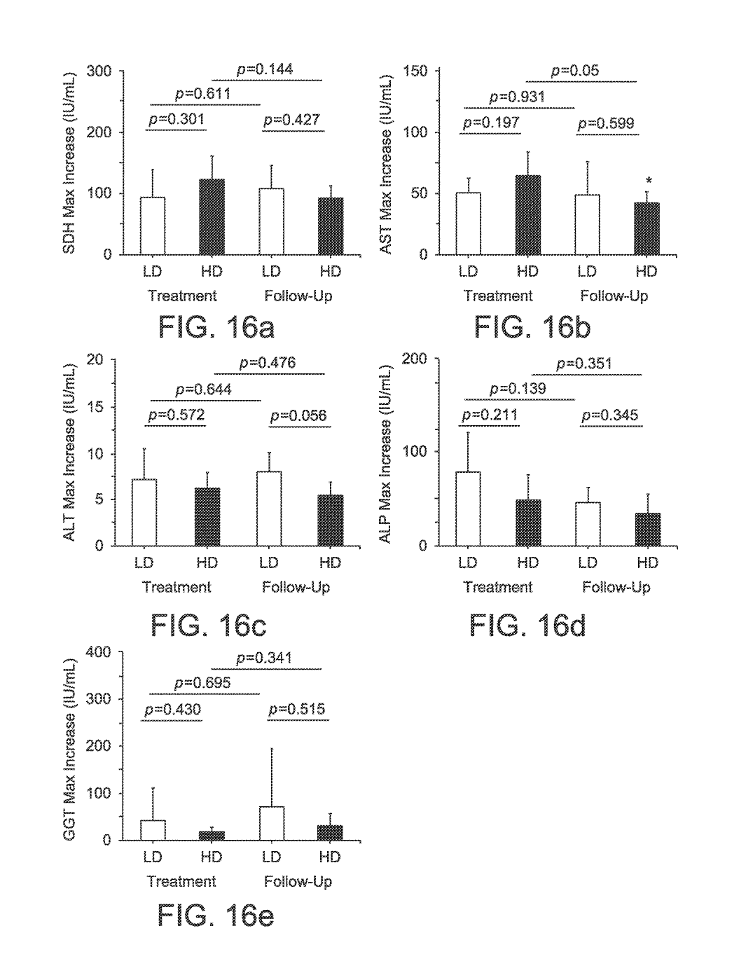

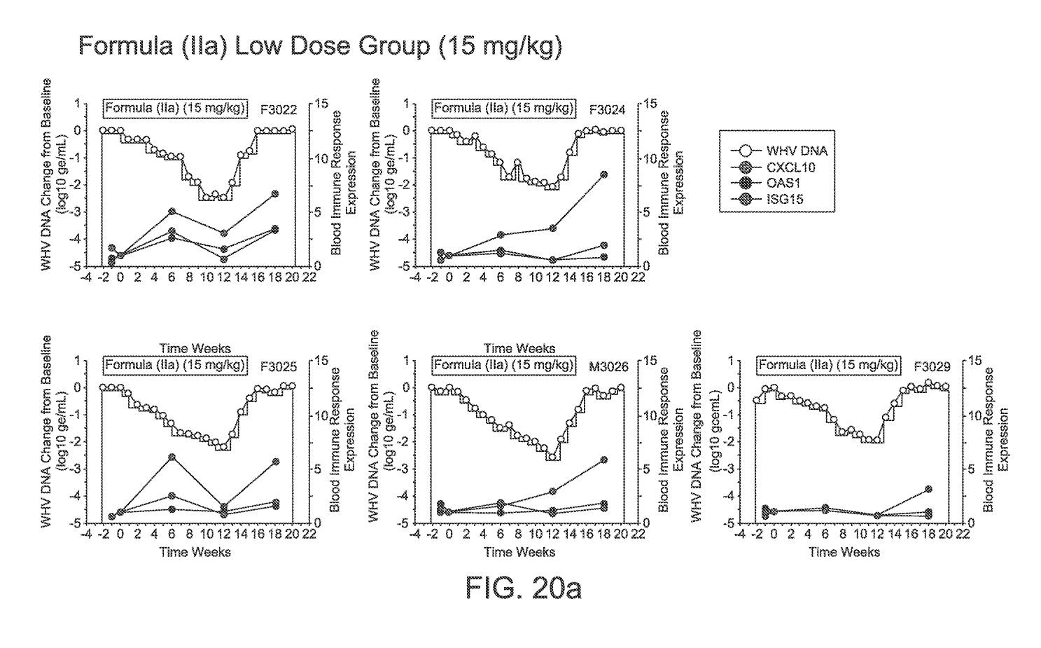

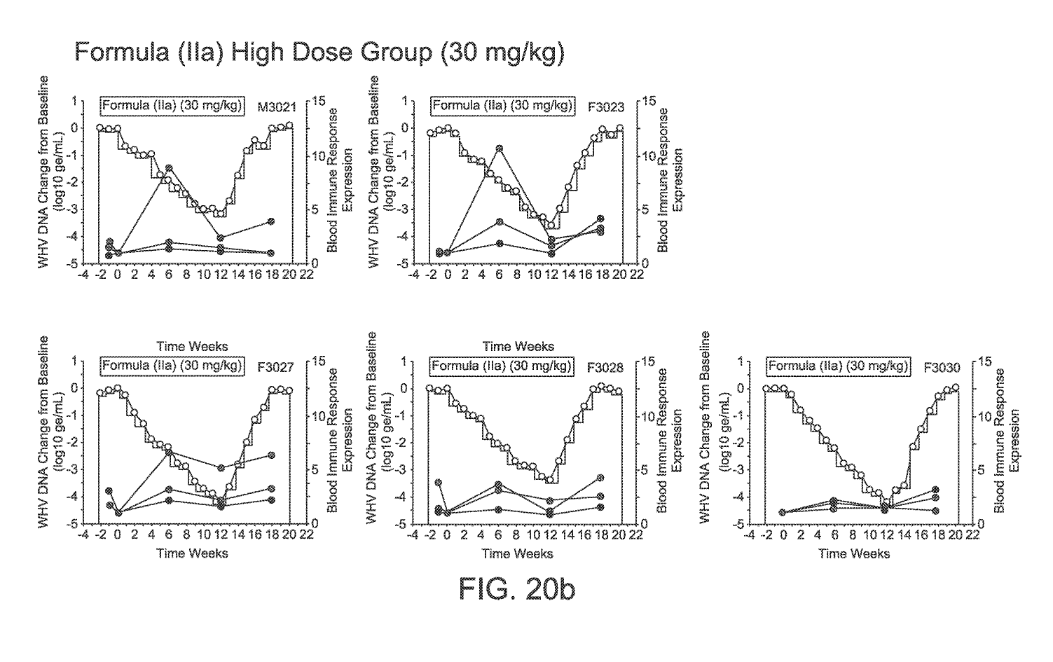

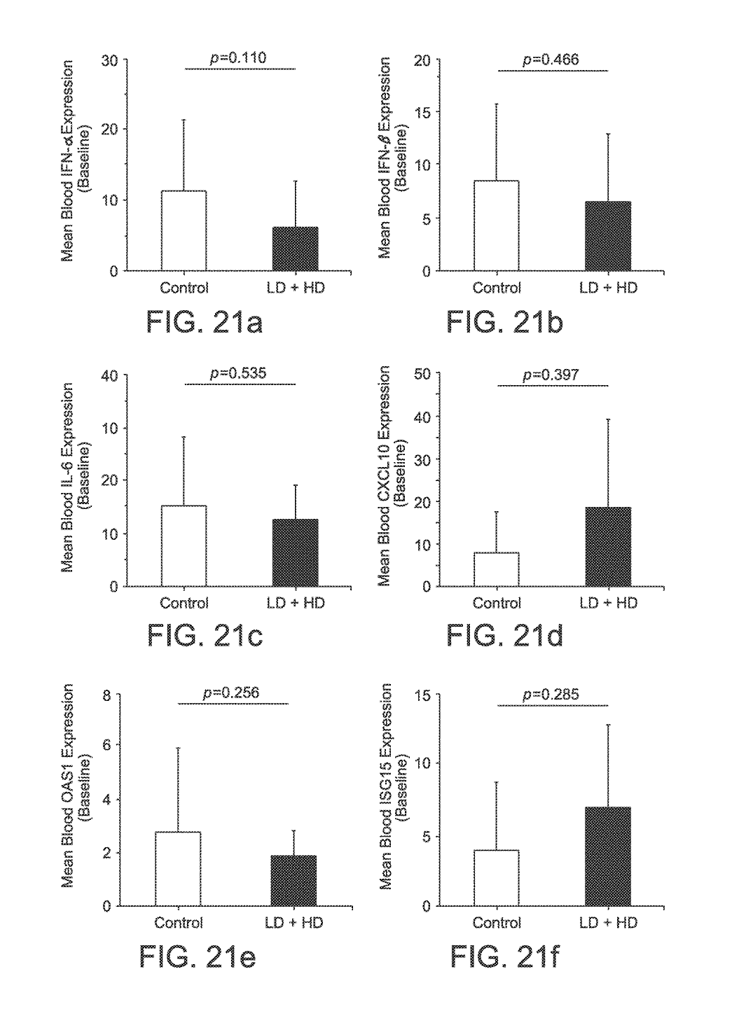

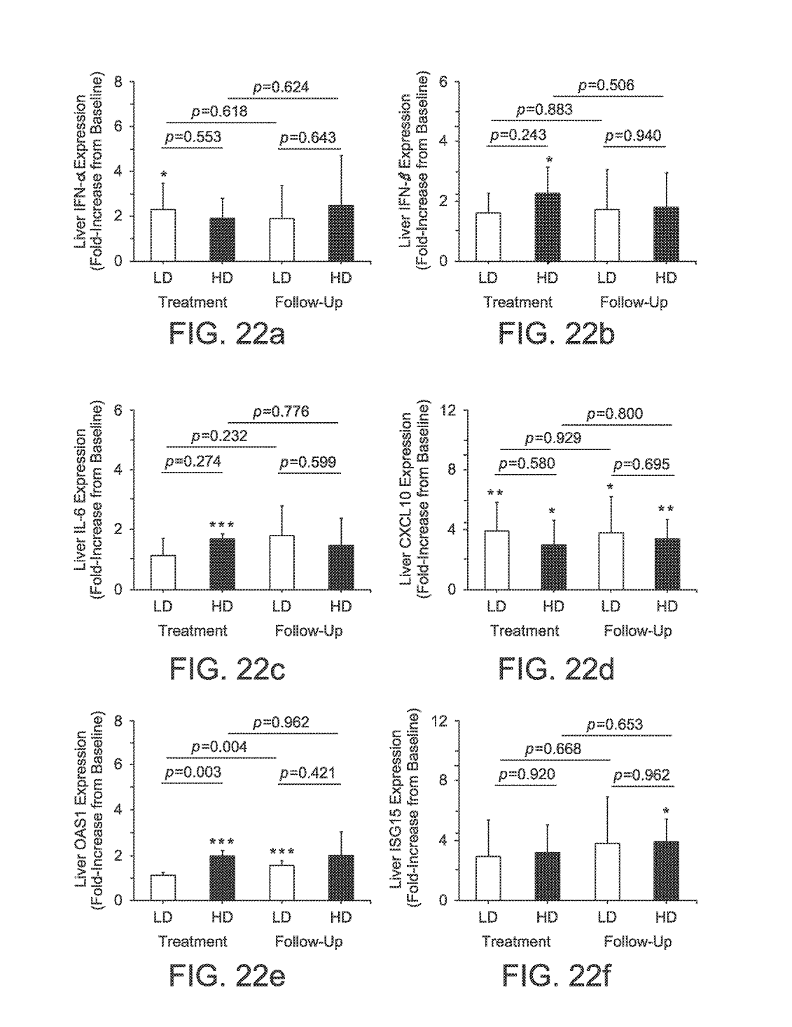

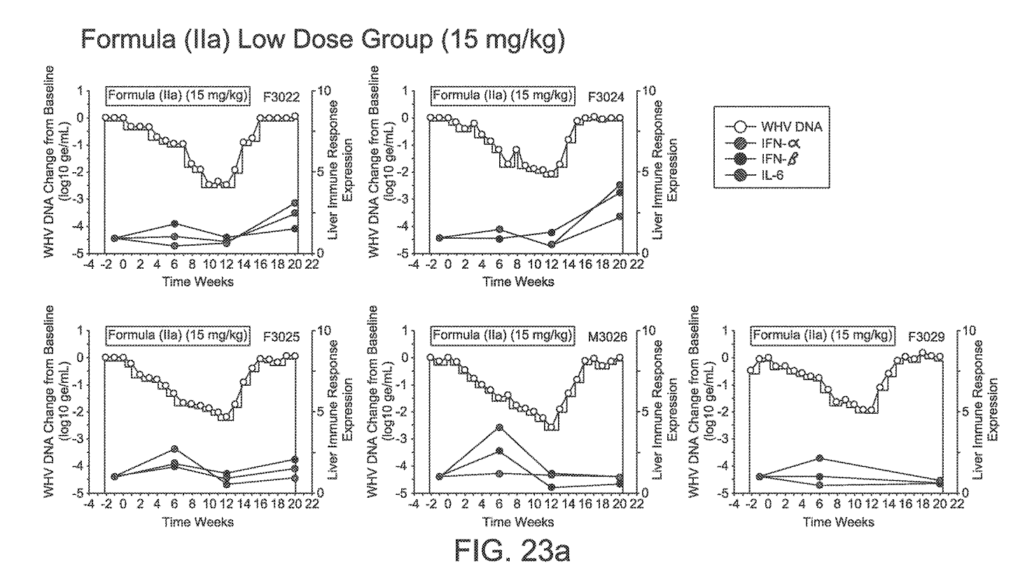

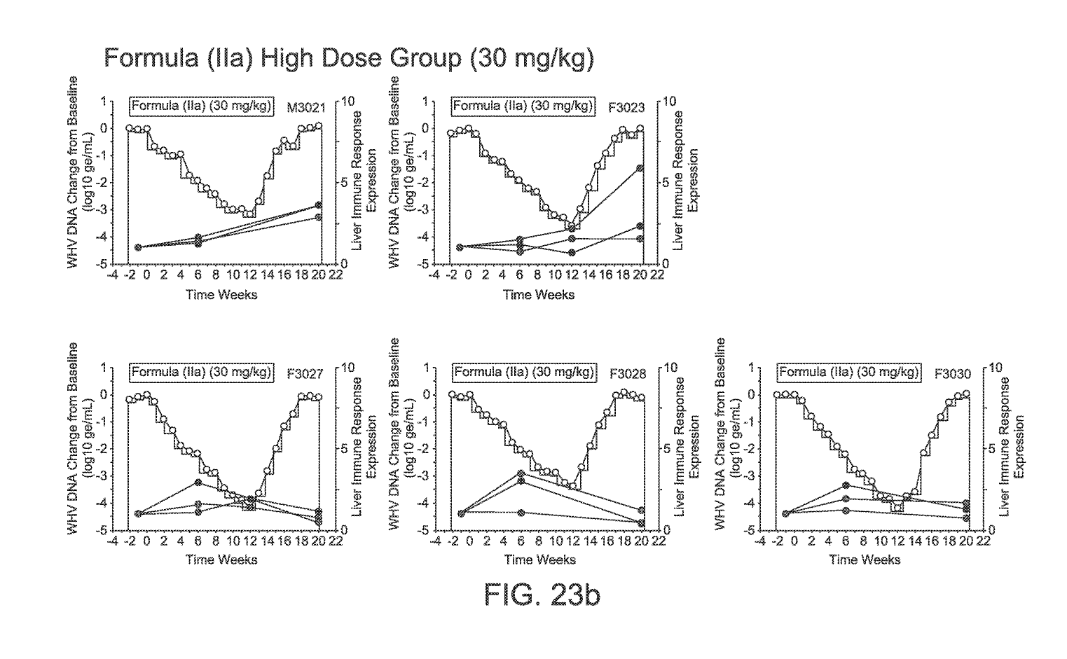

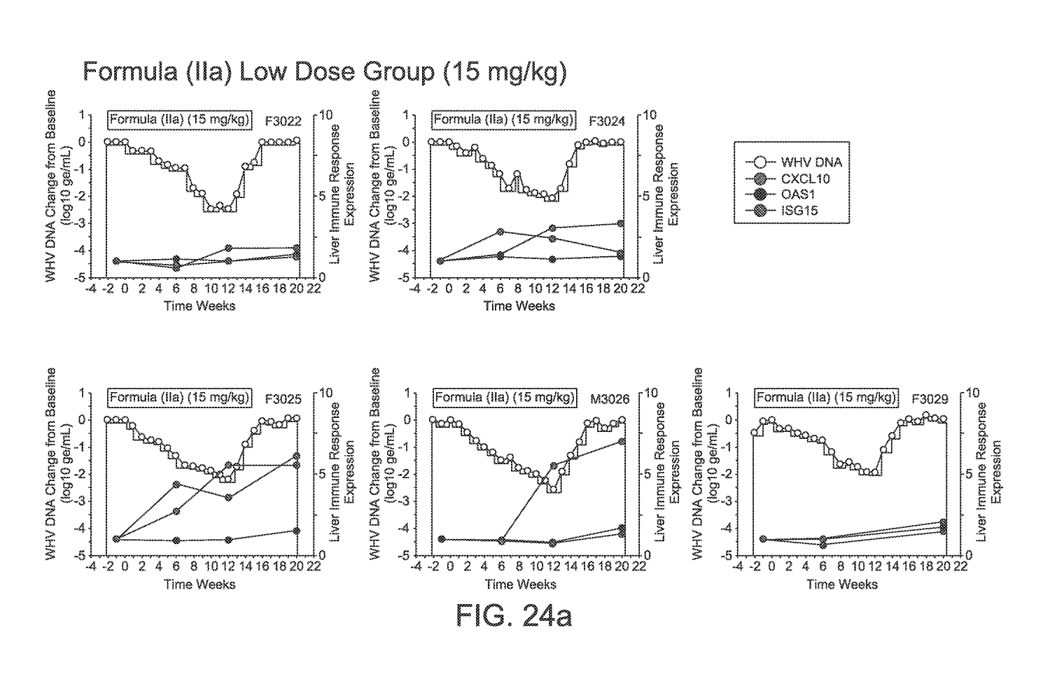

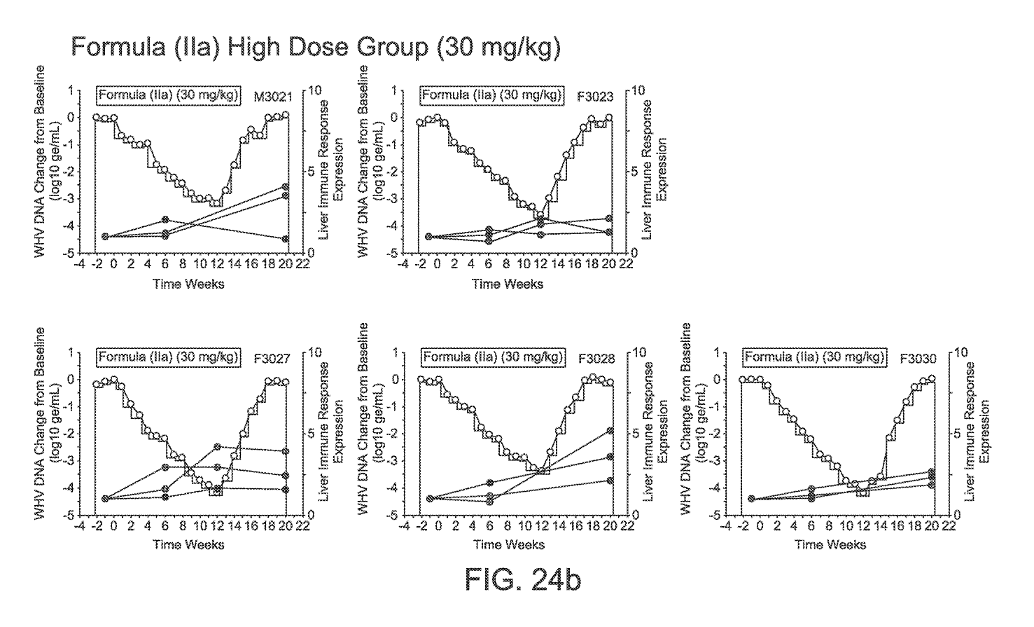

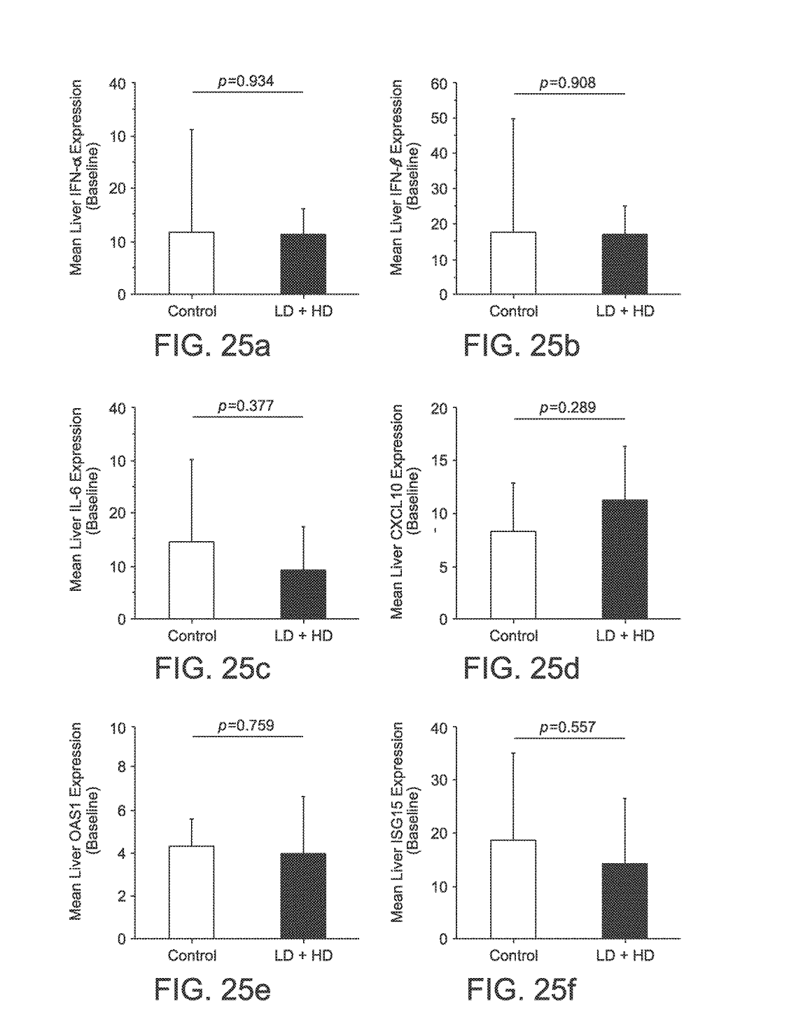

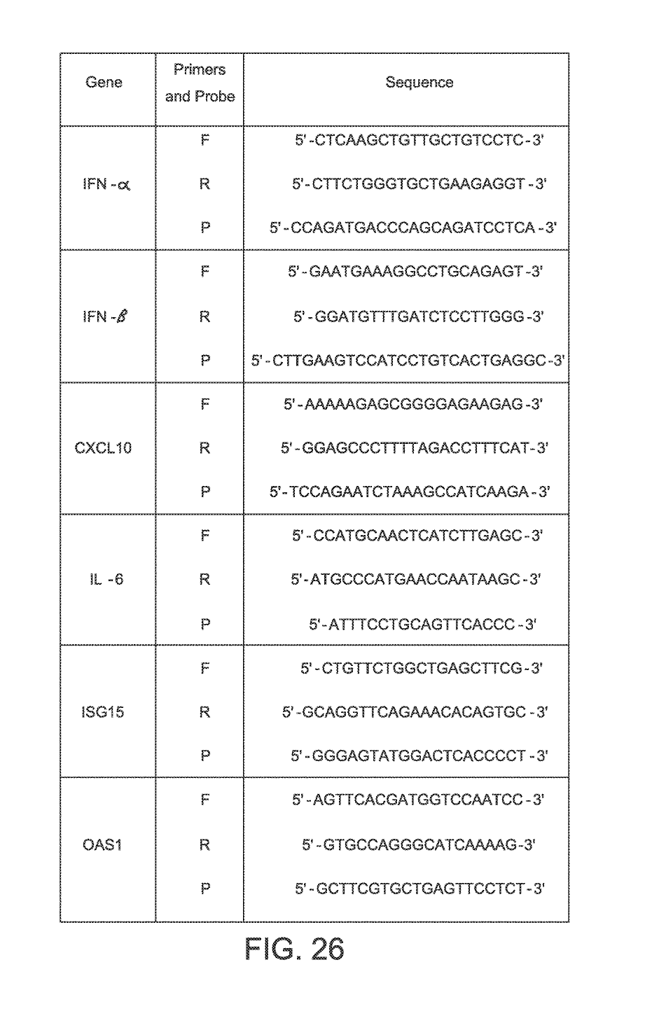

Formula (IIa) treatment induced dose-dependent and sometimes long-lasting expression of type I IFNs and ISGs in the blood of chronic WHV carrier woodchucks. There was a trend of Formula (IIa) treatment towards the induction of mRNA expression of type I IFNs (i.e., IFN-α and IFN-β) and select antiviral ISGs (i.e., OAS1 and ISG15) in blood, with significant induction at the high dose when compared to pretreatment (FIG. 7, FIG. 16). In contrast, both the low and high doses significantly induced the expression of the proinflammatory cytokine, IL-6, and of another ISG, CXCL10, when compared to pretreatment, but increases in transcript level were more pronounced in the high dose group. Transient increases in gene expression were observed at week 6 of treatment in both groups and in most individual animals, except for IFN-α and IFN-β in the high dose group as expression increased until the end of treatment at week 12, and increased further during the follow-up at week 18. Conversely, expression of all other genes declined at the end of treatment and stayed at comparable or lower levels during follow-up, except for the expression of CXCL 10 and OAS1 that increased in the low or high dose groups, respectively. However, these overall differences were not statistically significant when compared between both groups or between treatment and follow-up for individual groups. Given that Formula (IIa) treatment at two separate doses was associated with significant suppression of WHV replication, this suggests that dose-dependent induction of host innate immunity (and associated cellular immunity) plays a role in the antiviral response mediated by this compound.
Formula (IIa) treatment induced comparable and long-lasting expression of IFNs and ISGs in the liver of chronic WHV carrier woodchucks. Analogous to the observations in the periphery, Formula (IIa) treatment also induced mRNA expression of IFNs and ISGs in liver (FIG. 8, FIG. 11). Compared to pretreatment, expression of IFN-α was significantly induced in the low dose group whereas significantly increased expression of IFN-β, IL-6 and OAS1 was observed in the high dose group. The difference in expression of OAS1 (but not of other genes) was also statistically significant between low and high dose groups during treatment. Furthermore, CXCL10 expression was significantly induced by treatment in both groups. Conversely to the periphery, expression of CXCL10, OAS1 and ISG15 was not transient during treatment and increased further at the end of treatment and during follow-up in both groups and in most individual animals (FIG. 20, FIG. 21). Significant elevation compared to pretreatment was observed at the end of the study for CXCL10 in both groups, for OAS1 in the low dose group, and for ISG15 in the high dose group. OAS1 expression was also significantly elevated in the low dose group during follow-up when compared to treatment. Significantly increased expression was not observed for the other genes tested although there was a tendency of IL-6 and IFN-α in the low or high dose groups, respectively, towards increased level during follow-up. These results suggest that treatment with Formula (IIa) at two separate doses induces comparable expression of IFNs and ISGs in liver. As expression of ISGs lasted beyond the end of treatment and was still elevated during viral relapse, this indicates that additional antiviral immune mechanisms are involved in the treatment response to Formula (IIa).

### Discussion

For establishing efficacy, safety and pharmacodynamics associated with antiviral response against HBV, chronic WHV carrier woodchucks were treated with Formula (IIa) for 12 weeks at two separate doses. Treatment with Formula (IIa) was well tolerated and produced dose-dependent and uniform antiviral effects by inducing multi-log reduction in serum WHV DNA and WHsAg and marked declines in hepatic WHV cccDNA, DNA RI, RNA and cytoplasmic WHcAg in all animals. However, Formula (IIa)-induced suppression of WHV replication was transient and viral rebound was observed following cessation of treatment, although relapse was significantly delayed in animals administered the higher dose. As neither dose of Formula (IIa) was able to produce loss of WHsAg and WHeAg, seroconversion to anti-WHs and anti-WHe antibodies and reduced HCC incidence were not observed.

During viral infections, virus-derived nucleic acids (both DNA and RNA) are mainly sensed by certain pattern-recognition receptors such as RIG-1 and NOD2 which are located within the cytoplasm of cells, including hepatocytes. Binding of these viral sensor proteins to PAMP within the viral nucleic acids activates downstream signaling pathways which include the mitochondrial antiviral signaling protein (MAVS) leading to the induction of the IFN-regulatory factor-3 (IRF-3) and NF-κB dependent gene expression, and the subsequent production of type I and type III IFNs and inflammatory cytokines. Thus, sensing of viral nucleic acids is a crucial process to induce antiviral innate immune responses for limiting viral replication and for activation of adaptive immunity. For HBV it has been recently shown that RIG-1 sensing is mediated through recognition of the 5′-end ϵ region of the HBV pg RNA which leads to the induction of type III rather than type I IFNs in human primary hepatocytes in response to in vitro infection. In addition, activated RIG-1 is able to counter-act the interaction of the HBV polymerase protein with the HBV pgRNA in an IFN pathway independent manner resulting in suppressed viral replication. In this context, it is pertinent to mention that in in vitro and in vivo studies, Formula (IIa) has shown potent antiviral activity against a number of RNA viruses, including HCV, Norovirus and RSV. The Formula (IIa)-mediated activation of RIG-1 and NOD2 supports a broad-spectrum antiviral profile, including activity against resistant variants of HCV and HBV, suggesting that these host cytoplasmic sensors should be agnostic to the type of RNA virus and genotypes. In addition, in in vitro studies using HBV-infected primary human hepatocytes, Formula (IIa) induced significant reduction in HBV DNA levels as well as declines in secreted HBsAg and HBV e antigen (HBeAg) similar to that is caused by IFN-α treatment. Taken together, the overall data from these studies supports the induction of host innate immune responses by Formula (IIa) as a significant contributor to its antiviral activity.

Aside from the host immune modulating activity (see below), the antiviral response induced by Formula (IIa) in the present study (FIGS. 2 and 4) was in the range of those of nucleos(t)ides and immunonmodulators previously evaluated in the woodchuck model of CHB. The magnitude of viral load reduction with Formula (IIa) was comparable to Emtricitabine, Tenofovir and Adefovir after administration for 12 weeks or to recombinant woodchuck IFN-α administered for 15 weeks in partial responders. As elevations in SDH noted during Formula (IIa) treatment at week 6 were temporally associated with initial reductions in serum WHsAg and hepatic WHV cccDNA (FIGS. 2-4, 11), this rise in liver enzyme activity may indicate immune mediated viral clearance of infected hepatocytes by cytotoxic effector cells. As SDH activity in serum declined during the remainder of treatment and liver inflammation was reduced at the end of treatment (FIGS. 5 and 13), this may further indicate that other, non-cytolytic mechanism(s) contributed to the peak suppression of WHV replication, including apoptosis of WHV-infected hepatocytes (although not observed on a cell level during histopathological examination of liver biopsy tissues).

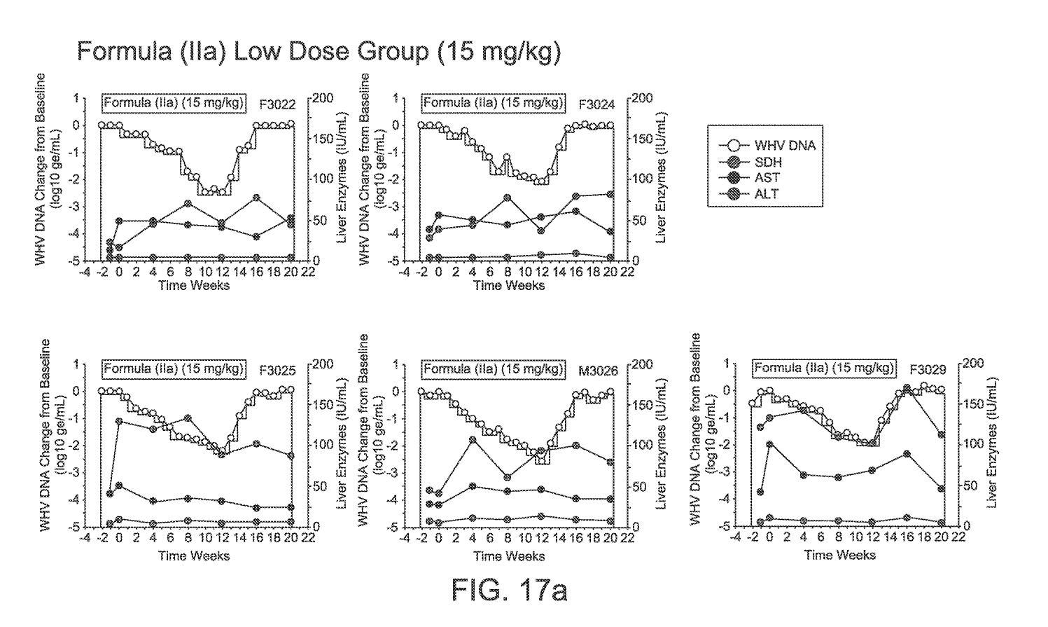

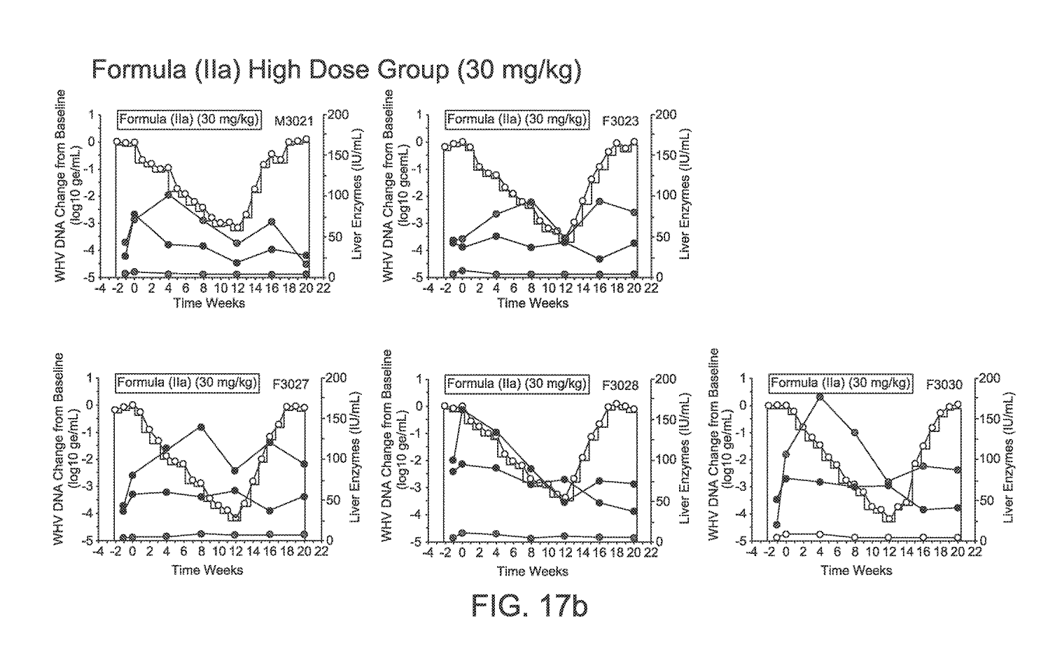

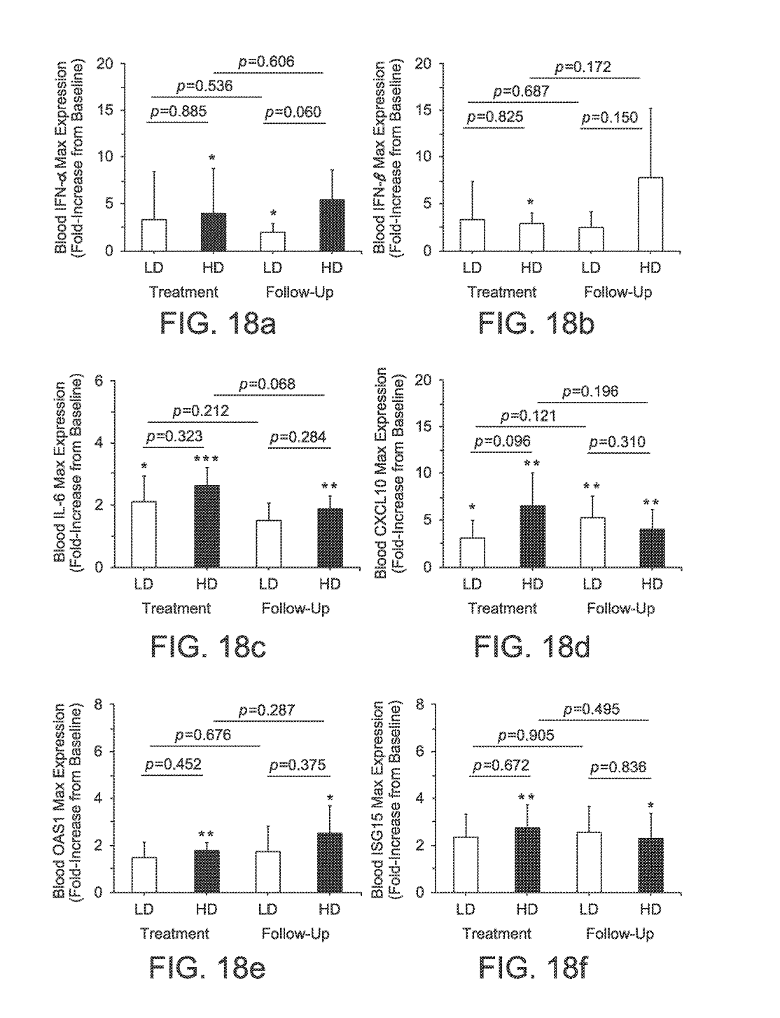

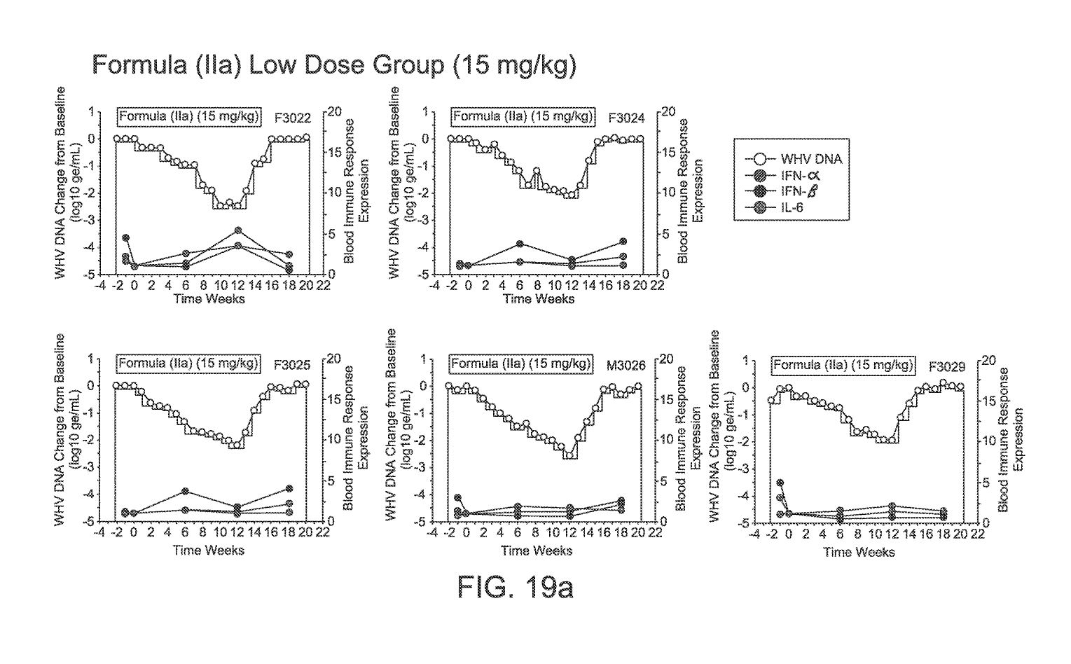

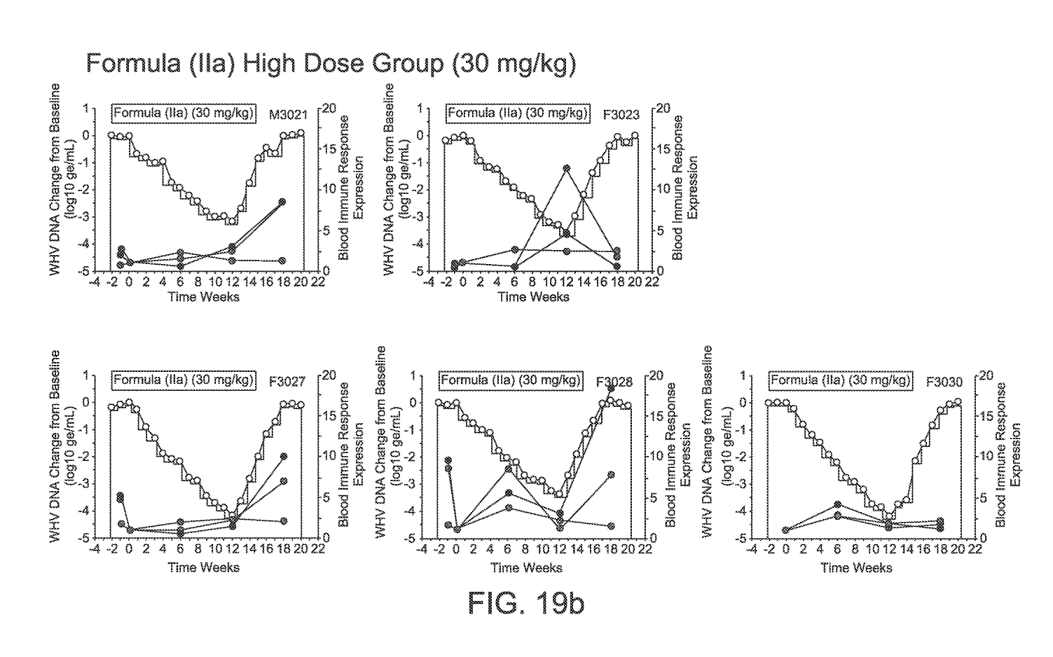

As mentioned above, type III rather than type I IFNs are predominantly induced in human primary hepatocytes in response to in vitro HBV infection through RIG-1 mediated sensing. Similarly, host innate immune response in chronic HBV and WHV infections is impaired and that the expression of type I IFNs and of IFN-α and IFN-β stimulated genes is limited in the virus-infected liver. One explanation of this finding is that the x antigen or the polymerase protein of HBV (and by analogy of WHV) interact with MAVS or competes for binding of DDX3 (a RNA helicase of the DEAD box family) with TBK1 (a serine/threonine protein kinase), respectively, and inhibits the RIG-1 mediated IFN pathway thereby possibly enabling HBV to evade the antiviral innate immune response. Considering the absent type I IFN response in chronic WHV infection, the peripheral and hepatic induction of IFN-α and IFN-β and of ISGs such as CXCL10, OAS1 and ISG15 as well as of the proinflammatory cytokine, IL-6, during and even beyond treatment with Formula (IIa) is important because it suggests that an antiviral innate immune response was induced by Formula (IIa) in a dose-dependent manner (FIGS. 7, 8, 16-21).

Since several studies have demonstrated that IFN (IFN-α) mediated antiviral effects can directly inhibit HBV and WHV, an interesting finding of this study was that the antiviral response to Formula (IIa) did not correlate well with the long-lasting hepatic expression of antiviral ISGs tested, suggesting that other immune response and/or antiviral mechanisms may play a role, especially in the peak antiviral response to treatment. Since peripheral and hepatic expression of ISGs during treatment was restricted to approximately 2-4 hours post-dose, it is likely that maximum expression of these ISGs was missed and that peak induction may be associated with the antiviral response to Formula (IIa). Finally, WHV may have limited (although not abrogate) type I IFN signaling in liver if considering a recent study demonstrating that HBV can inhibit IFN-α signaling. Considering all data derived from the present study, it appears that Formula (IIa) has also a direct antiviral component that involves interference of the WHV polymerase protein to engage with WHV pg RNA by Formula (IIa)-activated RIG-1 and NOD2) and that may have contributed to the overall, and especially peak treatment response. This assumption is consistent with the translocation studies of Formula (IIa) on dsRNA in the presence of RIG-I. Our data is also in agreement with the demonstrated potent efficacy of Formula (IIa) in HBV transgenic mice, an inherently immunotolerant animal model of chronic HBV infection. In a dose-ranging study of Formula (IIa) in the HBV transgenic mouse model once-daily oral administration of Formula (IIa) (1 to 100 mg/kg/day) for 14 days resulted in significant reduction in liver HBV DNA and the achieved antiviral effect at higher doses was comparable to that of Adefovir used as a positive control.

In summary, by establishing the efficacy, safety and pharmacodynamics of Formula (IIa) in an immunocompetent animal model of CHB, this study provided insights into the host immune stimulating and direct antiviral activities of this new class of anti-HBV compounds.

### Example 3

### Study and Efficacy of Formula (IIa) Treatment in Combination with Entecavir in the Woodchuck Model of Chronic Hepatitis B Virus Infection

### Study Objective

The objective of this study was to determine if prolonged treatment with Formula (IIa) in combination with entecavir (ETV) is safe and produces sustained antiviral activity in woodchucks chronically infected with the woodchuck hepatitis virus (WHV), an established animal model of chronic hepatitis B virus (HBV) infection. The study tested Formula (IIa) during a 12-week period of oral drug administration in combination with 4 weeks of oral treatment with the direct acting antiviral, ETV. One group of woodchucks received ETV during the initial 4 weeks of treatment, followed by 12 weeks of Formula (IIa) dosing. The other group of woodchucks was initially treated with Formula (IIa) for 12 weeks, followed by 4 weeks of ETV treatment. Treated woodchucks were followed for a total of 24 weeks and changes in viremia and antigenemia in serum and liver from pretreatment level were evaluated. In addition, Formula (IIa) concentrations in serum and liver, induction of host innate immune responses in peripheral blood and liver, seroconversion to antibodies against WHV surface antigen, and rebound viremia off Formula (IIa)/ETV treatment were be determined.

### Study Design

Ten (10) woodchucks chronically infected with WHV were used. Woodchucks were born in captivity and were inoculated at 3 days of age with cWHV7P2a inoculum, then reared until 11 months of age. Prior to the study, woodchucks were confirmed as established chronic carriers of WHV based on established criteria including the presence of WHV surface antigen (WHsAg), antibody against WHsAg (anti-WHs), and WHV DNA. Woodchucks used in the study were of both gender and approximately between 12 and 14 months of age at the start of the study.

### Experimental Plan:

Two to one weeks prior to the initiation of the study, chronic WHV carrier woodchucks were anesthetized and whole blood drawn for WHV serology (determinations of WHV surface antigen [WHsAg] and antibodies against WHsAg [anti-WHs]), for serum WHV DNA, for complete blood counts (CBCs), and for clinical biochemical profiles (see Summary Table below). An additional whole blood aliquot was drawn into PAXgene blood tubes (Qiagen) and stored for determinations of transcript expression changes of selected host innate immune response genes by real time PCR. Another whole blood aliquot was obtained for later determination of Formula (IIa) concentrations in plasma.

Body weights and body temperatures was determined (i.e., woodchucks were microchipped for accurate identification and the implant also records body temperature via a hand-held scanner). Using the parameters described above, woodchucks were stratified into two groups based on gender, body weight, serum viral and antigen loads, and serum levels of gamma-glutamyl transferase (GGT). If necessary, woodchucks were further stratified on the basis of hematological and other clinical biochemical data. Selected woodchucks were free of evidence for HCC based on low GGT levels that will be confirmed upon arrival at the study site by hepatic ultrasound examination.

Two (2) groups of five (5) chronic WHV carrier woodchucks each were used in the study:

Group 1 was orally treated for 4 weeks with ETV at a daily dose of 0.5 mg/kg starting at T₀, followed by add-on treatment with Formula (IIa) for 12 weeks at a daily dose of 30 mg/kg. Woodchucks were then followed for a total of 24 weeks.

Group 2 was orally treated for 12 weeks with Formula (IIa) at a daily dose of 30 mg/kg starting at T₀, followed by add-on treatment with ETV for 4 weeks at a daily dose of 0.5 mg/kg. Woodchucks were then also followed for a total of 24 weeks.

The dose of Formula (IIa) administered orally to woodchucks of Group 1 and 2 was selected by the based on the results of the results of Example 2 in which 12 weeks of Formula (IIa) treatment at a daily oral dose of 30 mg/kg was without toxicity but produced significant antiviral efficacy. The dose of ETV was based on previous short-and long-term treatment studies in woodchucks. It is expected that daily oral dosing with ETV at 0.5 mg/kg for 4 weeks will suppress serum viremia by approximately 3-4 log₁₀.

Formula (IIa) was supplied as a pre-weighed powder in individual containers. An amount of powder sufficient to treat all 10 pre-weighed woodchucks per day with Formula (IIa) was mixed directly with woodchuck diet powder (Dyets Inc., Bethlehem, Pa.) and then suspended with HPLC water. The drug was orally administered with a dose syringe at T₀ (Group 2) or after 4 weeks of treatment with ETV (Group 1), and thereafter every day for 12 weeks (84 days). Drug dosing was then followed by an extra 2 ml of woodchuck liquid diet to ensure complete consumption of Formula (IIa).

ETV was provided as powder. An amount of powder sufficient to treat all 10 woodchucks per day was suspended in isotonic saline at a concentration of 0.5 mg/ml. A volume (ml) of drug solution equivalent to the body weight (kg) of an individual woodchuck was then mixed with approximately 3-5 ml of woodchuck liquid diet and administered orally with a dose syringe at T₀ (Group 1) or after 12 weeks of treatment with Formula (IIa) (Group 2), and thereafter every day for 4 weeks (28 days). ETV dosing was followed by an extra 2 ml of woodchuck liquid diet to ensure complete consumption of drug.

Prior to administration of Formula (IIa) or ETV at T₀, then weekly until the end of the study at week 24, whole blood samples were drawn from each woodchuck under ketamine/xylazine anesthesia for determinations of serum WHV DNA, WHsAg, and anti-WHs (see Summary Table below). WHsAg and anti-WHs were measured quantitatively if indicated by significant reductions in serum. Blood samples were kept at room temperature following collection and serum harvested upon observation of clot retraction. Harvested serum was transferred to microcentrifuge tubes and stored frozen at −70° C. until use.

Whole blood samples for the determination of Formula (IIa) plasma concentrations were also obtained prior to drug treatment at T₀, and then bi-weekly throughout the study (see Summary Table below). Whole blood was collected into K3EDTA tubes and placed on wet ice for no longer than 30 minutes before processing. Thereafter, blood will be centrifuged at approximately 5000 g for 15 minutes at 4° C., and duplicate plasma samples (>0.1 ml each) transferred into fresh storage tubes. Plasma will be stored frozen at −70° C. until the end of the study.

Whole EDTA blood samples for CBCs and serum samples for biochemical profiles will be obtained prior to the start of treatment with Formula (IIa) or ETV at T₀, and then at weeks 4, 8, 12, 16, 20, and 24 (see Summary Table below). Additional CBCs and biochemical profiles were obtained if indicated by clinical abnormalities, especially during combination treatment with Formula (IIa) and ETV. Blood samples for CBC determinations were kept at 4° C. following collection and sent out the same day on cold packs to the Animal Health Diagnostic Center (AHDC) at Cornell University (Ithaca, N.Y.). Serum samples for biochemical tests that include the serum activities of liver enzymes were stored at −70° C. and sent out during the week of collection on dry ice to AHDC.

The induction of an innate immune response in peripheral blood and liver of woodchucks by combination treatment with Formula (IIa) and ETV were determined via real time PCR-based assays. Whole blood samples were obtained prior to the start of treatment with Formula (IIa) at T₀, and then at weeks 4, 10, 16, 20, and 24 (Group 1) or at weeks 6, 12, 16, 20, and 24 (Group 2) (see Summary Table below). Whole blood was drawn into PAXgene blood tubes at the indicated time points and stored at −70° C. until use. Total RNA was isolated from whole blood and treated with DNase using the PAXgene Blood miRNA Kit (Quiagen). This was followed by reverse transcription of mRNA into cDNA and amplification of host immune response genes by real time PCR. Woodchuck cytokine and interferon-stimulated genes included IFN-α, IFN-β, interleukin 6 (IL-6), 2′-5′-oligoadenylate synthetase 1 (OAS-1), interferon-induced 17 kDa protein (ISG15), and interferon gamma-induced protein 10 (IP-10or CXCL10). Woodchuck 18S rRNA expression was used to normalize target gene expression. Liver biopsies were obtained under anesthesia using 16 g disposable needles directed by ultrasound imaging at pretreatment (i.e., approximately 1 week prior to the start of treatment with Formula (IIa) or ETV at T₀). Additional liver biopsies were obtained at weeks 4, 16 and 20 (Group 1) or at weeks 12, 16 and 20 (Group 2). A final liver biopsy was performed at the end of the study at week 24 during necropsy (see Summary Table below). The needle was inserted dorsolaterally and somewhat cranially into the margin of the large, left lateral lobe of the liver. Assessments of the biopsy specimens included measurement of viral nucleic acids (i.e., WHV covalently closed circular DNA, WHV DNA replicative intermediates, and WHV RNA), histology for progression of liver disease and cancer (i.e., portal inflammation/hepatitis, bile duct proliferation, sinusoidal inflammation/hepatitis, necrosis, fibrosis, steatosis, and apoptosis (i.e., apoptotic bodies), and immunohistochemistry for hepatic expression of WHV antigens (i.e., surface and core). Liver tissue was stored frozen at −70° C. until the end of the study.

Prior to the start of treatment with Formula (IIa) or ETV at T₀, and then weekly until the end of the study at week 24, body weight and body temperature of woodchucks was recorded (see Summary Table below). Body temperatures were obtained via a microchip inserted subcutaneously that can be read with a hand-held scanner. Additional body temperatures were obtained during combination treatment with Formula (IIa) and ETV, if indicated by clinical abnormalities (i.e., fever spikes).

Whole blood samples for determination of host innate immune responses were obtained, as well as serum samples for determination of Formula (IIa) plasma concentrations. Liver samples for WHV nucleic acid determinations, histology, immunohistochemistry, host innate immune response measurements, and hepatic Formula (IIa) concentrations were acquired immediately following euthanasia and before complete postmortem examination.

TABLE 1
Summary Table of Experimental Procedures and Tests

| | | | | Test | | | |
|---|---|---|---|---|---|---|---|
| | | | | | | Plasma | |
| | | | | | | for | Whole Blood |
| | Experimental Procedure | | | | | Formula | for Host Innate |
| | | | Body | WHV | | (Ia) | Immune |
| Week of | | Liver | Weight | DNA & | CBC/Serum | Level | Response |
| Study | Bleed | Biopsy^{a,b} | &Temp. | Serology | Biochemistry^{c} | Analysis^{a} | Analysis^{b} |
| Week −2 to −1 | + | + | + | + | + | + | + |
| T₀* | + | | + | + | + | + | + |
| Week 1 | + | | + | + | | | |
| Week 2 | + | | + | + | | + | |
| Week 3 | + | | + | + | | | |
| Week 4 | + | +G1 | + | + | + | + | +G1G1 |
| Week 5 | + | | + | + | | | |
| Week 6 | + | | + | + | | + | +G2 |
| Week 7 | + | | + | + | | | |
| Week 8 | + | | + | + | + | + | |
| Week 9 | + | | + | + | | | |
| Week 10 | + | | + | + | | + | +G1 |
| Week 11 | + | | + | + | | | |
| Week 12 | + | +G2 | + | + | + | + | +G2 |
| Week 13 | + | | + | + | | | |
| Week 14 | + | | + | + | | + | |
| Week 15 | + | | + | + | | | |
| Week 16 | + | + | + | + | + | + | + |
| Week 17 | + | | + | + | | | |
| Week 18 | + | | + | + | | + | |
| Week 19 | + | | + | + | | | |
| Week 20 | + | + | + | + | + | + | + |
| Week 21 | + | | | + | | | |
| Week 22 | + | | + | + | | + | |
| Week 23 | + | | + | + | | | |
| Week 24 | + | + | + | + | + | + | + |

*Woodchucks of Group 1 (G1 = ETV +Formula (IIa) group) were orally treated for 4 weeks with ETV at a dose of 0.5 mg/kg starting at T₀, followed by add-on treatment with Formula (IIa) for 12 weeks at a dose of 30 mg/kg. Woodchucks of Group 2 (G2 = Formula (IIa) +ETV group) were orally treated for 12 weeks with Formula (IIa) at a dose of 30 mg/kg starting at T₀, followed by add-on treatment with ETV for 4 weeks at a dose of 0.5 mg/kg.
^{a}Levels of Formula (IIa) will be determined in liver and plasma.
^{b}Host innate immune response in liver and peripheral blood following treatment with Formula (IIa) and ETV will be determined using real time RT-PCR-based assays.
^{c}Hematology and clinical chemistry parameters will be determined at Cornell University (AHDC, Ithaca, NY).

Study results

Antiviral activity of treatment with Formula (IIa) and ETV was assessed by comparing serum WHV DNA and WHsAg loads and hepatic WHV nucleic acid levels of chronic WHV carrier woodchucks during/following combination treatment with those obtained at pretreatment. The immune modulating activity of Formula (IIa) was assessed by comparing the mRNA expression of selected host innate immune response genes in peripheral blood and liver during/following combination treatment with that observed at pretreatment. Seroconversion in chronic WHV carrier woodchucks following administration of Formula (IIa) and ETV was assessed by comparing the anti-WHs levels during/following combination treatment with those obtained at pretreatment. Progression of liver disease, hepatic expression of WHV antigens, and Formula (IIa) induced apoptosis during/following combination treatment were also compared to the same parameters observed at pretreatment. Possible toxicity associated with Formula (IIa) and ETV treatment was evaluated by clinical observations made daily and by comparing weekly body temperature and body weight measurements during/following combination treatment with those obtained at pretreatment, and by comparing hematological and clinical chemistry parameters during the course of the study. All parameters described above were also compared between woodchucks of Groups 1 and 2 for determination of sustained antiviral effects associated with both combination treatment regimens.

### Body Weights and Body Temperature

Woodchucks treated with Formula (IIa) in combination with ETV gained body weight throughout the study, especially during the treatment and follow up periods between T₀ and week 16 or weeks 17 to 19, respectively. Overall, the mean body weight was comparable between both groups during most of the study period but there was a tendency toward slightly lower body weight in woodchucks treated with Formula (IIa) plus ETV during the follow-up period between weeks 21 and 24, when compared to woodchucks that received ETV plus Formula (IIa). However, the changes in mean body weight between both groups were comparable throughout the study and were not statistically different (P>0.05), indicating that there was no evidence of overt toxicity related to Formula (IIa) treatment in combination with ETV for 16 weeks.

Mean body temperature for woodchucks of both groups fluctuated over time but remained essentially stable throughout most of the study. Overall, the changes in mean body temperature between woodchucks of both groups were comparable throughout the study and not statistically different (P>0.05), indicating that Formula (IIa) treatment in combination with ETV was not associated with any long-lasting fever spikes in individual woodchucks.

### Serum WHV DNA

There were consistent and significant (P<0.05) treatment regimen effects of oral dosing with Formula (IIa) in combination with ETV for 16 weeks on viral markers in serum, when compared to the pretreatment level at T₀. Antiviral effects were more pronounced and longer-lasting in woodchucks of the ‘Formula (IIa)+ETV’ group treated first with Formula (IIa) (i.e., 30 mg/kg) for 12 weeks followed by add-on administration of ETV (i.e., 0.5 mg/kg) for 4 weeks than in woodchucks of the ‘ETV+Formula (IIa)’ group administered first ETV for 4 weeks followed by add-on treatment with Formula (IIa) for 12 weeks. Antiviral effects mediated by Formula (IIa) on WHV DNA and WHsAg were uniform for most woodchucks of the ‘Formula (IIa)+ETV’ group, whereas woodchucks of the ‘ETV+Formula (IIa)’ group showed more variability in antiviral response, especially during rebound of serum WHV markers following cessation of ETV treatment and during viral suppression by continued treatment with Formula (IIa). Antiviral effects mediated by ETV on WHV DNA (and in part on WHsAg) were marked in woodchucks of both groups.

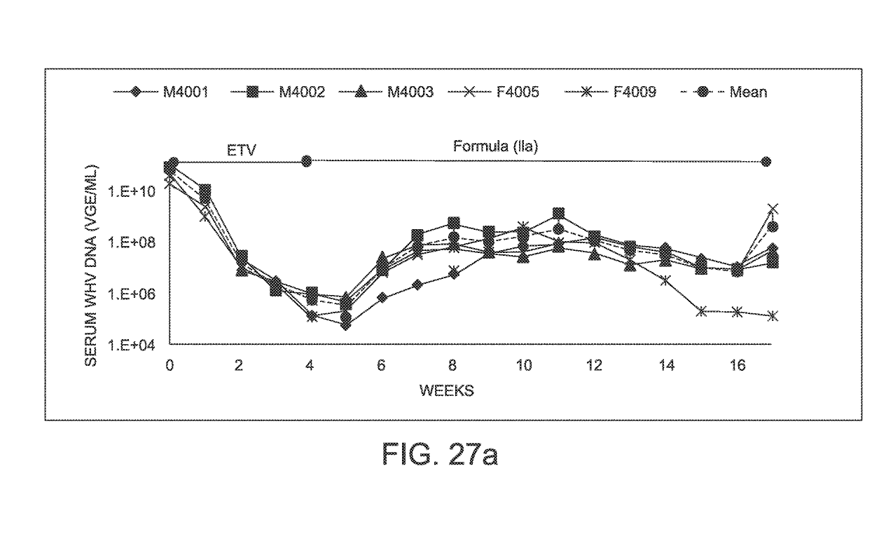

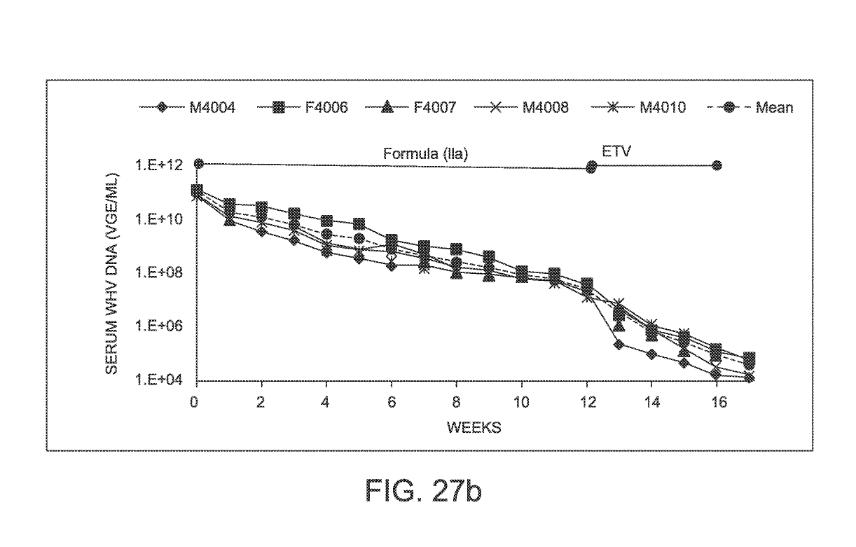

All woodchucks of the ‘ETV+Formula (IIa)’ group had marked reductions in serum WHV DNA from pretreatment level during the 4 weeks of ETV (FIGS. 27A-B). Serum WHV DNA became never undetectable in any of the woodchucks in this group, and variability in antiviral effect was observed during the treatment period (i.e., woodchuck F4009). After cessation of Formula (IIa) treatment, all woodchucks showed recrudescence of viral replication, and serum viremia increased immediately by 2.96 log₁₀ during the initial 2 weeks of the follow-up period, and then more gradually by another 1.0 log₁₀ until the end of the study at week 24. In woodchucks of this group, serum WHV DNA returned to pretreatment level within 2-6 weeks after Formula (IIa) withdrawal (i.e., by weeks 18-22 of the study). All woodchucks of the ‘Formula (IIa)+ETV’ group had pronounced reductions in serum WHV DNA during the 12 weeks of Formula (IIa) treatment that were noticed as early as week 1 after the start of dosing. Reductions in group mean serum viremia appeared to occur gradually as WHV DNA declined by approximately 1 log 10 every 3 weeks during the initial 6 weeks of treatment. At the end of Formula (IIa) treatment at week 12, mean serum WHV DNA was reduced from pretreatment level by 3.54 log 10. Following the add-on ETV treatment for 4 weeks, mean serum viremia declined further by 2.80 log 10 and was reduced from pretreatment level by 6.34 log 10 at week 17 (i.e., one week after cessation of ETV treatment, FIGS. 27A-B). Serum WHV DNA, however, became never undetectable in any of the woodchucks in this group. After cessation of ETV treatment, all woodchucks showed recrudescence of viral replication, and mean serum viremia increased immediately by 3.26 log 10 during the initial 3 weeks of the follow-up period. Thereafter, mean serum WHV DNA increased less quickly and only by 1.47 log 10 until the end of the study at week 24. Serum WHV DNA in the surviving woodchucks of this group never returned to pretreatment level during the follow-up period, and mean serum viremia stayed 1.62 log 10 below the baseline.

In summary, the salient observations for serum WHV DNA described above included: 1) uniform and significant (P<0.05) reductions in serum viremia from pretreatment level during initial ETV and Formula (IIa) treatment for 4 or 12 weeks, respectively; 2) more pronounced reductions in serum viremia from pretreatment level during Formula (IIa) treatment in combination with ETV in the ‘Formula (IIa)+ETV’ group than during ETV treatment in combination with Formula (IIa) in the ‘ETV+Formula (IIa) group; and 3) return of serum viremia to pretreatment level following cessation of Formula (IIa) treatment in the ‘ETV+Formula (IIa) group that was delayed following cessation of ETV treatment in the ‘Formula (IIa)+ETV’ group. Due to the above differences, the serum geometric WHV DNA concentration was significantly lower (P<0.05) in the ‘ETV+Formula (IIa) group than in the ‘Formula (IIa)+ETV’ group between weeks 1 and 7 of the study. However, the serum geometric WHV DNA concentration was significantly lower (P<0.05) in the ‘Formula (IIa)+ETV’ group than in the ‘ETV+Formula (IIa) group between weeks 12 and 24, demonstrating a treatment regimen dependent effect on serum viremia in regard to the magnitude and sustainability of serum WHV DNA reduction.

### Serum WHsAg

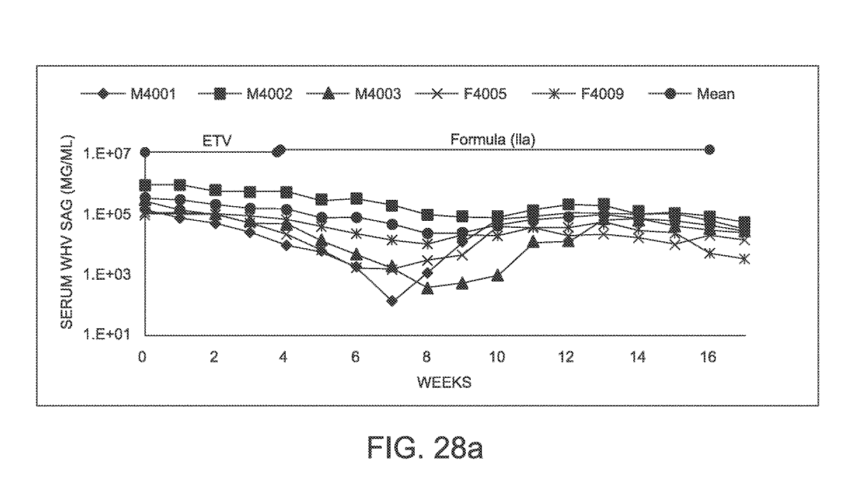

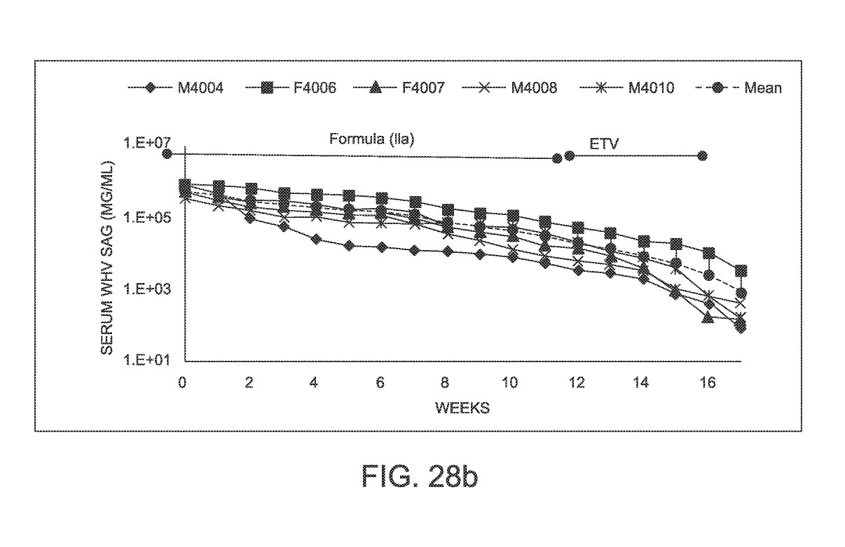

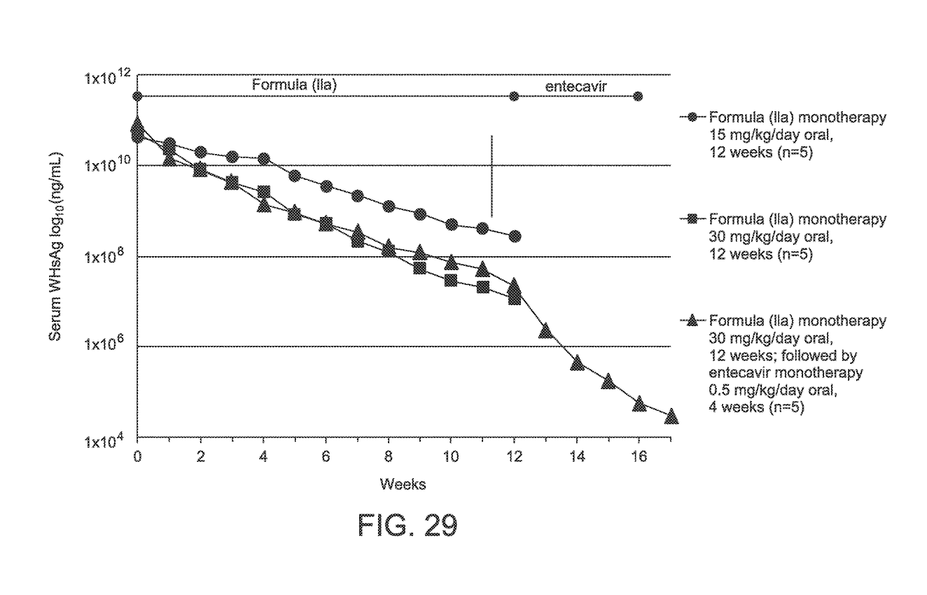

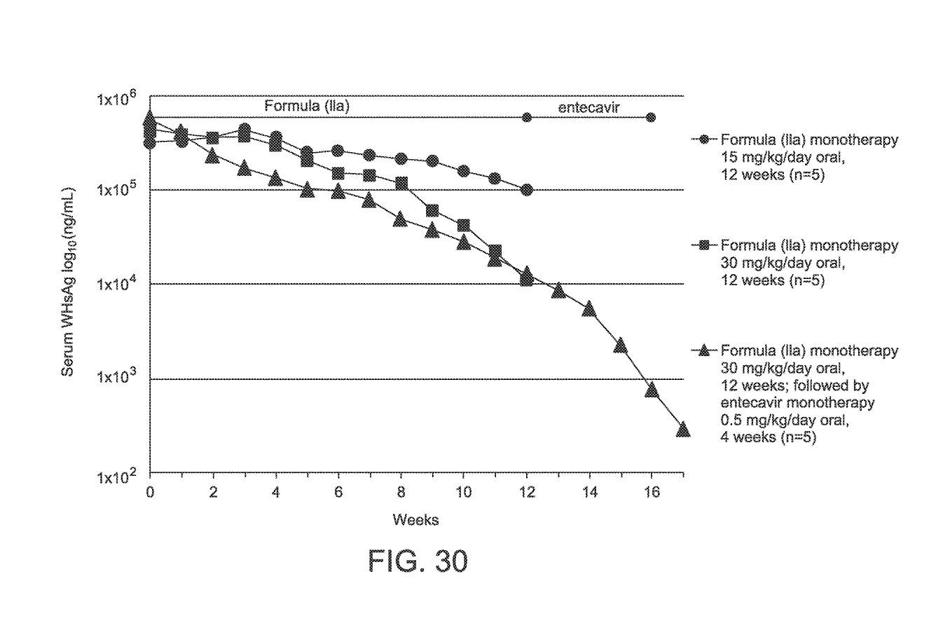

Pronounced changes were also observed in the serum WHsAg levels of woodchucks of the ‘ETV+Formula (IIa)’ and ‘Formula (IIa)+ETV’ groups during the 16 weeks of Formula (IIa) treatment in combination with ETV (FIGS. 28A-B). Most woodchucks of the ‘ETV+Formula (IIa)’ group had marked reductions in serum WHsAg from pretreatment level during the 4 weeks of ETV administration that were observed as early as 1-3 weeks after the start of treatment, except for woodchuck M4002 in which the decline in WHsAg was less pronounced. Reductions in group mean serum antigenemia appeared to occur more gradually when compared to the decline of mean serum viremia, but WHsAg declined by 1.17 log 10 until week 8 (i.e., four weeks after cessation of ETV treatment). Despite add-on Formula (IIa) treatment from week 5 onward, mean serum antigenemia increased by 0.62 log 10 at week 13 but still stayed 0.55 log 10 below the pretreatment level. During the remainder of Formula (IIa) treatment, the mean serum antigenemia declined again by 0.57 log 10 and was reduced from pretreatment level by 1.13 log 10 at week 17 (one week after cessation of Formula (IIa) treatment). After cessation of Formula (IIa) treatment, all woodchucks showed recrudescence of viral replication, and serum antigenemia increased gradually by 1.15 log 10 until the end of the study at week 24. In woodchucks of this group, serum WHsAg returned to pretreatment levels within 3-8 weeks after Formula (IIa) withdrawal (i.e., by weeks 19-24 of the study). All woodchucks of the ‘Formula (IIa)+ETV’ group had pronounced reductions in serum WHsAg during the 12 weeks of Formula (IIa) treatment that were noticed as early as 1-2 weeks after the start of dosing. Reductions in group mean serum viremia appeared to occur gradually, and WHsAg declined by 1.49 log 10 until the end of Formula (IIa) treatment in week 12. Following add-on ETV treatment for 4 weeks, mean serum antigenemia declined further by 1.38 log 10 and was reduced from pretreatment level by 2.87 log 10 at week 17 (i.e., one week after cessation of ETV treatment). After cessation of ETV treatment, all woodchucks showed recrudescence of viral replication and mean serum antigenemia increased by 2.28 log 10 until the end of the study at week 24. Serum WHsAg in the surviving woodchucks of this group never returned to pretreatment level and mean serum antigenemia stayed 0.59 log 10 below the baseline. Overall, compared to the pretreatment level at T0, the geometric mean WHsAg concentration in serum of woodchucks of the ‘ETV+Formula (IIa)’ group was maximal reduced by 1.76 log 10 at week 7 (after 4 weeks of ETV treatment), and by 1.10 log 10 at week 17 (after 12 weeks of Formula (IIa) treatment). At the end of the follow-up period at week 24, the serum geometric WHsAg concentration was slightly higher than that at pretreatment at T0. The geometric mean WHsAg concentration in serum of woodchucks of the ‘Formula (IIa)+ETV’ group was maximal reduced by 1.66.

In summary, salient observations for serum WHsAg described above included: 1) significant (P<0.05) reductions in serum antigenemia from pretreatment level during initial ETV and Formula (IIa) treatment for 4 or 12 weeks, respectively; 2) more pronounced reductions in serum antigenemia from pretreatment level during Formula (IIa) treatment in combination with ETV in the ‘Formula (IIa)+ETV’ group than during ETV treatment in combination with Formula (IIa) in the ‘ETV+Formula (IIa)’ group; and 3) return of serum antigenemia to pretreatment level following cessation of Formula (IIa) treatment in the ‘ETV+Formula (IIa)’ group that was delayed following cessation of ETV treatment in the ‘Formula (IIa)+ETV’ group. Due to the above differences, the serum geometric WHsAg concentration was significantly lower (P<0.05) in the ‘Formula (IIa)+ETV’ group than in the ‘ETV+Formula (IIa)’ group between weeks 13 and 19 and again between weeks 21 and 23 of the study, demonstrating a treatment regimen dependent effect on serum antigenemia in regard to the magnitude and sustainability of serum WHsAg reduction.

### Example 4

### EC₅₀ Determination of Formula (IIa) in Cells Chronically Infected with Resistant Variants of HBV

Antiviral assays were carried out in cells chronically infected with resistant variants of HBV. In order to gauge the efficacy of Formula (IIa) and the antiviral nucleoside analogs lamivudine (3TC) and adefovir dipivoxil (ADV) against the tested strains, the in vitro activity (EC₅₀, μM) was determined using six different cell samples chronically infected with HBV. Each cell sample was infected with either wild type HBV or a resistant variant of HBV, wherein the resistant variants comprise mutations in the HBV polymerase (P) at the following amino acid positions: M204V, M204I, L180M, L180M/M204V, and N236T. In each assay, Formula (IIa), 3TC, or ADV was added to the cells daily for nine consecutive days.

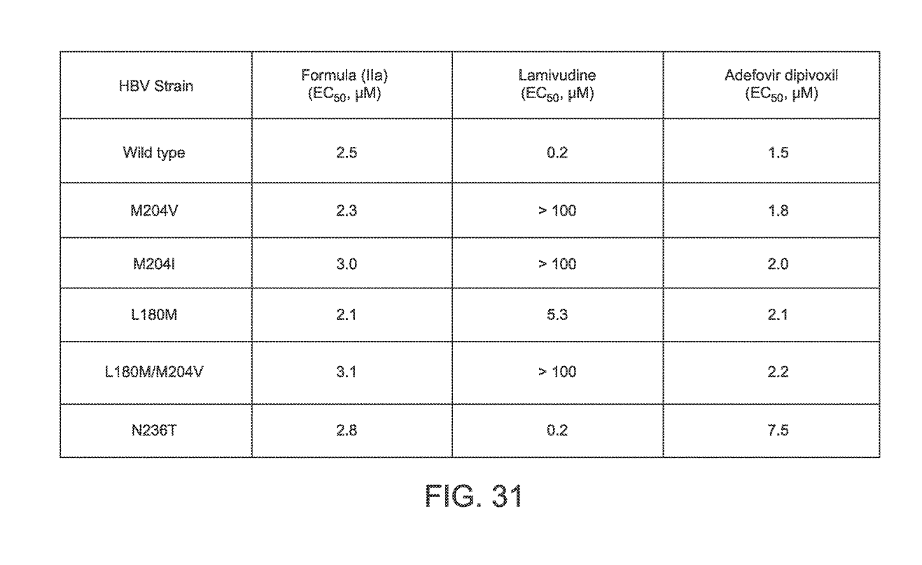

The data indicate that Formula (IIa) was efficacious against cells infected with all the HBV strains tested, both wild type and resistant variants, similar to ADV (FIG. 31). By contrast, 3TC was only effective against less than half of the resistant HBV strains tested.

### EQUIVALENTS

The disclosures of each and every patent, patent application, and publication cited herein are hereby incorporated herein by reference in their entirety. While this disclosure has been described with reference to specific aspects, it is apparent that other aspects and variations may be devised by others skilled in the art without departing from the true spirit and scope of the disclosure. The appended claims are intended to be construed to include all such aspects and equivalent variations. Any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference.

While this disclosure has been particularly shown and described with references to preferred embodiments thereof, it will be understood by those skilled in the art that various changes in form and details may be made therein without departing from the scope of the disclosure encompassed by the appended claims.

1. A method of treating a subject infected with the Hepatitis B virus, the method comprising administering to the subject a pharmaceutical composition comprising a compound of Formula (I) at a dosage of about 0.5 mg/kg to about 100 mg/kg, wherein the compound is selected from: 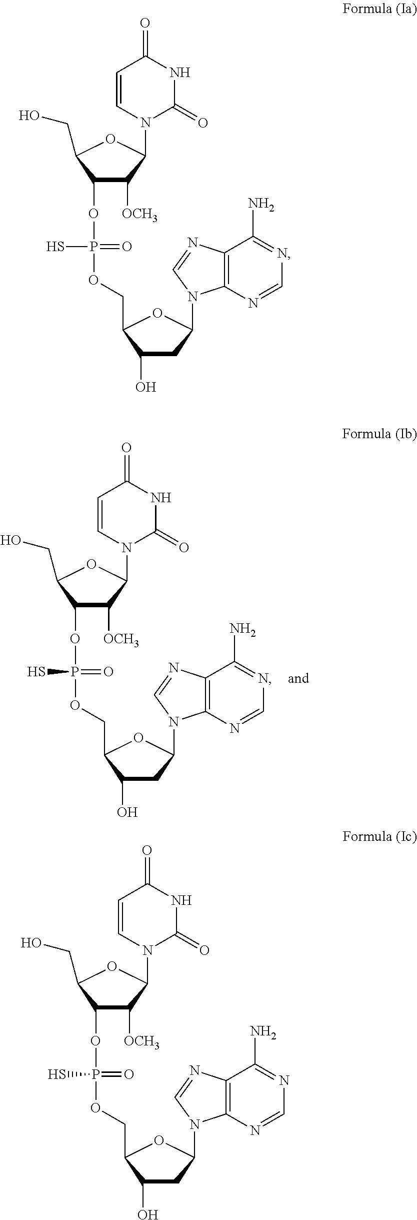
or a prodrug or pharmaceutically acceptable salt thereof to thereby treat the subject.

2. The method of claim 1, wherein the prodrug of Formula (I) is a compound of Formula (II), wherein the compound is selected from: 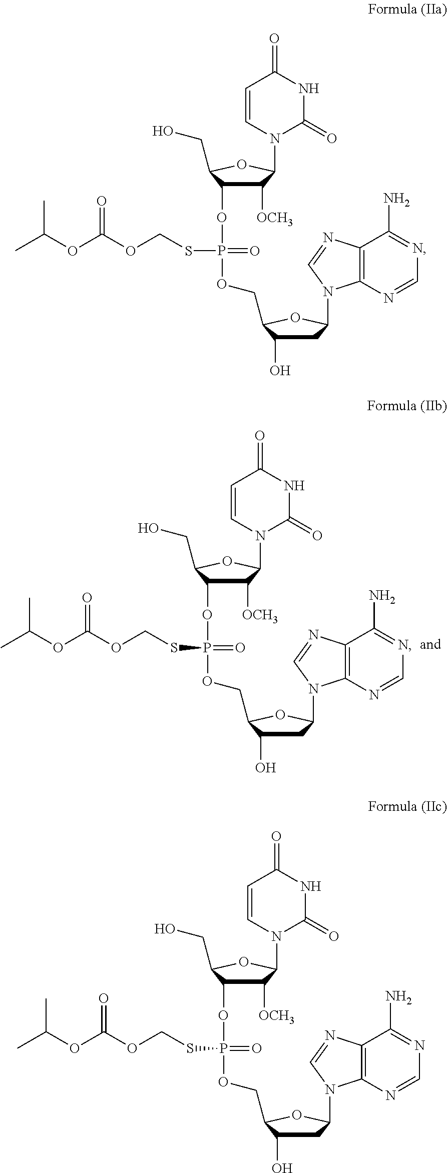
or a pharmaceutically acceptable salt thereof.

3. The method of claim 1, wherein the composition comprises a mixture of compounds of Formula (I), e.g., Formula (Ib) and Formula (Ic).

4-5. (canceled)

6. The method of claim 2, wherein the composition comprises a mixture of compounds of Formula (II), e.g., Formula (IIb) and Formula (IIc).

7-8. (canceled)

9. The method of claim 1, wherein the compound of Formula (I) or Formula (II) is administered orally (e.g., the compound of Formula (II) is administered orally).

10. The method claim 1, wherein the compound of Formula (I) or Formula (II) is administered parenterally (e.g., the compound of Formula (II) is administered parenterally).

11-19. (canceled)

20. The method of claim 1, further comprising the administration of a therapeutically effective amount of an additional agent.

21. The method of claim 20, wherein the additional agent is an antiviral agent or an anticancer agent.

22-24. (canceled)

25. The method of claim 21, wherein the antiviral agent is entecavir.

26-39. (canceled)

40. A method of treating Hepatitis B virus in a subject, the method comprising administering to the subject a compound of Formula (I), wherein the compound is selected from: 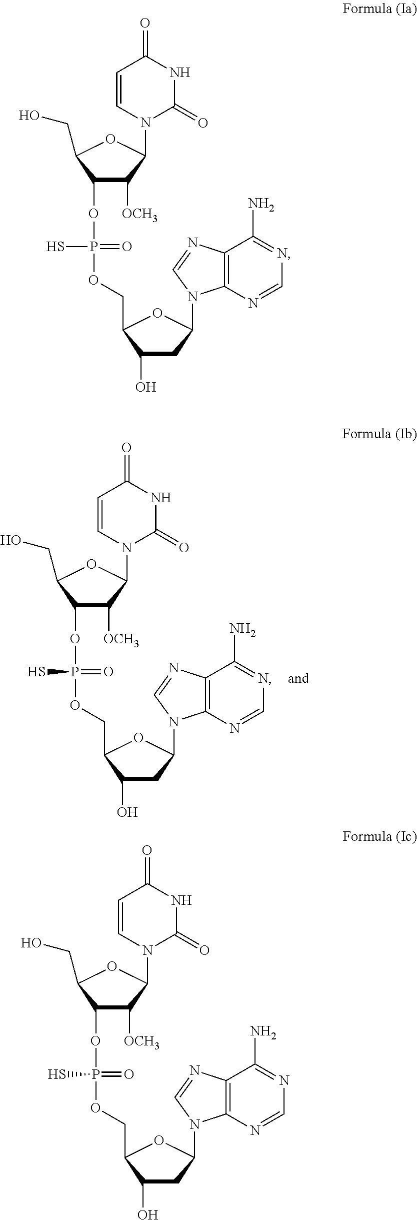
or a prodrug or pharmaceutically acceptable salt thereof in combination with entecavir or a pharmaceutically acceptable salt thereof to thereby treat the subject.

41. A method of treating Hepatitis B virus in a subject comprising administering to the subject a course of entecavir or a pharmaceutically acceptable salt thereof, wherein the subject has previously been treated with a course of compound of Formula (I), wherein the compound is selected from: 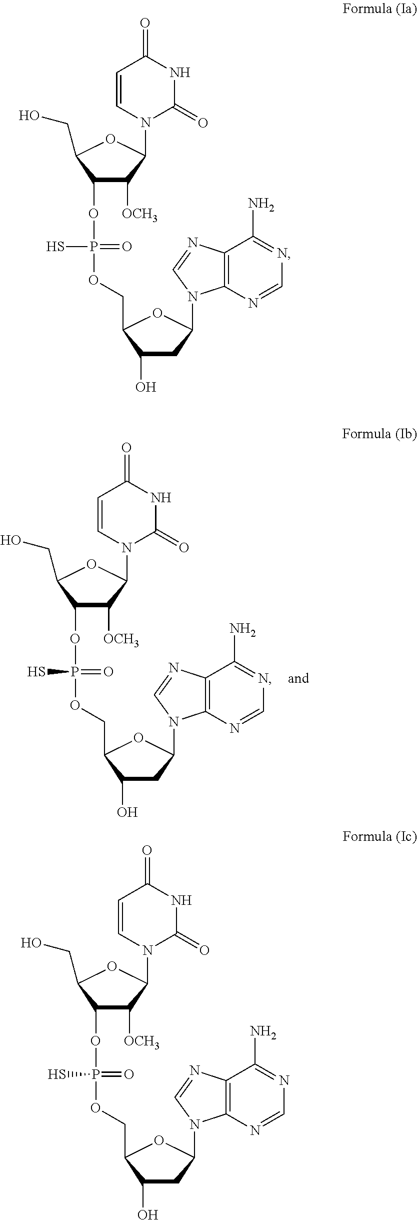
or a prodrug or pharmaceutically acceptable salt thereof to thereby treat the subject.

42-44. (canceled)

45. The method of claim 40, wherein the prodrug of Formula (I) is a compound of Formula (II), wherein the compound is selected from: 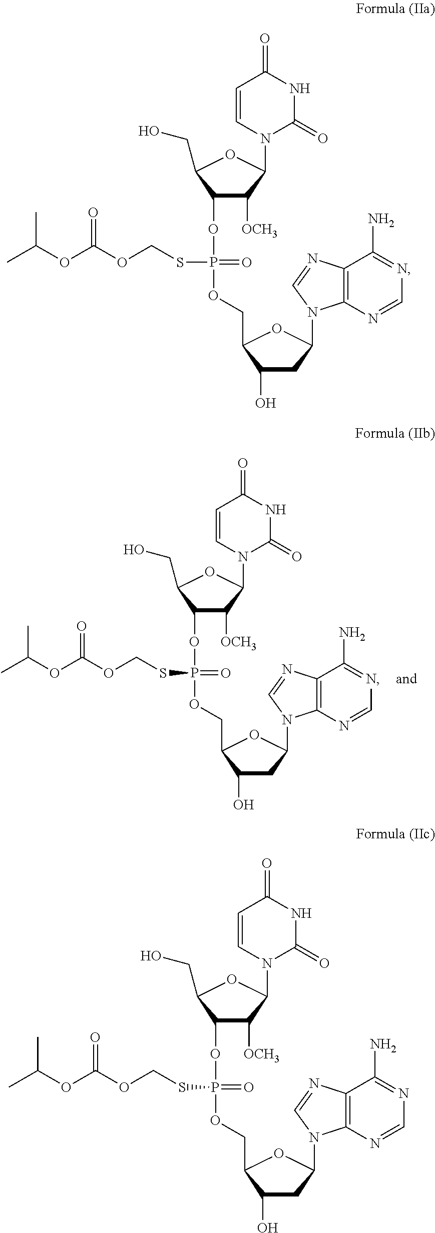
or a pharmaceutically acceptable salt thereof.

46-61. (canceled)

62. The method of claim 45, wherein the compound of Formula (I) or Formula (II) is formulated as a fixed dose combination with entecavir (e.g., as a liquid dosage form or solid dosage form, e.g., a capsule or tablet).

63. (canceled)

64. The method of claim 40, wherein the composition comprises a mixture of compounds of Formula (I), e.g., Formula (Ib) and Formula (Ic).

65-66. (canceled)

67. The method of claim 45, wherein the composition comprises a mixture of compounds of Formula (II), e.g., Formula (IIb) and Formula (IIc).

68-71. (canceled)

72. The method of claim 45, further comprising the administration of a therapeutically effective amount of an additional agent.

73-91. (canceled)

92. A method of treating Hepatitis D virus in a subject, the method comprising administering to the subject a compound of Formula (I), wherein the compound is selected from: 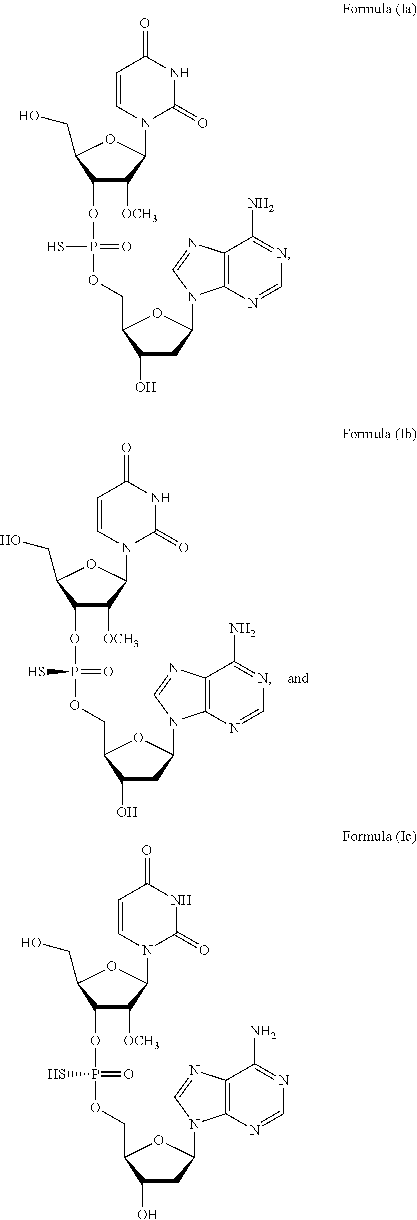
or a prodrug or pharmaceutically acceptable salt thereof in combination with entecavir or a pharmaceutically acceptable salt thereof to thereby treat the subject.

93. The method of claim 92, wherein the prodrug of Formula (I) is a compound of Formula (II), wherein the compound is selected from: 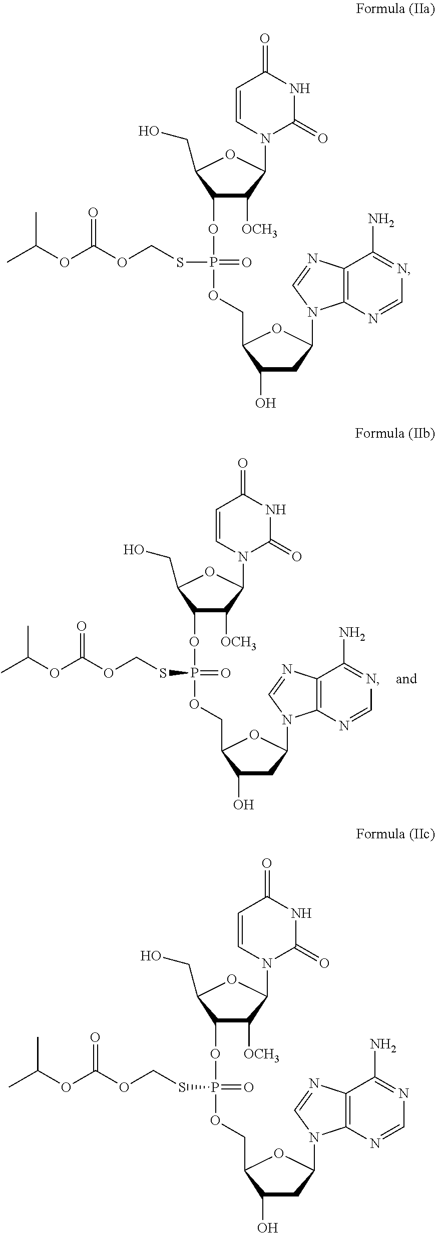
or a pharmaceutically acceptable salt thereof.

94-186. (canceled)

187. A pharmaceutical composition comprising a compound of Formula (I) wherein the compound is selected from: 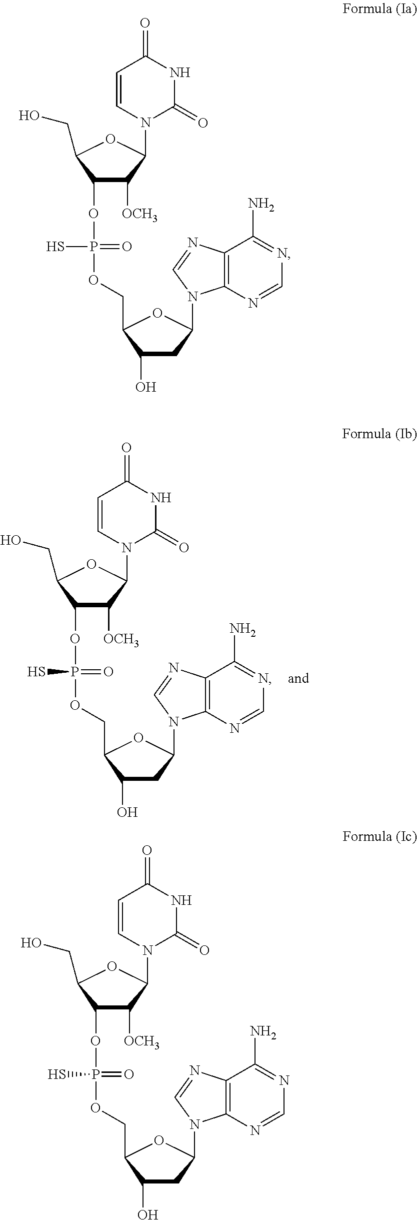
or a prodrug or pharmaceutically acceptable salt thereof and entecavir or a pharmaceutically acceptable salt thereof.

188. (canceled)

189. The composition of claim 187, wherein the prodrug of Formula (I) is a compound of Formula (II) and the compound is selected from: 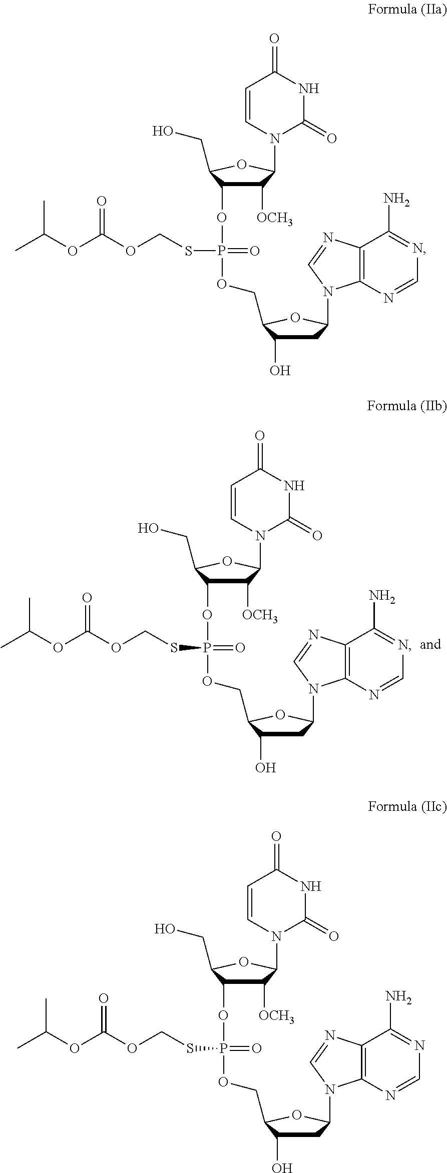
or a pharmaceutically acceptable salt thereof.

190-199. (canceled)


SEQUENCE LISTING (19 sequences)

SEQ ID NO: 1 - protein, 4 aa, Unknown
  Description of Unknown ′DEAD′ family peptide motif sequence
DEAD

SEQ ID NO: 2 - DNA, 20 bp, Artificial Sequence
  Description of Artificial Sequence Synthetic primer
ctcaagctgttgctgtcctc

SEQ ID NO: 3 - DNA, 20 bp, Artificial Sequence
  Description of Artificial Sequence Synthetic primer
cttctgggtgctgaagaggt

SEQ ID NO: 4 - DNA, 23 bp, Artificial Sequence
  Description of Artificial Sequence Synthetic probe
ccagatgacccagcagatcctca

SEQ ID NO: 5 - DNA, 20 bp, Artificial Sequence
  Description of Artificial Sequence Synthetic primer
gaatgaaaggcctgcagagt

SEQ ID NO: 6 - DNA, 20 bp, Artificial Sequence
  Description of Artificial Sequence Synthetic primer
ggatgtttgatctccttggg

SEQ ID NO: 7 - DNA, 26 bp, Artificial Sequence
  Description of Artificial Sequence Synthetic probe
cttgaagtccatcctgtcactgaggc

SEQ ID NO: 8 - DNA, 20 bp, Artificial Sequence
  Description of Artificial Sequence Synthetic primer
aaaaagagcggggagaagag

SEQ ID NO: 9 - DNA, 22 bp, Artificial Sequence
  Description of Artificial Sequence Synthetic primer
ggagcccttttagacctttcat

SEQ ID NO: 10 - DNA, 23 bp, Artificial Sequence
  Description of Artificial Sequence Synthetic probe
tccagaatctaaagccatcaaga

SEQ ID NO: 11 - DNA, 20 bp, Artificial Sequence
  Description of Artificial Sequence Synthetic primer
ccatgcaactcatcttgagc

SEQ ID NO: 12 - DNA, 20 bp, Artificial Sequence
  Description of Artificial Sequence Synthetic primer
atgcccatgaaccaataagc

SEQ ID NO: 13 - DNA, 18 bp, Artificial Sequence
  Description of Artificial Sequence Synthetic probe
atttcctgcagttcaccc

SEQ ID NO: 14 - DNA, 19 bp, Artificial Sequence
  Description of Artificial Sequence Synthetic primer
ctgttctggctgagcttcg

SEQ ID NO: 15 - DNA, 21 bp, Artificial Sequence
  Description of Artificial Sequence Synthetic primer
gcaggttcagaaacacagtgc

SEQ ID NO: 16 - DNA, 20 bp, Artificial Sequence
  Description of Artificial Sequence Synthetic probe
gggagtatggactcacccct

SEQ ID NO: 17 - DNA, 20 bp, Artificial Sequence
  Description of Artificial Sequence Synthetic primer
agttcacgatggtccaatcc

SEQ ID NO: 18 - DNA, 18 bp, Artificial Sequence
  Description of Artificial Sequence Synthetic primer
gtgccagggcatcaaaag

SEQ ID NO: 19 - DNA, 20 bp, Artificial Sequence
  Description of Artificial Sequence Synthetic probe
gcttcgtgctgagttcctct